(19) United States
(12) Patent Application Publication
(10) Pub. No.: US 2021/0121592 A1
(43) Pub. Date: Apr. 29, 2021

(54) DUAL DECONTAMINATION MECHANISM DEVICE

(71) Applicant: Mikael NAYGAUZ, Forrest Hills, NY (US)

(72) Inventor: Mikael NAYGAUZ, Forrest Hills, NY (US)

(21) Appl. No.: 16/751,552

(22) Filed: Jan. 24, 2020

Related U.S. Application Data
(60) Provisional application No. 62/925,240, filed on Oct. 24, 2019.
(60) Provisional application No. 62/930,711, filed on Nov. 5, 2019.
(60) Provisional application No. 62/938,774, filed on Nov. 21, 2019.

(51) Int. Cl. A61L 2/26; A61L 2/18

(52) U.S. Cl. CPC A61L 2/26; A61L 2/18; A61J 1/065; A61L 2202/121; A61L 2202/23; A61L 2202/123

(57) ABSTRACT

The present invention relates, in some embodiments thereof, to systems, devices, connection ports, and methods allowing for an engagement of two or more pharmaceutical vessels, containers, or pharmaceutical administration devices in a decontaminated and/or sterile manner. In some embodiments of the invention, the systems, devices, connection ports, and methods of the invention include a dual decontamination action device having a wiping member and a removable sealing member, a system comprising a device having a wiping member and a removable sealing member and a connection port having a removable sealing member. A first removable sealing member attached to a housing of a device is provided, the housing further comprising a wiping member configured to move across the housing; and a connection port having a removable sealing member, both removable sealing members configured to displace from the engagement between the device and the connection port while a hermetically sealed connection is maintained between the device and the connection port. In some embodiments of the invention, the systems, devices, connection ports and methods include a wiping member that is configured to wipe off the surface(s) of the engaged device and connection port, thus providing a decontaminated/sterile fluid communication between the device and the connection port.

### CROSS REFERENCE TO RELATED APPLICATIONS

This application claims the benefit of U.S. Provisional No. 62/925,240 entitled System, Device, and Method for Establishing Sterile Fluid Connections filed on Oct. 24, 2019; U.S. Provisional No. 62/930,711 entitled Dual Decontamination Mechanism Device and filed Nov. 5, 2019; U.S. Provisional No. 62/938,774 entitled Dual Decontamination Mechanism Device and filed Nov. 21, 2019.

This application claims the benefit of U.S. patent Ser. No. 16/100,964 entitled Decontamination Device for Pharmaceutical Vessels filed on Aug. 10, 2018; U.S. patent Ser. No. 16/100,594 entitled Devices and Systems with an External Displacement Mechanism for Contaminant-Free Engagement of Pharmaceutical Vessels and Pharmaceutical Administration Devices filed on Aug. 10, 2018; and U.S. patent Ser. No. 16/100,712 entitled Devices and Systems with an Internal Displacement Mechanism for Contaminant-Free Engagement of Medical Vessels and Devices.

The contents of the above applications are all incorporated by reference as if fully set forth herein in their entirety.

### FIELD OF THE INVENTION

The present invention relates, in some embodiments thereof, to systems, devices, connectors and methods allowing for an engagement of two or more pharmaceutical vessels or pharmaceutical administration devices in a decontaminated manner. More particularly, the present invention relates to devices, systems, and methods that provide a sterile fluid passageway between at least two containers.

### BACKGROUND OF THE INVENTION

The medicinal practice routinely involves administration of medical substances, such as, medicaments, fluids, nutritional substances and the alike, to patients or animals. The preparation and/or administration of such medicinal substances typically involves one or more transfers of those substances between pharmaceutical vessels or administration devices (such as, vials, bags, bottles, syringes, infusion lines, connectors, etc.). Each such act of transferring substances between vessels or devices exposes the vessel surfaces and accordingly the medical substances themselves to contaminants present in ambient air or ambient air particles (e.g., bacteria, viruses, funguses, spores, pyrogens, dirt). In addition, vessel surfaces are further prone to contaminations due to physical contact of the surfaces, for example, with nonsterile gloves, a user's hands, or devices.

Such contaminations are a major problem in the healthcare setting since contaminants, once invading into/inside the vessels and thus within the medicinal substances (such as medications, injectable medications, sterile fluids, etc.), may pose substantial danger if administered intracorporeally to patients. Contaminated substances if injected/infused into a patient may cause the patient to have an infection, a systemic infection, fever, etc. which may require hospitalization of the patient for treatment.

Typical vessel surfaces include, but are not limited to, rubber bungs and/or stoppers (optionally covered by a cap and/or seal that can be flicked off and/or are removed prior to usage thereof). These rubber bungs/stoppers are used to allow penetration by a needle attached to a syringe or by other medical connectors. When the cap and/or seal is flicked off and/or removed, the rubber bung and/or stopper is exposed to ambient air and to contaminants present therein. Accordingly, exposure of vessel surfaces to ambient air may involve contamination of the vessel surfaces by bacteria, fungus, viruses, etc. present in the ambient air and consequently contamination of a vessel surface and also contamination of a beneficial substance to be provided to a patient.

Existing systems include U.S. D720067; U.S. D717947; U.S. D703812; U.S. D690418; U.S. D639939; U.S. D637713; U.S. Pat. Nos. 9,790,011; 9,775,777; 9,561,326; 9,493,281; 9,492,353; 9,309,020; 9,173,816; 9,168,203; 9,162,803; 9,039,672; 8,926,583; 8,827,978; 8,790,330; 8,662,985; 8,657,803; 8,622,985; 8,562,583; 8,545,475; 8,523,838; 8,491,563; 8,480,646; 8,449,521; 8,381,776; 8,336,587; 8,328,772; 8,287,513; 8,225,826; 8,075,550; 8,029,747; 7,998,134; 7,975,733; 7,942,860; 7,867,215; 7,744,581; 7,731,678; 7,387,216; 7,306,584; 6,875,203; 6,729,370; 6,715,520; 6,602,239; 6,409,708; 6,343,629; 6,162,199; 6,113,583; 6,063,068; 5,893,397; 5,876,380; 5,832,971; 5,807,374; 5,746,733; 5,569,235; 5,462,535; 5,405,326; 5,292,318; 5,279,582; 4,944,723; 4,932,947; 4,932,937; 4,919,657; 4,915,701; 4,826,489; 4,673,404; 4,564,054; 3,610,241; 3,605,743; 3,587,575; 3,583,399; 3,578,037; 3,556,099; 3,552,387; 3,406,686; 3,380,450; 3,375,825; 3,342,180; 3,330,282; 3,330,281; 3,306,290; 3,255,752; 3,253,592; 3,076,456; 2,972,991; 2,922,419; US20160262982; US20160038373; US20150209568; US20140183196; US20140016570; US20140007973; US20140000754; US20130184672; US20130006200; US20120209238; US20120209218; US20120203194; US20110284561; US20110186177; US20110125128; US20110108158; US20110098647; US20100249745; US20100198182; US20100152669; US20100147402; US20100036319; US20100004602; US20090057258; US20080312634; US20080223484; US20080171981; US20060276759; US20050215976; US20030199847; US20030187420; US20020130100; US20020115981; US20020099354; ES2577377T3; EP2852367B1; EP2666513; EP2155141B1.

In order to overcome this obstacle, the current medical practice involves swabbing the surface of a connection interface with a disinfecting agent, such as 70% isopropyl alcohol, prior to accessing/piercing/puncturing the vessel surface. Other methods of ensuring sterility of injectable medicaments include i.v. (intravenous) rooms which are constructed and located inside hospitals and compounding pharmacies which are used for the sterile preparation of i.v. (intravenous) medications. Such i.v. rooms, to keep medicinal preparations as sterile as possible, are equipped with special instruments/equipment including, hoods with air filtration systems (e.g., HEPA filters), ventilation systems and air pressure systems. Additionally, those rooms necessitate that the medical staff working in these rooms are properly garmented, are properly trained, and require aseptic techniques, and employ quality control and validation processes. These systems require regular upkeep by certified personnel and require regular, typically daily, cleaning. Construction of such cleanrooms may cost approximately between $200,000 and $1,000,000 depending on the facility, size, location, and specifications of the cleanroom. These systems are therefore expensive to construct and maintain, are labor intensive, and require regular maintenance and testing (typically monthly sterility testing) to assure that they are operating effectively. The above described systems and methods are either cumbersome and expensive or inefficient in addressing the problem of reducing/eliminating contaminants on connection interfaces. Additionally, the mentioned cleanrooms, hoods, and systems are not foolproof and occasionally fail to pass monthly sterility tests performed by third parties in which case the cleanrooms and hoods must be re-cleaned, re-sanitized, and re-tested for sterility.

Many compounds for medical use are packaged separately from diluents used to facilitate administration of the compound to a patient. These medical compounds are packaged in a variety of known pharmaceutical containers (e.g., vials) in solid (e.g., lyophilized or spray-dried) form, in liquid form, in other forms. Prior to administration of these compounds to a patient, the compounds are mixed with a variety of known diluents in order to reconstitute, dilute, and/or facilitate intravenous or subcutaneous delivery to a patient. The diluents used can contain additional active compounds, if desired. In order to maintain the sterility of both the compound and the diluent in their respective containers, it is desirable to provide a system for intermixing that is substantially closed, i.e., one that does not expose the compound or diluent to the external environment. Such exposure could negatively affect the sterility of the resulting mixture of the compound and diluent, or, in the case of hazardous compounds, could expose healthcare workers to the hazardous compound.

Thus, there is a long felt and unmet need for pharmaceutical vessels, devices, systems and/or methods that afford transfer of medical substances in a decontaminated and/or sterile manner. There is a need for reliable, user-friendly and cost-effective solutions allowing contaminant-free and/or sterile engagement of vessels and/or devices for drug preparation and administration processes.

### SUMMARY OF THE INVENTION

Disclosed herein are various embodiments of a system, device, and a corresponding method that allow a user to establish a decontaminated and/or sterile fluid passageway between a device and a connection port which affords a user the ability to intermix at least two substances from at least two separate containers while ensuring sterility of the engagement and/or sterility of the fluidic communication between the at least two separate containers. Various embodiments of the device, connection port, system, and method are also disclosed.

Objects of the invention are achieved by providing systems, devices, vessels and methods for engaging medical devices, such as vessels, in a decontaminated manner.

Objects of the invention are achieved by providing systems, devices, vessels and methods which are directed to transferring medical substances in an efficient, user-friendly and sterile manner.

Objects of the invention are achieved by providing devices, systems, and methods for establishing decontaminated and/or sterile connections between a device and a connection port.

Objects of the invention are achieved by providing systems, devices, and methods for sterilizing connection ports.

Objects of the invention are achieved by providing a device having two mechanisms for providing a sterile and/or decontaminated connection between the device and a connection port.

Objects of the invention are achieved by providing a system having dual decontamination/sterilization functionality comprising at least one removable seal and a wiping member.

Objects of the invention are achieved by providing a system having a device with a wiping member and a removable sealing member, and a connection port having a removable sealing member.

Objects of the invention are achieved by providing devices, systems, and methods having dual decontamination mechanism of action in particular an external displacement decontamination mechanism for removable sealing members and an internal displacement decontamination mechanism for internally displaceable plugs.

Objects of the invention are achieved by providing devices, systems, and methods having guiding members, sealing members, and “O-rings” configured to align removable sealing members of devices, containers, and connection ports in a manner that facilitate the simultaneous displacement of at least two removable sealing members while maintaining hermetic coupling between devices, containers, connection ports and combinations thereof.

The present invention provides devices, systems, and methods that decontaminate and/or sterilize surfaces of medical and/or pharmaceutical vessels (such as vials, bags, bottles, devices, etc.) via two (a dual) mechanisms of action and thereafter allow decontaminated/sterile fluid passageway between the vessels. The present invention is also directed to transferring medical substances (such as medicaments, diluents, sterile fluids, etc.) in a sterile manner.

The present invention, in one or more embodiments, is based on a device having two (dual) decontamination mechanism structures and two (dual) decontamination mechanisms of action. The device having an externally displaceable removable sealing member which represents the first decontamination structure and mechanism, and the device further having a wiping member which provides the second decontamination structure and mechanism of action. A dual decontamination mechanism device is important in that if one of the decontamination structures (removable sealing member or wiping member) spontaneously fails then the second decontamination mechanism serves as a back-up mechanism to ensure decontamination/sterilization of the connection between the device and a connection port. The device and the connection port may form fluidic communication with each other after decontamination of the device to the connection port.

In a first aspect, the present invention provides a device for establishing a decontaminated fluid passageway, comprising: a housing defining a fluid passageway, a distal end portion of the housing configured to couple a connection port, the distal end portion of the housing having a securing mechanism configured to engage the connection port; a wiping member located inside the housing, the wiping member configured to move across the housing; an actuator connected to the wiping member, the actuator configured for moving the wiping member across the housing, the actuator extending through an opening in the wall of the housing; a removable sealing member for sealing the distal end portion of the housing, the removable sealing member positioned to seal the distal end portion of the fluid passageway, the removable sealing member configured to be movable between a first sealed position and a second unsealed position, the removable sealing member being moveable by manual manipulation of the removable sealing member, wherein the removable sealing member is displaced from the fluid passageway when the removable sealing member is in the second unsealed position.

In one or more embodiments, the removable sealing member is configured to substantially fluidly seal the fluid passageway when in a first sealed position, and wherein the removable sealing member is configured to allow the passage of fluid into the housing when the removable sealing member is in the second unsealed position.

In one or more embodiments, the removable sealing member is configured to hermetically seal an opening of the housing when the removable sealing member is in a first sealed position, and wherein the removable sealing member is configured to allow the passage of fluid into the housing when the removable sealing member is in the second unsealed position.

In one or more embodiments, the removable sealing member is further configured to be moved from the first sealed position to the second unsealed position via a displacement of the removable sealing member, wherein the displacement of the removable sealing member is external to the fluid passageway of the housing.

In one or more embodiments, the removable sealing member is in a folded configuration.

In one or more embodiments, a portion of the removable sealing member is attached to an interior surface of the housing.

In one or more embodiments, a portion of the removable sealing member is attached to an interior surface of the housing and a portion of the removable sealing member is attached to an exterior surface of the housing.

In one or more embodiments, a portion of the removable sealing member is attached to an interior of the housing and a portion of the removable sealing member is attached to an exterior of the housing.

In one or more embodiments, the wiping member is made of a rubber material and the removable sealing member is made of a plastic film.

In one or more embodiments, the first sealing member is characterized as being pierceable.

In one or more embodiments, the first sealing member is characterized as being frangible.

In one or more embodiments, the actuator is positioned through a side wall of the housing.

In one or more embodiments, the actuator is positioned through a top wall of the housing.

In one or more embodiments, the actuator is positioned through a bottom of the housing.

In one or more embodiments, the actuator comprises at least one of a handle, a rod, and a tab.

In one or more embodiments, the housing further comprising a locking member located on an interior wall of the housing, the locking member configured for locking the wiping member in an extended position to prevent the wiping member from returning to an original position.

In one or more embodiments, the housing further comprising a locking member, the locking member configured for locking the wiping member in an extended position after the wiping member has wiped off the first sealing member.

In one or more embodiments, the housing is made of a plastic material.

In one or more embodiments, the device further comprising a piercing member.

In one or more embodiments, the piercing member is configured to pierce through the first sealing member.

In one or more embodiments, the piercing member is configured to pierce through the first sealing member and the piercing member configured to pierce through the sealing member of a connection port.

In one or more embodiments, the piercing member is a needle.

In one or more embodiments, the piercing member is a hollowed needle.

In one or more embodiments, the device further comprising a piercing member actuator, the piercing member actuator configured to move the piercing member.

In one or more embodiments, the securing mechanism at the distal end portion of the housing is a thread configured to engage a complimentary thread of the connection port.

In one or more embodiments, the securing mechanism at the distal end portion of the housing are ratchet teeth configured to engage a complimentary retention member of the connection port.

In one or more embodiments, the securing mechanism at the distal end portion of the housing is a clamping member configured to engage a complimentary retention member of the connection port.

In one or more embodiments, the securing mechanism at the distal end portion of the housing is a retention member configured to engage a complimentary clamping member of the connection port.

In one or more embodiments, the securing mechanism at the distal end portion of the housing is an adhesive configured to engage the connection port.

In one or more embodiments, the proximal end portion of the housing further configured to couple with a container.

In one or more embodiments, the proximal end portion of the housing further comprising a securing mechanism selected from a group consisting of a thread, ratchet teeth, a clamping member, and an adhesive.

In one or more embodiments, the proximal end portion of the housing is attached to a container.

In one or more embodiments, the proximal end portion of the housing is integrally attached to a container.

In one or more embodiments, the proximal end portion of the housing forms a unitary structure with a container.

In one or more embodiments, the container is a bag or a bottle.

In one or more embodiments, the container is a vial.

In one or more embodiments, the proximal end portion of the housing is attached to a syringe.

In one or more embodiments, the proximal end portion of the housing is attached to a filter.

In one or more embodiments, the proximal end portion of the housing is attached to an infusion line.

In one or more embodiments, the proximal end portion of the housing is a thread.

In one or more embodiments, the proximal end portion of the housing is attached to a luer.

In one or more embodiments, the proximal end portion of the housing is a luer-lock.

In one or more embodiments, the locking member is a pin.

In one or more embodiments, at least a portion of the removable sealing member is located through a wall of the housing.

In one or more embodiments, at least one of an antibacterial substance, an antiviral substance, and an antifungal substance is located inside the housing

In one or more embodiments, at least one of an antibacterial substance, an antiviral substance, and an antifungal substance is located on a surface of the housing.

In one or more embodiments, at least one of an antibacterial substance, an antiviral substance, and an antifungal substance is located on an exterior surface of the housing.

In one or more embodiments, at least one of an antibacterial substance, an antiviral substance, and an antifungal substance is located on the removable sealing member.

In one or more embodiments, at least one of an antibacterial substance, an antiviral substance, and an antifungal substance is located on a surface of the removable sealing member.

In one or more embodiments, at least one of an antibacterial substance, an antiviral substance, and an antifungal substance is located on an exterior surface of the removable sealing member.

In one or more embodiments, the securing mechanism of the distal end portion of the housing is configured to be movable between a first extended position and a second contracted position.

In one or more embodiments, the distal end portion of the housing is further configured to be movable between a first extended position and a second contracted position.

In one or more embodiment, the securing mechanism is selected from a group consisting of a thread, ratchet teeth, a clamping member, a retention member, and an adhesive.

In one or more embodiments, the housing further comprising a guiding member configured to align the removable sealing member of the housing with a removable sealing member of a connection port.

In one or more embodiments, the guiding member is a rod.

In one or more embodiments, the guiding member is a dent.

In one or more embodiments, the guiding member is a slot.

In one or more embodiments, the guiding member is a protrusion.

In one or more embodiments, the guiding member is configured to align the removable sealing member of the housing of the device with a removable sealing member of a connection port, wherein upon alignment of the removable sealing member of the housing of the device with the removable sealing member of the connection port both the removable sealing member of the housing and the removable sealing member of the connection port will displace in about the same direction and at about the same time external to the location of fluidic communication between the housing and the connection port.

In one or more embodiments, the proximal end portion of the housing is integrally attached to a container and the distal end portion of the housing having a guiding member.

In one or more embodiments, the wiping member is configured to extend from the top wall of the housing to the bottom wall of the housing.

In one or more embodiments, the wiping member is configured to provide a hermetic seal between the top wall and the wiping member and the bottom wall and the wiping member when the wiping member moves across the housing.

In one or more embodiments, the wiping member is configured to wipe off a surface of the first sealing member of the housing when the wiping member moves across the housing.

In one or more embodiments, a filter is disposed in, on, or over an aperture in the housing for releasing air from the housing when actuating the wiping member.

In one or more embodiments, the housing is manufactured from a group consisting of plastic, rigid plastic, flexible plastic, polymer, and combinations thereof.

In one or more embodiments, the housing is made of a glass material, a metal material, and combinations thereof.

In one or more embodiments, the wiping member is made of a plastic substance, a rubber substance, an elastomeric substance, and combinations thereof.

In one or more embodiments, the wiping member is covered and/or saturated with an antibacterial substance, an antiviral substance, an anti-tuberculin substance, an anti-HIV substance, hydrogen peroxide, isopropyl alcohol, and combinations thereof.

In one or more embodiments, the device and/or housing is disposable and/or for single use.

In one or more embodiments, a surface of the removable sealing member has an adhesive on the surface of the removable sealing member.

In one or more embodiments, an exterior surface of the removable sealing member has an adhesive.

In one or more embodiments, the device further comprises a sealing ring.

In one or more embodiments, the sealing ring of the device is an “O-ring”.

In one or more embodiments, the sealing ring is a circumferential sealing ring.

In one or more embodiments, the “O-ring” is a circumferential “O-ring”.

In one or more embodiments, the sealing ring of the device is made of at least one of a plastic material and a rubber material.

In one or more embodiments, the sealing “O-ring” of the device is made of at least one of a plastic material and a rubber material.

In one or more embodiments, the device further comprising teeth configured to engage/couple holes/dents of a connection port.

In one or more embodiments, the teeth of the device are retractable teeth.

In one or more embodiments, the teeth are positioned at a distal end portion of the device.

In one or more embodiments, the retractable teeth are positioned at a distal end portion of the device.

In one or more embodiments, the teeth are positioned on an engagement mechanism of the device.

In one or more embodiments, the teeth are positioned on a securing mechanism of the device.

In one or more embodiments, the teeth are positioned on the device.

In one or more embodiments, the teeth are positioned on the housing of the device.

In one or more embodiments, the retractable teeth are positioned on the device.

In one or more embodiments, the retractable teeth are positioned on the housing of the device.

In one or more embodiments, the teeth are configured to engage/couple a hole/dent of a connection port.

In one or more embodiments, the retractable teeth are configured to engage/couple a hole/dent of a connection port.

In one or more embodiments, the teeth are configured to provide coupling of a connection port to the device, the teeth are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the device further comprising holes/dents configured to engage/couple with teeth of a connection port.

In one or more embodiments, the device further comprising holes/dents configured to engage/couple with retractable teeth of a connection port.

In one or more embodiments, the holes/debts are positioned at a distal end portion of the device.

In one or more embodiments, the holes/dents are positioned on an engagement mechanism of the device.

In one or more embodiments, the holes/dents are positioned on a securing mechanism of the device.

In one or more embodiments, the holes/dents are positioned on the device.

In one or more embodiments, the holes/dents are positioned on the housing of the device.

In one or more embodiments, the holes/dents are configured to engage/couple teeth of a connection port.

In one or more embodiments, the holes/dents are configured to engage/couple retractable teeth of a connection port.

In one or more embodiments, the holes/dents are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, a plurality of the devices are provided attached to a container.

In one or more embodiments, a proximal end portion of the devices is attached to the container.

In one or more embodiments, the proximal end portion of the devices is attached to the container.

In one or more embodiments, a plurality of the devices are provided integrally attached to a container.

In one or more embodiments, a proximal end portion of the plurality of the devices is attached to the container.

In one or more embodiments, the plurality of the devices form a unitary structure with the container.

In a second aspect, the present invention provides a device for establishing a decontaminated fluid passageway, comprising:
a housing having a side wall, a top and a bottom, the housing defining a fluid passageway, a distal end portion of the housing configured to couple a connection port, the distal end portion of the housing having a securing mechanism configured to engage the connection port;
a wiping member located inside the housing;
an actuator connected to the wiping member, the actuator configured for moving the wiping member across the housing, the actuator extending through an opening in the wall of the housing;
a first sealing member for sealing a proximal end portion of the housing, the first sealing member positioned to seal the proximal end portion of the fluid passageway, the first sealing member positioned to be wiped off by the wiping member when the wiping member moves across the housing; and
a removable sealing member for sealing the distal end portion of the housing, the removable sealing member positioned to seal the distal end portion of the fluid passageway, the removable sealing member configured to be movable between a first sealed position and a second unsealed position, the removable sealing member being moveable by manual manipulation of the removable sealing member, wherein the removable sealing member is displaced from the fluid passageway when the removable sealing member is in the second unsealed position.

In one or more embodiments, the removable sealing member is configured to substantially fluidly seal the fluid passageway when in a first sealed position, and wherein the removable sealing member is configured to allow the passage of fluid into the housing when the removable sealing member is in the second unsealed position.

In one or more embodiments, the removable sealing member is further configured to be moved from the first sealed position to the second unsealed position via a displacement of the removable sealing member, wherein the displacement of the removable sealing member is external to the fluid passageway of the housing.

In one or more embodiments, the removable sealing member is in a folded configuration.

In one or more embodiments, a portion of the removable sealing member is attached to an interior surface of the housing.

In one or more embodiments, a portion of the removable sealing member is attached to an interior surface of the housing and a portion of the removable sealing member is attached to an exterior surface of the housing.

In one or more embodiments, a portion of the removable sealing member is attached to an interior of the housing and a portion of the removable sealing member is attached to an exterior of the housing.

In one or more embodiments, the wiping member is made of a rubber material and the removable sealing member is made of a plastic film.

In one or more embodiments, the first sealing member is characterized as being pierceable.

In one or more embodiments, the first sealing member is characterized as being frangible.

In one or more embodiments, the actuator is positioned through a side wall of the housing.

In one or more embodiments, the actuator is positioned through a top wall of the housing.

In one or more embodiments, the actuator is positioned through a bottom of the housing.

In one or more embodiments, the actuator comprises at least one of a handle, a rod, and a tab.

In one or more embodiments, the housing further comprising a locking member located on an interior wall of the housing, the locking member configured for locking the wiping member in an extended position to prevent the wiping member from returning to an original position.

In one or more embodiments, the housing further comprising a locking member, the locking member configured for locking the wiping member in an extended position after the wiping member has wiped off the first sealing member.

In one or more embodiments, the housing is made of a plastic material.

In one or more embodiments, the device further comprising a piercing member.

In one or more embodiments, the piercing member is configured to pierce through the first sealing member.

In one or more embodiments, the piercing member is configured to pierce through the first sealing member and the piercing member configured to pierce through the sealing member of a connection port.

In one or more embodiments, the piercing member is a needle.

In one or more embodiments, the piercing member is a hollowed needle.

In one or more embodiments, the device further comprising a piercing member actuator, the piercing member actuator configured to move the piercing member.

In one or more embodiments, the securing mechanism at the distal end portion of the housing is a thread configured to engage a complimentary thread of the connection port.

In one or more embodiments, the securing mechanism at the distal end portion of the housing are ratchet teeth configured to engage a complimentary retention member of the connection port.

In one or more embodiments, the securing mechanism at the distal end portion of the housing is a clamping member configured to engage a complimentary retention member of the connection port.

In one or more embodiments, the securing mechanism at the distal end portion of the housing is a retention member configured to engage a complimentary clamping member of the connection port.

In one or more embodiments, the securing mechanism at the distal end portion of the housing is an adhesive configured to engage the connection port.

In one or more embodiments, the proximal end portion of the housing further configured to couple with a container.

In one or more embodiments, the proximal end portion of the housing further comprising a securing mechanism selected from a group consisting of a thread, ratchet teeth, a clamping member, and an adhesive.

In one or more embodiments, the proximal end portion of the housing is attached to a container.

In one or more embodiments, the proximal end portion of the housing is integrally attached to a container.

In one or more embodiments, the proximal end portion of the housing forms a unitary structure with a container.

In one or more embodiments, the container is a bag or a bottle.

In one or more embodiments, the container is a vial.

In one or more embodiments, the proximal end portion of the housing is attached to a syringe.

In one or more embodiments, the proximal end portion of the housing is attached to a filter.

In one or more embodiments, the proximal end portion of the housing is attached to an infusion line.

In one or more embodiments, the proximal end portion of the housing is a thread.

In one or more embodiments, the proximal end portion of the housing is attached to a luer.

In one or more embodiments, the proximal end portion of the housing is a luer-lock.

In one or more embodiments, the locking member is a pin.

In one or more embodiments, at least a portion of the removable sealing member is located through a wall of the housing.

In one or more embodiments, at least one of an antibacterial substance, an antiviral substance, and an antifungal substance is located inside the housing

In one or more embodiments, at least one of an antibacterial substance, an antiviral substance, and an antifungal substance is located on a surface of the housing.

In one or more embodiments, at least one of an antibacterial substance, an antiviral substance, and an antifungal substance is located on an exterior surface of the housing.

In one or more embodiments, at least one of an antibacterial substance, an antiviral substance, and an antifungal substance is located on the removable sealing member.

In one or more embodiments, at least one of an antibacterial substance, an antiviral substance, and an antifungal substance is located on a surface of the removable sealing member.

In one or more embodiments, at least one of an antibacterial substance, an antiviral substance, and an antifungal substance is located on an exterior surface of the removable sealing member.

In one or more embodiments, the securing mechanism of the distal end portion of the housing is configured to be movable between a first extended position and a second contracted position.

In one or more embodiments, the distal end portion of the housing is further configured to be movable between a first extended position and a second contracted position.

In one or more embodiment, the securing mechanism is selected from a group consisting of a thread, ratchet teeth, a clamping member, a retention member, and an adhesive.

In one or more embodiments, the device further comprises a sealing ring.

In one or more embodiments, the sealing ring of the device is an “O-ring”.

In one or more embodiments, the sealing ring is a circumferential sealing ring.

In one or more embodiments, the “O-ring” is a circumferential “O-ring”.

In one or more embodiments, the sealing ring of the device is made of at least one of a plastic material and a rubber material.

In one or more embodiments, the sealing “O-ring” of the device is made of at least one of a plastic material and a rubber material.

In one or more embodiments, the housing further comprising a guiding member configured to align the removable sealing member of the housing with a removable sealing member of a connection port.

In one or more embodiments, the guiding member is a rod.

In one or more embodiments, the guiding member is a dent.

In on or more embodiments, the guiding member is a slot.

In one or more embodiments, the guiding member is a protrusion.

In one or more embodiments, the guiding member is configured to align the removable sealing member of the housing of the device with a removable sealing member of a connection port, wherein upon alignment of the removable sealing member of the housing of the device with the removable sealing member of the connection port both the removable sealing member of the housing and the removable sealing member of the connection port will displace in about the same direction and at about the same time external to the location of fluidic communication between the housing and the connection port.

In one or more embodiments, the proximal end portion of the housing is integrally attached to a container and the distal end portion of the housing having a guiding member.

In one or more embodiments, the wiping member is configured to extend from the top wall of the housing to the bottom wall of the housing.

In one or more embodiments, the wiping member is configured to provide a hermetic seal between the top wall and the wiping member and the bottom wall and the wiping member when the wiping member moves across the housing.

In one or more embodiments, the wiping member is configured to wipe off a surface of the first sealing member of the housing when the wiping member moves across the housing.

In one or more embodiments, a filter is disposed in, on, or over an aperture in the housing for releasing air from the housing when actuating the wiping member.

In one or more embodiments, the housing is manufactured from a group consisting of plastic, rigid plastic, flexible plastic, polymer, and combinations thereof.

In one or more embodiments, the housing is made of a glass material, a metal material, and combinations thereof.

In one or more embodiments, the wiping member is made of a plastic substance, a rubber substance, an elastomeric substance, and combinations thereof.

In one or more embodiments, the wiping member is covered and/or saturated with an antibacterial substance, an antiviral substance, an anti-tuberculin substance, an anti-HIV substance, hydrogen peroxide, isopropyl alcohol, and combinations thereof.

In one or more embodiments, the device and/or housing is disposable and/or for single use.

In one or more embodiments, a surface of the removable sealing member has an adhesive on the surface of the removable sealing member.

In one or more embodiments, an exterior surface of the removable sealing member has an adhesive.

In one or more embodiments, the device further comprising teeth configured to engage/couple holes/dents of a connection port.

In one or more embodiments, the teeth of the device are retractable teeth.

In one or more embodiments, the teeth are positioned at a distal end portion of the device.

In one or more embodiments, the retractable teeth are positioned at a distal end portion of the device.

In one or more embodiments, the teeth are positioned on an engagement mechanism of the device.

In one or more embodiments, the teeth are positioned on a securing mechanism of the device.

In one or more embodiments, the teeth are positioned on the device.

In one or more embodiments, the teeth are positioned on the housing of the device.

In one or more embodiments, the retractable teeth are positioned on the device.

In one or more embodiments, the retractable teeth are positioned on the housing of the device.

In one or more embodiments, the teeth are configured to engage/couple a hole/dent of a connection port.

In one or more embodiments, the retractable teeth are configured to engage/couple a hole/dent of a connection port.

In one or more embodiments, the teeth are configured to provide coupling of a connection port to the device, the teeth are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the device further comprising holes/dents configured to engage/couple with teeth of a connection port.

In one or more embodiments, the device further comprising holes/dents configured to engage/couple with retractable teeth of a connection port.

In one or more embodiments, the holes/debts are positioned at a distal end portion of the device.

In one or more embodiments, the holes/dents are positioned on an engagement mechanism of the device.

In one or more embodiments, the holes/dents are positioned on a securing mechanism of the device.

In one or more embodiments, the holes/dents are positioned on the device.

In one or more embodiments, the holes/dents are positioned on the housing of the device.

In one or more embodiments, the holes/dents are configured to engage/couple teeth of a connection port.

In one or more embodiments, the holes/dents are configured to engage/couple retractable teeth of a connection port.

In one or more embodiments, the holes/dents are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

The present invention, in one or more embodiments, is based on a system comprising a device having two decontamination mechanisms, and a connection port having one decontamination mechanism. The device having a removable sealing member which provides a first decontaminating action when the removable sealing member is removed/displaced from the device and the device having a wiping member providing a second decontaminating action, the wiping member configured to move across the device thus wiping off/removing any remaining contaminants from the fluid passageway of the device. The wiping member of the device is further configured to wipe off a surface of the connection port. The wiping member of the device is intended to move across the housing and exert it's wiping action after the removable sealing member of the device and the removable sealing member of the connection port have been displaced external to the location of fluidic communication between the device and the connection port. The connection port having one decontamination mechanism being the removable sealing member of the connection port.

The device and the connection port of the system are intended to engage/couple with each other, preferably in an airtight/hermetic manner. At about the time and/or after said coupling, ambient air particles/contaminants are entrapped between the removable sealing member of the device and the removable sealing member of the connection port. The removable sealing member of the device and the removable sealing member of the connection port are then displaced at about the same time, optionally or alternately simultaneously, to a second unsealed position and after said displacement the wiping member of the device is moved across the device via actuation of the actuator, the movement of the wiping member across the device wipes off either or both a first sealing member of the device and a second sealing member of the connection port. Thus a system having a dual decontamination mechanism is provided, the first decontamination mechanism being the displacement to a second unsealed position of the removable sealing member of the device and the removable sealing member of the connection port, and the second decontamination mechanism being the wiping of at least one of, or both, the first sealing member of the device and the second sealing member of the connection port.

In an aspect, the present invention provides a system for establishing a decontaminated fluid passageway between a device and a connection port, comprising:
(i) a device comprising:
   (a) a housing having a side wall, a top and a bottom, the housing defining a fluid passageway, a distal end portion of the housing configured to couple a connection port, the distal end portion of the housing having a securing mechanism configured to engage the connection port;
   (b) a wiping member located inside the housing;
   (c) an actuator connected to the wiping member, the actuator configured for moving the wiping member across the housing, the actuator extending through an opening in the housing;
   (d) a first sealing member for sealing a proximal end portion of the housing, the first sealing member positioned to seal the proximal end portion of the fluid passageway, the first sealing member positioned to be wiped off by the wiping member when the wiping member moves across the housing: and
   (e) a removable sealing member for sealing the distal end portion of the housing, the removable sealing member positioned to seal the distal end portion of the fluid passageway, the removable sealing member configured to be movable between a first sealed position and a second unsealed position, the removable sealing member being moveable by manual manipulation of the removable sealing member, wherein the removable sealing member is displaced from the fluid passageway when the removable sealing member in the second unsealed position,
(ii) a connection port, comprising:
   (a) a proximal end portion defining a fluid passageway, the proximal end portion of the connection port configured to couple the distal end portion of the housing of the device, the proximal end portion of the connection port having a securing mechanism configured to engage the securing mechanism of the distal end portion of the housing of the device;
   (b) a removable sealing member for sealing the proximal end portion of the fluid passageway of the connection port, the removable sealing member of the connection port configured to be movable between a first sealed position and a second unsealed position, the removable sealing member of the connection port being movable by manual manipulation of the removable sealing member, wherein the removable sealing member is displaced from the fluid passageway of the connection port when the removable sealing member is in the second unsealed position.

In one or more embodiments, the removable sealing member of the device and the removable sealing member of the connection port are both configured to substantially seal the fluid passageway between the device and the connection port when both the removable sealing member of the device and the removable sealing member of the connection port are in a first sealed position, and wherein the removable sealing member of the device and the removable sealing member of the connection port are configured to allow passage of fluid between the device and the connection port when the removable sealing member of the device and the removable sealing member of the connection port are both in the second unsealed position.

In one or more embodiments, the removable sealing member of the device and the removable sealing member of the connection port are both configured to be moved from the first sealed position to the second unsealed position via a displacement of the removable sealing members of both the device and the connection port, wherein the displacement of both removable sealing members of the device and the connection port are external to the fluid passageway between the device and the connection port.

In one or more embodiments, the removable sealing member of the device is in a folded configuration and the removable sealing member of the connection port is in a folded configuration.

In one or more embodiments, the removable sealing member of the device is in a folded configuration or the removable sealing member of the connection port is in a folded configuration.

In one or more embodiments, at least one of the removable sealing members of the device and the removable sealing member of the connection port are in a folded configuration.

In one or more embodiments, a portion of the removable sealing member of the device is attached to an interior surface of the housing.

In one or more embodiments, a portion of the removable sealing member of the device is attached to an interior surface of the housing and a portion of the removable sealing member of the device is attached to an exterior surface of the housing.

In one or more embodiments, the wiping member is made of a rubber material, the removable sealing member of the device is made of a plastic material, and the removable sealing member of the connection port is made of a plastic material.

In one or more embodiments, the first sealing member of the device is characterized as being at least one of pierceable and frangible.

In one or more embodiments, the actuator of the device comprising at least one of a rod, a tab, and a handle.

In one or more embodiments, the actuator positioned through a wall of the housing.

In one or more embodiments, the actuator positioned through a top wall of the housing, a bottom wall of the housing, or a side wall of the housing.

In one or more embodiments, the actuator is a handle.

In one or more embodiments, the actuator is a tab.

In one or more embodiments, the housing further comprising a locking member configured for locking the wiping member in an extended position to prevent the wiping member from returning to an original position.

In one or more embodiments, the wiping member located on an interior wall of the housing.

In one or more embodiments, the locking member is a pin.

In one or more embodiments, the locking member is retractable pin.

In one or more embodiments, the device further comprising a piercing member.

In one or more embodiments, the housing further comprising a piercing member.

In one or more embodiments, the device further comprising a piercing member actuator.

In one or more embodiments, the piercing member actuator configured to move the piercing member through the first sealing member of the device.

In one or more embodiments, the piercing member is a needle.

In one or more embodiments, the piercing member is a hollowed needle.

In one or more embodiments, the piercing member is configured to pierce through the second sealing member of the connection port when the connection port is engaged to the housing of the device.

In one or more embodiments, the piercing member actuator is configured to move the piercing member through the second sealing member of the connection port when the connection port is coupled to the housing of the device.

In one or more embodiments, the connection port further comprising a second sealing member located at the proximal end portion of the connection port.

In one or more embodiments, the connection port further comprising a second sealing member located at a proximal end portion of the connection port.

In one or more embodiments, the removable sealing member of the connection port covers the second sealing member of the connection port when the removable sealing member of the connection port is in a first sealed position.

In one or more embodiments, the second sealing member is pierceable.

In one or more embodiments, the second sealing member is a rubber bung.

In one or more embodiments, the second sealing member is frangible.

In one or more embodiments, the second sealing member is a rubber stopper.

In one or more embodiments, the second sealing member is made of a plastic material, a metal alloy, and combinations thereof.

In one or more embodiments, the second sealing member is a plug that substantially seals the fluid passageway between the device and the connection port.

In one or more embodiments, the plug is displaceable.

In one or more embodiments, when the device is coupled to the connection port, and when the removable sealing member of the device and the removable sealing member of the connection port are both in the second unsealed position, a surface of the second sealing member of the connection port is exposed to an interior of the housing of the device.

In one or more embodiments, wherein the surface of the second sealing member of the connection port is characterized in that the surface of the second sealing member is positioned to be wiped off by the wiping member of the device when the wiping member moves across the housing.

In one or more embodiments, wherein when the device is coupled to the connection port, the surface of the second sealing member of the connection port and the wiping member of the device form an airtight seal when the wiping member moves across the housing of the device and wipes off the surface of the second sealing member.

In one or more embodiments, the wiping member of the device wipes off a surface of the second sealing member of the connection port via at least one of press and friction force(s).

In one or more embodiments, the device further comprising a piercing member configured to pierce through the first sealing member of the device and the second sealing member of the connection port.

In one or more embodiments, wherein the piercing member is a needle.

In one or more embodiments, wherein the piercing member is a hollowed needle.

In one or more embodiments, the securing mechanism at the distal end portion of the housing of the device is a thread and the securing mechanism at the proximal end portion of the connection port is a complimentary thread.

In one or more embodiments, the securing mechanism at the distal end portion of the housing of the device are ratchet teeth and the securing mechanism at the proximal end portion of the connection port is a complimentary retention member.

In one or more embodiments, the securing mechanism at the distal end portion of the device is a retention member and the securing mechanism at the proximal end portion of the connection port are complimentary ratchet teeth.

In one or more embodiments, the securing mechanism at the distal end portion of the housing of the device is a clamping member and the securing mechanism at the proximal end portion of the connection port is a complimentary retention member.

In one or more embodiments, the securing mechanism at the distal end portion of the housing of the device is a retention member and the securing mechanism at the proximal end portion of the connection port is a complimentary clamping member.

In one or more embodiments, at least one of the securing mechanism at the distal end portion of the housing and the securing mechanism at the proximal end portion of the connection port is an adhesive.

In one or more embodiments, the proximal end portion of the housing of the device is further configured to couple to a container and the distal end portion of the connection port is further configured to couple to a container.

In one or more embodiments, the proximal end portion of the housing of the device is further configured to couple to a bag and the distal end portion of the connection port is further configured to couple to a vial.

In one or more embodiments, wherein the distal end portion of the connection port is attached to a syringe and the proximal end portion of the housing of the device is attached to a container.

In one or more embodiments, the distal end portion of the connection port is integrally attached to a container and the proximal end portion of the housing of the device is integrally attached to a container.

In one or more embodiments of the system, the distal end portion of the connection port forms a unitary structure with a container and the proximal end portion of the housing of the device forms a unitary structure with a container.

In one or more embodiments of the system, the distal end portion of the connection port is attached to any of a syringe, a filter, an infusion line, and wherein the proximal end portion of the housing of the device is attached to any of a syringe, a filter, an infusion line, and a container.

In one or more embodiments of the system, wherein the removable sealing member of the device is attached to the housing of the device via a frangible seal and the removable sealing member of the connection port is attached to the connection port via a frangible seal.

In one or more embodiments of the system, wherein the removable sealing member of the device and the removable sealing member of the connection port are both configured to be displaced from the fluid passageway via a pulling motion.

In one or more embodiments of the system, wherein the removable sealing member of the device and the removable sealing member of the connection port are both configured to be displaced from the fluid passageway at about the same time via a pulling motion.

In one or more embodiments of the system, wherein at least a portion of the removable sealing member of the device is located through a wall of the housing.

In one or more embodiments, wherein at least a portion of the removable sealing member of the device is located through a side wall of the housing.

In one or more embodiments of the system, wherein at least one of an antibacterial substance, an antiviral substance, and an antifungal substance is located inside the housing of the device, on a surface of the removable sealing member, on the wiping member, and combinations thereof.

In one or more embodiments of the system, wherein the securing mechanism of the distal end portion of the housing of the device is further configured to be movable between a first extended position and a second contracted position.

In one or more embodiments of the system, wherein when the securing mechanism of the distal end portion of the housing of the device is in the second contracted position the second sealing member of the connection port is positioned to be wiped off by the wiping member of the device.

In one or more embodiments of the system, wherein when the device is coupled to the connection port and when the securing mechanism of the device is in a first extended position, an exterior surface of the second sealing member of the connection port is not aligned with an interior surface of the housing, and wherein when the device is coupled to the connection port and the securing mechanism of the device is in the second contracted position, an exterior surface of the second sealing member of the connection port is aligned with an interior surface of the housing.

In one or more embodiments of the system, wherein when the exterior surface of the second sealing member of the connection port is aligned with an interior surface of the housing, the wiping member is able to wipe off the exterior surface of the second sealing member of the connection port when the wiping member moves across the housing.

In one or more embodiments of the system, wherein the distal end portion of the housing of the device is further configured to be movable between a first extended position and a second contracted position.

In one or more embodiments, wherein the securing mechanism of the distal end portion of the housing of the device is selected from a group consisting of a thread, ratchet teeth, a clamping member, a retention member, and an adhesive.

In one or more embodiments of the system, the housing further comprising a guiding member configured to align the removable sealing member of the housing with the removable sealing member of the connection port.

In one or more embodiments, the guiding member is a rod.

In one or more embodiments, the guiding member is a dent.

In one or more embodiments, the guiding member is a slot.

In one or more embodiments, the guiding member is a protrusion.

In one or more embodiments of the system, the guiding member is configured to align the removable sealing member of the housing of the device with the removable sealing member of the connection port, wherein upon alignment of the removable sealing member of the housing of the device with the removable sealing member of the connection port both the removable sealing member of the housing and the removable sealing member of the connection port will displace in about the same direction and at about the same time external to the location of fluidic communication between the housing and the connection port.

In one or more embodiments of the system, the connection port has a guiding member configured to align the removable sealing member of the connection port with the removable sealing member of the device when the connection port couples with the device.

In one or more embodiments of the system, the guiding member of the connection port is a rod.

In one or more embodiments of the system, the guiding member of the connection port is a dent.

In one or more embodiments of the system, the guiding member of the connection port is a slot.

In one or more embodiments of the system, the guiding member of the connection port is a protrusion.

In one or more embodiments of the system, wherein the device further comprising a guiding member configured to align the removable sealing member of the device with the removable sealing member of the connection port.

In one or more embodiments of the system, wherein the connection port further comprising a guiding member configured to align the removable sealing member of the device with the removable sealing member of the connection port.

In one or more embodiments of the system, wherein the device further comprising a guiding member and wherein the connection port further comprising a guiding member, wherein both the guiding member of the device and the guiding member of the connection port are configured to engage with each other in such a manner as to align the removable sealing member of the device and the removable sealing member of the connection port so that both said removable sealing members are able to displace at about the same time and in the same direction when moved to a second unsealed position.

In one or more embodiments of the system, wherein the guiding member of the device is a rod and the guiding member of the connection port is a complimentary slot which allows the rod to enter into the slot.

In one or more embodiments of the system, wherein the guiding member of the connection port is a rod and the guiding member of the device is a complimentary slot which allows the rod to enter into the slot.

In one or more embodiments of the system, wherein the guiding member of the device is a protrusion and the complimentary guiding member of the connection port is a dent, wherein the dent allows the protrusion to engage and/or enter into the dent.

In one or more embodiments of the system, wherein the guiding member of the connection port is a protrusion and the complimentary guiding member of the device is a dent, wherein the dent allows the protrusion to engage and/or enter into the dent.

In one or more embodiments, the proximal end portion of the housing is integrally attached to a container and the distal end portion of the housing having a guiding member.

In one or more embodiments, the wiping member is configured to extend from the top wall of the housing to the bottom wall of the housing.

In one or more embodiments, the wiping member is configured to provide a hermetic seal between the top wall and the wiping member and the bottom wall and the wiping member when the wiping member moves across the housing.

In one or more embodiments, the wiping member is configured to wipe off a surface of the first sealing member of the housing when the wiping member moves across the housing.

In one or more embodiments, a filter is disposed in, on, or over an aperture in the housing for releasing air from the housing when actuating the wiping member.

In one or more embodiments, the housing is manufactured from a group consisting of plastic, rigid plastic, flexible plastic, polymer, and combinations thereof.

In one or more embodiments, the housing is made of a glass material, a metal material, and combinations thereof.

In one or more embodiments, the wiping member is made of a plastic substance, a rubber substance, an elastomeric substance, and combinations thereof.

In one or more embodiments, the wiping member is covered and/or saturated with an antibacterial substance, an antiviral substance, an anti-tuberculin substance, an anti-HIV substance, hydrogen peroxide, isopropyl alcohol, and combinations thereof.

In one or more embodiments, the device and/or housing is disposable and/or for single use.

In one or more embodiments, a surface of the removable sealing member has an adhesive on the surface of the removable sealing member.

In one or more embodiments, an exterior surface of the removable sealing member has an adhesive.

In one or more embodiments, the device further comprises a sealing ring.

In one or more embodiments, the sealing ring of the device is an “O-ring”.

In one or more embodiments, the sealing ring is a circumferential sealing ring.

In one or more embodiments, the “O-ring” is a circumferential “O-ring”.

In one or more embodiments, the sealing ring of the device is made of at least one of a plastic material and a rubber material.

In one or more embodiments, the sealing “O-ring” of the device is made of at least one of a plastic material and a rubber material.

In one or more embodiments, the connection port further comprises a sealing ring.

In one or more embodiments, the sealing ring of the connection port is an “O-ring”.

In one or more embodiments, the sealing ring of the connection port is a circumferential sealing ring.

In one or more embodiments, the “O-ring” of the connection port is a circumferential “O-ring”.

In one or more embodiments, the sealing ring of the connection port is made of at least one of a plastic material and a rubber material.

In one or more embodiments, the sealing “O-ring” of the connection port is made of at least one of a plastic material and a rubber material.

In one or more embodiments, both the device and the connection port further comprise a sealing ring.

In one or more embodiments, the sealing ring of the device and the sealing ring of the connection port is an “O-ring”.

In one or more embodiments, the sealing ring of the device is a circumferential sealing ring and the sealing ring of the connection port is a circumferential sealing ring.

In one or more embodiments, the “O-ring” of the device is a circumferential “O-ring” and the “O-ring of the connection port is a circumferential “O-ring”.

In one or more embodiments, the sealing ring of the connection port is made of at least one of a plastic material and a rubber material and the sealing ring of the device is made of at least one of a plastic material and a rubber material.

In one or more embodiments, the sealing “O-ring” of the device and the sealing “O-ring” of the connection port are made of at least one of a plastic material and a rubber material.

In one or more embodiments, the device further comprising teeth configured to engage/couple holes/dents of a connection port.

In one or more embodiments, the teeth of the device are retractable teeth.

In one or more embodiments, the teeth are positioned at a distal end portion of the device.

In one or more embodiments, the retractable teeth are positioned at a distal end portion of the device.

In one or more embodiments, the teeth are positioned on an engagement mechanism of the device.

In one or more embodiments, the teeth are positioned on a securing mechanism of the device.

In one or more embodiments, the teeth are positioned on the device.

In one or more embodiments, the teeth are positioned on the housing of the device.

In one or more embodiments, the retractable teeth are positioned on the device.

In one or more embodiments, the retractable teeth are positioned on the housing of the device.

In one or more embodiments, the teeth are configured to engage/couple a hole/dent of a connection port.

In one or more embodiments, the retractable teeth are configured to engage/couple a hole/dent of a connection port.

In one or more embodiments, the teeth are configured to provide coupling of a connection port to the device, the teeth are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the device further comprising holes/dents configured to engage/couple with teeth of a connection port.

In one or more embodiments, the device further comprising holes/dents configured to engage/couple with retractable teeth of a connection port.

In one or more embodiments, the holes/debts are positioned at a distal end portion of the device.

In one or more embodiments, the holes/dents are positioned on an engagement mechanism of the device.

In one or more embodiments, the holes/dents are positioned on a securing mechanism of the device.

In one or more embodiments, the holes/dents are positioned on the device.

In one or more embodiments, the holes/dents are positioned on the housing of the device.

In one or more embodiments, the holes/dents are configured to engage/couple teeth of a connection port.

In one or more embodiments, the holes/dents are configured to engage/couple retractable teeth of a connection port.

In one or more embodiments, the holes/dents are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments of the system, the device further comprising teeth and the connection port further comprising holes/dents.

In one or more embodiments of the system, the device further comprising retractable teeth and the connection port further comprising holes/dents configured to engage/couple the retractable teeth.

In one or more embodiments of the system, the device further comprising holes/dents and the connection port further comprising teeth.

In one or more embodiments of the system, the device further comprising holes/dents and the connection port further comprising retractable teeth.

In one or more embodiments, the holes/dents of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the holes/dents of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the teeth of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the retractable teeth of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the teeth of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the retractable teeth of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In a further aspect, the present invention provides a system for establishing a decontaminated fluid passageway between a device and a connection port, comprising: i.) a device comprising: a housing, the housing defining a fluid passageway, a distal end portion of the housing configured to couple a connection port, the distal end portion of the housing having a securing mechanism configured to engage the connection port; a wiping member located inside the housing; an actuator connected to the wiping member, the actuator configured for moving the wiping member across the housing, the actuator extending through an opening in the housing; a removable sealing member for sealing the distal end portion of the housing, the removable sealing member positioned to seal the distal end portion of the fluid passageway, the removable sealing member configured to be movable between a first sealed position and a second unsealed position, the removable sealing member being moveable by manual manipulation of the removable sealing member, wherein the removable sealing member is displaced from the fluid passageway when the removable sealing member in the second unsealed position; ii.) a connection port, comprising: a proximal end portion defining a fluid passageway, the proximal end portion of the connection port configured to couple the distal end portion of the housing of the device, the proximal end portion of the connection port having a securing mechanism configured to engage the securing mechanism of the distal end portion of the housing of the device; a removable sealing member for sealing the proximal end portion of the fluid passageway of the connection port, the removable sealing member of the connection port configured to be movable between a first sealed position and a second unsealed position, the removable sealing member of the connection port being movable by manual manipulation of the removable sealing member, wherein the removable sealing member is displaced from the fluid passageway of the connection port when the removable sealing member is in the second unsealed position.

In one or more embodiments, the removable sealing member of the device and the removable sealing member of the connection port are both configured to substantially seal the fluid passageway between the device and the connection port when both the removable sealing member of the device and the removable sealing member of the connection port are in a first sealed position, and wherein the removable sealing member of the device and the removable sealing member of the connection port are configured to allow passage of fluid between the device and the connection port when the removable sealing member of the device and the removable sealing member of the connection port are both in the second unsealed position.

In one or more embodiments, the removable sealing member of the device and the removable sealing member of the connection port are both configured to hermetically seal the fluid passageway between the device and the connection port when both the removable sealing member of the device and the removable sealing member of the connection port are in a first sealed position, and wherein the removable sealing member of the device and the removable sealing member of the connection port are configured to allow passage of fluid between the device and the connection port when the removable sealing member of the device and the removable sealing member of the connection port are both in the second unsealed position.

In one or more embodiments, the removable sealing member of the device and the removable sealing member of the connection port are both configured to hermetically seal an opening of the device and an opening of the connection port when both the removable sealing member of the device and the removable sealing member of the connection port are in a first sealed position, and wherein the removable sealing member of the device and the removable sealing member of the connection port are configured to allow passage of fluid between the device and the connection port when the removable sealing member of the device and the removable sealing member of the connection port are both in the second unsealed position.

In one or more embodiments, the removable sealing member of the device and the removable sealing member of the connection port are both configured to be moved from the first sealed position to the second unsealed position via a displacement of the removable sealing members of both the device and the connection port, wherein the displacement of both removable sealing members of the device and the connection port are external to the fluid passageway between the device and the connection port.

In one or more embodiments, the removable sealing member of the device is in a folded configuration and the removable sealing member of the connection port is in a folded configuration.

In one or more embodiments, the removable sealing member of the device is in a folded configuration or the removable sealing member of the connection port is in a folded configuration.

In one or more embodiments, at least one of the removable sealing members of the device and the removable sealing member of the connection port are in a folded configuration.

In one or more embodiments, a portion of the removable sealing member of the device is attached to an interior surface of the housing.

In one or more embodiments, a portion of the removable sealing member of the device is attached to an interior surface of the housing and a portion of the removable sealing member of the device is attached to an exterior surface of the housing.

In one or more embodiments, the wiping member is made of a rubber material, the removable sealing member of the device is made of a plastic material, and the removable sealing member of the connection port is made of a plastic material.

In one or more embodiments, the first sealing member of the device is characterized as being at least one of pierceable and frangible.

In one or more embodiments, the actuator of the device comprising at least one of a rod, a tab, and a handle.

In one or more embodiments, the actuator positioned through a wall of the housing.

In one or more embodiments, the actuator positioned through a top wall of the housing, a bottom wall of the housing, or a side wall of the housing.

In one or more embodiments, the actuator is a handle.

In one or more embodiments, the actuator is a tab.

In one or more embodiments, the housing further comprising a locking member configured for locking the wiping member in an extended position to prevent the wiping member from returning to an original position.

In one or more embodiments, the wiping member located on an interior wall of the housing.

In one or more embodiments, the locking member is a pin.

In one or more embodiments, the locking member is retractable pin.

In one or more embodiments, the device further comprising a piercing member.

In one or more embodiments, the housing further comprising a piercing member.

In one or more embodiments, the device further comprising a piercing member actuator.

In one or more embodiments, the piercing member actuator configured to move the piercing member through the first sealing member of the device.

In one or more embodiments, the piercing member is a needle.

In one or more embodiments, the piercing member is a hollowed needle.

In one or more embodiments, the piercing member is configured to pierce through the second sealing member of the connection port when the connection port is engaged to the housing of the device.

In one or more embodiments, the piercing member actuator is configured to move the piercing member through the second sealing member of the connection port when the connection port is coupled to the housing of the device.

In one or more embodiments, the connection port further comprising a second sealing member located at the proximal end portion of the connection port.

In one or more embodiments, the connection port further comprising a second sealing member located at a proximal end portion of the connection port.

In one or more embodiments, the removable sealing member of the connection port covers the second sealing member of the connection port when the removable sealing member of the connection port is in a first sealed position.

In one or more embodiments, the second sealing member is pierceable.

In one or more embodiments, the second sealing member is a rubber bung.

In one or more embodiments, the second sealing member is frangible.

In one or more embodiments, the second sealing member is a rubber stopper.

In one or more embodiments, the second sealing member is made of a plastic material, a metal alloy, and combinations thereof.

In one or more embodiments, the second sealing member is a plug that substantially seals the fluid passageway between the device and the connection port.

In one or more embodiments, the plug is displaceable.

In one or more embodiments, when the device is coupled to the connection port, and when the removable sealing member of the device and the removable sealing member of the connection port are both in the second unsealed position, a surface of the second sealing member of the connection port is exposed to an interior of the housing of the device.

In one or more embodiments, wherein the surface of the second sealing member of the connection port is characterized in that the surface of the second sealing member is positioned to be wiped off by the wiping member of the device when the wiping member moves across the housing.

In one or more embodiments, wherein when the device is coupled to the connection port, the surface of the second sealing member of the connection port and the wiping member of the device form an airtight seal when the wiping member moves across the housing of the device and wipes off the surface of the second sealing member.

In one or more embodiments, the wiping member of the device wipes off a surface of the second sealing member of the connection port via at least one of press and friction force(s).

In one or more embodiments, the device further comprising a piercing member configured to pierce through the first sealing member of the device and the second sealing member of the connection port.

In one or more embodiments, wherein the piercing member is a needle.

In one or more embodiments, wherein the piercing member is a hollowed needle.

In one or more embodiments, the securing mechanism at the distal end portion of the housing of the device is a thread and the securing mechanism at the proximal end portion of the connection port is a complimentary thread.

In one or more embodiments, the securing mechanism at the distal end portion of the housing of the device are ratchet teeth and the securing mechanism at the proximal end portion of the connection port is a complimentary retention member.

In one or more embodiments, the securing mechanism at the distal end portion of the device is a retention member and the securing mechanism at the proximal end portion of the connection port are complimentary ratchet teeth.

In one or more embodiments, the securing mechanism at the distal end portion of the housing of the device is a clamping member and the securing mechanism at the proximal end portion of the connection port is a complimentary retention member.

In one or more embodiments, the securing mechanism at the distal end portion of the housing of the device is a retention member and the securing mechanism at the proximal end portion of the connection port is a complimentary clamping member.

In one or more embodiments, at least one of the securing mechanism at the distal end portion of the housing and the securing mechanism at the proximal end portion of the connection port is an adhesive.

In one or more embodiments, the proximal end portion of the housing of the device is further configured to couple to a container and the distal end portion of the connection port is further configured to couple to a container.

In one or more embodiments, the proximal end portion of the housing of the device is further configured to couple to a bag and the distal end portion of the connection port is further configured to couple to a vial.

In one or more embodiments, wherein the distal end portion of the connection port is attached to a syringe and the proximal end portion of the housing of the device is attached to a container.

In one or more embodiments, the distal end portion of the connection port is integrally attached to a container and the proximal end portion of the housing of the device is integrally attached to a container.

In one or more embodiments of the system, the distal end portion of the connection port forms a unitary structure with a container and the proximal end portion of the housing of the device forms a unitary structure with a container.

In one or more embodiments of the system, the distal end portion of the connection port is attached to any of a syringe, a filter, an infusion line, and wherein the proximal end portion of the housing of the device is attached to any of a syringe, a filter, an infusion line, and a container.

In one or more embodiments of the system, wherein the removable sealing member of the device is attached to the housing of the device via a frangible seal and the removable sealing member of the connection port is attached to the connection port via a frangible seal.

In one or more embodiments of the system, wherein the removable sealing member of the device and the removable sealing member of the connection port are both configured to be displaced from the fluid passageway via a pulling motion.

In one or more embodiments of the system, wherein the removable sealing member of the device and the removable sealing member of the connection port are both configured to be displaced from the fluid passageway at about the same time via a pulling motion.

In one or more embodiments of the system, wherein at least a portion of the removable sealing member of the device is located through a wall of the housing.

In one or more embodiments, wherein at least a portion of the removable sealing member of the device is located through a side wall of the housing.

In one or more embodiments of the system, wherein at least one of an antibacterial substance, an antiviral substance, and an antifungal substance is located inside the housing of the device, on a surface of the removable sealing member, on the wiping member, and combinations thereof.

In one or more embodiments of the system, wherein the securing mechanism of the distal end portion of the housing of the device is further configured to be movable between a first extended position and a second contracted position.

In one or more embodiments of the system, wherein when the securing mechanism of the distal end portion of the housing of the device is in the second contracted position the second sealing member of the connection port is positioned to be wiped off by the wiping member of the device.

In one or more embodiments of the system, wherein when the device is coupled to the connection port and when the securing mechanism of the device is in a first extended position, an exterior surface of the second sealing member of the connection port is not aligned with an interior surface of the housing, and wherein when the device is coupled to the connection port and the securing mechanism of the device is in the second contracted position, an exterior surface of the second sealing member of the connection port is aligned with an interior surface of the housing.

In one or more embodiments of the system, wherein when the exterior surface of the second sealing member of the connection port is aligned with an interior surface of the housing, the wiping member is able to wipe off the exterior surface of the second sealing member of the connection port when the wiping member moves across the housing.

In one or more embodiments of the system, wherein the distal end portion of the housing of the device is further configured to be movable between a first extended position and a second contracted position.

In one or more embodiments, wherein the securing mechanism of the distal end portion of the housing of the device is selected from a group consisting of a thread, ratchet teeth, a clamping member, a retention member, and an adhesive.

In one or more embodiments of the system, the housing further comprising a guiding member configured to align the removable sealing member of the housing with the removable sealing member of the connection port.

In one or more embodiments, the guiding member is a rod.

In one or more embodiments, the guiding member is a dent.

In one or more embodiments, the guiding member is a slot.

In one or more embodiments, the guiding member is a protrusion.

In one or more embodiments of the system, the guiding member is configured to align the removable sealing member of the housing of the device with the removable sealing member of the connection port, wherein upon alignment of the removable sealing member of the housing of the device with the removable sealing member of the connection port both the removable sealing member of the housing and the removable sealing member of the connection port will displace in about the same direction and at about the same time external to the location of fluidic communication between the housing and the connection port.

In one or more embodiments of the system, the connection port has a guiding member configured to align the removable sealing member of the connection port with the removable sealing member of the device when the connection port couples with the device.

In one or more embodiments of the system, the guiding member of the connection port is a rod.

In one or more embodiments of the system, the guiding member of the connection port is a dent.

In one or more embodiments of the system, the guiding member of the connection port is a slot.

In one or more embodiments of the system, the guiding member of the connection port is a protrusion.

In one or more embodiments of the system, wherein the device further comprising a guiding member configured to align the removable sealing member of the device with the removable sealing member of the connection port.

In one or more embodiments of the system, wherein the connection port further comprising a guiding member configured to align the removable sealing member of the device with the removable sealing member of the connection port.

In one or more embodiments of the system, wherein the device further comprising a guiding member and wherein the connection port further comprising a guiding member, wherein both the guiding member of the device and the guiding member of the connection port are configured to engage with each other in such a manner as to align the removable sealing member of the device and the removable sealing member of the connection port so that both said removable sealing members are able to displace at about the same time and in the same direction when moved to a second unsealed position.

In one or more embodiments of the system, wherein the guiding member of the device is a rod and the guiding member of the connection port is a complimentary slot which allows the rod to enter into the slot.

In one or more embodiments of the system, wherein the guiding member of the connection port is a rod and the guiding member of the device is a complimentary slot which allows the rod to enter into the slot.

In one or more embodiments of the system, wherein the guiding member of the device is a protrusion and the complimentary guiding member of the connection port is a dent, wherein the dent allows the protrusion to engage and/or enter into the dent.

In one or more embodiments of the system, wherein the guiding member of the connection port is a protrusion and the complimentary guiding member of the device is a dent, wherein the dent allows the protrusion to engage and/or enter into the dent.

In one or more embodiments, the proximal end portion of the housing is integrally attached to a container and the distal end portion of the housing having a guiding member.

In one or more embodiments, the wiping member is configured to extend from the top wall of the housing to the bottom wall of the housing.

In one or more embodiments, the wiping member is configured to provide a hermetic seal between the top wall and the wiping member and the bottom wall and the wiping member when the wiping member moves across the housing.

In one or more embodiments, the wiping member is configured to wipe off a surface of the first sealing member of the housing when the wiping member moves across the housing.

In one or more embodiments, a filter is disposed in, on, or over an aperture in the housing for releasing air from the housing when actuating the wiping member.

In one or more embodiments, the housing is manufactured from a group consisting of plastic, rigid plastic, flexible plastic, polymer, and combinations thereof.

In one or more embodiments, the housing is made of a glass material, a metal material, and combinations thereof.

In one or more embodiments, the wiping member is made of a plastic substance, a rubber substance, an elastomeric substance, and combinations thereof.

In one or more embodiments, the wiping member is covered and/or saturated with an antibacterial substance, an antiviral substance, an anti-tuberculin substance, an anti-HIV substance, hydrogen peroxide, isopropyl alcohol, and combinations thereof.

In one or more embodiments, the device and/or housing is disposable and/or for single use.

In one or more embodiments, a surface of the removable sealing member has an adhesive on the surface of the removable sealing member.

In one or more embodiments, an exterior surface of the removable sealing member has an adhesive.

In one or more embodiments, the device further comprises a sealing ring.

In one or more embodiments, the sealing ring of the device is an “O-ring”.

In one or more embodiments, the sealing ring is a circumferential sealing ring.

In one or more embodiments, the “O-ring” is a circumferential “O-ring”.

In one or more embodiments, the sealing ring of the device is made of at least one of a plastic material and a rubber material.

In one or more embodiments, the sealing “O-ring” of the device is made of at least one of a plastic material and a rubber material.

In one or more embodiments, the connection port further comprises a sealing ring.

In one or more embodiments, the sealing ring of the connection port is an “O-ring”.

In one or more embodiments, the sealing ring of the connection port is a circumferential sealing ring.

In one or more embodiments, the “O-ring” of the connection port is a circumferential “O-ring”.

In one or more embodiments, the sealing ring of the connection port is made of at least one of a plastic material and a rubber material.

In one or more embodiments, the sealing “O-ring” of the connection port is made of at least one of a plastic material and a rubber material.

In one or more embodiments, both the device and the connection port further comprise a sealing ring.

In one or more embodiments, the sealing ring of the device and the sealing ring of the connection port is an “O-ring”.

In one or more embodiments, the sealing ring of the device is a circumferential sealing ring and the sealing ring of the connection port is a circumferential sealing ring.

In one or more embodiments, the “O-ring” of the device is a circumferential “O-ring” and the “O-ring of the connection port is a circumferential “O-ring”.

In one or more embodiments, the sealing ring of the connection port is made of at least one of a plastic material and a rubber material and the sealing ring of the device is made of at least one of a plastic material and a rubber material.

In one or more embodiments, the sealing “O-ring” of the device and the sealing “O-ring” of the connection port are made of at least one of a plastic material and a rubber material.

In one or more embodiments, the device further comprising teeth configured to engage/couple holes/dents of a connection port.

In one or more embodiments, the teeth of the device are retractable teeth.

In one or more embodiments, the teeth are positioned at a distal end portion of the device.

In one or more embodiments, the retractable teeth are positioned at a distal end portion of the device.

In one or more embodiments, the teeth are positioned on an engagement mechanism of the device.

In one or more embodiments, the teeth are positioned on a securing mechanism of the device.

In one or more embodiments, the teeth are positioned on the device.

In one or more embodiments, the teeth are positioned on the housing of the device.

In one or more embodiments, the retractable teeth are positioned on the device.

In one or more embodiments, the retractable teeth are positioned on the housing of the device.

In one or more embodiments, the teeth are configured to engage/couple a hole/dent of a connection port.

In one or more embodiments, the retractable teeth are configured to engage/couple a hole/dent of a connection port.

In one or more embodiments, the teeth are configured to provide coupling of a connection port to the device, the teeth are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the device further comprising holes/dents configured to engage/couple with teeth of a connection port.

In one or more embodiments, the device further comprising holes/dents configured to engage/couple with retractable teeth of a connection port.

In one or more embodiments, the holes/debts are positioned at a distal end portion of the device.

In one or more embodiments, the holes/dents are positioned on an engagement mechanism of the device.

In one or more embodiments, the holes/dents are positioned on a securing mechanism of the device.

In one or more embodiments, the holes/dents are positioned on the device.

In one or more embodiments, the holes/dents are positioned on the housing of the device.

In one or more embodiments, the holes/dents are configured to engage/couple teeth of a connection port.

In one or more embodiments, the holes/dents are configured to engage/couple retractable teeth of a connection port.

In one or more embodiments, the holes/dents are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments of the system, the device further comprising teeth and the connection port further comprising holes/dents.

In one or more embodiments of the system, the device further comprising retractable teeth and the connection port further comprising holes/dents configured to engage/couple the retractable teeth.

In one or more embodiments of the system, the device further comprising holes/dents and the connection port further comprising teeth.

In one or more embodiments of the system, the device further comprising holes/dents and the connection port further comprising retractable teeth.

In one or more embodiments, the holes/dents of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the holes/dents of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the teeth of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the retractable teeth of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the teeth of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the retractable teeth of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In a further aspect, the present invention provides a method for establishing a decontaminated fluid passageway between a device and a connection port, the method comprising:

i) providing a device comprising: a housing having a side wall, a top and a bottom, the housing defining a fluid passageway, a distal end portion of the housing configured to couple a connection port, the distal end portion of the housing having a securing mechanism configured to engage the connection port; a wiping member located inside the housing; an actuator connected to the wiping member, the actuator configured for moving the wiping member across the housing, the actuator extending through an opening in the housing; and a removable sealing member for sealing the distal end portion of the housing, the removable sealing member positioned to seal the distal end portion of the fluid passageway, the removable sealing member configured to be movable between a first sealed position and a second unsealed position, the removable sealing member being moveable by manual manipulation of the removable sealing member, wherein the removable sealing member is displaced from the fluid passageway when the removable sealing member in the second unsealed position, ii) providing a connection port, comprising: a proximal end portion defining a fluid passageway, the proximal end portion of the connection port configured to couple the distal end portion of the housing of the device, the proximal end portion of the connection port having a securing mechanism configured to engage the securing mechanism of the distal end portion of the housing of the device; a removable sealing member for sealing the proximal end portion of the fluid passageway of the connection port, the removable sealing member of the connection port configured to be movable between a first sealed position and a second unsealed position, the removable sealing member of the connection port being movable by manual manipulation of the removable sealing member, wherein the removable sealing member is displaced from the fluid passageway of the connection port when the removable sealing member is in the second unsealed position; iii) connecting the distal end portion of the housing of the device to the proximal end portion of the connection port; iv) moving the removable sealing member of the device to the second unsealed position by manually manipulation the removable sealing member of the device and moving the removable sealing member of the connection port to the second unsealed position by manually manipulating the removable sealing member of the connection port; and v) moving the wiping member of the device across the housing.

In one or more embodiments of the method, the moving of the removable sealing member of the device and the moving of the removable sealing member of the connection port displaces both the removable sealing member of the device and displaces the removable sealing member of the connection port from the fluid passageway of both the housing and the connection port.

In one or more embodiments of the method, the moving of the removable sealing member of the device and the moving of the removable sealing member of the connection port displaces both the removable sealing member of the device and displaces the removable sealing member of the connection port from the fluid passageway between the housing of the device and the connection port.

In one or more embodiments of the method, the moving of the removable sealing member of the device to the second unsealed position and moving the removable sealing member of the connection port to the second unsealed position occurs before moving of the wiping member across the housing.

In one or more embodiments of the method, the moving of the wiping member across the housing occurs after displacing the removable sealing members of the device and the connection port.

In one or more embodiments of the method, displacing the removable sealing member of the device and displacing the removable sealing member of the connection port occurs at about the same time.

In one or more embodiments of the method, displacing the removable sealing member of the device and the displacing the removable sealing member of the connection port occurs at the same time.

In one or more embodiments of the method, displacing the removable sealing member of the device and displacing the removable sealing member of the connection port occurs in the same direction.

In one or more embodiments of the method, the moving the removable sealing member of the device to the second unsealed position and the moving of the removable sealing member of the connection port to the second unsealed position decontaminates the connection between the device and the connection port.

In one or more embodiments of the method, the actuator comprises at least one of a tab, a rod, and a handle.

In one or more embodiments of the method, the actuator is a tab.

In one or more embodiments of the method, the actuator is a rod.

In one or more embodiments of the method, the actuator is a handle.

In one or more embodiments of the method, a sliding motion by a user moves the tab across the housing which results in the movement of the wiping member across the housing.

In one or more embodiments of the method, a sliding motion by a user moves the rod across the housing which results in the movement of the wiping member across the housing.

In one or more embodiments of the method, wherein the wiping member is moved across the housing of the device, the wiping member wipes off the first sealing member of the device.

In one or more embodiments of the method, wherein the connection port further comprises a second sealing member located at the proximal end portion of the connection port.

In one or more embodiments of the method, wherein the connection port further comprising a second sealing member located at the proximal end portion of the connection port.

In one or more embodiments of the method, wherein when the wiping member is moved across the housing of the device, the wiping member wipes off the first sealing member of the device and the wiping member wipes off the second sealing member of the connection port.

In one or more embodiments of the method, wherein when the wiping member wipes off the first sealing member of the device and the wiping member wipes off the second sealing member of the connection port, the wiping member decontaminates a surface of the first sealing member of the device and the wiping member decontaminates a surface of the second sealing member of the connection port.

In one or more embodiments of the method, wherein the device further comprising a piercing member configured to pierce through the first sealing member of the device and the piercing member configured to pierce through the second sealing member of the connection port.

In one or more embodiments of the method, the method further comprising the piercing member piercing through the first sealing member of the device and the second sealing member of the connection port.

In one or more embodiments of the method, wherein the piercing of the first sealing member and the piercing of the second sealing member by the piercing member establishes a fluidic communication between the device and the connection port.

In one or more embodiments of the method, wherein moving of the removable sealing member of the device and moving of the removable sealing member of the connection port occurs via a pulling motion by a user of both the removable sealing member of the device and the removable sealing member of the connection port.

In one or more embodiments of the method, wherein moving of the removable sealing member of the device to the second unsealed position and moving of the removable sealing member of the connection port to the second unsealed position exposes a surface of the second sealing member of the connection port to the interior of the housing of the device.

In one or more embodiments of the method, wherein when the wiping member is moved across the housing of the device, the wiping member wipes off the first sealing member of the device and the wiping member wipes off the second sealing member of the connection port, the wiping member further quarantines any contaminants located inside the housing away from the fluid passageway established between the device and the connection port.

In one or more embodiments of the method, wherein the housing further comprising a locking member located on an interior wall of the housing, the locking member configured for locking the wiping member in an extended position to prevent the wiping member from returning to an original position.

In one or more embodiments of the method, wherein the method further comprising the locking member locking the wiping member in an extended position.

In one or more embodiments of the method, wherein the locking member locks the wiping member in an extended position after the wiping member has moved across the housing.

In one or more embodiments of the method, the securing mechanism of the distal end portion of the housing of the device is further configured to be movable between a first extended position and a second contracted position.

In one or more embodiments of the method, wherein when the device is coupled to the connection port and when the securing mechanism of the device is in the first extended position, an exterior surface of the second sealing member of the connection port is not aligned with an interior surface of the housing, and wherein when the device is coupled to the connection port and the securing mechanism of the device is in the second contracted position, an exterior surface of the second sealing member of the connection port is aligned with an interior surface of the housing.

In one or more embodiments of the method, wherein when the exterior surface of the second sealing member of the connection port is aligned with an interior surface of the housing, the wiping member is able to wipe off the exterior surface of the second sealing member of the connection port when the wiping member moves across the housing.

In one or more embodiments of the method, wherein when the distal end portion of the housing of the device is further configured to be moveable between a first extended position and a second contracted position.

In one or more embodiments of the method, the securing mechanism of the device is a thread.

In one or more embodiments of the method, the securing mechanism of the device are ratchet teeth.

In one or more embodiments of the method, the securing mechanism of the device is an adhesive.

In one or more embodiments of the method, the securing mechanism of the device is a clamping member.

In one or more embodiments of the device, the securing mechanism of the device is a retention member.

In one or more embodiments of the method, the distal end portion of the housing of the device is further configured to be movable between a first extended position and a second contracted position.

In one or more embodiments of the method, the method further comprising moving the securing mechanism of the distal end portion of the housing of the device from a first extended position to a second contracted position.

In one or more embodiments of the method, wherein the moving of the securing mechanism of the distal end portion of the housing of the device from the first extended position to the second contracted position occurs after the moving of the removable sealing member of the device to the second unsealed position and after the moving of the removable sealing member of the connection port to the second unsealed position.

In one or more embodiments of the method, wherein the moving of the securing mechanism of the distal end portion of the housing from the first extended position to the second contracted position occurs before moving of the wiping member of the device across the housing.

In one or more embodiments of the method, wherein the moving of the securing mechanism of the distal end portion of the housing of the device from the first extended position to the second contracted position occurs after the moving of the removable sealing member of the device to the second unsealed position and after the moving of the removable sealing member of the connection port to the second unsealed position, and before moving of the wiping member across the housing.

In one or more embodiments of the method, wherein the securing mechanism of the distal end portion of the housing of the device is selected from a group consisting of a thread, ratchet teeth, a clamping member, a retention member, and an adhesive.

In one or more embodiments, the proximal end portion of the housing of the device is further configured to couple to a container and the distal end portion of the connection port is further configured to couple to a container.

In one or more embodiments, the proximal end portion of the housing of the device is further configured to couple to a bag and the distal end portion of the connection port is further configured to couple to a vial.

In one or more embodiments, wherein the distal end portion of the connection port is attached to a syringe and the proximal end portion of the housing of the device is attached to a container.

In one or more embodiments, the distal end portion of the connection port is integrally attached to a container and the proximal end portion of the housing of the device is integrally attached to a container.

In one or more embodiments of the system, the distal end portion of the connection port forms a unitary structure with a container and the proximal end portion of the housing of the device forms a unitary structure with a container.

In one or more embodiments of the system, the distal end portion of the connection port is attached to any of a syringe, a filter, an infusion line, and wherein the proximal end portion of the housing of the device is attached to any of a syringe, a filter, an infusion line, and a container.

In one or more embodiments, the device further comprises a sealing ring.

In one or more embodiments, the sealing ring of the device is an “O-ring”.

In one or more embodiments, the sealing ring is a circumferential sealing ring.

In one or more embodiments, the “O-ring” is a circumferential “O-ring”.

In one or more embodiments, the sealing ring of the device is made of at least one of a plastic material and a rubber material.

In one or more embodiments, the sealing “O-ring” of the device is made of at least one of a plastic material and a rubber material.

In one or more embodiments, the connection port further comprises a sealing ring.

In one or more embodiments, the sealing ring of the connection port is an “O-ring”.

In one or more embodiments, the sealing ring of the connection port is a circumferential sealing ring.

In one or more embodiments, the “O-ring” of the connection port is a circumferential “O-ring”.

In one or more embodiments, the sealing ring of the connection port is made of at least one of a plastic material and a rubber material.

In one or more embodiments, the sealing “O-ring” of the connection port is made of at least one of a plastic material and a rubber material.

In one or more embodiments, both the device and the connection port further comprise a sealing ring.

In one or more embodiments, the sealing ring of the device and the sealing ring of the connection port is an “O-ring”.

In one or more embodiments, the sealing ring of the device is a circumferential sealing ring and the sealing ring of the connection port is a circumferential sealing ring.

In one or more embodiments, the “O-ring” of the device is a circumferential “O-ring” and the “O-ring of the connection port is a circumferential “O-ring”.

In one or more embodiments, the sealing ring of the connection port is made of at least one of a plastic material and a rubber material and the sealing ring of the device is made of at least one of a plastic material and a rubber material.

In one or more embodiments, the sealing “O-ring” of the device and the sealing “O-ring” of the connection port are made of at least one of a plastic material and a rubber material.

In one or more embodiments, the device further comprising teeth configured to engage/couple holes/dents of a connection port.

In one or more embodiments, the teeth of the device are retractable teeth.

In one or more embodiments, the teeth are positioned at a distal end portion of the device.

In one or more embodiments, the retractable teeth are positioned at a distal end portion of the device.

In one or more embodiments, the teeth are positioned on an engagement mechanism of the device.

In one or more embodiments, the teeth are positioned on a securing mechanism of the device.

In one or more embodiments, the teeth are positioned on the device.

In one or more embodiments, the teeth are positioned on the housing of the device.

In one or more embodiments, the retractable teeth are positioned on the device.

In one or more embodiments, the retractable teeth are positioned on the housing of the device.

In one or more embodiments, the teeth are configured to engage/couple a hole/dent of a connection port.

In one or more embodiments, the retractable teeth are configured to engage/couple a hole/dent of a connection port.

In one or more embodiments, the teeth are configured to provide coupling of a connection port to the device, the teeth are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the device further comprising holes/dents configured to engage/couple with teeth of a connection port.

In one or more embodiments, the device further comprising holes/dents configured to engage/couple with retractable teeth of a connection port.

In one or more embodiments, the holes/debts are positioned at a distal end portion of the device.

In one or more embodiments, the holes/dents are positioned on an engagement mechanism of the device.

In one or more embodiments, the holes/dents are positioned on a securing mechanism of the device.

In one or more embodiments, the holes/dents are positioned on the device.

In one or more embodiments, the holes/dents are positioned on the housing of the device.

In one or more embodiments, the holes/dents are configured to engage/couple teeth of a connection port.

In one or more embodiments, the holes/dents are configured to engage/couple retractable teeth of a connection port.

In one or more embodiments, the holes/dents are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments of the system, the device further comprising teeth and the connection port further comprising holes/dents.

In one or more embodiments of the system, the device further comprising retractable teeth and the connection port further comprising holes/dents configured to engage/couple the retractable teeth.

In one or more embodiments of the system, the device further comprising holes/dents and the connection port further comprising teeth.

In one or more embodiments of the system, the device further comprising holes/dents and the connection port further comprising retractable teeth.

In one or more embodiments, the holes/dents of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the holes/dents of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the teeth of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the retractable teeth of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the teeth of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the retractable teeth of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the method further comprising moving the connection port from a first extended position to a second contracted position when the connection port is engaged/coupled to the device.

In one or more embodiments, the moving of the connection port from a first extended position to the second contracted positioned involves moving the connection port towards the device.

In one or more embodiments, the moving of the connection port from a first extended position to the second contracted positioned involves moving the device towards the connection port.

In one or more embodiments, the moving of the connection port from a first extended position to the second contracted positioned involves moving the connection port and the device towards each other.

In one or more embodiments, the moving of the connection port from a first extended position to the second contracted positioned involves pushing the proximal end portion of the connection port towards the distal end portion of the device and aligning a second sealing member of the connection port to an interior wall of the housing of the device.

In one or more embodiments, moving the connection port from the extended position to the contracted position when the connection port is coupled to the device occurs after displacement of the removable sealing members to the second unsealed position.

In one or more embodiments, moving the connection port from the extended position to the contracted position when the connection port is coupled to the device occurs prior to moving the wiping member across the interior of the device.

In one or more embodiments, moving the connection port from the extended position to the contracted position when the connection port is coupled to the device occurs after displacement of the removable sealing members to the second unsealed position and occurs prior to moving the wiping member across the device.

In a further aspect, the present invention provides a method for establishing a decontaminated fluid passageway between a device and a connection port, the method comprising:
i. providing a device comprising:
   a. a housing having a side wall, a top and a bottom, the housing defining a fluid passageway, a distal end portion of the housing configured to couple a connection port, the distal end portion of the housing having a securing mechanism configured to engage the connection port;
   b. a wiping member located inside the housing;
   c. an actuator connected to the wiping member, the actuator configured for moving the wiping member across the housing, the actuator extending through an opening in the housing;
   d. a first sealing member for sealing a proximal end portion of the housing, the first sealing member positioned to seal the proximal end portion of the fluid passageway, the first sealing member positioned to be wiped off by the wiping member when the wiping member moves across the housing: and
   e. a removable sealing member for sealing the distal end portion of the housing, the removable sealing member positioned to seal the distal end portion of the fluid passageway, the removable sealing member configured to be movable between a first sealed position and a second unsealed position, the removable sealing member being moveable by manual manipulation of the removable sealing member, wherein the removable sealing member is displaced from the fluid passageway when the removable sealing member in the second unsealed position,
ii. providing a connection port, comprising:
   a. a proximal end portion defining a fluid passageway, the proximal end portion of the connection port configured to couple the distal end portion of the housing of the device, the proximal end portion of the connection port having a securing mechanism configured to engage the securing mechanism of the distal end portion of the housing of the device;
   b. a removable sealing member for sealing the proximal end portion of the fluid passageway of the connection port, the removable sealing member of the connection port configured to be movable between a first sealed position and a second unsealed position, the removable sealing member of the connection port being movable by manual manipulation of the removable sealing member, wherein the removable sealing member is displaced from the fluid passageway of the connection port when the removable sealing member is in the second unsealed position;
iii. connecting the distal end portion of the housing of the device to the proximal end portion of the connection port;
iv. moving the removable sealing member of the device to the second unsealed position by manually manipulation the removable sealing member of the device and moving the removable sealing member of the connection port to the second unsealed position by manually manipulating the removable sealing member of the connection port; and
v. moving the wiping member of the device across the housing.

In one or more embodiments of the method, the moving of the removable sealing member of the device and the moving of the removable sealing member of the connection port displaces both the removable sealing member of the device and displaces the removable sealing member of the connection port from the fluid passageway of both the housing and the connection port.

In one or more embodiments of the method, the moving of the removable sealing member of the device and the moving of the removable sealing member of the connection port displaces both the removable sealing member of the device and displaces the removable sealing member of the connection port from the fluid passageway between the housing of the device and the connection port.

In one or more embodiments of the method, the moving of the removable sealing member of the device to the second unsealed position and moving the removable sealing member of the connection port to the second unsealed position occurs before moving of the wiping member across the housing.

In one or more embodiments of the method, the moving of the wiping member across the housing occurs after displacing the removable sealing members of the device and the connection port.

In one or more embodiments of the method, displacing the removable sealing member of the device and displacing the removable sealing member of the connection port occurs at about the same time.

In one or more embodiments of the method, displacing the removable sealing member of the device and the displacing the removable sealing member of the connection port occurs at the same time.

In one or more embodiments of the method, displacing the removable sealing member of the device and displacing the removable sealing member of the connection port occurs in the same direction.

In one or more embodiments of the method, the moving the removable sealing member of the device to the second unsealed position and the moving of the removable sealing member of the connection port to the second unsealed position decontaminates the connection between the device and the connection port.

In one or more embodiments of the method, the actuator comprises at least one of a tab, a rod, and a handle.

In one or more embodiments of the method, the actuator is a tab.

In one or more embodiments of the method, the actuator is a rod.

In one or more embodiments of the method, the actuator is a handle.

In one or more embodiments of the method, a sliding motion by a user moves the tab across the housing which results in the movement of the wiping member across the housing.

In one or more embodiments of the method, a sliding motion by a user moves the rod across the housing which results in the movement of the wiping member across the housing.

In one or more embodiments of the method, wherein the wiping member is moved across the housing of the device, the wiping member wipes off the first sealing member of the device.

In one or more embodiments of the method, wherein the connection port further comprises a second sealing member located at the proximal end portion of the connection port.

In one or more embodiments of the method, wherein the connection port further comprising a second sealing member located at the proximal end portion of the connection port.

In one or more embodiments of the method, wherein when the wiping member is moved across the housing of the device, the wiping member wipes off the first sealing member of the device and the wiping member wipes off the second sealing member of the connection port.

In one or more embodiments of the method, wherein when the wiping member wipes off the first sealing member of the device and the wiping member wipes off the second sealing member of the connection port, the wiping member decontaminates a surface of the first sealing member of the device and the wiping member decontaminates a surface of the second sealing member of the connection port.

In one or more embodiments of the method, wherein the device further comprising a piercing member configured to pierce through the first sealing member of the device and the piercing member configured to pierce through the second sealing member of the connection port.

In one or more embodiments of the method, the method further comprising the piercing member piercing through the first sealing member of the device and the second sealing member of the connection port.

In one or more embodiments of the method, wherein the piercing of the first sealing member and the piercing of the second sealing member by the piercing member establishes a fluidic communication between the device and the connection port.

In one or more embodiments of the method, wherein moving of the removable sealing member of the device and moving of the removable sealing member of the connection port occurs via a pulling motion by a user of both the removable sealing member of the device and the removable sealing member of the connection port.

In one or more embodiments of the method, wherein moving of the removable sealing member of the device to the second unsealed position and moving of the removable sealing member of the connection port to the second unsealed position exposes a surface of the second sealing member of the connection port to the interior of the housing of the device.

In one or more embodiments of the method, wherein when the wiping member is moved across the housing of the device, the wiping member wipes off the first sealing member of the device and the wiping member wipes off the second sealing member of the connection port, the wiping member further quarantines any contaminants located inside the housing away from the fluid passageway established between the device and the connection port.

In one or more embodiments of the method, wherein the housing further comprising a locking member located on an interior wall of the housing, the locking member configured for locking the wiping member in an extended position to prevent the wiping member from returning to an original position.

In one or more embodiments of the method, wherein the method further comprising the locking member locking the wiping member in an extended position.

In one or more embodiments of the method, wherein the locking member locks the wiping member in an extended position after the wiping member has moved across the housing.

In one or more embodiments of the method, the securing mechanism of the distal end portion of the housing of the device is further configured to be movable between a first extended position and a second contracted position.

In one or more embodiments of the method, wherein when the device is coupled to the connection port and when the securing mechanism of the device is in the first extended position, an exterior surface of the second sealing member of the connection port is not aligned with an interior surface of the housing, and wherein when the device is coupled to the connection port and the securing mechanism of the device is in the second contracted position, an exterior surface of the second sealing member of the connection port is aligned with an interior surface of the housing.

In one or more embodiments of the method, wherein when the exterior surface of the second sealing member of the connection port is aligned with an interior surface of the housing, the wiping member is able to wipe off the exterior surface of the second sealing member of the connection port when the wiping member moves across the housing.

In one or more embodiments of the method, wherein when the distal end portion of the housing of the device is further configured to be moveable between a first extended position and a second contracted position.

In one or more embodiments of the method, the securing mechanism of the device is a thread.

In one or more embodiments of the method, the securing mechanism of the device are ratchet teeth.

In one or more embodiments of the method, the securing mechanism of the device is an adhesive.

In one or more embodiments of the method, the securing mechanism of the device is a clamping member.

In one or more embodiments of the device, the securing mechanism of the device is a retention member.

In one or more embodiments of the method, the distal end portion of the housing of the device is further configured to be movable between a first extended position and a second contracted position.

In one or more embodiments of the method, the method further comprising moving the securing mechanism of the distal end portion of the housing of the device from a first extended position to a second contracted position.

In one or more embodiments of the method, wherein the moving of the securing mechanism of the distal end portion of the housing of the device from the first extended position to the second contracted position occurs after the moving of the removable sealing member of the device to the second unsealed position and after the moving of the removable sealing member of the connection port to the second unsealed position.

In one or more embodiments of the method, wherein the moving of the securing mechanism of the distal end portion of the housing from the first extended position to the second contracted position occurs before moving of the wiping member of the device across the housing.

In one or more embodiments of the method, wherein the moving of the securing mechanism of the distal end portion of the housing of the device from the first extended position to the second contracted position occurs after the moving of the removable sealing member of the device to the second unsealed position and after the moving of the removable sealing member of the connection port to the second unsealed position, and before moving of the wiping member across the housing.

In one or more embodiments of the method, wherein the securing mechanism of the distal end portion of the housing of the device is selected from a group consisting of a thread, ratchet teeth, a clamping member, a retention member, and an adhesive.

In one or more embodiments, the proximal end portion of the housing of the device is further configured to couple to a container and the distal end portion of the connection port is further configured to couple to a container.

In one or more embodiments, the proximal end portion of the housing of the device is further configured to couple to a bag and the distal end portion of the connection port is further configured to couple to a vial.

In one or more embodiments, wherein the distal end portion of the connection port is attached to a syringe and the proximal end portion of the housing of the device is attached to a container.

In one or more embodiments, the distal end portion of the connection port is integrally attached to a container and the proximal end portion of the housing of the device is integrally attached to a container.

In one or more embodiments of the system, the distal end portion of the connection port forms a unitary structure with a container and the proximal end portion of the housing of the device forms a unitary structure with a container.

In one or more embodiments of the system, the distal end portion of the connection port is attached to any of a syringe, a filter, an infusion line, and wherein the proximal end portion of the housing of the device is attached to any of a syringe, a filter, an infusion line, and a container.

In one or more embodiments, the device further comprises a sealing ring.

In one or more embodiments, the sealing ring of the device is an “O-ring”.

In one or more embodiments, the sealing ring is a circumferential sealing ring.

In one or more embodiments, the “O-ring” is a circumferential “O-ring”.

In one or more embodiments, the sealing ring of the device is made of at least one of a plastic material and a rubber material.

In one or more embodiments, the sealing “O-ring” of the device is made of at least one of a plastic material and a rubber material.

In one or more embodiments, the connection port further comprises a sealing ring.

In one or more embodiments, the sealing ring of the connection port is an “O-ring”.

In one or more embodiments, the sealing ring of the connection port is a circumferential sealing ring.

In one or more embodiments, the “O-ring” of the connection port is a circumferential “O-ring”.

In one or more embodiments, the sealing ring of the connection port is made of at least one of a plastic material and a rubber material.

In one or more embodiments, the sealing “O-ring” of the connection port is made of at least one of a plastic material and a rubber material.

In one or more embodiments, both the device and the connection port further comprise a sealing ring.

In one or more embodiments, the sealing ring of the device and the sealing ring of the connection port is an “O-ring”.

In one or more embodiments, the sealing ring of the device is a circumferential sealing ring and the sealing ring of the connection port is a circumferential sealing ring.

In one or more embodiments, the “O-ring” of the device is a circumferential “O-ring” and the “O-ring of the connection port is a circumferential “O-ring”.

In one or more embodiments, the sealing ring of the connection port is made of at least one of a plastic material and a rubber material and the sealing ring of the device is made of at least one of a plastic material and a rubber material.

In one or more embodiments, the sealing “O-ring” of the device and the sealing “O-ring” of the connection port are made of at least one of a plastic material and a rubber material.

In one or more embodiments, the device further comprising teeth configured to engage/couple holes/dents of a connection port.

In one or more embodiments, the teeth of the device are retractable teeth.

In one or more embodiments, the teeth are positioned at a distal end portion of the device.

In one or more embodiments, the retractable teeth are positioned at a distal end portion of the device.

In one or more embodiments, the teeth are positioned on an engagement mechanism of the device.

In one or more embodiments, the teeth are positioned on a securing mechanism of the device.

In one or more embodiments, the teeth are positioned on the device.

In one or more embodiments, the teeth are positioned on the housing of the device.

In one or more embodiments, the retractable teeth are positioned on the device.

In one or more embodiments, the retractable teeth are positioned on the housing of the device.

In one or more embodiments, the teeth are configured to engage/couple a hole/dent of a connection port.

In one or more embodiments, the retractable teeth are configured to engage/couple a hole/dent of a connection port.

In one or more embodiments, the teeth are configured to provide coupling of a connection port to the device, the teeth are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the device further comprising holes/dents configured to engage/couple with teeth of a connection port.

In one or more embodiments, the device further comprising holes/dents configured to engage/couple with retractable teeth of a connection port.

In one or more embodiments, the holes/debts are positioned at a distal end portion of the device.

In one or more embodiments, the holes/dents are positioned on an engagement mechanism of the device.

In one or more embodiments, the holes/dents are positioned on a securing mechanism of the device.

In one or more embodiments, the holes/dents are positioned on the device.

In one or more embodiments, the holes/dents are positioned on the housing of the device.

In one or more embodiments, the holes/dents are configured to engage/couple teeth of a connection port.

In one or more embodiments, the holes/dents are configured to engage/couple retractable teeth of a connection port.

In one or more embodiments, the holes/dents are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments of the system, the device further comprising teeth and the connection port further comprising holes/dents.

In one or more embodiments of the system, the device further comprising retractable teeth and the connection port further comprising holes/dents configured to engage/couple the retractable teeth.

In one or more embodiments of the system, the device further comprising holes/dents and the connection port further comprising teeth.

In one or more embodiments of the system, the device further comprising holes/dents and the connection port further comprising retractable teeth.

In one or more embodiments, the holes/dents of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the holes/dents of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the teeth of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the retractable teeth of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the teeth of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the retractable teeth of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the method further comprising moving the connection port from a first extended position to a second contracted position when the connection port is engaged/coupled to the device.

In one or more embodiments, the moving of the connection port from a first extended position to the second contracted positioned involves moving the connection port towards the device.

In one or more embodiments, the moving of the connection port from a first extended position to the second contracted positioned involves moving the device towards the connection port.

In one or more embodiments, the moving of the connection port from a first extended position to the second contracted positioned involves moving the connection port and the device towards each other.

In one or more embodiments, the moving of the connection port from a first extended position to the second contracted positioned involves pushing the proximal end portion of the connection port towards the distal end portion of the device and aligning a second sealing member of the connection port to an interior wall of the housing of the device.

In one or more embodiments, moving the connection port from the extended position to the contracted position when the connection port is coupled to the device occurs after displacement of the removable sealing members to the second unsealed position.

In one or more embodiments, moving the connection port from the extended position to the contracted position when the connection port is coupled to the device occurs prior to moving the wiping member across the interior of the device.

In one or more embodiments, moving the connection port from the extended position to the contracted position when the connection port is coupled to the device occurs after displacement of the removable sealing members to the second unsealed position and occurs prior to moving the wiping member across the device.

In yet a further aspect of the invention, the present invention provides a decontamination system for the engagement of vessels, the system comprising:

a first container;

a second container;

a housing connected to the first container;

a first connection interface attached to the housing; and

a second connection interface attached to the second container,

wherein the first connection interface and the second connection interface are configured to be coupled to each other, allowing for an engagement between the first vessel and the second vessel via the housing, wherein the first connection interface and the second connection interface are further configured to externally displace from the engagement while a hermetically sealed connection is maintained between the housing and the second container, and wherein the housing accommodates a wiping member configured to move within the housing and wipe off a surface of at least one of the second container and the housing, providing a decontaminated fluid passageway between the first container and the second container.

In one or more embodiments, at least one of the first connection interface and the second connection interface is a film. In one or more embodiments, the film is a plastic or a metal alloy.

In one or more embodiments, upon the engagement between the first container and the second container, the first connection interface and the second connection interface are configured to slide with respect to the first container and the second container, thereby removing contaminants between the first container and the second container.

In one or more embodiments, the first connection interface is configured to seal and/or cover a port or opening of the first container and/or a port or opening of the second container.

In one or more embodiments, the first connection interface and the second connection interface are configured to seal and/or cover an aperture of the first container and an aperture of the second container, respectively.

In one or more embodiments, the decontamination system results in air particle-free engagement of the first container and the second container, wherein air particles that contact the first and second interfaces are entrapped by the first connection interface and the second connection interface.

In one or more embodiments, the system further comprises an engagement mechanism configured to provide a secure engagement between the first container and the second container. In one or more embodiments, the engagement mechanism is located on the first container and/or, on the housing, and/or on the second container. In one or more embodiments, the engagement mechanism is selected from a thread, a luer, an adhesive, and a ratchet teeth mechanism. Optionally, a twisting, turning, and/or snapping motion is associated with the engagement. In an exemplary embodiment, a twisting motion is associated with a luer mechanism. In an exemplary embodiment, a snapping motion is associated with a ratchet teeth mechanism. In one or more embodiments, when the first contained and the second container engage via the engagement mechanism, an airtight seal is formed between the first container and the second container. In one or more embodiments, when the first container and the second container engage via the engagement mechanism a sealed aperture is formed between the first container and the second container.

In one or more embodiments, the sealed aperture formed between the first container and the second container allows for the displacement of at least one of the connection interfaces of the first container and the second container. In one or more embodiments, the sealed aperture is configured to displace at least one connection interface through the sealed aperture while maintaining the hermetic seal between the first container and the second container. In one or more embodiments, the system further comprises an engagement mechanism configured to provide a secure engagement between the first connection interface and the second connection interface. In one or more embodiments, the engagement mechanism is located on the first connection interface and/or on the second connection interface. In one or more embodiments, the engagement mechanism is selected from a thread, a luer, an adhesive, and a ratchet teeth mechanism. In one or more embodiments, the engagement mechanism includes a mechanical object that connects the first connection interface with the second connection interface providing for a simultaneous external displacement of the first and second connection interfaces. In one or more embodiments, the mechanical object is a protrusion, a dent, or a hook. In one or more embodiments, the engagement mechanism that secures the first connection interface to the second connection interface is an adhesive. In one or more embodiments, the engagement mechanism secures the first connection interface to the second connection interface forming an airtight seal between the first connection interface and the second connection interface. In one or more embodiments, the engagement mechanism is a thread on the first container configured to engage a complementary thread on the second container. In one or more embodiments, the engagement mechanism is a thread on the first connection interface configured to engage a complementary thread on the second connection interface. In one or more embodiments, the thread of at least one of the thread and complementary thread is a luer. In one or more embodiments, the system further comprising an engagement mechanism configured to provide a secure engagement between the housing and the first and/or second containers. In one or more embodiments, the engagement mechanism is located on the housing and/or on the first and/or second container. In one or more embodiments, the engagement mechanism is a thread on the housing configured to engage a complementary thread on the first container and/or second container.

In one or more embodiments, the system further comprises a first rail mechanism on the first container configured to provide external displacement of the first connection interface. In one or more embodiments, the system further comprises a second rail mechanism on the second container configured to provide external displacement of the second connection interface.

In one or more embodiments, the system further comprises a first rail mechanism on the first container configured to provide external displacement of the first connection interface and configured to provide external displacement of the second connection interface.

In one or more embodiments, the system further comprises a first slit mechanism on the first container configured to allow external displacement of the first connection interface and configured to provide external displacement of the second connection interface. In one or more embodiments, the first slit mechanism is an aperture. In one or more embodiments, the first slit mechanism is a sealed aperture. In one or more embodiments, the seal of the sealed aperture is a frangible seal. In one or more embodiments, the frangible seal is opened via a force of a user's hand.

In one or more embodiments, the system comprises a seal or a cover disposed between a container and a connection interface. In one or more embodiments, the seal or cover is frangible. In one or more embodiments, the seal or cover is pierceable.

In one or more embodiments, the system comprises a first seal or a first cover disposed between a first container and a first connection interface and a second seal or a second cover disposed between a second vessel and a second connection interface. In one or more embodiments, the first seal or first cover and a second seal or a second cover is frangible. In one or more embodiments, the first seal or first cover and a second seal or a second cover is frangible.

In one or more embodiments, the system further comprises a first hinge mechanism on a first container configured to provide external displacement of a first connection interface and comprising a second hinge mechanism on a second container configured to provide external displacement of a second connection interface.

In one or more embodiments, the system further comprises a piercing member on the first and/or second container and/or housing configured to pierce a sealed opening of the first and/or second container and/or housing, thereby allowing fluid to pass between the vessels. In one or more embodiments, the piercing member is a needle.

In one or more embodiments, the first and second connection interfaces are externally displaced via a sliding or a pulling motion.

In one or more embodiments, when the first connection interface and the second connection interface are displaced, the connection between the first container and the second container is decontaminated. In one or more embodiments, when the first and/or second connection interface slides off the first and/or second rail mechanism, the hermetic connection between the first vessel and the second vessel is maintained. In one or more embodiments, when the first and second connection interface slides off the first rail mechanism, the hermetic connection between the first container and the second container is maintained.

In one or more embodiments, the first connection interface and the second connection interface externally displace from the engagement between the first container and the second container through a slit or an aperture sealed by the force of the engagement between the first container and the second container.

In one or more embodiments, the first connection interface and the second connection interface externally displace from the engagement between the first container and the second container at the same time or about the same time.

In one or more embodiments, the interior of the housing may have a pressure less than the pressure of ambient air.

In one or more embodiments, circumferential ends of at least one of or both of the first and second connection interface ends are concealed within the hermetically sealed connection between the first container and the second container.

In one or more embodiments, the system further comprises a tab on at least one of the first connection interface and the second connection interface, the tab configured to allow for external displacement of the first connection interface and the second connection interface with respect to the first container and the second container. In one or more embodiments, the tab is configured to be pulled axially by a user, the axial pulling of the tab configured to displace the first connection interface and the second connection interface from the housing of the first container and the second container, while maintaining a hermetic seal between the first container and the second container.

In one or more embodiments, the first connection interface is coupled to the first container and wherein the second connection interface is coupled to the second container.

In one or more embodiments, the first connection interface has edges configured to isolate an entire surface of the first connection interface that is exposed to contaminants from contacting an internal compartment of the first container and/or the second container when the first connection interface is engaged with the second connection interface.

In one or more embodiments, the first connection interface has edges configured to isolate a portion of the surface of the first connection interface that is exposed to contaminants from contacting an internal compartment of the first vessel and/or the second vessel when the first connection interface is engaged with the second connection interface.

In one or more embodiments, the edges are circumferential edges. In one or more embodiments, the circumferential edges define the outer portion of the first connection interface and the second connection interface. In one or more embodiments, the edges are further configured so the sides of the edges are not exposed to contaminants. In one or more embodiments, the sides of the edges that are exposed to an internal compartment of the first container and/or the second container are not exposed to contaminants.

In one or more embodiments, the outer portion of the edges of the first connection interface are not exposed to contaminants. In one or more embodiments, the edges of the first connection interface are configured to fully entrap the contaminants between the first and second connection interfaces upon engagement of the first and second connection interfaces. In one or more embodiments, the edges of the first connection interface are flat.

In one or more embodiments, the edges of the first connection interface are configured to snuggly fit the edges of the second connection interface. In one or more embodiments, the edge is an exterior portion of the connection interface that comes in contact with an interior compartment of the housing and/or the container.

In one or more embodiments, the first connection interface is configured to seal and/or cover an opening of the housing and/or an opening of the second container.

In one or more embodiments, the system further comprising a locking-unlocking mechanism between the housing and the second container.

In one or more embodiments, the system further comprising an actuator configured to move the wiping member within the housing. In one or more embodiments, the actuator is selected from a group consisting of a handle, tab, a button, a touch button, a lever, a gear, a spring, a mechanical actuator, an electric actuator, and combinations thereof. In one or more embodiments, the actuator is connected to the wiping member and configured to move the wiping member. In one or more embodiments, the actuator moves the wiping member via a pushing, pulling and/or sliding motion. In one or more embodiments, the actuator manipulates the wiping member from the exterior of the housing. In one or more embodiments, the actuator is connected to the wiping member and configured to move the wiping member across the housing. In one or more embodiments, the actuator is spring loaded. In one or more embodiments, the actuator extends through an opening in a wall of the housing. In one or more embodiments, an airtight seal is maintained between the actuator and the opening in the wall. In one or more embodiments, the housing has a side wall, a top wall, and a bottom wall, wherein the top wall and the bottom wall each have an opening adapted for connection to the first and/or second container. In one or more embodiments, the wiping member is configured to extend from the top wall to the bottom wall of the housing.

In one or more embodiments, the housing further comprising a filter disposed in an aperture in the housing for releasing air from the housing when actuating the wiping member.

In one or more embodiments, the system further comprising a locking member disposed in the housing, the locking member being configured for locking the wiping member in an end position and to prevent the wiping member from returning to an initial position. In one or more embodiments, the wiping member is comprised of a rigid material, a rubber material, flexible material, expandable material, and combinations thereof.

In one or more embodiments, the housing is pre-attached to the first container. In one or more embodiments, the housing is integrally attached to the first container.

In one or more embodiments, the openings of the housing have vertical walls with internal threads, the internal threads being configured for cooperating with external threads on the at least one vessel to connect the vessel to the housing.

In one or more embodiments, the bottom wall of the housing contains a plurality of the openings configured for connecting a plurality of vessels to the housing, and wherein the device is configured so that actuating the wiping member wipes the surfaces of all vessels connected to the housing

In one or more embodiments, the side wall of the housing is open opposite the wiping member.

In one or more embodiments, the housing has a hermetic seal between the top wall and the bottom wall of the housing during actuation of the wiping member.

In one or more embodiments, the wiping member is covered by a sterilizing substance or a disinfecting substance. In one or more embodiments, the sterilizing substance is an antibacterial, an antiviral, and anti-tuberculin, and combinations thereof.

In one or more embodiments, the housing is disposable, intended for one-time use, or repeat use. In one or more embodiments, the wiping member is configured to move across the housing via a sliding motion. In one or more embodiments, the wiping member is made of a rubber.

In one or more embodiments, the housing is attached and abuts a wall of a container. In one or more embodiments, the housing is flush mounted to a wall of a container. In one or more embodiments, the housing is surface mounted to a wall of a container.

In one or more embodiments, the container is a bag.

In one or more embodiments, the container is a bottle.

In one or more embodiments, the container is a vial.

In one or more embodiments, the first container is a bag and the second container is a bottle.

In one or more embodiments, the first container is a bottle and the second container is a vial.

In one or more embodiments, the first container is a bag and the second container is a vial.

In one or more embodiments, the connection interface is a removable sealing member.

In one or more embodiments the first connection interface is a removable sealing member and the second connection interface is a removable sealing member.

In one or more embodiments, the device further comprises a sealing ring.

In one or more embodiments, the sealing ring of the device is an “O-ring”.

In one or more embodiments, the sealing ring is a circumferential sealing ring.

In one or more embodiments, the “O-ring” is a circumferential “O-ring”.

In one or more embodiments, the sealing ring of the device is made of at least one of a plastic material and a rubber material.

In one or more embodiments, the sealing “O-ring” of the device is made of at least one of a plastic material and a rubber material.

In one or more embodiments, the connection port further comprises a sealing ring.

In one or more embodiments, the sealing ring of the connection port is an “O-ring”.

In one or more embodiments, the sealing ring of the connection port is a circumferential sealing ring.

In one or more embodiments, the “O-ring” of the connection port is a circumferential “O-ring”.

In one or more embodiments, the sealing ring of the connection port is made of at least one of a plastic material and a rubber material.

In one or more embodiments, the sealing “O-ring” of the connection port is made of at least one of a plastic material and a rubber material.

In one or more embodiments, both the device and the connection port further comprise a sealing ring.

In one or more embodiments, the sealing ring of the device and the sealing ring of the connection port is an “O-ring”.

In one or more embodiments, the sealing ring of the device is a circumferential sealing ring and the sealing ring of the connection port is a circumferential sealing ring.

In one or more embodiments, the “O-ring” of the device is a circumferential “O-ring” and the “O-ring of the connection port is a circumferential “O-ring”.

In one or more embodiments, the sealing ring of the connection port is made of at least one of a plastic material and a rubber material and the sealing ring of the device is made of at least one of a plastic material and a rubber material.

In one or more embodiments, the sealing “O-ring” of the device and the sealing “O-ring” of the connection port are made of at least one of a plastic material and a rubber material.

In a further aspect, the present invention provides a method of decontaminating a connection between two vessels, the method comprising the steps of:

providing a housing connected to a first container, wherein the housing accommodates a wiping member configured to move within the housing, the housing further having a removable sealing member attached to the housing;

providing a second container having a removable sealing member;

engaging the housing to the second container via removable sealing members attached to the housing and the second container;

externally displacing the removable sealing member of the housing and the removable sealing member of the second container; and

moving the wiping member within the housing,

thereby providing a hermetically sealed and decontaminated connection between the first and second containers.

In one or more embodiments, the moving of the wiping member within the housing is moving the wiping member across the housing.

In one or more embodiments, the step of externally displacing the removable sealing member of the housing and externally displacing the removable sealing member of the second container occurs via a sliding motion.

In one or more embodiments, the step of externally displacing the removable sealing members of the housing and of the second container occurs via sliding the removable sealing member of the housing and the removable sealing member of the second container axially with respect to the first contained and the second container, thereby removing contaminants located between the removable sealing member of the housing and the removable sealing member of the second container.

In one or more embodiments, an airtight seal is formed between the removable sealing member of the housing and the removable sealing member of the second container prior to the external displacement of the removable sealing member of the housing and the removable sealing member of the second container.

In one or more embodiments, the displacement of the removable sealing member of the housing and the removable sealing member of the second container is via a rail mechanism, such that the airtight sealed compartment between the first connection interface and the second connection interface slides off the rail.

In one or more embodiments, when the airtight seal between the removable sealing member of the housing and the removable sealing member of the second container is displaced, the connection between the first container and the second container is decontaminated.

In one or more embodiments, the airtight seal between the removable sealing member of the housing and the removable sealing member of the second container slides off the rail while maintaining the hermetically sealed connection between the first and second containers.

In one or more embodiments, the step of externally displacing the removable sealing member of the housing and the removable sealing member of the second container occurs via peeling.

In one or more embodiments, the step of externally displacing the removable sealing member of the housing and the removable sealing member of the second container occurs at about the same time.

In one or more embodiments, circumferential ends of at least one of or both of the removable sealing members of the housing and removable sealing member of the second container ends are concealed within the airtight seal, allowing for sterile external displacement.

In one or more embodiments, the method further comprises connecting the removable sealing member of the housing to the removable sealing member of the second container via an engagement mechanism.

In one or more embodiments, connecting of the removable sealing member of the housing to the removable sealing member of the second container occurs prior to externally displacing the removable sealing member of the housing and the removable sealing member of the second container from the engagement between the first container and the second container.

In one or more embodiments, the connecting of the removable sealing member of the housing to the removable sealing member of the second container is selected from a twisting of a thread onto a complementary thread, attaching ratchet teeth to a complementary retention member, and adhering the removable sealing member of the housing to the removable sealing member of the second container via an adhesive.

In one or more embodiments, the sealed aperture is a valve. In one or more embodiments, the valve is a one-way valve.

In one or more embodiments, the device further comprises a sealing ring.

In one or more embodiments, the sealing ring of the device is an “O-ring”.

In one or more embodiments, the sealing ring is a circumferential sealing ring.

In one or more embodiments, the “O-ring” is a circumferential “O-ring”.

In one or more embodiments, the sealing ring of the device is made of at least one of a plastic material and a rubber material.

In one or more embodiments, the sealing “O-ring” of the device is made of at least one of a plastic material and a rubber material.

In one or more embodiments, the connection port further comprises a sealing ring.

In one or more embodiments, the sealing ring of the connection port is an “O-ring”.

In one or more embodiments, the sealing ring of the connection port is a circumferential sealing ring.

In one or more embodiments, the “O-ring” of the connection port is a circumferential “O-ring”.

In one or more embodiments, the sealing ring of the connection port is made of at least one of a plastic material and a rubber material.

In one or more embodiments, the sealing “O-ring” of the connection port is made of at least one of a plastic material and a rubber material.

In one or more embodiments, both the device and the connection port further comprise a sealing ring.

In one or more embodiments, the sealing ring of the device and the sealing ring of the connection port is an “O-ring”.

In one or more embodiments, the sealing ring of the device is a circumferential sealing ring and the sealing ring of the connection port is a circumferential sealing ring.

In one or more embodiments, the “O-ring” of the device is a circumferential “O-ring” and the “O-ring of the connection port is a circumferential “O-ring”.

In one or more embodiments, the sealing ring of the connection port is made of at least one of a plastic material and a rubber material and the sealing ring of the device is made of at least one of a plastic material and a rubber material.

In one or more embodiments, the sealing “O-ring” of the device and the sealing “O-ring” of the connection port are made of at least one of a plastic material and a rubber material.

In one or more embodiments, the device further comprising teeth configured to engage/couple holes/dents of a connection port.

In one or more embodiments, the teeth of the device are retractable teeth.

In one or more embodiments, the teeth are positioned at a distal end portion of the device.

In one or more embodiments, the retractable teeth are positioned at a distal end portion of the device.

In one or more embodiments, the teeth are positioned on an engagement mechanism of the device.

In one or more embodiments, the teeth are positioned on a securing mechanism of the device.

In one or more embodiments, the teeth are positioned on the device.

In one or more embodiments, the teeth are positioned on the housing of the device.

In one or more embodiments, the retractable teeth are positioned on the device.

In one or more embodiments, the retractable teeth are positioned on the housing of the device.

In one or more embodiments, the teeth are configured to engage/couple a hole/dent of a connection port.

In one or more embodiments, the retractable teeth are configured to engage/couple a hole/dent of a connection port.

In one or more embodiments, the teeth are configured to provide coupling of a connection port to the device, the teeth are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the device further comprising holes/dents configured to engage/couple with teeth of a connection port.

In one or more embodiments, the device further comprising holes/dents configured to engage/couple with retractable teeth of a connection port.

In one or more embodiments, the holes/debts are positioned at a distal end portion of the device.

In one or more embodiments, the holes/dents are positioned on an engagement mechanism of the device.

In one or more embodiments, the holes/dents are positioned on a securing mechanism of the device.

In one or more embodiments, the holes/dents are positioned on the device.

In one or more embodiments, the holes/dents are positioned on the housing of the device.

In one or more embodiments, the holes/dents are configured to engage/couple teeth of a connection port.

In one or more embodiments, the holes/dents are configured to engage/couple retractable teeth of a connection port.

In one or more embodiments, the holes/dents are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments of the system, the device further comprising teeth and the connection port further comprising holes/dents.

In one or more embodiments of the system, the device further comprising retractable teeth and the connection port further comprising holes/dents configured to engage/couple the retractable teeth.

In one or more embodiments of the system, the device further comprising holes/dents and the connection port further comprising teeth.

In one or more embodiments of the system, the device further comprising holes/dents and the connection port further comprising retractable teeth.

In one or more embodiments, the holes/dents of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the holes/dents of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the teeth of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the retractable teeth of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the teeth of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the retractable teeth of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the method further comprising moving the connection port from a first extended position to a second contracted position when the connection port is engaged/coupled to the device.

In one or more embodiments, the moving of the connection port from a first extended position to the second contracted positioned involves moving the connection port towards the device.

In one or more embodiments, the moving of the connection port from a first extended position to the second contracted positioned involves moving the device towards the connection port.

In one or more embodiments, the moving of the connection port from a first extended position to the second contracted positioned involves moving the connection port and the device towards each other.

In one or more embodiments, the moving of the connection port from a first extended position to the second contracted positioned involves pushing the proximal end portion of the connection port towards the distal end portion of the device and aligning a second sealing member of the connection port to an interior wall of the housing of the device.

In one or more embodiments, moving the connection port from the extended position to the contracted position when the connection port is coupled to the device occurs after displacement of the removable sealing members to the second unsealed position.

In one or more embodiments, moving the connection port from the extended position to the contracted position when the connection port is coupled to the device occurs prior to moving the wiping member across the interior of the device.

In one or more embodiments, moving the connection port from the extended position to the contracted position when the connection port is coupled to the device occurs after displacement of the removable sealing members to the second unsealed position and occurs prior to moving the wiping member across the device.

In yet a further aspect, the present invention provides a device for establishing a sterile fluid passageway, comprising:
a housing defining a fluid passageway;
a wiping member disposed inside the housing, the wiping member configured to move across the housing; and
a removable sealing member for sealing an end of the housing.

In one or more embodiments, the removable sealing member is configured to substantially fluidly seal the fluid passageway when in a first sealed position, and wherein the removable sealing member is configured to allow the passage of fluid into the housing when the removable sealing member is in the second unsealed position.

In one or more embodiments, the removable sealing member is further configured to be moved from the first sealed position to the second unsealed position via a displacement of the removable sealing member, wherein the displacement of the removable sealing member is external to the fluid passageway of the housing.

In one or more embodiments, the removable sealing member is in a folded configuration.

In one or more embodiments, a portion of the removable sealing member is attached to an interior surface of the housing.

In one or more embodiments, a portion of the removable sealing member is attached to an interior surface of the housing and a portion of the removable sealing member is attached to an exterior surface of the housing.

In one or more embodiments, a portion of the removable sealing member is attached to an interior of the housing and a portion of the removable sealing member is attached to an exterior of the housing.

In one or more embodiments, the wiping member is made of a rubber material and the removable sealing member is made of a plastic film.

In one or more embodiments, the first sealing member is characterized as being pierceable.

In one or more embodiments, the first sealing member is characterized as being frangible.

In one or more embodiments, the actuator is positioned through a side wall of the housing.

In one or more embodiments, the actuator is positioned through a top wall of the housing.

In one or more embodiments, the actuator is positioned through a bottom of the housing.

In one or more embodiments, the actuator comprises at least one of a handle, a rod, and a tab.

In one or more embodiments, the housing further comprising a locking member located on an interior wall of the housing, the locking member configured for locking the wiping member in an extended position to prevent the wiping member from returning to an original position.

In one or more embodiments, the housing further comprising a locking member, the locking member configured for locking the wiping member in an extended position after the wiping member has wiped off the first sealing member.

In one or more embodiments, the housing is made of a plastic material.

In one or more embodiments, the device further comprising a piercing member.

In one or more embodiments, the piercing member is configured to pierce through the first sealing member.

In one or more embodiments, the piercing member is configured to pierce through the first sealing member and the piercing member configured to pierce through the sealing member of a connection port.

In one or more embodiments, the piercing member is a needle.

In one or more embodiments, the piercing member is a hollowed needle.

In one or more embodiments, the device further comprising a piercing member actuator, the piercing member actuator configured to move the piercing member.

In one or more embodiments, the securing mechanism at the distal end portion of the housing is a thread configured to engage a complimentary thread of the connection port.

In one or more embodiments, the securing mechanism at the distal end portion of the housing are ratchet teeth configured to engage a complimentary retention member of the connection port.

In one or more embodiments, the securing mechanism at the distal end portion of the housing is a clamping member configured to engage a complimentary retention member of the connection port.

In one or more embodiments, the securing mechanism at the distal end portion of the housing is a retention member configured to engage a complimentary clamping member of the connection port.

In one or more embodiments, the securing mechanism at the distal end portion of the housing is an adhesive configured to engage the connection port.

In one or more embodiments, the proximal end portion of the housing further configured to couple with a container.

In one or more embodiments, the proximal end portion of the housing further comprising a securing mechanism selected from a group consisting of a thread, ratchet teeth, a clamping member, and an adhesive.

In one or more embodiments, the proximal end portion of the housing is attached to a container.

In one or more embodiments, the proximal end portion of the housing is integrally attached to a container.

In one or more embodiments, the proximal end portion of the housing forms a unitary structure with a container.

In one or more embodiments, the container is a bag or a bottle.

In one or more embodiments, the container is a vial.

In one or more embodiments, the proximal end portion of the housing is attached to a syringe.

In one or more embodiments, the proximal end portion of the housing is attached to a filter.

In one or more embodiments, the proximal end portion of the housing is attached to an infusion line.

In one or more embodiments, the proximal end portion of the housing is a thread.

In one or more embodiments, the proximal end portion of the housing is attached to a luer.

In one or more embodiments, the proximal end portion of the housing is a luer-lock.

In one or more embodiments, the locking member is a pin.

In one or more embodiments, at least a portion of the removable sealing member is located through a wall of the housing.

In one or more embodiments, at least one of an antibacterial substance, an antiviral substance, and an antifungal substance is located inside the housing

In one or more embodiments, at least one of an antibacterial substance, an antiviral substance, and an antifungal substance is located on a surface of the housing.

In one or more embodiments, at least one of an antibacterial substance, an antiviral substance, and an antifungal substance is located on an exterior surface of the housing.

In one or more embodiments, at least one of an antibacterial substance, an antiviral substance, and an antifungal substance is located on the removable sealing member.

In one or more embodiments, at least one of an antibacterial substance, an antiviral substance, and an antifungal substance is located on a surface of the removable sealing member.

In one or more embodiments, at least one of an antibacterial substance, an antiviral substance, and an antifungal substance is located on an exterior surface of the removable sealing member.

In one or more embodiments, the securing mechanism of the distal end portion of the housing is configured to be movable between a first extended position and a second contracted position.

In one or more embodiments, the distal end portion of the housing is further configured to be movable between a first extended position and a second contracted position.

In one or more embodiment, the securing mechanism is selected from a group consisting of a thread, ratchet teeth, a clamping member, a retention member, and an adhesive.

In one or more embodiments, the housing further comprising a guiding member configured to align the removable sealing member of the housing with a removable sealing member of a connection port.

In one or more embodiments, the guiding member is a rod.

In one or more embodiments, the guiding member is a dent.

In one or more embodiments, the guiding member is a slot.

In one or more embodiments, the guiding member is a protrusion.

In one or more embodiments, the guiding member is configured to align the removable sealing member of the housing of the device with a removable sealing member of a connection port, wherein upon alignment of the removable sealing member of the housing of the device with the removable sealing member of the connection port both the removable sealing member of the housing and the removable sealing member of the connection port will displace in about the same direction and at about the same time external to the location of fluidic communication between the housing and the connection port.

In one or more embodiments, the proximal end portion of the housing is integrally attached to a container and the distal end portion of the housing having a guiding member.

In one or more embodiments, the wiping member is configured to extend from the top wall of the housing to the bottom wall of the housing.

In one or more embodiments, the wiping member is configured to provide a hermetic seal between the top wall and the wiping member and the bottom wall and the wiping member when the wiping member moves across the housing.

In one or more embodiments, the wiping member is configured to wipe off a surface of the first sealing member of the housing when the wiping member moves across the housing.

In one or more embodiments, a filter is disposed in, on, or over an aperture in the housing for releasing air from the housing when actuating the wiping member.

In one or more embodiments, the housing is manufactured from a group consisting of plastic, rigid plastic, flexible plastic, polymer, and combinations thereof.

In one or more embodiments, the housing is made of a glass material, a metal material, and combinations thereof.

In one or more embodiments, the wiping member is made of a plastic substance, a rubber substance, an elastomeric substance, and combinations thereof.

In one or more embodiments, the wiping member is covered and/or saturated with an antibacterial substance, an antiviral substance, an anti-tuberculin substance, an anti-HIV substance, hydrogen peroxide, isopropyl alcohol, and combinations thereof.

In one or more embodiments, the device and/or housing is disposable and/or for single use.

In one or more embodiments, a surface of the removable sealing member has an adhesive on the surface of the removable sealing member.

In one or more embodiments, an exterior surface of the removable sealing member has an adhesive.

In one or more embodiments, the device further comprises a sealing ring.

In one or more embodiments, the sealing ring of the device is an “O-ring”.

In one or more embodiments, the sealing ring of the device is a circumferential sealing ring.

In one or more embodiments, the “O-ring” of the device is a circumferential “O-ring”.

In one or more embodiments, the sealing ring of the device is made of at least one of a plastic material and a rubber material.

In one or more embodiments, the sealing “O-ring” of the device is made of at least one of a plastic material and a rubber material.

The present invention, in one or more embodiments, is based on a system comprising a device having two decontamination mechanisms, and a connection port having one decontamination mechanism. The device having a removable sealing member which provides a first decontaminating action when the removable sealing member is removed/displaced from the device and the device having a wiping member providing a second decontaminating action, the wiping member configured to move across the device thus wiping off/removing any remaining contaminants from the fluid passageway of the device. The wiping member of the device is further configured to wipe off a surface of the connection port. The wiping member of the device is intended to move across the housing and exert it's wiping action after the removable sealing member of the device and the removable sealing member of the connection port have been displaced external to the location of fluidic communication between the device and the connection port. The connection port having one decontamination mechanism being the removable sealing member of the connection port.

The device and the connection port of the system are intended to engage/couple with each other, preferably in an airtight/hermetic manner. At about the time and/or after said coupling, ambient air particles/contaminants are entrapped between the removable sealing member of the device and the removable sealing member of the connection port. The removable sealing member of the device and the removable sealing member of the connection port are then displaced at about the same time, optionally or alternately simultaneously, to a second unsealed position and after said displacement the wiping member of the device is moved across the device via actuation of the actuator, the movement of the wiping member across the device wipes off either or both a first sealing member of the device and a second sealing member of the connection port. Thus a system having a dual decontamination mechanism is provided, the first decontamination mechanism being the displacement to a second unsealed position of the removable sealing member of the device and the removable sealing member of the connection port, and the second decontamination mechanism being the wiping of at least one of, or both, the first sealing member of the device and the second sealing member of the connection port.

In a further aspect the present invention provides a device comprising: a housing having a wiping member disposed inside the housing, the wiping member configured to move across the housing; an actuator connected to the wiping member, the actuator configured for moving the wiping member across the housing, the actuator extending through an opening in the wall of the housing; a removable sealing member for sealing a distal end portion of the housing, the removable sealing member positioned to seal the distal end portion of the fluid passageway, the removable sealing member configured to be movable between a first sealed position and a second unsealed position, the removable sealing member being moveable by manual manipulation of the removable sealing member, wherein the removable sealing member is displaced from the fluid passageway when the removable sealing member is in the second unsealed position; and a plug disposed at the distal end portion of the device, the plug being covered by the removable sealing member when the removable sealing member is in the first sealed position and wherein the plug is exposed to ambient air particles/contaminants when the removable sealing member is in the second unsealed position, the plug configured to displace from the distal end portion of the device into the interior of the device or exterior to the device by manual manipulation of the plug.

In one or more embodiments, the plug is made of a plastic material.

In one or more embodiments, the plug is made of a rubber material.

In one or more embodiments, the plug is a bung.

In one or more embodiments, the plug is a rubber bung.

In one or more embodiments, the plug is a stopper.

In one or more embodiments, the plug is a rubber stopper.

In one or more embodiments, the plug is pushed outside the device to an exterior of the device by a user.

In one or more embodiments, the plug is pushed exterior to the device by application of a force of at least one of an actuator and a piercing member.

In one or more embodiments, the plug is pushed exterior to the device by application of a force to the device.

In one or more embodiments, the plug is further configured to engage/couple a second plug of a connection port.

In one or more embodiments, the plug has an engagement mechanism in the form of any of a clamping member, a clasping member, a retention member, a thread, and a ratchet teeth mechanism.

In one or more embodiments, the device further comprises a sealing ring.

In one or more embodiments, the sealing ring of the device is an “O-ring”.

In one or more embodiments, the sealing ring of the device is a circumferential sealing ring.

In one or more embodiments, the “O-ring” of the device is a circumferential “O-ring”.

In one or more embodiments, the sealing ring of the device is made of at least one of a plastic material and a rubber material.

In one or more embodiments, the sealing “O-ring” of the device is made of at least one of a plastic material and a rubber material.

In a further aspect the present invention provides a system comprising: a device comprising: a housing having a wiping member disposed inside the housing, the wiping member configured to move across the housing; an actuator connected to the wiping member, the actuator configured for moving the wiping member across the housing, the actuator extending through an opening in the wall of the housing; a removable sealing member for sealing a distal end portion of the housing, the removable sealing member positioned to seal the distal end portion of the fluid passageway, the removable sealing member configured to be movable between a first sealed position and a second unsealed position, the removable sealing member being moveable by manual manipulation of the removable sealing member, wherein the removable sealing member is displaced from the fluid passageway when the removable sealing member is in the second unsealed position; and a plug disposed at the distal end portion of the device, the plug being covered by the removable sealing member when the removable sealing member is in the first sealed position, the plug configured to displace from the distal end portion of the device into the interior of the device or exterior to the device by manual manipulation of the plug; and a connection port comprising: a proximal end portion defining a fluid passageway, the proximal end portion of the connection port configured to couple the distal end portion of the housing of the device, the proximal end portion of the connection port having a securing mechanism configured to engage the securing mechanism of a distal end portion of the housing of the device; a removable sealing member for sealing the proximal end portion of the fluid passageway of the connection port, the removable sealing member of the connection port configured to be movable between a first sealed position and a second unsealed position, the removable sealing member of the connection port being movable by manual manipulation of the removable sealing member, wherein the removable sealing member is displaced from the fluid passageway of the connection port when the removable sealing member is in the second unsealed position; and a second sealing member disposed at the proximal end portion of the connection port, the second sealing member being covered by the removable sealing member of the connection port when the removable sealing member is in the first sealed position, the second sealing member configured to displace from the proximal end portion of the device into the interior of the connection port or exterior to the connection port by manual manipulation of the second sealing member.

In one or more embodiments, the second sealing member is a plug.

In one or more embodiments, the second sealing member is configured to internally displace into the fluid passageway of the connection port or into the interior of the housing of the device.

In one or more embodiments, the second sealing member is made of a plastic material.

In one or more embodiments, the second sealing member is made of a rubber material.

In one or more embodiments, the second sealing member is a bung.

In one or more embodiments, the second sealing member is a rubber bung.

In one or more embodiments, the second sealing member is a stopper.

In one or more embodiments, the second sealing member is a rubber stopper.

In one or more embodiments, the second sealing member is configured to be pushed exterior to the connection port by a force of a user's hand.

In one or more embodiments, the second sealing member is configured to be pushed into the interior of the connection port by application of a force of at least one of an actuator and a piercing member.

In one or more embodiments, the second sealing member is configured to be pushed into the interior of the connection port by a force applied to the second sealing member.

In one or more embodiments, the second sealing member is further configured to engage/couple the plug of the housing.

In one or more embodiments, the second sealing member is further configured to engage the plug of the device.

In one or more embodiments, the second sealing member has an engagement mechanism in the form of any of a clamping member, a clasping member, a retention member, a thread, and a ratchet teeth mechanism.

In one or more embodiments, the second sealing member of the connection port engages the plug of the device, and after said engagement both the second sealing member and the plug displace into the interior of the device.

In one or more embodiments, the second sealing member of the connection port engages the plug of the device, and after said engagement both the second sealing member and the plug displace into the interior of the connection port.

In one or more embodiments, the second sealing member of the connection port engages/couples the plug of the device, and after said engagement both the second sealing member and the plug displace into the interior of the device or into the interior of the connection port.

In one or more embodiments the removable sealing member of the device and the removable sealing member of the connection port displace external to the device and the connection port prior to engagement and displacement of the plug and the second sealing member.

In one or more embodiments, the plug engages/couples the second sealing member and displaces into the interior of the device or into the interior of the connection port after external displacement of the removable sealing member of the device and the removable sealing member of the connection port.

In one or more embodiments, the wiping member moves across the device after the internal displacement of the plug and the second sealing member.

In one or more embodiments, the plug engages/couples the second sealing member and displaces into the interior of the device or into the interior of the connection port prior to movement of the wiping member across the housing of the device.

In one or more embodiments, the second sealing member of the connection port and the plug of the device are both configured to be displaced into the interior of the device when the connection port is engaged/coupled to the device.

In one or more embodiments, the second sealing member of the connection port and the plug of the device are both configured to be displaced into the interior of the connection port when the connection port is engaged/coupled to the device.

In one or more embodiments, the device further comprises a sealing ring.

In one or more embodiments, the sealing ring of the device is an “O-ring”.

In one or more embodiments, the sealing ring of the device is a circumferential sealing ring.

In one or more embodiments, the “O-ring” of the device is a circumferential “O-ring”.

In one or more embodiments, the sealing ring of the device is made of at least one of a plastic material and a rubber material.

In one or more embodiments, the sealing “O-ring” of the device is made of at least one of a plastic material and a rubber material.

In one or more embodiments, the connection port further comprises a sealing ring.

In one or more embodiments, the sealing ring of the connection port is an “O-ring”.

In one or more embodiments, the sealing ring of the connection port is a circumferential sealing ring.

In one or more embodiments, the “O-ring” of the connection port is a circumferential “O-ring”.

In one or more embodiments, the sealing ring of the connection port is made of at least one of a plastic material and a rubber material.

In one or more embodiments, the sealing “O-ring” of the connection port is made of at least one of a plastic material and a rubber material.

In one or more embodiments, both the device and connection port further comprising a sealing ring.

In one or more embodiments, both the sealing ring(s) of the device and the connection port are an “O-ring”.

In one or more embodiments, the sealing ring of the device and the connection port are a circumferential sealing ring(s).

In one or more embodiments, the “O-ring” of the device and the connection port are a circumferential “O-ring”.

In one or more embodiments, the sealing ring of the device and the connection port are made of at least one of a plastic material and a rubber material.

In one or more embodiments, the sealing “O-ring” of both the device and the connection port are made of at least one of a plastic material and a rubber material.

In a further aspect the present invention provides a method for establishing a decontaminated fluid passageway between a device and a connection port, comprising: providing a device having a housing, the housing having a wiping member disposed inside the housing, the wiping member configured to move across the housing, the housing further having an actuator connected to the wiping member, the actuator configured for moving the wiping member across the housing, the actuator extending through an opening in the wall of the housing, the housing further having a removable sealing member for sealing a distal end portion of the housing, the removable sealing member positioned to seal the distal end portion of the fluid passageway, the removable sealing member configured to be movable between a first sealed position and a second unsealed position, the removable sealing member being moveable by manual manipulation of the removable sealing member, wherein the removable sealing member is displaced from the fluid passageway when the removable sealing member is in the second unsealed position, the housing further having a plug disposed at the distal end portion of the housing of the device, the plug being covered by the removable sealing member when the removable sealing member is in the first sealed position, the plug configured to displace from the distal end portion of the device into the interior of the device or exterior to the device by manual manipulation of the plug; providing a connection port having a proximal end portion defining a fluid passageway, the proximal end portion of the connection port configured to couple the distal end portion of the housing of the device, the proximal end portion of the connection port having a securing mechanism configured to engage the securing mechanism of a distal end portion of the housing of the device, the connection port further having a removable sealing member for sealing the proximal end portion of the fluid passageway of the connection port, the removable sealing member of the connection port configured to be movable between a first sealed position and a second unsealed position, the removable sealing member of the connection port being movable by manual manipulation of the removable sealing member, wherein the removable sealing member is displaced from the fluid passageway of the connection port when the removable sealing member is in the second unsealed position, the connection port further having a second sealing member disposed at the proximal end portion of the connection port, the second sealing member being covered by the removable sealing member of the connection port when the removable sealing member is in the first sealed position, the second sealing member configured to displace from the proximal end portion of the device into the interior of the connection port or exterior to the connection port by manual manipulation of the second sealing member; connecting the distal end portion of the housing of the device to the proximal end portion of the connection port; moving the removable sealing member of the device to the second unsealed position by manually manipulating the removable sealing member of the device and moving the removable sealing member of the connection port to the second unsealed position by manually manipulating the removable sealing member of the connection port; engaging the plug of the device to the second sealing member of the connection port via an engagement mechanism on at least one of the plug and second sealing member; displacing both the plug and the second sealing member into the interior of the device or into the interior of the connection port; and moving the wiping member of the device across the housing.

In one or more embodiments, the second sealing member is a plug.

In one or more embodiments, the second sealing member is configured to internally displace into the fluid passageway of the connection port or into the interior of the housing of the device.

In one or more embodiments, the second sealing member is made of a plastic material.

In one or more embodiments, the second sealing member is made of a rubber material.

In one or more embodiments, the second sealing member is a bung.

In one or more embodiments, the second sealing member is a rubber bung.

In one or more embodiments, the second sealing member is a stopper.

In one or more embodiments, the second sealing member is a rubber stopper.

In one or more embodiments, the second sealing member is configured to be pushed exterior to the connection port by a force of a user's hand.

In one or more embodiments, the second sealing member is configured to be pushed into the interior of the connection port by application of a force of at least one of an actuator and a piercing member.

In one or more embodiments, the second sealing member is configured to be pushed into the interior of the connection port by a force applied to the second sealing member.

In one or more embodiments, the second sealing member is further configured to engage/couple the plug of the housing.

In one or more embodiments, the second sealing member is further configured to engage the plug of the device.

In one or more embodiments, the second sealing member has an engagement mechanism in the form of any of a clamping member, a clasping member, a retention member, a thread, and a ratchet teeth mechanism.

In one or more embodiments, the second sealing member of the connection port engages the plug of the device, and after said engagement both the second sealing member and the plug displace into the interior of the device.

In one or more embodiments, the second sealing member of the connection port engages the plug of the device, and after said engagement both the second sealing member and the plug displace into the interior of the connection port.

In one or more embodiments, the second sealing member of the connection port engages/couples the plug of the device, and after said engagement both the second sealing member and the plug displace into the interior of the device or into the interior of the connection port.

In one or more embodiments the removable sealing member of the device and the removable sealing member of the connection port displace external to the device and the connection port prior to engagement and displacement of the plug and the second sealing member.

In one or more embodiments, the plug engages/couples the second sealing member and displaces into the interior of the device or into the interior of the connection port after external displacement of the removable sealing member of the device and the removable sealing member of the connection port.

In one or more embodiments, the wiping member moves across the device after the internal displacement of the plug and the second sealing member.

In one or more embodiments, the plug engages/couples the second sealing member and displaces into the interior of the device or into the interior of the connection port prior to movement of the wiping member across the housing of the device.

In one or more embodiments, the second sealing member of the connection port and the plug of the device are both configured to be displaced into the interior of the device when the connection port is engaged/coupled to the device.

In one or more embodiments, the second sealing member of the connection port and the plug of the device are both configured to be displaced into the interior of the connection port when the connection port is engaged/coupled to the device.

In one or more embodiments of the method, the moving of the removable sealing member of the device and the moving of the removable sealing member of the connection port displaces both the removable sealing member of the device and displaces the removable sealing member of the connection port from the fluid passageway of both the housing and the connection port.

In one or more embodiments of the method, the moving of the removable sealing member of the device and the moving of the removable sealing member of the connection port displaces both the removable sealing member of the device and displaces the removable sealing member of the connection port from the fluid passageway between the housing of the device and the connection port.

In one or more embodiments of the method, the moving of the removable sealing member of the device to the second unsealed position and moving the removable sealing member of the connection port to the second unsealed position occurs before moving of the wiping member across the housing.

In one or more embodiments of the method, the moving of the wiping member across the housing occurs after displacing the removable sealing members of the device and the connection port.

In one or more embodiments of the method, displacing the removable sealing member of the device and displacing the removable sealing member of the connection port occurs at about the same time.

In one or more embodiments of the method, displacing the removable sealing member of the device and the displacing the removable sealing member of the connection port occurs at the same time.

In one or more embodiments of the method, displacing the removable sealing member of the device and displacing the removable sealing member of the connection port occurs in the same direction.

In one or more embodiments of the method, the moving the removable sealing member of the device to the second unsealed position and the moving of the removable sealing member of the connection port to the second unsealed position decontaminates the connection between the device and the connection port.

In one or more embodiments of the method, the actuator comprises at least one of a tab, a rod, and a handle.

In one or more embodiments of the method, the actuator is a tab.

In one or more embodiments of the method, the actuator is a rod.

In one or more embodiments of the method, the actuator is a handle.

In one or more embodiments of the method, a sliding motion by a user moves the tab across the housing which results in the movement of the wiping member across the housing.

In one or more embodiments of the method, a sliding motion by a user moves the rod across the housing which results in the movement of the wiping member across the housing.

In one or more embodiments of the method, wherein the wiping member is moved across the housing of the device, the wiping member wipes off the first sealing member of the device.

In one or more embodiments of the method, wherein the connection port further comprises a second sealing member located at the proximal end portion of the connection port.

In one or more embodiments of the method, wherein the connection port further comprising a second sealing member located at the proximal end portion of the connection port.

In one or more embodiments of the method, wherein when the wiping member is moved across the housing of the device, the wiping member wipes off the first sealing member of the device and the wiping member wipes off the second sealing member of the connection port.

In one or more embodiments of the method, wherein when the wiping member wipes off the first sealing member of the device and the wiping member wipes off the second sealing member of the connection port, the wiping member decontaminates a surface of the first sealing member of the device and the wiping member decontaminates a surface of the second sealing member of the connection port.

In one or more embodiments of the method, wherein the device further comprising a piercing member configured to pierce through the first sealing member of the device and the piercing member configured to pierce through the second sealing member of the connection port.

In one or more embodiments of the method, the method further comprising the piercing member piercing through the first sealing member of the device and the second sealing member of the connection port.

In one or more embodiments of the method, wherein the piercing of the first sealing member and the piercing of the second sealing member by the piercing member establishes a fluidic communication between the device and the connection port.

In one or more embodiments of the method, wherein moving of the removable sealing member of the device and moving of the removable sealing member of the connection port occurs via a pulling motion by a user of both the removable sealing member of the device and the removable sealing member of the connection port.

In one or more embodiments of the method, wherein moving of the removable sealing member of the device to the second unsealed position and moving of the removable sealing member of the connection port to the second unsealed position exposes a surface of the second sealing member of the connection port to the interior of the housing of the device.

In one or more embodiments of the method, wherein when the wiping member is moved across the housing of the device, the wiping member wipes off the first sealing member of the device and the wiping member wipes off the second sealing member of the connection port, the wiping member further quarantines any contaminants located inside the housing away from the fluid passageway established between the device and the connection port.

In one or more embodiments of the method, wherein the housing further comprising a locking member located on an interior wall of the housing, the locking member configured for locking the wiping member in an extended position to prevent the wiping member from returning to an original position.

In one or more embodiments of the method, wherein the method further comprising the locking member locking the wiping member in an extended position.

In one or more embodiments of the method, wherein the locking member locks the wiping member in an extended position after the wiping member has moved across the housing.

In one or more embodiments of the method, the securing mechanism of the distal end portion of the housing of the device is further configured to be movable between a first extended position and a second contracted position.

In one or more embodiments of the method, wherein when the device is coupled to the connection port and when the securing mechanism of the device is in the first extended position, an exterior surface of the second sealing member of the connection port is not aligned with an interior surface of the housing, and wherein when the device is coupled to the connection port and the securing mechanism of the device is in the second contracted position, an exterior surface of the second sealing member of the connection port is aligned with an interior surface of the housing.

In one or more embodiments of the method, wherein when the exterior surface of the second sealing member of the connection port is aligned with an interior surface of the housing, the wiping member is able to wipe off the exterior surface of the second sealing member of the connection port when the wiping member moves across the housing.

In one or more embodiments of the method, wherein when the distal end portion of the housing of the device is further configured to be moveable between a first extended position and a second contracted position.

In one or more embodiments of the method, the securing mechanism of the device is a thread.

In one or more embodiments of the method, the securing mechanism of the device are ratchet teeth.

In one or more embodiments of the method, the securing mechanism of the device is an adhesive.

In one or more embodiments of the method, the securing mechanism of the device is a clamping member.

In one or more embodiments of the device, the securing mechanism of the device is a retention member.

In one or more embodiments of the method, the distal end portion of the housing of the device is further configured to be movable between a first extended position and a second contracted position.

In one or more embodiments of the method, the method further comprising moving the securing mechanism of the distal end portion of the housing of the device from a first extended position to a second contracted position.

In one or more embodiments of the method, wherein the moving of the securing mechanism of the distal end portion of the housing of the device from the first extended position to the second contracted position occurs after the moving of the removable sealing member of the device to the second unsealed position and after the moving of the removable sealing member of the connection port to the second unsealed position.

In one or more embodiments of the method, wherein the moving of the securing mechanism of the distal end portion of the housing from the first extended position to the second contracted position occurs before moving of the wiping member of the device across the housing.

In one or more embodiments of the method, wherein the moving of the securing mechanism of the distal end portion of the housing of the device from the first extended position to the second contracted position occurs after the moving of the removable sealing member of the device to the second unsealed position and after the moving of the removable sealing member of the connection port to the second unsealed position, and before moving of the wiping member across the housing.

In one or more embodiments of the method, wherein the securing mechanism of the distal end portion of the housing of the device is selected from a group consisting of a thread, ratchet teeth, a clamping member, a retention member, and an adhesive.

In one or more embodiments, the proximal end portion of the housing of the device is further configured to couple to a container and the distal end portion of the connection port is further configured to couple to a container.

In one or more embodiments, the proximal end portion of the housing of the device is further configured to couple to a bag and the distal end portion of the connection port is further configured to couple to a vial.

In one or more embodiments, wherein the distal end portion of the connection port is attached to a syringe and the proximal end portion of the housing of the device is attached to a container.

In one or more embodiments, the distal end portion of the connection port is integrally attached to a container and the proximal end portion of the housing of the device is integrally attached to a container.

In one or more embodiments of the system, the distal end portion of the connection port forms a unitary structure with a container and the proximal end portion of the housing of the device forms a unitary structure with a container.

In one or more embodiments of the system, the distal end portion of the connection port is attached to any of a syringe, a filter, an infusion line, and wherein the proximal end portion of the housing of the device is attached to any of a syringe, a filter, an infusion line, and a container.

In one or more embodiments, the device further comprises a sealing ring.

In one or more embodiments, the sealing ring of the device is an “O-ring”.

In one or more embodiments, the sealing ring of the device is a circumferential sealing ring.

In one or more embodiments, the “O-ring” of the device is a circumferential “O-ring”.

In one or more embodiments, the sealing ring of the device is made of at least one of a plastic material and a rubber material.

In one or more embodiments, the sealing “O-ring” of the device is made of at least one of a plastic material and a rubber material.

In one or more embodiments, the connection port further comprises a sealing ring.

In one or more embodiments, the sealing ring of the connection port is an “O-ring”.

In one or more embodiments, the sealing ring of the connection port is a circumferential sealing ring.

In one or more embodiments, the “O-ring” of the connection port is a circumferential “O-ring”.

In one or more embodiments, the sealing ring of the connection port is made of at least one of a plastic material and a rubber material.

In one or more embodiments, the sealing “O-ring” of the connection port is made of at least one of a plastic material and a rubber material.

In one or more embodiments, both the device and connection port further comprising a sealing ring.

In one or more embodiments, both the sealing ring(s) of the device and the connection port are an “O-ring”.

In one or more embodiments, the sealing ring of the device and the connection port are a circumferential sealing ring(s).

In one or more embodiments, the “O-ring” of the device and the connection port are a circumferential “O-ring”.

In one or more embodiments, the sealing ring of the device and the connection port are made of at least one of a plastic material and a rubber material.

In one or more embodiments, the sealing “O-ring” of both the device and the connection port are made of at least one of a plastic material and a rubber material.

In one or more embodiments, the sealing ring of at least one of the device and the connection port maintains a hermetic seal between the device and the connection port at about the time of displacement of the removable sealing members of the device and the connection port to a second unsealed position.

In one or more embodiments, the sealing ring of at least one of the device and the connection port maintains a hermetic seal between the device and the connection port and reseals the aperture through which the removable sealing members displace during the displacement of the removable sealing members of the device and the connection port to a second unsealed position.

In one or more embodiments, the “O-ring of at least one of the device and the connection port maintains a hermetic seal between the device and the connection port at about the time of displacement of the removable sealing members of the device and the connection port to a second unsealed position.

In one or more embodiments, the “O-ring of at least one of the device and the connection port maintains a hermetic seal between the device and the connection port and reseals the aperture through which the removable sealing members displace during the displacement of the removable sealing members of the device and the connection port to a second unsealed position.

In one or more embodiments, the sealing “O-ring” of at least one of the device and the connection port maintains a hermetic seal between the device and the connection port at about the time of displacement of the removable sealing members of the device and the connection port to a second unsealed position.

In one or more embodiments, the sealing “O-ring” of at least one of the device and the connection port maintains a hermetic seal between the device and the connection port and reseals the aperture through which the removable sealing members displace during the displacement of the removable sealing members of the device and the connection port to a second unsealed position.

In a further aspect, the present invention provides a device for decontaminated engagement between the device and a connection port, the device comprising: a housing defining a fluid passageway, a distal end portion of the housing configured to couple a connection port, the distal end portion of the housing having a securing mechanism configured to engage the connection port; a removable sealing member for sealing the distal end portion of the housing, the removable sealing member positioned to seal the distal end portion of the fluid passageway, the removable sealing member configured to be movable between a first sealed position and a second unsealed position, the removable sealing member being movable by manual manipulation of the removable sealing member, wherein the removable sealing member is displaced from the fluid passageway when the removable sealing member is in the second unsealed position; and a plug located in an opening at the distal end portion of the housing, the plug when in a first position is positioned to fluidly seal the opening of the distal end portion of the housing, the plug configured to be displaced from the first position by manual manipulation of the plug, wherein when the plug is displaced and in a second position, the plug is located inside an interior of the housing or the plug is located exterior to the housing.

In one or more embodiments, the plug further comprising an engagement/coupling mechanism configured to engage/couple a second sealing member of a connection port.

In one or more embodiments, the engagement/coupling mechanism of the plug are selected from the group consisting of ratchet teeth, a clamping member, a retention member, a thread, and combinations thereof.

In one or more embodiments, the plug is a stopper.

In one or more embodiments, the plug is a rubber stopper.

In one or more embodiments, the plug is a bung.

In one or more embodiments, the plug is a rubber bung.

In one or more embodiments, the plug engages/couples a second sealing member of a connection port and thereafter displaces into the housing of the device or exterior to the housing of the device.

In one or more embodiments, the device further comprising a guiding member configured to align the removable sealing member of the device with a removable sealing member of a connection port in such a manner in which the removable sealing members of both the device and the connection port lye flat against each other.

In one or more embodiments, the device further comprising a sealing ring.

In one or more embodiments, the device further comprising and “O-ring”.

In a further aspect, the present invention provides a system for decontaminated engagement between a device and a connection port, the system comprising: 1.) a device having a housing defining a fluid passageway, a distal end portion of the housing configured to couple a connection port, the distal end portion of the housing having a securing mechanism configured to engage the connection port; a removable sealing member for sealing the distal end portion of the housing, the removable sealing member positioned to seal the distal end portion of the fluid passageway, the removable sealing member configured to be movable between a first sealed position and a second unsealed position, the removable sealing member being movable by manual manipulation of the removable sealing member, wherein the removable sealing member is displaced from the fluid passageway when the removable sealing member is in the second unsealed position; and a plug located in an opening at the distal end portion of the housing, the plug when in a first position is positioned to fluidly seal the opening of the distal end portion of the housing, the plug configured to be displaced from the first position by manual manipulation of the plug, wherein when the plug is displaced and in a second position, the plug is located inside an interior of the housing or the plug is located exterior to the housing; and 2.) a connection port comprising a proximal end portion defining a fluid passageway, the proximal end portion of the connection port configured to couple the distal end portion of the housing of the device, the proximal end portion of the connection port having a securing mechanism configured to engage the securing mechanism of the distal end portion of the housing of the device; a removable sealing member for sealing the proximal end portion of the fluid passageway of the connection port, the removable sealing member of the connection port configured to be movable between a first sealed position and a second unsealed position, the removable sealing member of the connection port being movable by manual manipulation of the removable sealing member, wherein the removable sealing member is displaced from the fluid passageway of the connection port when the removable sealing member is in the second unsealed position; and a plug located in an opening at the proximal end portion of the connection port, the plug when in a first position is positioned to fluidly seal the opening of the proximal end portion of the connection port, the plug configured to be displaced from the first position by manual manipulation of the plug, wherein when the plug is displaced to a second position, the plug is located inside an interior of the connection port or the plug is located exterior to the connection port.

In one or more embodiments, at least one of the plug(s) of the device and the connection port further comprising an engagement/coupling mechanism.

In one or more embodiments, the engagement/coupling mechanism of the plug of the device and the plug of the connection port are selected from the group consisting of ratchet teeth, a clamping member, a retention member, a thread, and combinations thereof.

In one or more embodiments, at least one of the plug(s) of the device and the connection port is a stopper.

In one or more embodiments, at least one of the plug(s) of the device and the connection port is a rubber stopper.

In one or more embodiments, at least one of the plug(s) of the device and the connection port is a bung.

In one or more embodiments, at least one of the plug(s) of the device and the connection port is a rubber bung.

In one or more embodiments, the plug of the housing of the device is configured to engage the plug of the connection port.

In one or more embodiments, the plug of the connection port is configured to couple with the plug of the device.

In one or more embodiments, the plug of the device and the plug of the connection port are configured to engage/couple to each other and then both the plug of the device and the plug of the connection port are configured to simultaneously displace into the interior of the device or into the interior of the connection port.

In one or more embodiments, at least one of the device and the connection port further comprising a guiding member configured to align the removable sealing member of the device with a removable sealing member of a connection port in such a manner in which the removable sealing members of both the device and the connection port lie flat against each other.

In one or more embodiments, at least one of the device and the connection port further comprising a sealing ring.

In one or more embodiments, at least one of the device and the connection port further comprising and “O-ring”.

In one or more embodiments, the plug of the device and the plug of the connection port are intended to displace into the interior of the device or into the interior of the connection port after the removable sealing member of the device and the removable sealing member of the connection port have been displaced to a second unsealed position.

In a further aspect, the present invention provides a method for decontaminating a fluidic passageway between a device and a connection port, the method comprising the steps of: providing 1.) a device having a housing defining a fluid passageway, a distal end portion of the housing configured to couple a connection port, the distal end portion of the housing having a securing mechanism configured to engage the connection port; a removable sealing member for sealing the distal end portion of the housing, the removable sealing member positioned to seal the distal end portion of the fluid passageway, the removable sealing member configured to be movable between a first sealed position and a second unsealed position, the removable sealing member being movable by manual manipulation of the removable sealing member, wherein the removable sealing member is displaced from the fluid passageway when the removable sealing member is in the second unsealed position; and a plug located in an opening at the distal end portion of the housing, the plug when in a first position is positioned to fluidly seal the opening of the distal end portion of the housing, the plug configured to be displaced from the first position by manual manipulation of the plug, wherein when the plug is displaced and in a second position, the plug is located inside an interior of the housing or the plug is located exterior to the housing; providing 2.) a connection port comprising a proximal end portion defining a fluid passageway, the proximal end portion of the connection port configured to couple the distal end portion of the housing of the device, the proximal end portion of the connection port having a securing mechanism configured to engage the securing mechanism of the distal end portion of the housing of the device; a removable sealing member for sealing the proximal end portion of the fluid passageway of the connection port, the removable sealing member of the connection port configured to be movable between a first sealed position and a second unsealed position, the removable sealing member of the connection port being movable by manual manipulation of the removable sealing member, wherein the removable sealing member is displaced from the fluid passageway of the connection port when the removable sealing member is in the second unsealed position; and a plug located in an opening at the proximal end portion of the connection port, the plug when in a first position is positioned to fluidly seal the opening of the proximal end portion of the connection port, the plug configured to be displaced from the first position by manual manipulation of the plug, wherein when the plug is displaced to a second position, the plug is located inside an interior of the connection port or the plug is located exterior to the connection port; 3.) connecting/engaging/coupling the distal end portion of the device to the proximal end portion of the connection port; 4.) displacing the removable sealing member of the device and displacing the removable sealing member of the connection port to a second unsealed position; 5.) engaging/coupling the plug of the device to the plug of the connection port; and 6.) displacing both the plug of the device and the plug of the connection port into an interior of the device or into the interior of the connection port.

In one or more embodiments, at least one of the plug(s) of the device and the connection port further comprising an engagement/coupling mechanism.

In one or more embodiments, the engagement/coupling mechanism of the plug of the device and the plug of the connection port are selected from the group consisting of ratchet teeth, a clamping member, a retention member, a thread, and combinations thereof.

In one or more embodiments, at least one of the plug(s) of the device and the connection port is a stopper.

In one or more embodiments, at least one of the plug(s) of the device and the connection port is a rubber stopper.

In one or more embodiments, at least one of the plug(s) of the device and the connection port is a bung.

In one or more embodiments, at least one of the plug(s) of the device and the connection port is a rubber bung.

In one or more embodiments, the plug of the housing of the device is configured to engage the plug of the connection port.

In one or more embodiments, the plug of the connection port is configured to couple with the plug of the device.

In one or more embodiments, the plug of the device and the plug of the connection port are configured to engage/couple to each other and then both the plug of the device and the plug of the connection port are configured to simultaneously displace into the interior of the device or into the interior of the connection port.

In one or more embodiments, at least one of the device and the connection port further comprising a guiding member configured to align the removable sealing member of the device with a removable sealing member of a connection port in such a manner in which the removable sealing members of both the device and the connection port lie flat against each other.

In one or more embodiments, at least one of the device and the connection port further comprising a sealing ring.

In one or more embodiments, at least one of the device and the connection port further comprising and “O-ring”.

In one or more embodiments, the plug of the device and the plug of the connection port are intended to displace into the interior of the device or into the interior of the connection port after the removable sealing member of the device and the removable sealing member of the connection port have been displaced to a second unsealed position.

In a further aspect, the present invention provides a container having a removable sealing member and a guiding member, the container further having an engagement mechanism configured to engage/couple a second container, the removable sealing member positioned to seal an opening of the container when the removable sealing member is in a first sealed position, the removable sealing member configured to be movable between the first sealed position and a second unsealed position, the removable sealing member being moveable by manual manipulation of the removable sealing member, wherein the removable sealing member is displaced from the opening of the container when the removable sealing member is in the second unsealed position, the guiding member configured to align the removable sealing member of the container with a removable sealing member of a second container in a manner such that the removable sealing member of the container and the removable sealing member of a second container lye flat against each other when the container is engaged/coupled to the second container.

In one or more embodiments, the container further comprising an sealing ring.

In one or more embodiments, the container further comprising an “O-ring” configured to maintain a hermetic coupling between the container and a second container when the removable sealing member of the container is displaced to the second unsealed position when the container is engaged/coupled to a second container.

In one or more embodiments, the container is a bag or a bottle.

In one or more embodiments, the container is a vial.

In a further aspect, the present invention provides a system for decontaminated engagement between two containers comprising: a first container having a removable sealing member, the first container further having an engagement mechanism configured to engage/couple a second container, the removable sealing member positioned to seal an opening of the first container when the removable sealing member is in a first sealed position, the removable sealing member configured to be movable between the first sealed position and a second unsealed position, the removable sealing member being moveable by manual manipulation of the removable sealing member, wherein the removable sealing member is displaced from the opening of the first container when the removable sealing member is in the second unsealed position; a second container having a removable sealing member, the second container further having an engagement mechanism configured to engage/couple the first container, the removable sealing member positioned to seal an opening of the second container when the removable sealing member is in a first sealed position, the removable sealing member configured to be movable between the first sealed position and a second unsealed position, the removable sealing member being moveable by manual manipulation of the removable sealing member, wherein the removable sealing member is displaced from the opening of the second container when the removable sealing member is in the second unsealed position.

In one or more embodiments, the removable sealing member of the first container and the removable sealing member of the second container are configured to substantially fluidly seal an opening of the first container and an opening of the second container.

In one or more embodiments, the removable sealing member of the first container and the removable sealing member of the second container are configured to hermetically seal an opening of the first container and an opening of the second container.

In one or more embodiments, at least one of the first and second containers having at least one guiding member configured to align the removable sealing member of the first container with the removable sealing member of the second container so that the removable sealing members of the first and second containers lye flat against each other (one another).

In one or more embodiments, at least one of the first and second containers having a sealing ring.

In one or more embodiments, at least one of the first and second containers having a sealing ring disposed on an external surface of the containers.

In one or more embodiments, the sealing ring is a circumferential sealing ring.

In one or more embodiments, at least one of the first and second containers having an “O-ring”.

In one or more embodiments, at least one of the first and second containers having an “O-ring” disposed on an external surface of either or both the first and second containers.

In one or more embodiments, the “O-ring” is a circumferential “O-ring” configured to maintain a hermetic/airtight seal between the first container and the second container during and after displacement of the removable sealing members of both containers to a second unsealed position.

In one or more embodiments, at least one of the first and second containers having a plug positioned in an opening of the either or both the first and second container(s).

In or more embodiments, the plug(s) is/are pierceable.

In one or more embodiments, the plug(s) is/are frangible.

In one or more embodiments, the plug(s) is/are configured to displace into an interior of either the first container or the second container.

In still a further aspect, the present invention provides a system for establishing a decontaminated fluid passageway between a device and a connection port, comprising: i) a device comprising: a housing defining a fluid passageway, a distal end portion of the housing configured to couple a connection port, the distal end portion of the housing having a securing mechanism configured to engage the connection port; a removable sealing member for sealing the distal end portion of the housing, the removable sealing member positioned to seal the distal end portion of the fluid passageway, the removable sealing member configured to be movable between a first sealed position and a second unsealed position, the removable sealing member being moveable by manual manipulation of the removable sealing member, wherein the removable sealing member is displaced from the fluid passageway when the removable sealing member in the second unsealed position; ii) a connection port, comprising: a proximal end portion defining a fluid passageway, the proximal end portion of the connection port configured to couple the distal end portion of the housing of the device, the proximal end portion of the connection port having a securing mechanism configured to engage the securing mechanism of the distal end portion of the housing of the device; a removable sealing member for sealing the proximal end portion of the fluid passageway of the connection port, the removable sealing member of the connection port configured to be movable between a first sealed position and a second unsealed position, the removable sealing member of the connection port being movable by manual manipulation of the removable sealing member, wherein the removable sealing member is displaced from the fluid passageway of the connection port when the removable sealing member is in the second unsealed position; and a second sealing member positioned to seal an opening of the connection port, the second sealing member positioned inside and/or over the opening of the connection port, the second sealing member covered by the removable sealing member of the connection port when the removable sealing member of the connection port is in a first sealed position and the second sealing member being uncovered when the removable sealing member of the connection port is displaced to a second unsealed position.

In one or more embodiments, the second sealing member is at least one of pierceable and frangible.

In one or more embodiments, the second sealing member is a stopper.

In one or more embodiments, the second sealing member is a rubber stopper.

In one or more embodiments, the second sealing member is a bung.

In one or more embodiments, the second sealing member is a rubber bung.

In one or more embodiments, the second sealing member prevents escape/leakage of fluid out of the connection port in the event of spontaneous disengagement between the connection port and the device.

In one or more embodiments, the second sealing member is configured to be pierced by a piercing member of the device.

In one or more embodiments, the second sealing member is configured to be pierced by a piercing member of the connection port.

In one or more embodiments, at least one of the device and the connection port further comprising a piercing member configured to pierce through the second sealing member of the connection port.

In one or more embodiments, the device further comprising a plug positioned in an opening at the distal end portion of the device, the plug configured to prevent escape/leakage of fluid out of the device in the event of spontaneous disengagement between the connection port and the device.

In one or more embodiments, the plug of the device is at least one of pierceable and frangible.

In one or more embodiments, the plug of the device is configured to be pierced by a piercing member of the device or by a piercing member of the connection port.

In one or more embodiments, both the plug of the device and the second sealing member of the connection port serve as a means to prevent spillage of the contents held/transferred between the device and the connection port in the event of spontaneous/accidental disengagement of the device from the connection port.

In one or more embodiments, the removable sealing member of the device and the removable sealing member of the connection port are both configured to substantially seal the fluid passageway between the device and the connection port when both the removable sealing member of the device and the removable sealing member of the connection port are in a first sealed position, and wherein the removable sealing member of the device and the removable sealing member of the connection port are configured to allow passage of fluid between the device and the connection port when the removable sealing member of the device and the removable sealing member of the connection port are both in the second unsealed position.

In one or more embodiments, the removable sealing member of the device and the removable sealing member of the connection port are both configured to be moved from the first sealed position to the second unsealed position via a displacement of the removable sealing members of both the device and the connection port, wherein the displacement of both removable sealing members of the device and the connection port are external to the fluid passageway between the device and the connection port.

In one or more embodiments, the removable sealing member of the device is in a folded configuration and the removable sealing member of the connection port is in a folded configuration.

In one or more embodiments, the removable sealing member of the device is in a folded configuration or the removable sealing member of the connection port is in a folded configuration.

In one or more embodiments, at least one of the removable sealing members of the device and the removable sealing member of the connection port are in a folded configuration.

In one or more embodiments, a portion of the removable sealing member of the device is attached to an interior surface of the housing.

In one or more embodiments, a portion of the removable sealing member of the device is attached to an interior surface of the housing and a portion of the removable sealing member of the device is attached to an exterior surface of the housing.

In one or more embodiments, the first sealing member of the device is characterized as being at least one of pierceable and frangible.

In one or more embodiments, the device further comprising a piercing member.

In one or more embodiments, the housing further comprising a piercing member.

In one or more embodiments, the device further comprising a piercing member actuator.

In one or more embodiments, the piercing member actuator configured to move the piercing member through the first sealing member of the device.

In one or more embodiments, the piercing member is a needle.

In one or more embodiments, the piercing member is a hollowed needle.

In one or more embodiments, the piercing member is configured to pierce through the second sealing member of the connection port when the connection port is engaged to the housing of the device.

In one or more embodiments, the piercing member actuator is configured to move the piercing member through the second sealing member of the connection port when the connection port is coupled to the housing of the device.

In one or more embodiments, the connection port further comprising a second sealing member located at the proximal end portion of the connection port.

In one or more embodiments, the removable sealing member of the connection port covers the second sealing member of the connection port when the removable sealing member of the connection port is in a first sealed position.

In one or more embodiments, the second sealing member is pierceable.

In one or more embodiments, the second sealing member is a rubber bung.

In one or more embodiments, the second sealing member is frangible.

In one or more embodiments, the second sealing member is a rubber stopper.

In one or more embodiments, the second sealing member is made of a plastic material, a metal alloy, and combinations thereof.

In one or more embodiments, the second sealing member is a plug that substantially seals the fluid passageway between the device and the connection port.

In one or more embodiments, the plug is displaceable.

In one or more embodiments, when the device is coupled to the connection port, and when the removable sealing member of the device and the removable sealing member of the connection port are both in the second unsealed position, a surface of the second sealing member of the connection port is exposed to an interior of the housing of the device.

In one or more embodiments, wherein the surface of the second sealing member of the connection port is characterized in that the surface of the second sealing member is positioned to be wiped off by the wiping member of the device when the wiping member moves across the housing.

In one or more embodiments, wherein when the device is coupled to the connection port, the surface of the second sealing member of the connection port and the wiping member of the device form an airtight seal when the wiping member moves across the housing of the device and wipes off the surface of the second sealing member.

In one or more embodiments, the wiping member of the device wipes off a surface of the second sealing member of the connection port via at least one of press and friction force(s).

In one or more embodiments, the device further comprising a piercing member configured to pierce through the first sealing member of the device and the second sealing member of the connection port.

In one or more embodiments, wherein the piercing member is a needle.

In one or more embodiments, wherein the piercing member is a hollowed needle.

In one or more embodiments, the securing mechanism at the distal end portion of the housing of the device is a thread and the securing mechanism at the proximal end portion of the connection port is a complimentary thread.

In one or more embodiments, the securing mechanism at the distal end portion of the housing of the device are ratchet teeth and the securing mechanism at the proximal end portion of the connection port is a complimentary retention member.

In one or more embodiments, the securing mechanism at the distal end portion of the device is a retention member and the securing mechanism at the proximal end portion of the connection port are complimentary ratchet teeth.

In one or more embodiments, the securing mechanism at the distal end portion of the housing of the device is a clamping member and the securing mechanism at the proximal end portion of the connection port is a complimentary retention member.

In one or more embodiments, the securing mechanism at the distal end portion of the housing of the device is a retention member and the securing mechanism at the proximal end portion of the connection port is a complimentary clamping member.

In one or more embodiments, at least one of the securing mechanism at the distal end portion of the housing and the securing mechanism at the proximal end portion of the connection port is an adhesive.

In one or more embodiments, the proximal end portion of the housing of the device is further configured to couple to a container and the distal end portion of the connection port is further configured to couple to a container.

In one or more embodiments, the proximal end portion of the housing of the device is further configured to couple to a bag and the distal end portion of the connection port is further configured to couple to a vial.

In one or more embodiments, wherein the distal end portion of the connection port is attached to a syringe and the proximal end portion of the housing of the device is attached to a container.

In one or more embodiments, the distal end portion of the connection port is integrally attached to a container and the proximal end portion of the housing of the device is integrally attached to a container.

In one or more embodiments of the system, the distal end portion of the connection port forms a unitary structure with a container and the proximal end portion of the housing of the device forms a unitary structure with a container.

In one or more embodiments of the system, the distal end portion of the connection port is attached to any of a syringe, a filter, an infusion line, and wherein the proximal end portion of the housing of the device is attached to any of a syringe, a filter, an infusion line, and a container.

In one or more embodiments of the system, wherein the removable sealing member of the device is attached to the housing of the device via a frangible seal and the removable sealing member of the connection port is attached to the connection port via a frangible seal.

In one or more embodiments of the system, wherein the removable sealing member of the device and the removable sealing member of the connection port are both configured to be displaced from the fluid passageway via a pulling motion.

In one or more embodiments of the system, wherein the removable sealing member of the device and the removable sealing member of the connection port are both configured to be displaced from the fluid passageway at about the same time via a pulling motion.

In one or more embodiments of the system, wherein at least a portion of the removable sealing member of the device is located through a wall of the housing.

In one or more embodiments, wherein at least a portion of the removable sealing member of the device is located through a side wall of the housing.

In one or more embodiments of the system, wherein at least one of an antibacterial substance, an antiviral substance, and an antifungal substance is located inside the housing of the device, on a surface of the removable sealing member, on the wiping member, and combinations thereof.

In one or more embodiments of the system, wherein the securing mechanism of the distal end portion of the housing of the device is further configured to be movable between a first extended position and a second contracted position.

In one or more embodiments of the system, wherein when the securing mechanism of the distal end portion of the housing of the device is in the second contracted position the second sealing member of the connection port is positioned to be wiped off by the wiping member of the device.

In one or more embodiments of the system, wherein when the device is coupled to the connection port and when the securing mechanism of the device is in a first extended position, an exterior surface of the second sealing member of the connection port is not aligned with an interior surface of the housing, and wherein when the device is coupled to the connection port and the securing mechanism of the device is in the second contracted position, an exterior surface of the second sealing member of the connection port is aligned with an interior surface of the housing.

In one or more embodiments of the system, wherein when the exterior surface of the second sealing member of the connection port is aligned with an interior surface of the housing, the wiping member is able to wipe off the exterior surface of the second sealing member of the connection port when the wiping member moves across the housing.

In one or more embodiments of the system, wherein the distal end portion of the housing of the device is further configured to be movable between a first extended position and a second contracted position.

In one or more embodiments, wherein the securing mechanism of the distal end portion of the housing of the device is selected from a group consisting of a thread, ratchet teeth, a clamping member, a retention member, and an adhesive.

In one or more embodiments of the system, the housing further comprising a guiding member configured to align the removable sealing member of the housing with the removable sealing member of the connection port.

In one or more embodiments, the guiding member is a rod.

In one or more embodiments, the guiding member is a dent.

In one or more embodiments, the guiding member is a slot.

In one or more embodiments, the guiding member is a protrusion.

In one or more embodiments of the system, the guiding member is configured to align the removable sealing member of the housing of the device with the removable sealing member of the connection port, wherein upon alignment of the removable sealing member of the housing of the device with the removable sealing member of the connection port both the removable sealing member of the housing and the removable sealing member of the connection port will displace in about the same direction and at about the same time external to the location of fluidic communication between the housing and the connection port.

In one or more embodiments of the system, the connection port has a guiding member configured to align the removable sealing member of the connection port with the removable sealing member of the device when the connection port couples with the device.

In one or more embodiments of the system, the guiding member of the connection port is a rod.

In one or more embodiments of the system, the guiding member of the connection port is a dent.

In one or more embodiments of the system, the guiding member of the connection port is a slot.

In one or more embodiments of the system, the guiding member of the connection port is a protrusion.

In one or more embodiments of the system, wherein the device further comprising a guiding member configured to align the removable sealing member of the device with the removable sealing member of the connection port.

In one or more embodiments of the system, wherein the connection port further comprising a guiding member configured to align the removable sealing member of the device with the removable sealing member of the connection port.

In one or more embodiments of the system, wherein the device further comprising a guiding member and wherein the connection port further comprising a guiding member, wherein both the guiding member of the device and the guiding member of the connection port are configured to engage with each other in such a manner as to align the removable sealing member of the device and the removable sealing member of the connection port so that both said removable sealing members are able to displace at about the same time and in the same direction when moved to a second unsealed position.

In one or more embodiments of the system, wherein the guiding member of the device is a rod and the guiding member of the connection port is a complimentary slot which allows the rod to enter into the slot.

In one or more embodiments of the system, wherein the guiding member of the connection port is a rod and the guiding member of the device is a complimentary slot which allows the rod to enter into the slot.

In one or more embodiments of the system, wherein the guiding member of the device is a protrusion and the complimentary guiding member of the connection port is a dent, wherein the dent allows the protrusion to engage and/or enter into the dent.

In one or more embodiments of the system, wherein the guiding member of the connection port is a protrusion and the complimentary guiding member of the device is a dent, wherein the dent allows the protrusion to engage and/or enter into the dent.

In one or more embodiments, the proximal end portion of the housing is integrally attached to a container and the distal end portion of the housing having a guiding member.

In one or more embodiments, the wiping member is configured to extend from the top wall of the housing to the bottom wall of the housing.

In one or more embodiments, the wiping member is configured to provide a hermetic seal between the top wall and the wiping member and the bottom wall and the wiping member when the wiping member moves across the housing.

In one or more embodiments, the wiping member is configured to wipe off a surface of the first sealing member of the housing when the wiping member moves across the housing.

In one or more embodiments, a filter is disposed in, on, or over an aperture in the housing for releasing air from the housing when actuating the wiping member.

In one or more embodiments, the housing is manufactured from a group consisting of plastic, rigid plastic, flexible plastic, polymer, and combinations thereof.

In one or more embodiments, the housing is made of a glass material, a metal material, and combinations thereof.

In one or more embodiments, the wiping member is made of a plastic substance, a rubber substance, an elastomeric substance, and combinations thereof.

In one or more embodiments, the wiping member is covered and/or saturated with an antibacterial substance, an antiviral substance, an anti-tuberculin substance, an anti-HIV substance, hydrogen peroxide, isopropyl alcohol, and combinations thereof.

In one or more embodiments, the device and/or housing is disposable and/or for single use.

In one or more embodiments, a surface of the removable sealing member has an adhesive on the surface of the removable sealing member.

In one or more embodiments, an exterior surface of the removable sealing member has an adhesive.

In one or more embodiments, the device further comprises a sealing ring.

In one or more embodiments, the sealing ring of the device is an “O-ring”.

In one or more embodiments, the sealing ring is a circumferential sealing ring.

In one or more embodiments, the “O-ring” is a circumferential “O-ring”.

In one or more embodiments, the sealing ring of the device is made of at least one of a plastic material and a rubber material.

In one or more embodiments, the sealing “O-ring” of the device is made of at least one of a plastic material and a rubber material.

In one or more embodiments, the connection port further comprises a sealing ring.

In one or more embodiments, the sealing ring of the connection port is an “O-ring”.

In one or more embodiments, the sealing ring of the connection port is a circumferential sealing ring.

In one or more embodiments, the “O-ring” of the connection port is a circumferential “O-ring”.

In one or more embodiments, the sealing ring of the connection port is made of at least one of a plastic material and a rubber material.

In one or more embodiments, the sealing “O-ring” of the connection port is made of at least one of a plastic material and a rubber material.

In one or more embodiments, both the device and the connection port further comprise a sealing ring.

In one or more embodiments, the sealing ring of the device and the sealing ring of the connection port is an “O-ring”.

In one or more embodiments, the sealing ring of the device is a circumferential sealing ring and the sealing ring of the connection port is a circumferential sealing ring.

In one or more embodiments, the “O-ring” of the device is a circumferential “O-ring” and the “O-ring of the connection port is a circumferential “O-ring”.

In one or more embodiments, the sealing ring of the connection port is made of at least one of a plastic material and a rubber material and the sealing ring of the device is made of at least one of a plastic material and a rubber material.

In one or more embodiments, the sealing “O-ring” of the device and the sealing “O-ring” of the connection port are made of at least one of a plastic material and a rubber material.

In one or more embodiments, the device further comprising teeth configured to engage/couple holes/dents of a connection port.

In one or more embodiments, the teeth of the device are retractable teeth.

In one or more embodiments, the teeth are positioned at a distal end portion of the device.

In one or more embodiments, the retractable teeth are positioned at a distal end portion of the device.

In one or more embodiments, the teeth are positioned on an engagement mechanism of the device.

In one or more embodiments, the teeth are positioned on a securing mechanism of the device.

In one or more embodiments, the teeth are positioned on the device.

In one or more embodiments, the teeth are positioned on the housing of the device.

In one or more embodiments, the retractable teeth are positioned on the device.

In one or more embodiments, the retractable teeth are positioned on the housing of the device.

In one or more embodiments, the teeth are configured to engage/couple a hole/dent of a connection port.

In one or more embodiments, the retractable teeth are configured to engage/couple a hole/dent of a connection port.

In one or more embodiments, the teeth are configured to provide coupling of a connection port to the device, the teeth are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the device further comprising holes/dents configured to engage/couple with teeth of a connection port.

In one or more embodiments, the device further comprising holes/dents configured to engage/couple with retractable teeth of a connection port.

In one or more embodiments, the holes/debts are positioned at a distal end portion of the device.

In one or more embodiments, the holes/dents are positioned on an engagement mechanism of the device.

In one or more embodiments, the holes/dents are positioned on a securing mechanism of the device.

In one or more embodiments, the holes/dents are positioned on the device.

In one or more embodiments, the holes/dents are positioned on the housing of the device.

In one or more embodiments, the holes/dents are configured to engage/couple teeth of a connection port.

In one or more embodiments, the holes/dents are configured to engage/couple retractable teeth of a connection port.

In one or more embodiments, the holes/dents are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments of the system, the device further comprising teeth and the connection port further comprising holes/dents.

In one or more embodiments of the system, the device further comprising retractable teeth and the connection port further comprising holes/dents configured to engage/couple the retractable teeth.

In one or more embodiments of the system, the device further comprising holes/dents and the connection port further comprising teeth.

In one or more embodiments of the system, the device further comprising holes/dents and the connection port further comprising retractable teeth.

In one or more embodiments, the holes/dents of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the holes/dents of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the teeth of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the retractable teeth of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the teeth of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the retractable teeth of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In still a further aspect, the present invention provides a system for establishing a decontaminated fluid passageway between a device and a connection port, comprising: i) a device comprising: a housing defining a fluid passageway, a distal end portion of the housing configured to couple a connection port, the distal end portion of the housing having a securing mechanism configured to engage the connection port; a removable sealing member for sealing the distal end portion of the housing, the removable sealing member positioned to seal the distal end portion of the fluid passageway, the removable sealing member configured to be movable between a first sealed position and a second unsealed position, the removable sealing member being moveable by manual manipulation of the removable sealing member, wherein the removable sealing member is displaced from the fluid passageway when the removable sealing member in the second unsealed position, the housing further comprising a guiding member configured to engage a connection port when the connection port is attached/coupled to the device, the guiding member characterized in that is allows the connection port to couple device in a manner that aligns the removable sealing member of the device with the removable sealing member of a connection port so that both removable sealing members of the device and the connection port lie flat against one another; ii) a connection port, comprising: a proximal end portion defining a fluid passageway, the proximal end portion of the connection port configured to couple the distal end portion of the housing of the device, the proximal end portion of the connection port having a securing mechanism configured to engage the securing mechanism of the distal end portion of the housing of the device; a removable sealing member for sealing the proximal end portion of the fluid passageway of the connection port, the removable sealing member of the connection port configured to be movable between a first sealed position and a second unsealed position, the removable sealing member of the connection port being movable by manual manipulation of the removable sealing member, wherein the removable sealing member is displaced from the fluid passageway of the connection port when the removable sealing member is in the second unsealed position; and a second sealing member positioned to seal an opening of the connection port, the second sealing member positioned inside and/or over the opening of the connection port, the second sealing member covered by the removable sealing member of the connection port when the removable sealing member of the connection port is in a first sealed position and the second sealing member being uncovered when the removable sealing member of the connection port is displaced to a second unsealed position, the connection port further comprising an engagement mechanism configured to engage the guiding member of the device.

In one or more embodiments, the second sealing member is at least one of pierceable and frangible.

In one or more embodiments, the second sealing member is a stopper.

In one or more embodiments, the second sealing member is a rubber stopper.

In one or more embodiments, the second sealing member is a bung.

In one or more embodiments, the second sealing member is a rubber bung.

In one or more embodiments, the second sealing member prevents escape/leakage of fluid out of the connection port in the event of spontaneous disengagement between the connection port and the device.

In one or more embodiments, the second sealing member is configured to be pierced by a piercing member of the device.

In one or more embodiments, the second sealing member is configured to be pierced by a piercing member of the connection port.

In one or more embodiments, at least one of the device and the connection port further comprising a piercing member configured to pierce through the second sealing member of the connection port.

In one or more embodiments, the device further comprising a plug positioned in an opening at the distal end portion of the device, the plug configured to prevent escape/leakage of fluid out of the device in the event of spontaneous disengagement between the connection port and the device.

In one or more embodiments, the plug of the device is at least one of pierceable and frangible.

In one or more embodiments, the plug of the device is configured to be pierced by a piercing member of the device or by a piercing member of the connection port.

In one or more embodiments, both the plug of the device and the second sealing member of the connection port serve as a means to prevent spillage of the contents held/transferred between the device and the connection port in the event of spontaneous/accidental disengagement of the device from the connection port.

In one or more embodiments, the removable sealing member of the device and the removable sealing member of the connection port are both configured to substantially seal the fluid passageway between the device and the connection port when both the removable sealing member of the device and the removable sealing member of the connection port are in a first sealed position, and wherein the removable sealing member of the device and the removable sealing member of the connection port are configured to allow passage of fluid between the device and the connection port when the removable sealing member of the device and the removable sealing member of the connection port are both in the second unsealed position.

In one or more embodiments, the removable sealing member of the device and the removable sealing member of the connection port are both configured to be moved from the first sealed position to the second unsealed position via a displacement of the removable sealing members of both the device and the connection port, wherein the displacement of both removable sealing members of the device and the connection port are external to the fluid passageway between the device and the connection port.

In one or more embodiments, the removable sealing member of the device is in a folded configuration and the removable sealing member of the connection port is in a folded configuration.

In one or more embodiments, the removable sealing member of the device is in a folded configuration or the removable sealing member of the connection port is in a folded configuration.

In one or more embodiments, at least one of the removable sealing members of the device and the removable sealing member of the connection port are in a folded configuration.

In one or more embodiments, a portion of the removable sealing member of the device is attached to an interior surface of the housing.

In one or more embodiments, a portion of the removable sealing member of the device is attached to an interior surface of the housing and a portion of the removable sealing member of the device is attached to an exterior surface of the housing.

In one or more embodiments, the first sealing member of the device is characterized as being at least one of pierceable and frangible.

In one or more embodiments, the device further comprising a piercing member.

In one or more embodiments, the housing further comprising a piercing member.

In one or more embodiments, the device further comprising a piercing member actuator.

In one or more embodiments, the piercing member actuator configured to move the piercing member through the first sealing member of the device.

In one or more embodiments, the piercing member is a needle.

In one or more embodiments, the piercing member is a hollowed needle.

In one or more embodiments, the piercing member is configured to pierce through the second sealing member of the connection port when the connection port is engaged to the housing of the device.

In one or more embodiments, the piercing member actuator is configured to move the piercing member through the second sealing member of the connection port when the connection port is coupled to the housing of the device.

In one or more embodiments, the connection port further comprising a second sealing member located at the proximal end portion of the connection port.

In one or more embodiments, the removable sealing member of the connection port covers the second sealing member of the connection port when the removable sealing member of the connection port is in a first sealed position.

In one or more embodiments, the second sealing member is pierceable.

In one or more embodiments, the second sealing member is a rubber bung.

In one or more embodiments, the second sealing member is frangible.

In one or more embodiments, the second sealing member is a rubber stopper.

In one or more embodiments, the second sealing member is made of a plastic material, a metal alloy, and combinations thereof.

In one or more embodiments, the second sealing member is a plug that substantially seals the fluid passageway between the device and the connection port.

In one or more embodiments, the plug is displaceable.

In one or more embodiments, when the device is coupled to the connection port, and when the removable sealing member of the device and the removable sealing member of the connection port are both in the second unsealed position, a surface of the second sealing member of the connection port is exposed to an interior of the housing of the device.

In one or more embodiments, wherein the surface of the second sealing member of the connection port is characterized in that the surface of the second sealing member is positioned to be wiped off by the wiping member of the device when the wiping member moves across the housing.

In one or more embodiments, wherein when the device is coupled to the connection port, the surface of the second sealing member of the connection port and the wiping member of the device form an airtight seal when the wiping member moves across the housing of the device and wipes off the surface of the second sealing member.

In one or more embodiments, the wiping member of the device wipes off a surface of the second sealing member of the connection port via at least one of press and friction force(s).

In one or more embodiments, the device further comprising a piercing member configured to pierce through the first sealing member of the device and the second sealing member of the connection port.

In one or more embodiments, wherein the piercing member is a needle.

In one or more embodiments, wherein the piercing member is a hollowed needle.

In one or more embodiments, the securing mechanism at the distal end portion of the housing of the device is a thread and the securing mechanism at the proximal end portion of the connection port is a complimentary thread.

In one or more embodiments, the securing mechanism at the distal end portion of the housing of the device are ratchet teeth and the securing mechanism at the proximal end portion of the connection port is a complimentary retention member.

In one or more embodiments, the securing mechanism at the distal end portion of the device is a retention member and the securing mechanism at the proximal end portion of the connection port are complimentary ratchet teeth.

In one or more embodiments, the securing mechanism at the distal end portion of the housing of the device is a clamping member and the securing mechanism at the proximal end portion of the connection port is a complimentary retention member.

In one or more embodiments, the securing mechanism at the distal end portion of the housing of the device is a retention member and the securing mechanism at the proximal end portion of the connection port is a complimentary clamping member.

In one or more embodiments, at least one of the securing mechanism at the distal end portion of the housing and the securing mechanism at the proximal end portion of the connection port is an adhesive.

In one or more embodiments, the proximal end portion of the housing of the device is further configured to couple to a container and the distal end portion of the connection port is further configured to couple to a container.

In one or more embodiments, the proximal end portion of the housing of the device is further configured to couple to a bag and the distal end portion of the connection port is further configured to couple to a vial.

In one or more embodiments, wherein the distal end portion of the connection port is attached to a syringe and the proximal end portion of the housing of the device is attached to a container.

In one or more embodiments, the distal end portion of the connection port is integrally attached to a container and the proximal end portion of the housing of the device is integrally attached to a container.

In one or more embodiments of the system, the distal end portion of the connection port forms a unitary structure with a container and the proximal end portion of the housing of the device forms a unitary structure with a container.

In one or more embodiments of the system, the distal end portion of the connection port is attached to any of a syringe, a filter, an infusion line, and wherein the proximal end portion of the housing of the device is attached to any of a syringe, a filter, an infusion line, and a container.

In one or more embodiments of the system, wherein the removable sealing member of the device is attached to the housing of the device via a frangible seal and the removable sealing member of the connection port is attached to the connection port via a frangible seal.

In one or more embodiments of the system, wherein the removable sealing member of the device and the removable sealing member of the connection port are both configured to be displaced from the fluid passageway via a pulling motion.

In one or more embodiments of the system, wherein the removable sealing member of the device and the removable sealing member of the connection port are both configured to be displaced from the fluid passageway at about the same time via a pulling motion.

In one or more embodiments of the system, wherein at least a portion of the removable sealing member of the device is located through a wall of the housing.

In one or more embodiments, wherein at least a portion of the removable sealing member of the device is located through a side wall of the housing.

In one or more embodiments of the system, wherein at least one of an antibacterial substance, an antiviral substance, and an antifungal substance is located inside the housing of the device, on a surface of the removable sealing member, on the wiping member, and combinations thereof.

In one or more embodiments of the system, wherein the securing mechanism of the distal end portion of the housing of the device is further configured to be movable between a first extended position and a second contracted position.

In one or more embodiments of the system, wherein when the securing mechanism of the distal end portion of the housing of the device is in the second contracted position the second sealing member of the connection port is positioned to be wiped off by the wiping member of the device.

In one or more embodiments of the system, wherein when the device is coupled to the connection port and when the securing mechanism of the device is in a first extended position, an exterior surface of the second sealing member of the connection port is not aligned with an interior surface of the housing, and wherein when the device is coupled to the connection port and the securing mechanism of the device is in the second contracted position, an exterior surface of the second sealing member of the connection port is aligned with an interior surface of the housing.

In one or more embodiments of the system, wherein when the exterior surface of the second sealing member of the connection port is aligned with an interior surface of the housing, the wiping member is able to wipe off the exterior surface of the second sealing member of the connection port when the wiping member moves across the housing.

In one or more embodiments of the system, wherein the distal end portion of the housing of the device is further configured to be movable between a first extended position and a second contracted position.

In one or more embodiments, wherein the securing mechanism of the distal end portion of the housing of the device is selected from a group consisting of a thread, ratchet teeth, a clamping member, a retention member, and an adhesive.

In one or more embodiments of the system, the housing further comprising a guiding member configured to align the removable sealing member of the housing with the removable sealing member of the connection port.

In one or more embodiments, the guiding member is a rod.

In one or more embodiments, the guiding member is a dent.

In one or more embodiments, the guiding member is a slot.

In one or more embodiments, the guiding member is a protrusion.

In one or more embodiments of the system, the guiding member is configured to align the removable sealing member of the housing of the device with the removable sealing member of the connection port, wherein upon alignment of the removable sealing member of the housing of the device with the removable sealing member of the connection port both the removable sealing member of the housing and the removable sealing member of the connection port will displace in about the same direction and at about the same time external to the location of fluidic communication between the housing and the connection port.

In one or more embodiments of the system, the connection port has a guiding member configured to align the removable sealing member of the connection port with the removable sealing member of the device when the connection port couples with the device.

In one or more embodiments of the system, the guiding member of the connection port is a rod.

In one or more embodiments of the system, the guiding member of the connection port is a dent.

In one or more embodiments of the system, the guiding member of the connection port is a slot.

In one or more embodiments of the system, the guiding member of the connection port is a protrusion.

In one or more embodiments of the system, wherein the device further comprising a guiding member configured to align the removable sealing member of the device with the removable sealing member of the connection port.

In one or more embodiments of the system, wherein the connection port further comprising a guiding member configured to align the removable sealing member of the device with the removable sealing member of the connection port.

In one or more embodiments of the system, wherein the device further comprising a guiding member and wherein the connection port further comprising a guiding member, wherein both the guiding member of the device and the guiding member of the connection port are configured to engage with each other in such a manner as to align the removable sealing member of the device and the removable sealing member of the connection port so that both said removable sealing members are able to displace at about the same time and in the same direction when moved to a second unsealed position.

In one or more embodiments of the system, wherein the guiding member of the device is a rod and the guiding member of the connection port is a complimentary slot which allows the rod to enter into the slot.

In one or more embodiments of the system, wherein the guiding member of the connection port is a rod and the guiding member of the device is a complimentary slot which allows the rod to enter into the slot.

In one or more embodiments of the system, wherein the guiding member of the device is a protrusion and the complimentary guiding member of the connection port is a dent, wherein the dent allows the protrusion to engage and/or enter into the dent.

In one or more embodiments of the system, wherein the guiding member of the connection port is a protrusion and the complimentary guiding member of the device is a dent, wherein the dent allows the protrusion to engage and/or enter into the dent.

In one or more embodiments, the proximal end portion of the housing is integrally attached to a container and the distal end portion of the housing having a guiding member.

In one or more embodiments, the wiping member is configured to extend from the top wall of the housing to the bottom wall of the housing.

In one or more embodiments, the wiping member is configured to provide a hermetic seal between the top wall and the wiping member and the bottom wall and the wiping member when the wiping member moves across the housing.

In one or more embodiments, the wiping member is configured to wipe off a surface of the first sealing member of the housing when the wiping member moves across the housing.

In one or more embodiments, a filter is disposed in, on, or over an aperture in the housing for releasing air from the housing when actuating the wiping member.

In one or more embodiments, the housing is manufactured from a group consisting of plastic, rigid plastic, flexible plastic, polymer, and combinations thereof.

In one or more embodiments, the housing is made of a glass material, a metal material, and combinations thereof.

In one or more embodiments, the wiping member is made of a plastic substance, a rubber substance, an elastomeric substance, and combinations thereof.

In one or more embodiments, the wiping member is covered and/or saturated with an antibacterial substance, an antiviral substance, an anti-tuberculin substance, an anti-HIV substance, hydrogen peroxide, isopropyl alcohol, and combinations thereof.

In one or more embodiments, the device and/or housing is disposable and/or for single use.

In one or more embodiments, a surface of the removable sealing member has an adhesive on the surface of the removable sealing member.

In one or more embodiments, an exterior surface of the removable sealing member has an adhesive.

In one or more embodiments, the device further comprises a sealing ring.

In one or more embodiments, the sealing ring of the device is an “O-ring”.

In one or more embodiments, the sealing ring is a circumferential sealing ring.

In one or more embodiments, the “O-ring” is a circumferential “O-ring”.

In one or more embodiments, the sealing ring of the device is made of at least one of a plastic material and a rubber material.

In one or more embodiments, the sealing “O-ring” of the device is made of at least one of a plastic material and a rubber material.

In one or more embodiments, the connection port further comprises a sealing ring.

In one or more embodiments, the sealing ring of the connection port is an “O-ring”.

In one or more embodiments, the sealing ring of the connection port is a circumferential sealing ring.

In one or more embodiments, the “O-ring” of the connection port is a circumferential “O-ring”.

In one or more embodiments, the sealing ring of the connection port is made of at least one of a plastic material and a rubber material.

In one or more embodiments, the sealing “O-ring” of the connection port is made of at least one of a plastic material and a rubber material.

In one or more embodiments, both the device and the connection port further comprise a sealing ring.

In one or more embodiments, the sealing ring of the device and the sealing ring of the connection port is an “O-ring”.

In one or more embodiments, the sealing ring of the device is a circumferential sealing ring and the sealing ring of the connection port is a circumferential sealing ring.

In one or more embodiments, the “O-ring” of the device is a circumferential “O-ring” and the “O-ring of the connection port is a circumferential “O-ring”.

In one or more embodiments, the sealing ring of the connection port is made of at least one of a plastic material and a rubber material and the sealing ring of the device is made of at least one of a plastic material and a rubber material.

In one or more embodiments, the sealing “O-ring” of the device and the sealing “O-ring” of the connection port are made of at least one of a plastic material and a rubber material.

In one or more embodiments, the device further comprising teeth configured to engage/couple holes/dents of a connection port.

In one or more embodiments, the teeth of the device are retractable teeth.

In one or more embodiments, the teeth are positioned at a distal end portion of the device.

In one or more embodiments, the retractable teeth are positioned at a distal end portion of the device.

In one or more embodiments, the teeth are positioned on an engagement mechanism of the device.

In one or more embodiments, the teeth are positioned on a securing mechanism of the device.

In one or more embodiments, the teeth are positioned on the device.

In one or more embodiments, the teeth are positioned on the housing of the device.

In one or more embodiments, the retractable teeth are positioned on the device.

In one or more embodiments, the retractable teeth are positioned on the housing of the device.

In one or more embodiments, the teeth are configured to engage/couple a hole/dent of a connection port.

In one or more embodiments, the retractable teeth are configured to engage/couple a hole/dent of a connection port.

In one or more embodiments, the teeth are configured to provide coupling of a connection port to the device, the teeth are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the device further comprising holes/dents configured to engage/couple with teeth of a connection port.

In one or more embodiments, the device further comprising holes/dents configured to engage/couple with retractable teeth of a connection port.

In one or more embodiments, the holes/debts are positioned at a distal end portion of the device.

In one or more embodiments, the holes/dents are positioned on an engagement mechanism of the device.

In one or more embodiments, the holes/dents are positioned on a securing mechanism of the device.

In one or more embodiments, the holes/dents are positioned on the device.

In one or more embodiments, the holes/dents are positioned on the housing of the device.

In one or more embodiments, the holes/dents are configured to engage/couple teeth of a connection port.

In one or more embodiments, the holes/dents are configured to engage/couple retractable teeth of a connection port.

In one or more embodiments, the holes/dents are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments of the system, the device further comprising teeth and the connection port further comprising holes/dents.

In one or more embodiments of the system, the device further comprising retractable teeth and the connection port further comprising holes/dents configured to engage/couple the retractable teeth.

In one or more embodiments of the system, the device further comprising holes/dents and the connection port further comprising teeth.

In one or more embodiments of the system, the device further comprising holes/dents and the connection port further comprising retractable teeth.

In one or more embodiments, the holes/dents of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the holes/dents of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the teeth of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the retractable teeth of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the teeth of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the retractable teeth of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In still a further aspect, the present invention provides a system for establishing a decontaminated fluid passageway between a device and a connection port, comprising: i) a device comprising: a housing defining a fluid passageway, a distal end portion of the housing configured to couple a connection port, the distal end portion of the housing having a securing mechanism configured to engage the connection port; a removable sealing member for sealing the distal end portion of the housing, the removable sealing member positioned to seal the distal end portion of the fluid passageway, the removable sealing member configured to be movable between a first sealed position and a second unsealed position, the removable sealing member being moveable by manual manipulation of the removable sealing member, wherein the removable sealing member is displaced from the fluid passageway when the removable sealing member in the second unsealed position, the device further comprising an engagement mechanism configured to engage a guiding member of a connection port; ii) a connection port, comprising: a proximal end portion defining a fluid passageway, the proximal end portion of the connection port configured to couple the distal end portion of the housing of the device, the proximal end portion of the connection port having a securing mechanism configured to engage the securing mechanism of the distal end portion of the housing of the device; a removable sealing member for sealing the proximal end portion of the fluid passageway of the connection port, the removable sealing member of the connection port configured to be movable between a first sealed position and a second unsealed position, the removable sealing member of the connection port being movable by manual manipulation of the removable sealing member, wherein the removable sealing member is displaced from the fluid passageway of the connection port when the removable sealing member is in the second unsealed position; and a second sealing member positioned to seal an opening of the connection port, the second sealing member positioned inside and/or over the opening of the connection port, the second sealing member covered by the removable sealing member of the connection port when the removable sealing member of the connection port is in a first sealed position and the second sealing member being uncovered when the removable sealing member of the connection port is displaced to a second unsealed position, the connection port further comprising a guiding member configured to engage the device when the connection port is attached/coupled to the device, the guiding member characterized in that is allows the connection port to couple to the device in a manner that aligns the removable sealing member of the device with the removable sealing member of a connection port so that both removable sealing members of the device and the connection port lie flat against one another.

In one or more embodiments, the guiding member is selected from a rod, a hole, and a dent.

In one or more embodiments, the guiding member having a thread.

In one or more embodiments, the guiding member protrudes out of the connection port to an exterior of the connection port.

In one or more embodiments, the second sealing member is at least one of pierceable and frangible.

In one or more embodiments, the second sealing member is a stopper.

In one or more embodiments, the second sealing member is a rubber stopper.

In one or more embodiments, the second sealing member is a bung.

In one or more embodiments, the second sealing member is a rubber bung.

In one or more embodiments, the second sealing member prevents escape/leakage of fluid out of the connection port in the event of spontaneous disengagement between the connection port and the device.

In one or more embodiments, the second sealing member is configured to be pierced by a piercing member of the device.

In one or more embodiments, the second sealing member is configured to be pierced by a piercing member of the connection port.

In one or more embodiments, at least one of the device and the connection port further comprising a piercing member configured to pierce through the second sealing member of the connection port.

In one or more embodiments, the device further comprising a plug positioned in an opening at the distal end portion of the device, the plug configured to prevent escape/leakage of fluid out of the device in the event of spontaneous disengagement between the connection port and the device.

In one or more embodiments, the plug of the device is at least one of pierceable and frangible.

In one or more embodiments, the plug of the device is configured to be pierced by a piercing member of the device or by a piercing member of the connection port.

In one or more embodiments, both the plug of the device and the second sealing member of the connection port serve as a means to prevent spillage of the contents held/transferred between the device and the connection port in the event of spontaneous/accidental disengagement of the device from the connection port.

In one or more embodiments, the removable sealing member of the device and the removable sealing member of the connection port are both configured to substantially seal the fluid passageway between the device and the connection port when both the removable sealing member of the device and the removable sealing member of the connection port are in a first sealed position, and wherein the removable sealing member of the device and the removable sealing member of the connection port are configured to allow passage of fluid between the device and the connection port when the removable sealing member of the device and the removable sealing member of the connection port are both in the second unsealed position.

In one or more embodiments, the removable sealing member of the device and the removable sealing member of the connection port are both configured to be moved from the first sealed position to the second unsealed position via a displacement of the removable sealing members of both the device and the connection port, wherein the displacement of both removable sealing members of the device and the connection port are external to the fluid passageway between the device and the connection port.

In one or more embodiments, the removable sealing member of the device is in a folded configuration and the removable sealing member of the connection port is in a folded configuration.

In one or more embodiments, the removable sealing member of the device is in a folded configuration or the removable sealing member of the connection port is in a folded configuration.

In one or more embodiments, at least one of the removable sealing members of the device and the removable sealing member of the connection port are in a folded configuration.

In one or more embodiments, a portion of the removable sealing member of the device is attached to an interior surface of the housing.

In one or more embodiments, a portion of the removable sealing member of the device is attached to an interior surface of the housing and a portion of the removable sealing member of the device is attached to an exterior surface of the housing.

In one or more embodiments, the first sealing member of the device is characterized as being at least one of pierceable and frangible.

In one or more embodiments, the device further comprising a piercing member.

In one or more embodiments, the housing further comprising a piercing member.

In one or more embodiments, the device further comprising a piercing member actuator.

In one or more embodiments, the piercing member actuator configured to move the piercing member through the first sealing member of the device.

In one or more embodiments, the piercing member is a needle.

In one or more embodiments, the piercing member is a hollowed needle.

In one or more embodiments, the piercing member is configured to pierce through the second sealing member of the connection port when the connection port is engaged to the housing of the device.

In one or more embodiments, the piercing member actuator is configured to move the piercing member through the second sealing member of the connection port when the connection port is coupled to the housing of the device.

In one or more embodiments, the connection port further comprising a second sealing member located at the proximal end portion of the connection port.

In one or more embodiments, the removable sealing member of the connection port covers the second sealing member of the connection port when the removable sealing member of the connection port is in a first sealed position.

In one or more embodiments, the second sealing member is pierceable.

In one or more embodiments, the second sealing member is a rubber bung.

In one or more embodiments, the second sealing member is frangible.

In one or more embodiments, the second sealing member is a rubber stopper.

In one or more embodiments, the second sealing member is made of a plastic material, a metal alloy, and combinations thereof.

In one or more embodiments, the second sealing member is a plug that substantially seals the fluid passageway between the device and the connection port.

In one or more embodiments, the plug is displaceable.

In one or more embodiments, when the device is coupled to the connection port, and when the removable sealing member of the device and the removable sealing member of the connection port are both in the second unsealed position, a surface of the second sealing member of the connection port is exposed to an interior of the housing of the device.

In one or more embodiments, wherein the surface of the second sealing member of the connection port is characterized in that the surface of the second sealing member is positioned to be wiped off by the wiping member of the device when the wiping member moves across the housing.

In one or more embodiments, wherein when the device is coupled to the connection port, the surface of the second sealing member of the connection port and the wiping member of the device form an airtight seal when the wiping member moves across the housing of the device and wipes off the surface of the second sealing member.

In one or more embodiments, the wiping member of the device wipes off a surface of the second sealing member of the connection port via at least one of press and friction force(s).

In one or more embodiments, the device further comprising a piercing member configured to pierce through the first sealing member of the device and the second sealing member of the connection port.

In one or more embodiments, wherein the piercing member is a needle.

In one or more embodiments, wherein the piercing member is a hollowed needle.

In one or more embodiments, the securing mechanism at the distal end portion of the housing of the device is a thread and the securing mechanism at the proximal end portion of the connection port is a complimentary thread.

In one or more embodiments, the securing mechanism at the distal end portion of the housing of the device are ratchet teeth and the securing mechanism at the proximal end portion of the connection port is a complimentary retention member.

In one or more embodiments, the securing mechanism at the distal end portion of the device is a retention member and the securing mechanism at the proximal end portion of the connection port are complimentary ratchet teeth.

In one or more embodiments, the securing mechanism at the distal end portion of the housing of the device is a clamping member and the securing mechanism at the proximal end portion of the connection port is a complimentary retention member.

In one or more embodiments, the securing mechanism at the distal end portion of the housing of the device is a retention member and the securing mechanism at the proximal end portion of the connection port is a complimentary clamping member.

In one or more embodiments, at least one of the securing mechanism at the distal end portion of the housing and the securing mechanism at the proximal end portion of the connection port is an adhesive.

In one or more embodiments, the proximal end portion of the housing of the device is further configured to couple to a container and the distal end portion of the connection port is further configured to couple to a container.

In one or more embodiments, the proximal end portion of the housing of the device is further configured to couple to a bag and the distal end portion of the connection port is further configured to couple to a vial.

In one or more embodiments, wherein the distal end portion of the connection port is attached to a syringe and the proximal end portion of the housing of the device is attached to a container.

In one or more embodiments, the distal end portion of the connection port is integrally attached to a container and the proximal end portion of the housing of the device is integrally attached to a container.

In one or more embodiments of the system, the distal end portion of the connection port forms a unitary structure with a container and the proximal end portion of the housing of the device forms a unitary structure with a container.

In one or more embodiments of the system, the distal end portion of the connection port is attached to any of a syringe, a filter, an infusion line, and wherein the proximal end portion of the housing of the device is attached to any of a syringe, a filter, an infusion line, and a container.

In one or more embodiments of the system, wherein the removable sealing member of the device is attached to the housing of the device via a frangible seal and the removable sealing member of the connection port is attached to the connection port via a frangible seal.

In one or more embodiments of the system, wherein the removable sealing member of the device and the removable sealing member of the connection port are both configured to be displaced from the fluid passageway via a pulling motion.

In one or more embodiments of the system, wherein the removable sealing member of the device and the removable sealing member of the connection port are both configured to be displaced from the fluid passageway at about the same time via a pulling motion.

In one or more embodiments of the system, wherein at least a portion of the removable sealing member of the device is located through a wall of the housing.

In one or more embodiments, wherein at least a portion of the removable sealing member of the device is located through a side wall of the housing.

In one or more embodiments of the system, wherein at least one of an antibacterial substance, an antiviral substance, and an antifungal substance is located inside the housing of the device, on a surface of the removable sealing member, on the wiping member, and combinations thereof.

In one or more embodiments of the system, wherein the securing mechanism of the distal end portion of the housing of the device is further configured to be movable between a first extended position and a second contracted position.

In one or more embodiments of the system, wherein when the securing mechanism of the distal end portion of the housing of the device is in the second contracted position the second sealing member of the connection port is positioned to be wiped off by the wiping member of the device.

In one or more embodiments of the system, wherein when the device is coupled to the connection port and when the securing mechanism of the device is in a first extended position, an exterior surface of the second sealing member of the connection port is not aligned with an interior surface of the housing, and wherein when the device is coupled to the connection port and the securing mechanism of the device is in the second contracted position, an exterior surface of the second sealing member of the connection port is aligned with an interior surface of the housing.

In one or more embodiments of the system, wherein when the exterior surface of the second sealing member of the connection port is aligned with an interior surface of the housing, the wiping member is able to wipe off the exterior surface of the second sealing member of the connection port when the wiping member moves across the housing.

In one or more embodiments of the system, wherein the distal end portion of the housing of the device is further configured to be movable between a first extended position and a second contracted position.

In one or more embodiments, wherein the securing mechanism of the distal end portion of the housing of the device is selected from a group consisting of a thread, ratchet teeth, a clamping member, a retention member, and an adhesive.

In one or more embodiments of the system, the housing further comprising a guiding member configured to align the removable sealing member of the housing with the removable sealing member of the connection port.

In one or more embodiments, the guiding member is a rod.

In one or more embodiments, the guiding member is a dent.

In one or more embodiments, the guiding member is a slot.

In one or more embodiments, the guiding member is a protrusion.

In one or more embodiments of the system, the guiding member is configured to align the removable sealing member of the housing of the device with the removable sealing member of the connection port, wherein upon alignment of the removable sealing member of the housing of the device with the removable sealing member of the connection port both the removable sealing member of the housing and the removable sealing member of the connection port will displace in about the same direction and at about the same time external to the location of fluidic communication between the housing and the connection port.

In one or more embodiments of the system, the connection port has a guiding member configured to align the removable sealing member of the connection port with the removable sealing member of the device when the connection port couples with the device.

In one or more embodiments of the system, the guiding member of the connection port is a rod.

In one or more embodiments of the system, the guiding member of the connection port is a dent.

In one or more embodiments of the system, the guiding member of the connection port is a slot.

In one or more embodiments of the system, the guiding member of the connection port is a protrusion.

In one or more embodiments of the system, wherein the device further comprising a guiding member configured to align the removable sealing member of the device with the removable sealing member of the connection port.

In one or more embodiments of the system, wherein the connection port further comprising a guiding member configured to align the removable sealing member of the device with the removable sealing member of the connection port.

In one or more embodiments of the system, wherein the device further comprising a guiding member and wherein the connection port further comprising a guiding member, wherein both the guiding member of the device and the guiding member of the connection port are configured to engage with each other in such a manner as to align the removable sealing member of the device and the removable sealing member of the connection port so that both said removable sealing members are able to displace at about the same time and in the same direction when moved to a second unsealed position.

In one or more embodiments of the system, wherein the guiding member of the device is a rod and the guiding member of the connection port is a complimentary slot which allows the rod to enter into the slot.

In one or more embodiments of the system, wherein the guiding member of the connection port is a rod and the guiding member of the device is a complimentary slot which allows the rod to enter into the slot.

In one or more embodiments of the system, wherein the guiding member of the device is a protrusion and the complimentary guiding member of the connection port is a dent, wherein the dent allows the protrusion to engage and/or enter into the dent.

In one or more embodiments of the system, wherein the guiding member of the connection port is a protrusion and the complimentary guiding member of the device is a dent, wherein the dent allows the protrusion to engage and/or enter into the dent.

In one or more embodiments, the proximal end portion of the housing is integrally attached to a container and the distal end portion of the housing having a guiding member.

In one or more embodiments, the wiping member is configured to extend from the top wall of the housing to the bottom wall of the housing.

In one or more embodiments, the wiping member is configured to provide a hermetic seal between the top wall and the wiping member and the bottom wall and the wiping member when the wiping member moves across the housing.

In one or more embodiments, the wiping member is configured to wipe off a surface of the first sealing member of the housing when the wiping member moves across the housing.

In one or more embodiments, a filter is disposed in, on, or over an aperture in the housing for releasing air from the housing when actuating the wiping member.

In one or more embodiments, the housing is manufactured from a group consisting of plastic, rigid plastic, flexible plastic, polymer, and combinations thereof.

In one or more embodiments, the housing is made of a glass material, a metal material, and combinations thereof.

In one or more embodiments, the wiping member is made of a plastic substance, a rubber substance, an elastomeric substance, and combinations thereof.

In one or more embodiments, the wiping member is covered and/or saturated with an antibacterial substance, an antiviral substance, an anti-tuberculin substance, an anti-HIV substance, hydrogen peroxide, isopropyl alcohol, and combinations thereof.

In one or more embodiments, the device and/or housing is disposable and/or for single use.

In one or more embodiments, a surface of the removable sealing member has an adhesive on the surface of the removable sealing member.

In one or more embodiments, an exterior surface of the removable sealing member has an adhesive.

In one or more embodiments, the device further comprises a sealing ring.

In one or more embodiments, the sealing ring of the device is an “O-ring”.

In one or more embodiments, the sealing ring is a circumferential sealing ring.

In one or more embodiments, the “O-ring” is a circumferential “O-ring”.

In one or more embodiments, the sealing ring of the device is made of at least one of a plastic material and a rubber material.

In one or more embodiments, the sealing “O-ring” of the device is made of at least one of a plastic material and a rubber material.

In one or more embodiments, the connection port further comprises a sealing ring.

In one or more embodiments, the sealing ring of the connection port is an “O-ring”.

In one or more embodiments, the sealing ring of the connection port is a circumferential sealing ring.

In one or more embodiments, the “O-ring” of the connection port is a circumferential “O-ring”.

In one or more embodiments, the sealing ring of the connection port is made of at least one of a plastic material and a rubber material.

In one or more embodiments, the sealing “O-ring” of the connection port is made of at least one of a plastic material and a rubber material.

In one or more embodiments, both the device and the connection port further comprise a sealing ring.

In one or more embodiments, the sealing ring of the device and the sealing ring of the connection port is an “O-ring”.

In one or more embodiments, the sealing ring of the device is a circumferential sealing ring and the sealing ring of the connection port is a circumferential sealing ring.

In one or more embodiments, the “O-ring” of the device is a circumferential “O-ring” and the “O-ring of the connection port is a circumferential “O-ring”.

In one or more embodiments, the sealing ring of the connection port is made of at least one of a plastic material and a rubber material and the sealing ring of the device is made of at least one of a plastic material and a rubber material.

In one or more embodiments, the sealing “O-ring” of the device and the sealing “O-ring” of the connection port are made of at least one of a plastic material and a rubber material.

In one or more embodiments, the device further comprising teeth configured to engage/couple holes/dents of a connection port.

In one or more embodiments, the teeth of the device are retractable teeth.

In one or more embodiments, the teeth are positioned at a distal end portion of the device.

In one or more embodiments, the retractable teeth are positioned at a distal end portion of the device.

In one or more embodiments, the teeth are positioned on an engagement mechanism of the device.

In one or more embodiments, the teeth are positioned on a securing mechanism of the device.

In one or more embodiments, the teeth are positioned on the device.

In one or more embodiments, the teeth are positioned on the housing of the device.

In one or more embodiments, the retractable teeth are positioned on the device.

In one or more embodiments, the retractable teeth are positioned on the housing of the device.

In one or more embodiments, the teeth are configured to engage/couple a hole/dent of a connection port.

In one or more embodiments, the retractable teeth are configured to engage/couple a hole/dent of a connection port.

In one or more embodiments, the teeth are configured to provide coupling of a connection port to the device, the teeth are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the device further comprising holes/dents configured to engage/couple with teeth of a connection port.

In one or more embodiments, the device further comprising holes/dents configured to engage/couple with retractable teeth of a connection port.

In one or more embodiments, the holes/debts are positioned at a distal end portion of the device.

In one or more embodiments, the holes/dents are positioned on an engagement mechanism of the device.

In one or more embodiments, the holes/dents are positioned on a securing mechanism of the device.

In one or more embodiments, the holes/dents are positioned on the device.

In one or more embodiments, the holes/dents are positioned on the housing of the device.

In one or more embodiments, the holes/dents are configured to engage/couple teeth of a connection port.

In one or more embodiments, the holes/dents are configured to engage/couple retractable teeth of a connection port.

In one or more embodiments, the holes/dents are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments of the system, the device further comprising teeth and the connection port further comprising holes/dents.

In one or more embodiments of the system, the device further comprising retractable teeth and the connection port further comprising holes/dents configured to engage/couple the retractable teeth.

In one or more embodiments of the system, the device further comprising holes/dents and the connection port further comprising teeth.

In one or more embodiments of the system, the device further comprising holes/dents and the connection port further comprising retractable teeth.

In one or more embodiments, the holes/dents of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the holes/dents of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the teeth of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the retractable teeth of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the teeth of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the retractable teeth of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In still a further aspect, the present invention provides a system for establishing a decontaminated fluid passageway between a device and a connection port, comprising: i) a device comprising: a housing defining a fluid passageway, a proximal end portion of the housing attached to a container and a distal end portion of the housing configured to couple a connection port, the distal end portion of the housing having a securing mechanism configured to engage the connection port; a removable sealing member for sealing the distal end portion of the housing, the removable sealing member positioned to seal the distal end portion of the fluid passageway, the removable sealing member configured to be movable between a first sealed position and a second unsealed position, the removable sealing member being moveable by manual manipulation of the removable sealing member, wherein the removable sealing member is displaced from the fluid passageway when the removable sealing member in the second unsealed position, the device further comprising an engagement mechanism configured to engage a guiding member of a connection port; ii) a connection port, comprising: a distal end portion of the connection port attached to a container and a proximal end portion defining a fluid passageway, the proximal end portion of the connection port configured to couple the distal end portion of the housing of the device, the proximal end portion of the connection port having a securing mechanism configured to engage the securing mechanism of the distal end portion of the housing of the device; a removable sealing member for sealing the proximal end portion of the fluid passageway of the connection port, the removable sealing member of the connection port configured to be movable between a first sealed position and a second unsealed position, the removable sealing member of the connection port being movable by manual manipulation of the removable sealing member, wherein the removable sealing member is displaced from the fluid passageway of the connection port when the removable sealing member is in the second unsealed position; and a second sealing member positioned to seal an opening of the connection port, the second sealing member positioned inside and/or over the opening of the connection port, the second sealing member covered by the removable sealing member of the connection port when the removable sealing member of the connection port is in a first sealed position and the second sealing member being uncovered when the removable sealing member of the connection port is displaced to a second unsealed position, the connection port further comprising a guiding member configured to engage the device when the connection port is attached/coupled to the device, the guiding member characterized in that is allows the connection port to couple to the device in a manner that aligns the removable sealing member of the device with the removable sealing member of a connection port so that both removable sealing members of the device and the connection port lie flat against one another.

In one or more embodiments, the container attached to the proximal end portion of the device is a bag or a bottle.

In one or more embodiments, the container attached to the proximal end portion of the device is a vial.

In one or more embodiments, the container attached to the distal end portion of the connection port is a bag or a bottle.

In one or more embodiments, the container attached to the distal end portion of the connection port is a vial.

In one or more embodiments, the container attached to the proximal end portion of the housing of the device is a bag and the container attached to the distal end portion of the connection port is a bottle.

In one or more embodiments, the container attached to the proximal end portion of the housing of the device is a bag and the container attached to the distal end portion of the connection port is a vial.

In one or more embodiments, the container attached to the proximal end portion of the housing of the device is a bottle and the container attached to the distal end portion of the connection port is a bag.

In one or more embodiments, the container attached to the proximal end portion of the housing of the device is a vial and the container attached to the distal end portion of the connection port is a bag.

In one or more embodiments, the container attached to the proximal end portion of the housing of the device is a vial and the container attached to the distal end portion of the connection port is a vial.

In one or more embodiments, the container attached to the proximal end portion of the housing of the device is a bottle and the container attached to the distal end portion of the connection port is a bottle.

In one or more embodiments, the container attached to the proximal end portion of the housing is integrally attached to the housing of the device.

In one or more embodiments, the container attached to the proximal end portion of the housing of the device forms a unitary structure with the device.

In one or more embodiments, the container attached to the distal end portion of the connection port is integrally attached to the connection port.

In one or more embodiments, the container attached to the distal end portion of the connection port forms a unitary structure with the connection port.

In one or more embodiments, the guiding member is selected from a rod, a hole, and a dent.

In one or more embodiments, the guiding member having a thread.

In one or more embodiments, the guiding member protrudes out of the connection port to an exterior of the connection port.

In one or more embodiments, the second sealing member is at least one of pierceable and frangible.

In one or more embodiments, the second sealing member is a stopper.

In one or more embodiments, the second sealing member is a rubber stopper.

In one or more embodiments, the second sealing member is a bung.

In one or more embodiments, the second sealing member is a rubber bung.

In one or more embodiments, the second sealing member prevents escape/leakage of fluid out of the connection port in the event of spontaneous disengagement between the connection port and the device.

In one or more embodiments, the second sealing member is configured to be pierced by a piercing member of the device.

In one or more embodiments, the second sealing member is configured to be pierced by a piercing member of the connection port.

In one or more embodiments, at least one of the device and the connection port further comprising a piercing member configured to pierce through the second sealing member of the connection port.

In one or more embodiments, the device further comprising a plug positioned in an opening at the distal end portion of the device, the plug configured to prevent escape/leakage of fluid out of the device in the event of spontaneous disengagement between the connection port and the device.

In one or more embodiments, the plug of the device is at least one of pierceable and frangible.

In one or more embodiments, the plug of the device is configured to be pierced by a piercing member of the device or by a piercing member of the connection port.

In one or more embodiments, both the plug of the device and the second sealing member of the connection port serve as a means to prevent spillage of the contents held/transferred between the device and the connection port in the event of spontaneous/accidental disengagement of the device from the connection port.

In one or more embodiments, the removable sealing member of the device and the removable sealing member of the connection port are both configured to substantially seal the fluid passageway between the device and the connection port when both the removable sealing member of the device and the removable sealing member of the connection port are in a first sealed position, and wherein the removable sealing member of the device and the removable sealing member of the connection port are configured to allow passage of fluid between the device and the connection port when the removable sealing member of the device and the removable sealing member of the connection port are both in the second unsealed position.

In one or more embodiments, the removable sealing member of the device and the removable sealing member of the connection port are both configured to be moved from the first sealed position to the second unsealed position via a displacement of the removable sealing members of both the device and the connection port, wherein the displacement of both removable sealing members of the device and the connection port are external to the fluid passageway between the device and the connection port.

In one or more embodiments, the removable sealing member of the device is in a folded configuration and the removable sealing member of the connection port is in a folded configuration.

In one or more embodiments, the removable sealing member of the device is in a folded configuration or the removable sealing member of the connection port is in a folded configuration.

In one or more embodiments, at least one of the removable sealing members of the device and the removable sealing member of the connection port are in a folded configuration.

In one or more embodiments, a portion of the removable sealing member of the device is attached to an interior surface of the housing.

In one or more embodiments, a portion of the removable sealing member of the device is attached to an interior surface of the housing and a portion of the removable sealing member of the device is attached to an exterior surface of the housing.

In one or more embodiments, the first sealing member of the device is characterized as being at least one of pierceable and frangible.

In one or more embodiments, the device further comprising a piercing member.

In one or more embodiments, the housing further comprising a piercing member.

In one or more embodiments, the device further comprising a piercing member actuator.

In one or more embodiments, the piercing member actuator configured to move the piercing member through the first sealing member of the device.

In one or more embodiments, the piercing member is a needle.

In one or more embodiments, the piercing member is a hollowed needle.

In one or more embodiments, the piercing member is configured to pierce through the second sealing member of the connection port when the connection port is engaged to the housing of the device.

In one or more embodiments, the piercing member actuator is configured to move the piercing member through the second sealing member of the connection port when the connection port is coupled to the housing of the device.

In one or more embodiments, the connection port further comprising a second sealing member located at the proximal end portion of the connection port.

In one or more embodiments, the removable sealing member of the connection port covers the second sealing member of the connection port when the removable sealing member of the connection port is in a first sealed position.

In one or more embodiments, the second sealing member is pierceable.

In one or more embodiments, the second sealing member is a rubber bung.

In one or more embodiments, the second sealing member is frangible.

In one or more embodiments, the second sealing member is a rubber stopper.

In one or more embodiments, the second sealing member is made of a plastic material, a metal alloy, and combinations thereof.

In one or more embodiments, the second sealing member is a plug that substantially seals the fluid passageway between the device and the connection port.

In one or more embodiments, the plug is displaceable.

In one or more embodiments, when the device is coupled to the connection port, and when the removable sealing member of the device and the removable sealing member of the connection port are both in the second unsealed position, a surface of the second sealing member of the connection port is exposed to an interior of the housing of the device.

In one or more embodiments, wherein the surface of the second sealing member of the connection port is characterized in that the surface of the second sealing member is positioned to be wiped off by the wiping member of the device when the wiping member moves across the housing.

In one or more embodiments, wherein when the device is coupled to the connection port, the surface of the second sealing member of the connection port and the wiping member of the device form an airtight seal when the wiping member moves across the housing of the device and wipes off the surface of the second sealing member.

In one or more embodiments, the wiping member of the device wipes off a surface of the second sealing member of the connection port via at least one of press and friction force(s).

In one or more embodiments, the device further comprising a piercing member configured to pierce through the first sealing member of the device and the second sealing member of the connection port.

In one or more embodiments, wherein the piercing member is a needle.

In one or more embodiments, wherein the piercing member is a hollowed needle.

In one or more embodiments, the securing mechanism at the distal end portion of the housing of the device is a thread and the securing mechanism at the proximal end portion of the connection port is a complimentary thread.

In one or more embodiments, the securing mechanism at the distal end portion of the housing of the device are ratchet teeth and the securing mechanism at the proximal end portion of the connection port is a complimentary retention member.

In one or more embodiments, the securing mechanism at the distal end portion of the device is a retention member and the securing mechanism at the proximal end portion of the connection port are complimentary ratchet teeth.

In one or more embodiments, the securing mechanism at the distal end portion of the housing of the device is a clamping member and the securing mechanism at the proximal end portion of the connection port is a complimentary retention member.

In one or more embodiments, the securing mechanism at the distal end portion of the housing of the device is a retention member and the securing mechanism at the proximal end portion of the connection port is a complimentary clamping member.

In one or more embodiments, at least one of the securing mechanism at the distal end portion of the housing and the securing mechanism at the proximal end portion of the connection port is an adhesive.

In one or more embodiments, the proximal end portion of the housing of the device is further configured to couple to a container and the distal end portion of the connection port is further configured to couple to a container.

In one or more embodiments, the proximal end portion of the housing of the device is further configured to couple to a bag and the distal end portion of the connection port is further configured to couple to a vial.

In one or more embodiments, wherein the distal end portion of the connection port is attached to a syringe and the proximal end portion of the housing of the device is attached to a container.

In one or more embodiments, the distal end portion of the connection port is integrally attached to a container and the proximal end portion of the housing of the device is integrally attached to a container.

In one or more embodiments of the system, the distal end portion of the connection port forms a unitary structure with a container and the proximal end portion of the housing of the device forms a unitary structure with a container.

In one or more embodiments of the system, the distal end portion of the connection port is attached to any of a syringe, a filter, an infusion line, and wherein the proximal end portion of the housing of the device is attached to any of a syringe, a filter, an infusion line, and a container.

In one or more embodiments of the system, wherein the removable sealing member of the device is attached to the housing of the device via a frangible seal and the removable sealing member of the connection port is attached to the connection port via a frangible seal.

In one or more embodiments of the system, wherein the removable sealing member of the device and the removable sealing member of the connection port are both configured to be displaced from the fluid passageway via a pulling motion.

In one or more embodiments of the system, wherein the removable sealing member of the device and the removable sealing member of the connection port are both configured to be displaced from the fluid passageway at about the same time via a pulling motion.

In one or more embodiments of the system, wherein at least a portion of the removable sealing member of the device is located through a wall of the housing.

In one or more embodiments, wherein at least a portion of the removable sealing member of the device is located through a side wall of the housing.

In one or more embodiments of the system, wherein at least one of an antibacterial substance, an antiviral substance, and an antifungal substance is located inside the housing of the device, on a surface of the removable sealing member, on the wiping member, and combinations thereof.

In one or more embodiments of the system, wherein the securing mechanism of the distal end portion of the housing of the device is further configured to be movable between a first extended position and a second contracted position.

In one or more embodiments of the system, wherein when the securing mechanism of the distal end portion of the housing of the device is in the second contracted position the second sealing member of the connection port is positioned to be wiped off by the wiping member of the device.

In one or more embodiments of the system, wherein when the device is coupled to the connection port and when the securing mechanism of the device is in a first extended position, an exterior surface of the second sealing member of the connection port is not aligned with an interior surface of the housing, and wherein when the device is coupled to the connection port and the securing mechanism of the device is in the second contracted position, an exterior surface of the second sealing member of the connection port is aligned with an interior surface of the housing.

In one or more embodiments of the system, wherein when the exterior surface of the second sealing member of the connection port is aligned with an interior surface of the housing, the wiping member is able to wipe off the exterior surface of the second sealing member of the connection port when the wiping member moves across the housing.

In one or more embodiments of the system, wherein the distal end portion of the housing of the device is further configured to be movable between a first extended position and a second contracted position.

In one or more embodiments, wherein the securing mechanism of the distal end portion of the housing of the device is selected from a group consisting of a thread, ratchet teeth, a clamping member, a retention member, and an adhesive.

In one or more embodiments of the system, the housing further comprising a guiding member configured to align the removable sealing member of the housing with the removable sealing member of the connection port.

In one or more embodiments, the guiding member is a rod.

In one or more embodiments, the guiding member is a dent.

In one or more embodiments, the guiding member is a slot.

In one or more embodiments, the guiding member is a protrusion.

In one or more embodiments of the system, the guiding member is configured to align the removable sealing member of the housing of the device with the removable sealing member of the connection port, wherein upon alignment of the removable sealing member of the housing of the device with the removable sealing member of the connection port both the removable sealing member of the housing and the removable sealing member of the connection port will displace in about the same direction and at about the same time external to the location of fluidic communication between the housing and the connection port.

In one or more embodiments of the system, the connection port has a guiding member configured to align the removable sealing member of the connection port with the removable sealing member of the device when the connection port couples with the device.

In one or more embodiments of the system, the guiding member of the connection port is a rod.

In one or more embodiments of the system, the guiding member of the connection port is a dent.

In one or more embodiments of the system, the guiding member of the connection port is a slot.

In one or more embodiments of the system, the guiding member of the connection port is a protrusion.

In one or more embodiments of the system, wherein the device further comprising a guiding member configured to align the removable sealing member of the device with the removable sealing member of the connection port.

In one or more embodiments of the system, wherein the connection port further comprising a guiding member configured to align the removable sealing member of the device with the removable sealing member of the connection port.

In one or more embodiments of the system, wherein the device further comprising a guiding member and wherein the connection port further comprising a guiding member, wherein both the guiding member of the device and the guiding member of the connection port are configured to engage with each other in such a manner as to align the removable sealing member of the device and the removable sealing member of the connection port so that both said removable sealing members are able to displace at about the same time and in the same direction when moved to a second unsealed position.

In one or more embodiments of the system, wherein the guiding member of the device is a rod and the guiding member of the connection port is a complimentary slot which allows the rod to enter into the slot.

In one or more embodiments of the system, wherein the guiding member of the connection port is a rod and the guiding member of the device is a complimentary slot which allows the rod to enter into the slot.

In one or more embodiments of the system, wherein the guiding member of the device is a protrusion and the complimentary guiding member of the connection port is a dent, wherein the dent allows the protrusion to engage and/or enter into the dent.

In one or more embodiments of the system, wherein the guiding member of the connection port is a protrusion and the complimentary guiding member of the device is a dent, wherein the dent allows the protrusion to engage and/or enter into the dent.

In one or more embodiments, the proximal end portion of the housing is integrally attached to a container and the distal end portion of the housing having a guiding member.

In one or more embodiments, the wiping member is configured to extend from the top wall of the housing to the bottom wall of the housing.

In one or more embodiments, the wiping member is configured to provide a hermetic seal between the top wall and the wiping member and the bottom wall and the wiping member when the wiping member moves across the housing.

In one or more embodiments, the wiping member is configured to wipe off a surface of the first sealing member of the housing when the wiping member moves across the housing.

In one or more embodiments, a filter is disposed in, on, or over an aperture in the housing for releasing air from the housing when actuating the wiping member.

In one or more embodiments, the housing is manufactured from a group consisting of plastic, rigid plastic, flexible plastic, polymer, and combinations thereof.

In one or more embodiments, the housing is made of a glass material, a metal material, and combinations thereof.

In one or more embodiments, the wiping member is made of a plastic substance, a rubber substance, an elastomeric substance, and combinations thereof.

In one or more embodiments, the wiping member is covered and/or saturated with an antibacterial substance, an antiviral substance, an anti-tuberculin substance, an anti-HIV substance, hydrogen peroxide, isopropyl alcohol, and combinations thereof.

In one or more embodiments, the device and/or housing is disposable and/or for single use.

In one or more embodiments, a surface of the removable sealing member has an adhesive on the surface of the removable sealing member.

In one or more embodiments, an exterior surface of the removable sealing member has an adhesive.

In one or more embodiments, the device further comprises a sealing ring.

In one or more embodiments, the sealing ring of the device is an “O-ring”.

In one or more embodiments, the sealing ring is a circumferential sealing ring.

In one or more embodiments, the “O-ring” is a circumferential “O-ring”.

In one or more embodiments, the sealing ring of the device is made of at least one of a plastic material and a rubber material.

In one or more embodiments, the sealing “O-ring” of the device is made of at least one of a plastic material and a rubber material.

In one or more embodiments, the connection port further comprises a sealing ring.

In one or more embodiments, the sealing ring of the connection port is an “O-ring”.

In one or more embodiments, the sealing ring of the connection port is a circumferential sealing ring.

In one or more embodiments, the “O-ring” of the connection port is a circumferential “O-ring”.

In one or more embodiments, the sealing ring of the connection port is made of at least one of a plastic material and a rubber material.

In one or more embodiments, the sealing “O-ring” of the connection port is made of at least one of a plastic material and a rubber material.

In one or more embodiments, both the device and the connection port further comprise a sealing ring.

In one or more embodiments, the sealing ring of the device and the sealing ring of the connection port is an “O-ring”.

In one or more embodiments, the sealing ring of the device is a circumferential sealing ring and the sealing ring of the connection port is a circumferential sealing ring.

In one or more embodiments, the “O-ring” of the device is a circumferential “O-ring” and the “O-ring of the connection port is a circumferential “O-ring”.

In one or more embodiments, the sealing ring of the connection port is made of at least one of a plastic material and a rubber material and the sealing ring of the device is made of at least one of a plastic material and a rubber material.

In one or more embodiments, the sealing “O-ring” of the device and the sealing “O-ring” of the connection port are made of at least one of a plastic material and a rubber material.

In one or more embodiments, the device further comprising teeth configured to engage/couple holes/dents of a connection port.

In one or more embodiments, the teeth of the device are retractable teeth.

In one or more embodiments, the teeth are positioned at a distal end portion of the device.

In one or more embodiments, the retractable teeth are positioned at a distal end portion of the device.

In one or more embodiments, the teeth are positioned on an engagement mechanism of the device.

In one or more embodiments, the teeth are positioned on a securing mechanism of the device.

In one or more embodiments, the teeth are positioned on the device.

In one or more embodiments, the teeth are positioned on the housing of the device.

In one or more embodiments, the retractable teeth are positioned on the device.

In one or more embodiments, the retractable teeth are positioned on the housing of the device.

In one or more embodiments, the teeth are configured to engage/couple a hole/dent of a connection port.

In one or more embodiments, the retractable teeth are configured to engage/couple a hole/dent of a connection port.

In one or more embodiments, the teeth are configured to provide coupling of a connection port to the device, the teeth are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the device further comprising holes/dents configured to engage/couple with teeth of a connection port.

In one or more embodiments, the device further comprising holes/dents configured to engage/couple with retractable teeth of a connection port.

In one or more embodiments, the holes/debts are positioned at a distal end portion of the device.

In one or more embodiments, the holes/dents are positioned on an engagement mechanism of the device.

In one or more embodiments, the holes/dents are positioned on a securing mechanism of the device.

In one or more embodiments, the holes/dents are positioned on the device.

In one or more embodiments, the holes/dents are positioned on the housing of the device.

In one or more embodiments, the holes/dents are configured to engage/couple teeth of a connection port.

In one or more embodiments, the holes/dents are configured to engage/couple retractable teeth of a connection port.

In one or more embodiments, the holes/dents are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments of the system, the device further comprising teeth and the connection port further comprising holes/dents.

In one or more embodiments of the system, the device further comprising retractable teeth and the connection port further comprising holes/dents configured to engage/couple the retractable teeth.

In one or more embodiments of the system, the device further comprising holes/dents and the connection port further comprising teeth.

In one or more embodiments of the system, the device further comprising holes/dents and the connection port further comprising retractable teeth.

In one or more embodiments, the holes/dents of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the holes/dents of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the teeth of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the retractable teeth of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the teeth of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the retractable teeth of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In still a further aspect, the present invention provides a system for establishing a decontaminated fluid passageway between a device and a connection port, comprising: i) a device comprising: a housing defining a fluid passageway, a proximal end portion of the housing attached to a container and a distal end portion of the housing configured to couple a connection port, the distal end portion of the housing having a securing mechanism configured to engage the connection port; a removable sealing member for sealing the distal end portion of the housing, the removable sealing member positioned to seal the distal end portion of the fluid passageway, the removable sealing member configured to be movable between a first sealed position and a second unsealed position, the removable sealing member being moveable by manual manipulation of the removable sealing member, wherein the removable sealing member is displaced from the fluid passageway when the removable sealing member in the second unsealed position, the device further comprising a guiding member configured to engage the connection port when the connection port is attached/coupled to the device, the guiding member characterized in that is allows the device to couple to the connection port in a manner that aligns the removable sealing member of the device with the removable sealing member of a connection port so that both removable sealing members of the device and the connection port lie flat against one another; ii) a connection port, comprising: a distal end portion of the connection port attached to a container and a proximal end portion defining a fluid passageway, the proximal end portion of the connection port configured to couple the distal end portion of the housing of the device, the proximal end portion of the connection port having a securing mechanism configured to engage the securing mechanism of the distal end portion of the housing of the device; a removable sealing member for sealing the proximal end portion of the fluid passageway of the connection port, the removable sealing member of the connection port configured to be movable between a first sealed position and a second unsealed position, the removable sealing member of the connection port being movable by manual manipulation of the removable sealing member, wherein the removable sealing member is displaced from the fluid passageway of the connection port when the removable sealing member is in the second unsealed position; and a second sealing member positioned to seal an opening of the connection port, the second sealing member positioned inside and/or over the opening of the connection port, the second sealing member covered by the removable sealing member of the connection port when the removable sealing member of the connection port is in a first sealed position and the second sealing member being uncovered when the removable sealing member of the connection port is displaced to a second unsealed position.

In one or more embodiments, the connection port further comprising an engagement mechanism configured to engage the guiding member of the device.

In one or more embodiments, the engagement mechanism of the connection port configured to engage the guiding member of the device is selected from a rod, a dent, and a thread.

In one or more embodiments, the container attached to the proximal end portion of the device is a bag or a bottle.

In one or more embodiments, the container attached to the proximal end portion of the device is a vial.

In one or more embodiments, the container attached to the distal end portion of the connection port is a bag or a bottle.

In one or more embodiments, the container attached to the distal end portion of the connection port is a vial.

In one or more embodiments, the container attached to the proximal end portion of the housing of the device is a bag and the container attached to the distal end portion of the connection port is a bottle.

In one or more embodiments, the container attached to the proximal end portion of the housing of the device is a bag and the container attached to the distal end portion of the connection port is a vial.

In one or more embodiments, the container attached to the proximal end portion of the housing of the device is a bottle and the container attached to the distal end portion of the connection port is a bag.

In one or more embodiments, the container attached to the proximal end portion of the housing of the device is a vial and the container attached to the distal end portion of the connection port is a bag.

In one or more embodiments, the container attached to the proximal end portion of the housing of the device is a vial and the container attached to the distal end portion of the connection port is a vial.

In one or more embodiments, the container attached to the proximal end portion of the housing of the device is a bottle and the container attached to the distal end portion of the connection port is a bottle.

In one or more embodiments, the container attached to the proximal end portion of the housing is integrally attached to the housing of the device.

In one or more embodiments, the container attached to the proximal end portion of the housing of the device forms a unitary structure with the device.

In one or more embodiments, the container attached to the distal end portion of the connection port is integrally attached to the connection port.

In one or more embodiments, the container attached to the distal end portion of the connection port forms a unitary structure with the connection port.

In one or more embodiments, the guiding member is selected from a rod, a hole, and a dent.

In one or more embodiments, the guiding member having a thread.

In one or more embodiments, the guiding member protrudes out of the connection port to an exterior of the connection port.

In one or more embodiments, the second sealing member is at least one of pierceable and frangible.

In one or more embodiments, the second sealing member is a stopper.

In one or more embodiments, the second sealing member is a rubber stopper.

In one or more embodiments, the second sealing member is a bung.

In one or more embodiments, the second sealing member is a rubber bung.

In one or more embodiments, the second sealing member prevents escape/leakage of fluid out of the connection port in the event of spontaneous disengagement between the connection port and the device.

In one or more embodiments, the second sealing member is configured to be pierced by a piercing member of the device.

In one or more embodiments, the second sealing member is configured to be pierced by a piercing member of the connection port.

In one or more embodiments, at least one of the device and the connection port further comprising a piercing member configured to pierce through the second sealing member of the connection port.

In one or more embodiments, the device further comprising a plug positioned in an opening at the distal end portion of the device, the plug configured to prevent escape/leakage of fluid out of the device in the event of spontaneous disengagement between the connection port and the device.

In one or more embodiments, the plug of the device is at least one of pierceable and frangible.

In one or more embodiments, the plug of the device is configured to be pierced by a piercing member of the device or by a piercing member of the connection port.

In one or more embodiments, both the plug of the device and the second sealing member of the connection port serve as a means to prevent spillage of the contents held/transferred between the device and the connection port in the event of spontaneous/accidental disengagement of the device from the connection port.

In one or more embodiments, the removable sealing member of the device and the removable sealing member of the connection port are both configured to substantially seal the fluid passageway between the device and the connection port when both the removable sealing member of the device and the removable sealing member of the connection port are in a first sealed position, and wherein the removable sealing member of the device and the removable sealing member of the connection port are configured to allow passage of fluid between the device and the connection port when the removable sealing member of the device and the removable sealing member of the connection port are both in the second unsealed position.

In one or more embodiments, the removable sealing member of the device and the removable sealing member of the connection port are both configured to be moved from the first sealed position to the second unsealed position via a displacement of the removable sealing members of both the device and the connection port, wherein the displacement of both removable sealing members of the device and the connection port are external to the fluid passageway between the device and the connection port.

In one or more embodiments, the removable sealing member of the device is in a folded configuration and the removable sealing member of the connection port is in a folded configuration.

In one or more embodiments, the removable sealing member of the device is in a folded configuration or the removable sealing member of the connection port is in a folded configuration.

In one or more embodiments, at least one of the removable sealing members of the device and the removable sealing member of the connection port are in a folded configuration.

In one or more embodiments, a portion of the removable sealing member of the device is attached to an interior surface of the housing.

In one or more embodiments, a portion of the removable sealing member of the device is attached to an interior surface of the housing and a portion of the removable sealing member of the device is attached to an exterior surface of the housing.

In one or more embodiments, the first sealing member of the device is characterized as being at least one of pierceable and frangible.

In one or more embodiments, the device further comprising a piercing member.

In one or more embodiments, the housing further comprising a piercing member.

In one or more embodiments, the device further comprising a piercing member actuator.

In one or more embodiments, the piercing member actuator configured to move the piercing member through the first sealing member of the device.

In one or more embodiments, the piercing member is a needle.

In one or more embodiments, the piercing member is a hollowed needle.

In one or more embodiments, the piercing member is configured to pierce through the second sealing member of the connection port when the connection port is engaged to the housing of the device.

In one or more embodiments, the piercing member actuator is configured to move the piercing member through the second sealing member of the connection port when the connection port is coupled to the housing of the device.

In one or more embodiments, the connection port further comprising a second sealing member located at the proximal end portion of the connection port.

In one or more embodiments, the removable sealing member of the connection port covers the second sealing member of the connection port when the removable sealing member of the connection port is in a first sealed position.

In one or more embodiments, the second sealing member is pierceable.

In one or more embodiments, the second sealing member is a rubber bung.

In one or more embodiments, the second sealing member is frangible.

In one or more embodiments, the second sealing member is a rubber stopper.

In one or more embodiments, the second sealing member is made of a plastic material, a metal alloy, and combinations thereof.

In one or more embodiments, the second sealing member is a plug that substantially seals the fluid passageway between the device and the connection port.

In one or more embodiments, the plug is displaceable.

In one or more embodiments, when the device is coupled to the connection port, and when the removable sealing member of the device and the removable sealing member of the connection port are both in the second unsealed position, a surface of the second sealing member of the connection port is exposed to an interior of the housing of the device.

In one or more embodiments, wherein the surface of the second sealing member of the connection port is characterized in that the surface of the second sealing member is positioned to be wiped off by the wiping member of the device when the wiping member moves across the housing.

In one or more embodiments, wherein when the device is coupled to the connection port, the surface of the second sealing member of the connection port and the wiping member of the device form an airtight seal when the wiping member moves across the housing of the device and wipes off the surface of the second sealing member.

In one or more embodiments, the wiping member of the device wipes off a surface of the second sealing member of the connection port via at least one of press and friction force(s).

In one or more embodiments, the device further comprising a piercing member configured to pierce through the first sealing member of the device and the second sealing member of the connection port.

In one or more embodiments, wherein the piercing member is a needle.

In one or more embodiments, wherein the piercing member is a hollowed needle.

In one or more embodiments, the securing mechanism at the distal end portion of the housing of the device is a thread and the securing mechanism at the proximal end portion of the connection port is a complimentary thread.

In one or more embodiments, the securing mechanism at the distal end portion of the housing of the device are ratchet teeth and the securing mechanism at the proximal end portion of the connection port is a complimentary retention member.

In one or more embodiments, the securing mechanism at the distal end portion of the device is a retention member and the securing mechanism at the proximal end portion of the connection port are complimentary ratchet teeth.

In one or more embodiments, the securing mechanism at the distal end portion of the housing of the device is a clamping member and the securing mechanism at the proximal end portion of the connection port is a complimentary retention member.

In one or more embodiments, the securing mechanism at the distal end portion of the housing of the device is a retention member and the securing mechanism at the proximal end portion of the connection port is a complimentary clamping member.

In one or more embodiments, at least one of the securing mechanism at the distal end portion of the housing and the securing mechanism at the proximal end portion of the connection port is an adhesive.

In one or more embodiments, the proximal end portion of the housing of the device is further configured to couple to a container and the distal end portion of the connection port is further configured to couple to a container.

In one or more embodiments, the proximal end portion of the housing of the device is further configured to couple to a bag and the distal end portion of the connection port is further configured to couple to a vial.

In one or more embodiments, wherein the distal end portion of the connection port is attached to a syringe and the proximal end portion of the housing of the device is attached to a container.

In one or more embodiments, the distal end portion of the connection port is integrally attached to a container and the proximal end portion of the housing of the device is integrally attached to a container.

In one or more embodiments of the system, the distal end portion of the connection port forms a unitary structure with a container and the proximal end portion of the housing of the device forms a unitary structure with a container.

In one or more embodiments of the system, the distal end portion of the connection port is attached to any of a syringe, a filter, an infusion line, and wherein the proximal end portion of the housing of the device is attached to any of a syringe, a filter, an infusion line, and a container.

In one or more embodiments of the system, wherein the removable sealing member of the device is attached to the housing of the device via a frangible seal and the removable sealing member of the connection port is attached to the connection port via a frangible seal.

In one or more embodiments of the system, wherein the removable sealing member of the device and the removable sealing member of the connection port are both configured to be displaced from the fluid passageway via a pulling motion.

In one or more embodiments of the system, wherein the removable sealing member of the device and the removable sealing member of the connection port are both configured to be displaced from the fluid passageway at about the same time via a pulling motion.

In one or more embodiments of the system, wherein at least a portion of the removable sealing member of the device is located through a wall of the housing.

In one or more embodiments, wherein at least a portion of the removable sealing member of the device is located through a side wall of the housing.

In one or more embodiments of the system, wherein at least one of an antibacterial substance, an antiviral substance, and an antifungal substance is located inside the housing of the device, on a surface of the removable sealing member, on the wiping member, and combinations thereof.

In one or more embodiments of the system, wherein the securing mechanism of the distal end portion of the housing of the device is further configured to be movable between a first extended position and a second contracted position.

In one or more embodiments of the system, wherein when the securing mechanism of the distal end portion of the housing of the device is in the second contracted position the second sealing member of the connection port is positioned to be wiped off by the wiping member of the device.

In one or more embodiments of the system, wherein when the device is coupled to the connection port and when the securing mechanism of the device is in a first extended position, an exterior surface of the second sealing member of the connection port is not aligned with an interior surface of the housing, and wherein when the device is coupled to the connection port and the securing mechanism of the device is in the second contracted position, an exterior surface of the second sealing member of the connection port is aligned with an interior surface of the housing.

In one or more embodiments of the system, wherein when the exterior surface of the second sealing member of the connection port is aligned with an interior surface of the housing, the wiping member is able to wipe off the exterior surface of the second sealing member of the connection port when the wiping member moves across the housing.

In one or more embodiments of the system, wherein the distal end portion of the housing of the device is further configured to be movable between a first extended position and a second contracted position.

In one or more embodiments, wherein the securing mechanism of the distal end portion of the housing of the device is selected from a group consisting of a thread, ratchet teeth, a clamping member, a retention member, and an adhesive.

In one or more embodiments of the system, the housing further comprising a guiding member configured to align the removable sealing member of the housing with the removable sealing member of the connection port.

In one or more embodiments, the guiding member is a rod.

In one or more embodiments, the guiding member is a dent.

In one or more embodiments, the guiding member is a slot.

In one or more embodiments, the guiding member is a protrusion.

In one or more embodiments of the system, the guiding member is configured to align the removable sealing member of the housing of the device with the removable sealing member of the connection port, wherein upon alignment of the removable sealing member of the housing of the device with the removable sealing member of the connection port both the removable sealing member of the housing and the removable sealing member of the connection port will displace in about the same direction and at about the same time external to the location of fluidic communication between the housing and the connection port.

In one or more embodiments of the system, the connection port has a guiding member configured to align the removable sealing member of the connection port with the removable sealing member of the device when the connection port couples with the device.

In one or more embodiments of the system, the guiding member of the connection port is a rod.

In one or more embodiments of the system, the guiding member of the connection port is a dent.

In one or more embodiments of the system, the guiding member of the connection port is a slot.

In one or more embodiments of the system, the guiding member of the connection port is a protrusion.

In one or more embodiments of the system, wherein the device further comprising a guiding member configured to align the removable sealing member of the device with the removable sealing member of the connection port.

In one or more embodiments of the system, wherein the connection port further comprising a guiding member configured to align the removable sealing member of the device with the removable sealing member of the connection port.

In one or more embodiments of the system, wherein the device further comprising a guiding member and wherein the connection port further comprising a guiding member, wherein both the guiding member of the device and the guiding member of the connection port are configured to engage with each other in such a manner as to align the removable sealing member of the device and the removable sealing member of the connection port so that both said removable sealing members are able to displace at about the same time and in the same direction when moved to a second unsealed position.

In one or more embodiments of the system, wherein the guiding member of the device is a rod and the guiding member of the connection port is a complimentary slot which allows the rod to enter into the slot.

In one or more embodiments of the system, wherein the guiding member of the connection port is a rod and the guiding member of the device is a complimentary slot which allows the rod to enter into the slot.

In one or more embodiments of the system, wherein the guiding member of the device is a protrusion and the complimentary guiding member of the connection port is a dent, wherein the dent allows the protrusion to engage and/or enter into the dent.

In one or more embodiments of the system, wherein the guiding member of the connection port is a protrusion and the complimentary guiding member of the device is a dent, wherein the dent allows the protrusion to engage and/or enter into the dent.

In one or more embodiments, the proximal end portion of the housing is integrally attached to a container and the distal end portion of the housing having a guiding member.

In one or more embodiments, the wiping member is configured to extend from the top wall of the housing to the bottom wall of the housing.

In one or more embodiments, the wiping member is configured to provide a hermetic seal between the top wall and the wiping member and the bottom wall and the wiping member when the wiping member moves across the housing.

In one or more embodiments, the wiping member is configured to wipe off a surface of the first sealing member of the housing when the wiping member moves across the housing.

In one or more embodiments, a filter is disposed in, on, or over an aperture in the housing for releasing air from the housing when actuating the wiping member.

In one or more embodiments, the housing is manufactured from a group consisting of plastic, rigid plastic, flexible plastic, polymer, and combinations thereof.

In one or more embodiments, the housing is made of a glass material, a metal material, and combinations thereof.

In one or more embodiments, the wiping member is made of a plastic substance, a rubber substance, an elastomeric substance, and combinations thereof.

In one or more embodiments, the wiping member is covered and/or saturated with an antibacterial substance, an antiviral substance, an anti-tuberculin substance, an anti-HIV substance, hydrogen peroxide, isopropyl alcohol, and combinations thereof.

In one or more embodiments, the device and/or housing is disposable and/or for single use.

In one or more embodiments, a surface of the removable sealing member has an adhesive on the surface of the removable sealing member.

In one or more embodiments, an exterior surface of the removable sealing member has an adhesive.

In one or more embodiments, the device further comprises a sealing ring.

In one or more embodiments, the sealing ring of the device is an “O-ring”.

In one or more embodiments, the sealing ring is a circumferential sealing ring.

In one or more embodiments, the “O-ring” is a circumferential “O-ring”.

In one or more embodiments, the sealing ring of the device is made of at least one of a plastic material and a rubber material.

In one or more embodiments, the sealing “O-ring” of the device is made of at least one of a plastic material and a rubber material.

In one or more embodiments, the connection port further comprises a sealing ring.

In one or more embodiments, the sealing ring of the connection port is an “O-ring”.

In one or more embodiments, the sealing ring of the connection port is a circumferential sealing ring.

In one or more embodiments, the “O-ring” of the connection port is a circumferential “O-ring”.

In one or more embodiments, the sealing ring of the connection port is made of at least one of a plastic material and a rubber material.

In one or more embodiments, the sealing “O-ring” of the connection port is made of at least one of a plastic material and a rubber material.

In one or more embodiments, both the device and the connection port further comprise a sealing ring.

In one or more embodiments, the sealing ring of the device and the sealing ring of the connection port is an “O-ring”.

In one or more embodiments, the sealing ring of the device is a circumferential sealing ring and the sealing ring of the connection port is a circumferential sealing ring.

In one or more embodiments, the “O-ring” of the device is a circumferential “O-ring” and the “O-ring of the connection port is a circumferential “O-ring”.

In one or more embodiments, the sealing ring of the connection port is made of at least one of a plastic material and a rubber material and the sealing ring of the device is made of at least one of a plastic material and a rubber material.

In one or more embodiments, the sealing “O-ring” of the device and the sealing “O-ring” of the connection port are made of at least one of a plastic material and a rubber material.

In one or more embodiments, the device further comprising teeth configured to engage/couple holes/dents of a connection port.

In one or more embodiments, the teeth of the device are retractable teeth.

In one or more embodiments, the teeth are positioned at a distal end portion of the device.

In one or more embodiments, the retractable teeth are positioned at a distal end portion of the device.

In one or more embodiments, the teeth are positioned on an engagement mechanism of the device.

In one or more embodiments, the teeth are positioned on a securing mechanism of the device.

In one or more embodiments, the teeth are positioned on the device.

In one or more embodiments, the teeth are positioned on the housing of the device.

In one or more embodiments, the retractable teeth are positioned on the device.

In one or more embodiments, the retractable teeth are positioned on the housing of the device.

In one or more embodiments, the teeth are configured to engage/couple a hole/dent of a connection port.

In one or more embodiments, the retractable teeth are configured to engage/couple a hole/dent of a connection port.

In one or more embodiments, the teeth are configured to provide coupling of a connection port to the device, the teeth are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the device further comprising holes/dents configured to engage/couple with teeth of a connection port.

In one or more embodiments, the device further comprising holes/dents configured to engage/couple with retractable teeth of a connection port.

In one or more embodiments, the holes/debts are positioned at a distal end portion of the device.

In one or more embodiments, the holes/dents are positioned on an engagement mechanism of the device.

In one or more embodiments, the holes/dents are positioned on a securing mechanism of the device.

In one or more embodiments, the holes/dents are positioned on the device.

In one or more embodiments, the holes/dents are positioned on the housing of the device.

In one or more embodiments, the holes/dents are configured to engage/couple teeth of a connection port.

In one or more embodiments, the holes/dents are configured to engage/couple retractable teeth of a connection port.

In one or more embodiments, the holes/dents are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments of the system, the device further comprising teeth and the connection port further comprising holes/dents.

In one or more embodiments of the system, the device further comprising retractable teeth and the connection port further comprising holes/dents configured to engage/couple the retractable teeth.

In one or more embodiments of the system, the device further comprising holes/dents and the connection port further comprising teeth.

In one or more embodiments of the system, the device further comprising holes/dents and the connection port further comprising retractable teeth.

In one or more embodiments, the holes/dents of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the holes/dents of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the teeth of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the retractable teeth of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the teeth of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the retractable teeth of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In still a further aspect, the present invention provides a system for establishing a decontaminated fluid passageway between a device and a connection port, comprising: i) a device comprising: a housing defining a fluid passageway, a proximal end portion of the housing attached to a container and a distal end portion of the housing configured to couple a connection port, the distal end portion of the housing having a securing mechanism configured to engage the connection port; a removable sealing member for sealing the distal end portion of the housing, the removable sealing member positioned to seal the distal end portion of the fluid passageway, the removable sealing member configured to be movable between a first sealed position and a second unsealed position, the removable sealing member being moveable by manual manipulation of the removable sealing member, wherein the removable sealing member is displaced from the fluid passageway when the removable sealing member in the second unsealed position, the device further comprising a guiding member configured to engage the connection port when the connection port is attached/coupled to the device, the guiding member characterized in that is allows the device to couple to the connection port in a manner that aligns the removable sealing member of the device with the removable sealing member of a connection port so that both removable sealing members of the device and the connection port lie flat against one another, the device further comprising a piercing member configured to pierce through a second sealing member of a connection port; ii) a connection port, comprising: a distal end portion of the connection port attached to a container and a proximal end portion defining a fluid passageway, the proximal end portion of the connection port configured to couple the distal end portion of the housing of the device, the proximal end portion of the connection port having a securing mechanism configured to engage the securing mechanism of the distal end portion of the housing of the device; a removable sealing member for sealing the proximal end portion of the fluid passageway of the connection port, the removable sealing member of the connection port configured to be movable between a first sealed position and a second unsealed position, the removable sealing member of the connection port being movable by manual manipulation of the removable sealing member, wherein the removable sealing member is displaced from the fluid passageway of the connection port when the removable sealing member is in the second unsealed position; and a second sealing member positioned to seal an opening of the connection port, the second sealing member positioned inside and/or over the opening of the connection port, the second sealing member covered by the removable sealing member of the connection port when the removable sealing member of the connection port is in a first sealed position and the second sealing member being uncovered when the removable sealing member of the connection port is displaced to a second unsealed position.

In one or more embodiments, the device further comprising a piercing member actuator configured to move the piercing member through the second sealing member of the connection port thus piercing the second sealing member of the connection port to establish a fluidic communication between the device and the connection port.

In one or more embodiments, the piercing member actuator is in the form of a button.

In one or more embodiments, the piercing member actuator is in the form of a tab.

In one or more embodiments, the piercing member actuator is a needle.

In one or more embodiments, the piercing member actuator is a hollowed needle.

In one or more embodiments, the device further comprising a first sealing member disposed on a proximal end portion of the housing of the device, the first sealing member characterized in that it is positioned to fluidly seal the proximal end portion of the housing of the device.

In one or more embodiments, the piercing member is configured to pierce through both the first sealing member located at the proximal end portion of the device and pierce through the second sealing member of the connection port when the device and the connection port are engaged/coupled to each other.

In one or more embodiments, the connection port further comprising an engagement mechanism configured to engage the guiding member of the device.

In one or more embodiments, the engagement mechanism of the connection port configured to engage the guiding member of the device is selected from a rod, a dent, and a thread.

In one or more embodiments, the container attached to the proximal end portion of the device is a bag or a bottle.

In one or more embodiments, the container attached to the proximal end portion of the device is a vial.

In one or more embodiments, the container attached to the distal end portion of the connection port is a bag or a bottle.

In one or more embodiments, the container attached to the distal end portion of the connection port is a vial.

In one or more embodiments, the container attached to the proximal end portion of the housing of the device is a bag and the container attached to the distal end portion of the connection port is a bottle.

In one or more embodiments, the container attached to the proximal end portion of the housing of the device is a bag and the container attached to the distal end portion of the connection port is a vial.

In one or more embodiments, the container attached to the proximal end portion of the housing of the device is a bottle and the container attached to the distal end portion of the connection port is a bag.

In one or more embodiments, the container attached to the proximal end portion of the housing of the device is a vial and the container attached to the distal end portion of the connection port is a bag.

In one or more embodiments, the container attached to the proximal end portion of the housing of the device is a vial and the container attached to the distal end portion of the connection port is a vial.

In one or more embodiments, the container attached to the proximal end portion of the housing of the device is a bottle and the container attached to the distal end portion of the connection port is a bottle.

In one or more embodiments, the container attached to the proximal end portion of the housing is integrally attached to the housing of the device.

In one or more embodiments, the container attached to the proximal end portion of the housing of the device forms a unitary structure with the device.

In one or more embodiments, the container attached to the distal end portion of the connection port is integrally attached to the connection port.

In one or more embodiments, the container attached to the distal end portion of the connection port forms a unitary structure with the connection port.

In one or more embodiments, the guiding member is selected from a rod, a hole, and a dent.

In one or more embodiments, the guiding member having a thread.

In one or more embodiments, the guiding member protrudes out of the connection port to an exterior of the connection port.

In one or more embodiments, the second sealing member is at least one of pierceable and frangible.

In one or more embodiments, the second sealing member is a stopper.

In one or more embodiments, the second sealing member is a rubber stopper.

In one or more embodiments, the second sealing member is a bung.

In one or more embodiments, the second sealing member is a rubber bung.

In one or more embodiments, the second sealing member prevents escape/leakage of fluid out of the connection port in the event of spontaneous disengagement between the connection port and the device.

In one or more embodiments, the second sealing member is configured to be pierced by a piercing member of the device.

In one or more embodiments, the second sealing member is configured to be pierced by a piercing member of the connection port.

In one or more embodiments, at least one of the device and the connection port further comprising a piercing member configured to pierce through the second sealing member of the connection port.

In one or more embodiments, the device further comprising a plug positioned in an opening at the distal end portion of the device, the plug configured to prevent escape/leakage of fluid out of the device in the event of spontaneous disengagement between the connection port and the device.

In one or more embodiments, the plug of the device is at least one of pierceable and frangible.

In one or more embodiments, the plug of the device is configured to be pierced by a piercing member of the device or by a piercing member of the connection port.

In one or more embodiments, both the plug of the device and the second sealing member of the connection port serve as a means to prevent spillage of the contents held/transferred between the device and the connection port in the event of spontaneous/accidental disengagement of the device from the connection port.

In one or more embodiments, the removable sealing member of the device and the removable sealing member of the connection port are both configured to substantially seal the fluid passageway between the device and the connection port when both the removable sealing member of the device and the removable sealing member of the connection port are in a first sealed position, and wherein the removable sealing member of the device and the removable sealing member of the connection port are configured to allow passage of fluid between the device and the connection port when the removable sealing member of the device and the removable sealing member of the connection port are both in the second unsealed position.

In one or more embodiments, the removable sealing member of the device and the removable sealing member of the connection port are both configured to be moved from the first sealed position to the second unsealed position via a displacement of the removable sealing members of both the device and the connection port, wherein the displacement of both removable sealing members of the device and the connection port are external to the fluid passageway between the device and the connection port.

In one or more embodiments, the removable sealing member of the device is in a folded configuration and the removable sealing member of the connection port is in a folded configuration.

In one or more embodiments, the removable sealing member of the device is in a folded configuration or the removable sealing member of the connection port is in a folded configuration.

In one or more embodiments, at least one of the removable sealing members of the device and the removable sealing member of the connection port are in a folded configuration.

In one or more embodiments, a portion of the removable sealing member of the device is attached to an interior surface of the housing.

In one or more embodiments, a portion of the removable sealing member of the device is attached to an interior surface of the housing and a portion of the removable sealing member of the device is attached to an exterior surface of the housing.

In one or more embodiments, the first sealing member of the device is characterized as being at least one of pierceable and frangible.

In one or more embodiments, the device further comprising a piercing member.

In one or more embodiments, the housing further comprising a piercing member.

In one or more embodiments, the device further comprising a piercing member actuator.

In one or more embodiments, the piercing member actuator configured to move the piercing member through the first sealing member of the device.

In one or more embodiments, the piercing member is a needle.

In one or more embodiments, the piercing member is a hollowed needle.

In one or more embodiments, the piercing member is configured to pierce through the second sealing member of the connection port when the connection port is engaged to the housing of the device.

In one or more embodiments, the piercing member actuator is configured to move the piercing member through the second sealing member of the connection port when the connection port is coupled to the housing of the device.

In one or more embodiments, the connection port further comprising a second sealing member located at the proximal end portion of the connection port.

In one or more embodiments, the removable sealing member of the connection port covers the second sealing member of the connection port when the removable sealing member of the connection port is in a first sealed position.

In one or more embodiments, the second sealing member is pierceable.

In one or more embodiments, the second sealing member is a rubber bung.

In one or more embodiments, the second sealing member is frangible.

In one or more embodiments, the second sealing member is a rubber stopper.

In one or more embodiments, the second sealing member is made of a plastic material, a metal alloy, and combinations thereof.

In one or more embodiments, the second sealing member is a plug that substantially seals the fluid passageway between the device and the connection port.

In one or more embodiments, the plug is displaceable.

In one or more embodiments, when the device is coupled to the connection port, and when the removable sealing member of the device and the removable sealing member of the connection port are both in the second unsealed position, a surface of the second sealing member of the connection port is exposed to an interior of the housing of the device.

In one or more embodiments, wherein the surface of the second sealing member of the connection port is characterized in that the surface of the second sealing member is positioned to be wiped off by the wiping member of the device when the wiping member moves across the housing.

In one or more embodiments, wherein when the device is coupled to the connection port, the surface of the second sealing member of the connection port and the wiping member of the device form an airtight seal when the wiping member moves across the housing of the device and wipes off the surface of the second sealing member.

In one or more embodiments, the wiping member of the device wipes off a surface of the second sealing member of the connection port via at least one of press and friction force(s).

In one or more embodiments, the device further comprising a piercing member configured to pierce through the first sealing member of the device and the second sealing member of the connection port.

In one or more embodiments, wherein the piercing member is a needle.

In one or more embodiments, wherein the piercing member is a hollowed needle.

In one or more embodiments, the securing mechanism at the distal end portion of the housing of the device is a thread and the securing mechanism at the proximal end portion of the connection port is a complimentary thread.

In one or more embodiments, the securing mechanism at the distal end portion of the housing of the device are ratchet teeth and the securing mechanism at the proximal end portion of the connection port is a complimentary retention member.

In one or more embodiments, the securing mechanism at the distal end portion of the device is a retention member and the securing mechanism at the proximal end portion of the connection port are complimentary ratchet teeth.

In one or more embodiments, the securing mechanism at the distal end portion of the housing of the device is a clamping member and the securing mechanism at the proximal end portion of the connection port is a complimentary retention member.

In one or more embodiments, the securing mechanism at the distal end portion of the housing of the device is a retention member and the securing mechanism at the proximal end portion of the connection port is a complimentary clamping member.

In one or more embodiments, at least one of the securing mechanism at the distal end portion of the housing and the securing mechanism at the proximal end portion of the connection port is an adhesive.

In one or more embodiments, the proximal end portion of the housing of the device is further configured to couple to a container and the distal end portion of the connection port is further configured to couple to a container.

In one or more embodiments, the proximal end portion of the housing of the device is further configured to couple to a bag and the distal end portion of the connection port is further configured to couple to a vial.

In one or more embodiments, wherein the distal end portion of the connection port is attached to a syringe and the proximal end portion of the housing of the device is attached to a container.

In one or more embodiments, the distal end portion of the connection port is integrally attached to a container and the proximal end portion of the housing of the device is integrally attached to a container.

In one or more embodiments of the system, the distal end portion of the connection port forms a unitary structure with a container and the proximal end portion of the housing of the device forms a unitary structure with a container.

In one or more embodiments of the system, the distal end portion of the connection port is attached to any of a syringe, a filter, an infusion line, and wherein the proximal end portion of the housing of the device is attached to any of a syringe, a filter, an infusion line, and a container.

In one or more embodiments of the system, wherein the removable sealing member of the device is attached to the housing of the device via a frangible seal and the removable sealing member of the connection port is attached to the connection port via a frangible seal.

In one or more embodiments of the system, wherein the removable sealing member of the device and the removable sealing member of the connection port are both configured to be displaced from the fluid passageway via a pulling motion.

In one or more embodiments of the system, wherein the removable sealing member of the device and the removable sealing member of the connection port are both configured to be displaced from the fluid passageway at about the same time via a pulling motion.

In one or more embodiments of the system, wherein at least a portion of the removable sealing member of the device is located through a wall of the housing.

In one or more embodiments, wherein at least a portion of the removable sealing member of the device is located through a side wall of the housing.

In one or more embodiments of the system, wherein at least one of an antibacterial substance, an antiviral substance, and an antifungal substance is located inside the housing of the device, on a surface of the removable sealing member, on the wiping member, and combinations thereof.

In one or more embodiments of the system, wherein the securing mechanism of the distal end portion of the housing of the device is further configured to be movable between a first extended position and a second contracted position.

In one or more embodiments of the system, wherein when the securing mechanism of the distal end portion of the housing of the device is in the second contracted position the second sealing member of the connection port is positioned to be wiped off by the wiping member of the device.

In one or more embodiments of the system, wherein when the device is coupled to the connection port and when the securing mechanism of the device is in a first extended position, an exterior surface of the second sealing member of the connection port is not aligned with an interior surface of the housing, and wherein when the device is coupled to the connection port and the securing mechanism of the device is in the second contracted position, an exterior surface of the second sealing member of the connection port is aligned with an interior surface of the housing.

In one or more embodiments of the system, wherein when the exterior surface of the second sealing member of the connection port is aligned with an interior surface of the housing, the wiping member is able to wipe off the exterior surface of the second sealing member of the connection port when the wiping member moves across the housing.

In one or more embodiments of the system, wherein the distal end portion of the housing of the device is further configured to be movable between a first extended position and a second contracted position.

In one or more embodiments, wherein the securing mechanism of the distal end portion of the housing of the device is selected from a group consisting of a thread, ratchet teeth, a clamping member, a retention member, and an adhesive.

In one or more embodiments of the system, the housing further comprising a guiding member configured to align the removable sealing member of the housing with the removable sealing member of the connection port.

In one or more embodiments, the guiding member is a rod.

In one or more embodiments, the guiding member is a dent.

In one or more embodiments, the guiding member is a slot.

In one or more embodiments, the guiding member is a protrusion.

In one or more embodiments of the system, the guiding member is configured to align the removable sealing member of the housing of the device with the removable sealing member of the connection port, wherein upon alignment of the removable sealing member of the housing of the device with the removable sealing member of the connection port both the removable sealing member of the housing and the removable sealing member of the connection port will displace in about the same direction and at about the same time external to the location of fluidic communication between the housing and the connection port.

In one or more embodiments of the system, the connection port has a guiding member configured to align the removable sealing member of the connection port with the removable sealing member of the device when the connection port couples with the device.

In one or more embodiments of the system, the guiding member of the connection port is a rod.

In one or more embodiments of the system, the guiding member of the connection port is a dent.

In one or more embodiments of the system, the guiding member of the connection port is a slot.

In one or more embodiments of the system, the guiding member of the connection port is a protrusion.

In one or more embodiments of the system, wherein the device further comprising a guiding member configured to align the removable sealing member of the device with the removable sealing member of the connection port.

In one or more embodiments of the system, wherein the connection port further comprising a guiding member configured to align the removable sealing member of the device with the removable sealing member of the connection port.

In one or more embodiments of the system, wherein the device further comprising a guiding member and wherein the connection port further comprising a guiding member, wherein both the guiding member of the device and the guiding member of the connection port are configured to engage with each other in such a manner as to align the removable sealing member of the device and the removable sealing member of the connection port so that both said removable sealing members are able to displace at about the same time and in the same direction when moved to a second unsealed position.

In one or more embodiments of the system, wherein the guiding member of the device is a rod and the guiding member of the connection port is a complimentary slot which allows the rod to enter into the slot.

In one or more embodiments of the system, wherein the guiding member of the connection port is a rod and the guiding member of the device is a complimentary slot which allows the rod to enter into the slot.

In one or more embodiments of the system, wherein the guiding member of the device is a protrusion and the complimentary guiding member of the connection port is a dent, wherein the dent allows the protrusion to engage and/or enter into the dent.

In one or more embodiments of the system, wherein the guiding member of the connection port is a protrusion and the complimentary guiding member of the device is a dent, wherein the dent allows the protrusion to engage and/or enter into the dent.

In one or more embodiments, the proximal end portion of the housing is integrally attached to a container and the distal end portion of the housing having a guiding member.

In one or more embodiments, the wiping member is configured to extend from the top wall of the housing to the bottom wall of the housing.

In one or more embodiments, the wiping member is configured to provide a hermetic seal between the top wall and the wiping member and the bottom wall and the wiping member when the wiping member moves across the housing.

In one or more embodiments, the wiping member is configured to wipe off a surface of the first sealing member of the housing when the wiping member moves across the housing.

In one or more embodiments, a filter is disposed in, on, or over an aperture in the housing for releasing air from the housing when actuating the wiping member.

In one or more embodiments, the housing is manufactured from a group consisting of plastic, rigid plastic, flexible plastic, polymer, and combinations thereof.

In one or more embodiments, the housing is made of a glass material, a metal material, and combinations thereof.

In one or more embodiments, the wiping member is made of a plastic substance, a rubber substance, an elastomeric substance, and combinations thereof.

In one or more embodiments, the wiping member is covered and/or saturated with an antibacterial substance, an antiviral substance, an anti-tuberculin substance, an anti-HIV substance, hydrogen peroxide, isopropyl alcohol, and combinations thereof.

In one or more embodiments, the device and/or housing is disposable and/or for single use.

In one or more embodiments, a surface of the removable sealing member has an adhesive on the surface of the removable sealing member.

In one or more embodiments, an exterior surface of the removable sealing member has an adhesive.

In one or more embodiments, the device further comprises a sealing ring.

In one or more embodiments, the sealing ring of the device is an “O-ring”.

In one or more embodiments, the sealing ring is a circumferential sealing ring.

In one or more embodiments, the “O-ring” is a circumferential “O-ring”.

In one or more embodiments, the sealing ring of the device is made of at least one of a plastic material and a rubber material.

In one or more embodiments, the sealing “O-ring” of the device is made of at least one of a plastic material and a rubber material.

In one or more embodiments, the connection port further comprises a sealing ring.

In one or more embodiments, the sealing ring of the connection port is an “O-ring”.

In one or more embodiments, the sealing ring of the connection port is a circumferential sealing ring.

In one or more embodiments, the “O-ring” of the connection port is a circumferential “O-ring”.

In one or more embodiments, the sealing ring of the connection port is made of at least one of a plastic material and a rubber material.

In one or more embodiments, the sealing “O-ring” of the connection port is made of at least one of a plastic material and a rubber material.

In one or more embodiments, both the device and the connection port further comprise a sealing ring.

In one or more embodiments, the sealing ring of the device and the sealing ring of the connection port is an “O-ring”.

In one or more embodiments, the sealing ring of the device is a circumferential sealing ring and the sealing ring of the connection port is a circumferential sealing ring.

In one or more embodiments, the “O-ring” of the device is a circumferential “O-ring” and the “O-ring of the connection port is a circumferential “O-ring”.

In one or more embodiments, the sealing ring of the connection port is made of at least one of a plastic material and a rubber material and the sealing ring of the device is made of at least one of a plastic material and a rubber material.

In one or more embodiments, the sealing “O-ring” of the device and the sealing “O-ring” of the connection port are made of at least one of a plastic material and a rubber material.

In one or more embodiments, the device further comprising teeth configured to engage/couple holes/dents of a connection port.

In one or more embodiments, the teeth of the device are retractable teeth.

In one or more embodiments, the teeth are positioned at a distal end portion of the device.

In one or more embodiments, the retractable teeth are positioned at a distal end portion of the device.

In one or more embodiments, the teeth are positioned on an engagement mechanism of the device.

In one or more embodiments, the teeth are positioned on a securing mechanism of the device.

In one or more embodiments, the teeth are positioned on the device.

In one or more embodiments, the teeth are positioned on the housing of the device.

In one or more embodiments, the retractable teeth are positioned on the device.

In one or more embodiments, the retractable teeth are positioned on the housing of the device.

In one or more embodiments, the teeth are configured to engage/couple a hole/dent of a connection port.

In one or more embodiments, the retractable teeth are configured to engage/couple a hole/dent of a connection port.

In one or more embodiments, the teeth are configured to provide coupling of a connection port to the device, the teeth are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the device further comprising holes/dents configured to engage/couple with teeth of a connection port.

In one or more embodiments, the device further comprising holes/dents configured to engage/couple with retractable teeth of a connection port.

In one or more embodiments, the holes/debts are positioned at a distal end portion of the device.

In one or more embodiments, the holes/dents are positioned on an engagement mechanism of the device.

In one or more embodiments, the holes/dents are positioned on a securing mechanism of the device.

In one or more embodiments, the holes/dents are positioned on the device.

In one or more embodiments, the holes/dents are positioned on the housing of the device.

In one or more embodiments, the holes/dents are configured to engage/couple teeth of a connection port.

In one or more embodiments, the holes/dents are configured to engage/couple retractable teeth of a connection port.

In one or more embodiments, the holes/dents are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments of the system, the device further comprising teeth and the connection port further comprising holes/dents.

In one or more embodiments of the system, the device further comprising retractable teeth and the connection port further comprising holes/dents configured to engage/couple the retractable teeth.

In one or more embodiments of the system, the device further comprising holes/dents and the connection port further comprising teeth.

In one or more embodiments of the system, the device further comprising holes/dents and the connection port further comprising retractable teeth.

In one or more embodiments, the holes/dents of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the holes/dents of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the teeth of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the retractable teeth of the device are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the teeth of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the retractable teeth of the connection port are configured to provide coupling of a connection port to the device, the holes/dents are further configured to provide a connection port to couple to the device in a first extended position in which a second sealing member of the connection port is not aligned with an interior wall of the device and a second contracted position in which the second sealing member of the connection is aligned with an interior wall of the device.

In one or more embodiments, when the connection port is in a second contracted position when attached to the device, the second sealing member of the connection port is aligned with an interior wall of the device and the second sealing member is positioned to be wiped off by the wiping member of the device when the wiping member is moved across the device.

In one or more embodiments, the housing of the device further comprising a plug disposed in an opening located at the distal end portion of the device, the plug configured to fluidly seal the opening of the distal end portion of the device.

In one or more embodiments, the plug is selected from a stopper, a bung, a rubber stopper, and a rubber bung.

In one or more embodiments, the plug is pierceable.

In one or more embodiments, the plug is frangible.

Unless otherwise defined, all technical or/and scientific terms used herein have the same meaning as commonly understood by one of ordinary skill in the art to which the invention pertains. Although methods and materials similar or equivalent to those described herein can be used in the practice or testing of embodiments of the invention, exemplary methods or/and materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1F are schematic illustrations presenting an exemplary decontamination system which comprises a device having a removable sealing member and a wiping member, and a connection port having a removable sealing member for allowing a decontaminated and/or sterile communication between the device and the connection port, according to some embodiments of the invention.

FIGS. 2A-2D are schematic illustrations presenting an exemplary decontamination system with an engagement mechanism in the form of ratchet teeth for connecting between vessels, the system further comprises removable sealing members and a wiping member, for allowing a decontaminated communication between a first vessel and a second vessel, according to some embodiments of the invention.

FIGS. 3A-3D are schematic illustrations presenting an exemplary decontamination system with an engagement mechanism in the form of clamping arms for connecting between vessels, the system further comprises removable sealing members and a wiping member, for allowing a decontaminated communication between a device attached to a first vessel and a connection port attached to a second vessel, according to some embodiments of the invention.

FIGS. 4A-4D are schematic illustrations presenting an exemplary decontamination system which comprises connection interfaces that extend between an internal location within a housing of a device and an external position, and a wiping member, for allowing a decontaminated communication between a device and a second vessel, according to some embodiments of the invention.

FIGS. 5A-5D are schematic illustrations presenting an exemplary decontamination system which comprises an engagement mechanism in the form of threads for connecting between vessels, the system further comprises removable sealing members that extend between an internal location within a device and an external position, and a wiping member disposed inside the device, for allowing a decontaminated communication between a the device and a connection port, according to some embodiments of the invention.

FIGS. 6A-6D are schematic illustrations presenting an exemplary decontamination system which comprises a housing having a removable sealing member and a wiping member, and a locking mechanism for locking a wiping member, according to some embodiments of the invention.

FIGS. 7A-7F are schematic illustrations presenting an exemplary decontamination system which comprises removable sealing members and a wiping member located inside a housing of a device, for allowing a decontaminated communication between a first vessel and a second vessel via the device, the vessels can engage with each other via a push motion of one or both vessels towards each other, according to some embodiments of the invention.

FIGS. 8A-8F are schematic illustrations presenting an exemplary decontamination system which comprises removable sealing members located on a device having a wiping member and also located on a connection port of a vial/bottle, for allowing a decontaminated communication between a first vessel and the connection port of the vial/bottle. The vessels can engage with each other via a twist or push-snap motion of one or both vessels towards each other, according to some embodiments of the invention.

FIGS. 9A-9B are schematic illustrations presenting an exemplary decontamination system which comprises a lock mechanism for locking a first and a second vessels, and removable sealing members and a wiping member, for allowing a decontaminated and/or sterilized communication between the vessels, according to some embodiments of the invention.

FIGS. 10A-10F are schematic illustrations presenting an exemplary decontamination system which comprises a locking member for preventing return of a wiping member to its original location, according to some embodiments of the invention.

FIGS. 11A-11F are schematic illustrations presenting an exemplary decontamination system which comprises folded removable sealing members and a wiping member, for allowing a decontaminated communication between a device attached to a first vessel and a connection port attached to a second vessel, according to some embodiments of the invention.

FIG. 12 is a schematic illustration presenting an exemplary connector which comprises two removable sealing members and a wiping member, for allowing a decontaminated communication between any of a variety of first vessels and an of a variety of second vessels, according to some embodiments of the invention.

FIGS. 13A-13F are schematic illustrations presenting an exemplary decontamination system which comprises removable sealing members and a wiping member, for allowing a decontaminated communication between a device and a connection port attached to a second vessel, the system further includes a sealing ring for maintaining a sealed communication between the vessels following an external displacement of the removable sealing members, according to some embodiments of the invention.

FIGS. 14A-14B are schematic top view illustrations presenting a removable sealing member which covers an opening of a device and/or connection port with a sealing ring of the device and/or the connection port, according to some embodiments of the invention.

FIGS. 15A-15F are schematic illustrations presenting an exemplary decontamination system which comprises folded removable sealing members and a wiping member located inside a device, for allowing a decontaminated communication between a device attached to a first vessel and a connection port attached to a second vessel, according to some embodiments of the invention.

### DETAILED DESCRIPTION OF THE INVENTION

It is understood that the invention is not limited to the particular methodology, devices, items or products etc., described herein, as these may vary as the skilled artisan will recognize. It is also to be understood that the terminology used herein is used for the purpose of describing particular embodiments only and is not intended to limit the scope of the invention. The following exemplary embodiments may be described in the context of exemplary decontamination devices for ease of description and understanding. However, the invention is not limited to the specifically described products and methods and may be adapted to various applications without departing from the overall scope of the invention. All ranges disclosed herein include the endpoints. The use of the term “or” shall be construed to mean “and/or” unless the specific context indicates otherwise.

The system, device, and corresponding method disclosed herein allow a user (e.g., a pharmacist, nurse, doctor or other healthcare provider) to intermix at least two substances while maintaining sterility of vessel surfaces and sterility of a fluid passageway between the vessels while preventing unwanted release of the substance into the environment.

The type, size, shape, and material of the devices and connection ports are not critical features of the invention. Nor are the containers' contents. The invention is appropriate for all types of contents//medicaments/beneficial substances that require sterility of the contents/medicaments/beneficial substances be preserved. As described more fully below, the containers, devices, housings, and connection ports should have structures and/or features that allow for the sterile engagement between a device and a connection port to provide an airtight and decontaminated/sterile fluid communication/passageway between the device and the connection port.

The present invention relates to devices, methods and systems allowing an engagement of medical containers, devices, and connection ports in a sterile manner. In one embodiment, the present invention provides removable sealing members, that may be coupled to, or integrally formed with a container, a decontamination device, a device having a wiping member, and/or other vessels (such as vials, syringes, filters, infusion lines, etc.). The invention provides a solution to an unmet and long felt need in the medical setting and allows connecting in a sterile manner a device and connection port. The herein disclosed containers, devices, connection ports, systems, and methods are user friendly, cost effective and abolish the need for complicated and expensive known methods for transferring medical substances in a sterile manner. In an embodiment of the invention, the herein disclosed devices, connection ports, and systems are disposable. In an embodiment of the invention, the herein disclosed devices, connection ports, and systems are non-disposable. In an embodiment of the invention, the herein disclosed devices and connection ports are intended for one-time use. In an embodiment of the invention, the herein disclosed devices and connection ports are intended to be reused.

In an aspect of the invention, the devices and systems include a first removable sealing member attached to a first vessel and a second removable sealing member attached to a second vessel. A removable sealing member may be connected to a decontamination device having a housing with a wiping member. The first and second removable sealing members are configured to allow an engagement therebetween the removable sealing members. When the removable sealing members engage each other, ambient air particles present at the vicinity thereof are entrapped within/between the engagement formed by the removable sealing members. The engagement between the removable sealing members further establishes an airtight engagement of the vessels. The removable sealing members are further configured to externally displace from the vessels, thereby allowing fluid flow between the vessels and contaminants-free connection of the vessels. In one or more embodiments, either or both of the vessels are container(s). The containers may be a bag, a bottle, a vial and/or a syringe. In one or more embodiments the vessel may be a filter, an infusion line, or any other medicinal/medical device that stores, holds, or transfers beneficial substances/fluids.

The invention further provides a decontamination device having a housing with a wiping member operable to wipe off a surface of a vessel and decontaminate thereof. The decontamination devices may include a first removable sealing member and may be connected to a first vessel, allowing an engagement with a second vessel via a second removable sealing member. Once engaged, the removable sealing members may be pulled out by a user, maintaining a hermetically sealed connection between the vessels and via the housing of the decontamination device. The wiping member may be further actuated to wipe off a surface of the second vessel and of the housing, rendering a decontaminated fluid passageway between two vessels.

Accordingly, the herein disclosed invention allows transferring medical substances in a contaminants-free, or in a substantially contaminants-free manner. In one or more embodiments, the herein disclosed invention allows connecting vessels in a contaminants-free, or in a substantially contaminants-free manner.

In one or more embodiments, the herein disclosed invention provides a fluidic passageway or communication between medical containers in a contaminants-free, or in a substantially contaminants-free manner.

In one or more embodiments, the herein disclosed invention affords to isolate and/or entrap ambient air particles present between two, or more, vessels. In one or more embodiments, the herein disclosed invention, allows to substantially decrease the chances to introduce a contamination within a medical substance, when preparing or administering medical substances.

In one or more embodiments, the herein disclosed invention affords to isolate and/or entrap ambient air particles present between a device having a wiping member disposed inside the device and a connection port. In one or more embodiments, the device may be attached to a vessel such as a container, a bag, a bottle, a vial, a syringe, and the alike and the connection port may be attached to a vessel such as a container, a bag, a bottle, a vial, a syringe, and the a like. In one or more embodiments, the herein disclosed invention, allows to substantially decrease the chances to introduce contamination within a medical substance (such as a medicament, diluent), when preparing, or transferring, or administering medical/medicinal substances. In one or more embodiments, the herein disclosed invention allows the sterile fluid transfer of a beneficial substance between a device and a connection port.

As used herein, the term “substances” refers to various types of materials that should be kept sterile. The substances may be liquid, solid, semi-solid, or gas. In one or more embodiments, the substances are “medical substances”. As used herein the term “medical substances” refers to and encompasses any of the various medical drugs, fluids, nutritional products, solid powders, injectable medicaments, suspensions, liquids solutions and the like.

As used herein, the term “contaminant-free” is interchangeable with the term “sterile”, “disinfected”, and “decontaminated”. The term refers to substances that are free or substantially free of ambient air particles and/or pathogens, including but not limited to dirt, bacteria, viruses, funguses, spores, and/or pyrogens. Typically, when less or no air is introduced within medical substances, the chances of contamination by pathogens, such as, bacteria, viruses, funguses, spores, pyrogens or the alike is completely abolished or significantly reduced.

As used herein, “substantially contaminants-free” means significantly less ambient air present when transferring medical substances with the herein disclosed vessels and systems, as compared to comparable conditions for transferring medical substances without the herein disclosed vessels and systems.

As used herein, the term “ambient air particles” is interchangeable with the term “environmental air particles” and refers to air particles present in a non-filtered or filtered environment. For example, air can be purified by filters, such as a High Efficiency Particulate Air (HEPA) filter.

As used herein, the term “fluidly seal” and/or “fluidly sealing” is interchangeable with the term “substantially fluidly seal” and/or “substantially fluidly sealing” and refers to the sealing of an opening of a housing of a device and/or sealing an opening of a connection port in a manner that prevents a fluid from entering, exiting, and/or entering and exiting the interior of the housing of the device and/or the interior of the connection port. In one example, “fluidly sealing” and/or the term “substantially fluidly sealing” means covering an entire opening of a device thus preventing entry and/or exit of ambient air particles/contaminants into and/or out of the interior of the device. The device may have a wiping member disposed inside the device. In another example, “fluidly sealing” and/or the term “substantially fluidly sealing” means covering an entire opening of a connection port thus preventing entry and/or exit of ambient air particles/contaminants into and/or out of the connection port. In still another example, the term “fluidly seal”, “fluidly sealing”, “substantially fluidly seal”, and/or “substantially fluidly sealing” refers to hermetically/airtight sealing an opening of a device and/or hermetically/airtight sealing an opening of a connection port.

As used herein, the term “removable sealing member” is used interchangeably with the term “connection interface” and encompasses any surface, layer plane or the alike that can be attached to a vessel/device/connection port and removed from a vessel/device/connection port. The term may encompass a structure that can be coupled to a vessel/device/connection port and that can engage with a complementary connection interface/removable sealing member. As used herein, the term “removable sealing member” is meant to mean “connection interface”. As used herein, the term “connection interface” is meant to mean “removable sealing member”. In an embodiment of the invention, the connection interface/removable sealing member may seal or cover an aperture of a vessel. In an embodiment of the invention, the connection interface may seal or cover an opening for fluid passageway of a vessel or a device or a connection port. In an embodiment of the invention, the connection interface may be a surface of a container. In an embodiment of the invention, the connection interface may be a surface of a device. In an embodiment of the invention, the connection interface may be a surface of a vessel. In an embodiment of the invention, the connection interface/removable sealing member may cover another sealing member. In an embodiment of the invention, the connection interface abuts a container, a device, and/or a connection port. In an embodiment of the invention, the connection interface is surface mounted to a container, a device, and/or a connection port. In an embodiment of the invention, the connection interface is surface mounted to a wall of the container, a device, and/or a connection port. In an embodiment of the invention the connection interface is flush mounted to a container, a device, and/or a connection port.

In an embodiment of the invention, the connection interface abuts a device and/or a connection port. In an embodiment of the invention, the connection interface is surface mounted to a device and/or a connection port. In an embodiment of the invention, the connection interface is surface mounted to a wall of a device and/or a connection port. In an embodiment of the invention the connection interface is flush mounted to a device and/or a connection port.

In an embodiment of the invention, the connection interface abuts a vessel. In an embodiment of the invention, the connection interface is surface mounted to a vessel. In an embodiment of the invention, the connection interface is surface mounted to a wall of a vessel. In an embodiment of the invention the connection interface is flush mounted to a vessel. In an embodiment of the invention, the connection interface is flush mounted to a wall of a vessel.

In an embodiment of the invention, the present invention discloses a system comprising a removable sealing member attached to a device, the device having a wiping member disposed inside the device and the wiping member configured to move across the device, and a second removable sealing member attached to a connection port. The first removable sealing member and the second removable sealing member may be similar or may be different in size and/or shape. In one or more embodiments, the removable sealing member of the device and the removable sealing member of the connection port may be about the same size/surface area/dimensions. The exterior surface area of the removable sealing member faces ambient air particles/contaminants. In one or more embodiments, the exterior surface area facing ambient air, of the removable sealing members may be non-equal. In one or more embodiments, the difference in surface area between the exterior surface area of a plurality of connection interfaces is less than 0.3 inches squared. For example, less than 0.2 inches squared, or less than 0.1 inches squared.

In an embodiment of the invention, the first removable sealing member of the device and the second removable sealing member of the connection port when engaged with each other (together) entrap ambient air particles therebetween. In an embodiment of the invention, the first removable sealing member of the device and the second removable sealing member of the connection port when engaged with each other externally displace from the device and the connection port. In one or more embodiments, the term externally displace means displacing from either or both the device and the connection port.

In one or more embodiments, the removable sealing member(s) has a thin film layer. In an exemplary embodiment, the removable sealing member may present a thickness of about 1 mm or below/less. Alternatively, the removable sealing member may present a thickness of above 1 mm. The removable sealing member may be manufactured from various materials, including, an elastic polymer. The removable sealing member may be made from a plastic material. The removable sealing member may be a rubber or a polymer. The removable sealing member may be formed of a metal material (e.g., a metal alloy). In yet a further exemplary embodiment, the removable sealing member may preset the form of a thin film made of an aluminum foil, cling wrap, or the alike. The metal alloy may include, for example, aluminum. In yet a further exemplary embodiment, the removable sealing member may preset the form of a thin film made of an aluminum foil, or the alike. In one or more embodiments, the removable sealing member is attached to, or is integrally formed with a device, a connection port, a container, and/or a vessel. In one or more embodiments, the removable sealing member is peelable from the vessels. In one or more embodiments, the peelable removable sealing member is attached to a vessel via an adhesive. In one or more embodiments, the peelable removable sealing member may be detached from a vessel surface by the force of a user's hand. In one or more embodiments, the removable sealing member is movable or slidable along a rail located along a length of the aperture of a vessel. In one or more embodiments, the first and second removable sealing members may be attached to or engage each other via various mechanisms. For example, the removable sealing members may be engaged via an engagement mechanism, such as a thread, a luer, an adhesive, a mechanical object, a dent and a ratchet teeth mechanism. In one or more embodiments, the removable sealing member may be covered by a cover (not shown). In one or more embodiments, the cover may be a peelable cover. In one or more embodiments, a sterilizing or disinfecting substance may be located between the removable sealing member and the cover. In one or more embodiments, the sterilizing or disinfecting substance may be an alcohol. In one or more embodiments, the alcohol may be 70% isopropyl alcohol.

The removable sealing member may be coupled within any portion of the device and/or connection port. For example, the removable sealing member may be connected onto the device's body or aperture, the device's wall or port thereof. In another example, the removable sealing member may be connection onto the connection port's body or aperture, the connection port's wall or port thereof. The removable sealing member may be attached/coupled to an exterior surface of the device or connection port, to an interior surface of the device or connection port, and/or to both an exterior and interior surface of the device or connection port. In one or more embodiments, the removable sealing member of the device and/or connection port may be configured in a folded position that facilitates the peeling off of the removable sealing member from the device and/or the connection port.

In one or more embodiments, the first removable sealing member and the second removable sealing member are configured to prevent contaminants from entering the fluid passageway between a device and a connection port. In one or more embodiments, the first removable sealing member attached to the device and the second removable sealing member attached to the connection port are configured to prevent contaminants from entering the interior of the device, the interior of the housing of the device, and/or the interior of the connection port. In one or more embodiments, the first removable sealing member and the second removable sealing member are configured to prevent contaminants from entering a fluid housed inside a vessel's body, wherein the vessel is connected/attached to either or both the proximal end of the device and/or the distal end of the connection port.

In one or more embodiments, a first removable sealing member and is configured to prevent contaminants from contacting an internal compartment of the device, the inside/interior of the housing, and/or the interior of the first vessel attached to the proximal end portion of the device. In one or more embodiments, a second removable sealing member is configured to prevent contaminants from contacting an internal compartment of the connection port, the inside/interior of a second vessel attached to the distal end portion of the connection port.

As used herein the term “external displacement” refers to a displacement (i.e., dislocation) of the herein disclosed first and/or second, and/or any additional removable sealing member/connection interfaces. In an embodiment of the invention, the displacement is external, namely, outside the device and/or connection port and/or container(s) and/or vessels being connected by the herein disclosed system. In an embodiment of the invention, the displacement is external to the fluid communication established following engagement of the device with the connection port being connected by the herein disclosed system, methods, devices, and connection ports. In an embodiment of the invention, the displacement maintains a hermetic seal between the device and the connection port. In an embodiment of the invention, the external displacement maintains an airtight seal between the housing of the device and the connection port. The displacement may occur via a sliding motion, or by a pulling out motion, or by peeling the removable sealing members. In an embodiment of the invention, the displacement occurs for both the removable sealing member of the housing of the device and the removable sealing member of the connection port. The external displacement may optionally occur simultaneously for both removable sealing members or may at about the same time for both the removable sealing members. In an embodiment of the invention, the external displacement establishes a fluid passageway between two or more vessels. In one or more embodiments, the removable sealing member is configured to hermetically seal an aperture present in a housing of the device. In one or more embodiments, the removable sealing member is configured to hermetically seal an aperture present in a connection port. In one or more embodiments, the removable sealing member is configured to cover a second sealing member of the connection port. In one or more embodiments, the removable sealing member of the connection port is configured to hermetically seal the second sealing member of the connection port within the connection port. In one or more embodiments, at about the time of external displacement of the removable sealing members, the aperture through which the removable sealing members pass through reseals, allowing a hermetic airtight connection between the housing of the device and the connection port. In one or more embodiments, after external displacment of the removable sealing members, the aperture through which the removable sealing members pass through reseals, allowing a hermetic airtight connection between the device and the connection port. In one or more embodiments, the aperture through which the removable sealing members pass through reseals prior to detachment of the removable sealing members from the housing of the device and the connection port.

As used herein the term “vessel” refers to any device utilized for containing, housing or transferring substances as herein disclosed. In one or more embodiments, the vessel may be used for containing medical substances. In an embodiment of the invention, the vessel is a medical vessel. In an embodiment of the invention, the vessel is a medical device. In an embodiment of the invention, the vessels are used for, and adapted to allow connection to another vessel(s). In an embodiment of the invention, the vessel may be a medical container utilized for accommodating medical substances. Various types of medical containers are contemplated. The medical container may be selected, without limitation, from a vial, a bag, a chamber, a housing, a bottle, and the alike. In an embodiment of the invention, the term vessel further encompasses elements that can be used to connect between vessels. In accordance with this embodiment, the vessel may be selected, without limitation, from a connector, a port, a syringe, an infusion line, a tubing, a syringe, a filter, a spike, a port and a manifold. In an embodiment of the invention, one or more connection interfaces (for example, two or more, three or more, etc.) may be coupled to a first vessel and each of those connection interfaces may be coupled and engage with a one or more connection interfaces present on a one or more vessels. In one or more embodiments, the vessel may be a container, a bag, a bottle, a connector, an infusion line or a syringe having a plurality of externally displaceable connection interfaces. In one or more embodiments, the vessels to be engaged may have similar surface area or similar contact surface area (i.e., surface onto which the connection interface is coupled to). For example, a first vessel and a second vessel may have similar surface area or similar contact surface area. In one or more embodiments, the vessels to be engaged may have different surface area or different contact surface area. For example, a first removable sealing member attached to a first vessel may have a greater surface area or contact surface area than a second removable sealing member attached to a second vessel. As used herein the term “fluid communication” is used interchangeably with the term “fluidic passageway” and refers to a device and a connection port in which substances may pass therethrough either directly or indirectly. The fluid communication may occur via a fluid passageway through the device and the connection port that allows for the flow/transfer of a medical substance from the device to the connection port, flow/transfer of a medical substance from the connection port to the device, and/or flow/transfer of a medical substance between the connection port and the device.

As used herein, the term “guiding member” may be any type of mechanical object that guides either or both a connection port and a device to engage/couple each other (one another) in a manner in which a removable sealing member of the connection port aligns with the removable sealing member of the device so that the removable sealing member of the connection port lies against, optionally lies flat against, the removable sealing member of the device. The “guiding member” further aligns in the same direction at least one of the removable sealing member of the device and the removable sealing member of the connection port so that the tabs, optionally the portion of the removable sealing member(s) that a user pulls, will remove both removable sealing members (of the device and the connection port) at about the same time, optionally at the same time. The guiding member is characterized in that it directs an engagement between a device and a connection port in a manner that aligns the removable sealing member of the device with the removable sealing member of the connection port with the purpose of facilitating at about the same time, optionally at the same time, the external displacement of both the removable sealing member of the device and the removable sealing member of the connection port. The size, shape, and configuration of the guiding member and corresponding engagement member that engages/couples the guiding member is not important. The focus is the alignment of removable sealing members wherein the tabs are positioned in about the same direction, optionally in the same direction, that facilitates concurrent displacement of two removable sealing member from a first sealed position to a second unsealed position. The guiding member in one example may be a rod that engages/couples a complimentary hole/aperture in a on a device or connection port. Conversely, the guiding member may be a hole/aperture that engages/couples a complimentary rod. Either or both a device and a connection port may have the guiding member or complimentary engagement/coupling mechanism. The guiding member may be located on a device, on a device having a removable sealing member, on a device having a removable sealing member and a wiping member, on a connection port, on a vessel attached to any of said devices, on a container, on a container attached to any of said device, on a container attached to the connection port, and combinations thereof. The guiding member may have any type of geometric shape that will engage/couple a complimentary geometric shape of an engagement/coupling member configured to engage/couple the guiding member. The guiding member may be a thread, a luer-lock, a luer, a snap-on mechanism, a slide-on mechanism, etc. A complimentary engagement mechanism is provided to engage/couple the guiding member. In one example, the guiding member is located on an exterior of a device and the complimentary engagement mechanism is located on a connection port. In another example, the guiding member is located on an exterior of a connection port and the complimentary engagement mechanism is located on the device. In another example, both a device and a connection port may both have guiding members configured to couple a complimentary engagement/coupling member located on both the device and the connection port.

In an aspect of the invention, a container is provided having an external displacement mechanism and an internal displacement mechanism. The external displacement mechanism being a removable sealing member configured to be displaced form a first sealed position to a second unsealed position and a plug/stopper/bung configured to displace into the interior of the container or configured to displace to an exterior of the container.

In a further aspect of the invention, a system is provided comprising a first container and a second container. The first container is provided having an external displacement mechanism in the form of a removable sealing member configured to be displaced to a second unsealed position (displaced external to the fluid passageway that is formed between the first and second containers), the first container also having a displaceable plug/bung/stopper with an engagement mechanism, the engagement mechanism configured to engage/couple a second engagement mechanism of a displaceable plug/bung/stopper of a second container; and a second container configured to engage the first container, the second container having an external displacement mechanism in the form of a removable sealing member configured to be displaced to a second unsealed position (displaced external to the fluid passageway that is formed between the first and second container), the second container also having a displaceable plug/bung/stopper with an engagement mechanism configured to engage/couple with the displaceable plug/bung/stopper of the first container, wherein when the first container engages/couples with the second container, the removable sealing members of both the first and second containers are displaced from a first sealed position to a second unsealed position and the displaceable plugs of both the first and second containers engage/couple with each other and displace into the interior of the first container or into the interior of the second container. In accordance with this embodiment, the displacement of both removable sealing members to a second unsealed position decontaminates the connection between the first and second containers after which the displaceable plugs/bungs/stopper of the first and second container engage with each other and displace internally into either the first or second container thus establishing a decontaminated and sterile fluidic passageway between the first and second containers. The first container can be a bag, bottle, vial, etc. The second container can be a bag, bottle, vial, etc. The first container can be a bag and the second container can be a bottle/vial.

In an aspect of the invention, the devices and systems of the invention include a housing having a side wall, a top wall and a bottom wall, and a wiping member disposed within the housing. In an embodiment of the invention, the top wall and/or bottom wall of the housing have at least one opening(s) for receiving the vessels. The opening(s) may be formed by vertical walls extending from the top and/or bottom walls, forming an inner channel via which medical substances may be transferred following engagement between, and decontamination of the vessel surfaces. In one or more embodiments, the openings have non-vertical walls. In one or more embodiments, the openings may be flush mounted to the housing. In one or more embodiments, the openings may be surface mounted to the housing. In one or more embodiments, the openings may be concealed within at least a portion of the housing. In one or more embodiments, the openings may be concealed within the housing.

In an embodiment of the invention, the wiping member is operable to move across and wipe off any contaminates present on the one or more surfaces of the vessels via an actuator extending through an opening of the housing. In an embodiments of the invention, the wiping member is operable to move across the housing of the device and wipe off contaminants present on one or more surfaces of the interior of the housing of the device and/or a surface of a connection port which is attached/coupled to the housing of the device. The actuator is configured to manipulate movement of the wiping member across the housing. In one or more embodiments, the wiping member is operable to move across and wipe off any contaminants present on the one or more surfaces of the housing and the connection port via an actuator extending through an opening in a wall of the housing. The actuator may extend through a side wall of the housing, through a top wall of the housing, and/or through a bottom wall of the housing. In one or more embodiments, the actuator may extend through a top or bottom wall of the housing. In one or more embodiments, the wiping member is operable to move within the housing to wipe off any contaminants present on the one or more surfaces of the vessels. In one or more embodiments, the wiping member quarantines any contaminants on one side/end of the housing of the device thus providing a decontaminated and/or sterile fluid passageway within the housing of the device.

Various types of actuators are contemplated for allowing manipulation of the wiping member to decontaminate a surface of a housing, connection port, and/or a vessel. In one or more embodiments, the actuator is connected to the wiping member and extends externally via an opening within the housing. Exemplary actuators include, without limitation, a handle, a tab, a button, a touch button, a lever, a gear, a spring, a mechanical actuator, an electric actuator, an air pressure actuator, and combinations thereof. In one or more embodiments, the wiping member is operable to move across and wipe off any contaminates present on the one or more surfaces of the housing, of the connection port, and/or of the vessels without any actuator for mediating the wiping action. In accordance with this embodiment, a user may, for example, apply force onto the housing to thereby forcibly manipulate the wiping member to slide across the housing. In accordance with this embodiment, the housing may be flexible, so the user can manipulate the wiping member from the outside and by applying for example press forces onto the housing. In one or more embodiments, the wiping member is moveable from an initial position which precedes the wiping action to an end position which follows the wiping action of the wiping member. In one or more embodiments, the movement of the wiping member between the positions is reversible. In one or more embodiments, the movement of the wiping member between the positions is non-reversible and the wiping member is prevented from returning to the initial position. In one or more embodiments, a locking member may be disposed in or on the housing to prevent the wiping member to return to its initial position. In one or more embodiments, the locking member locks the wiping member in a second position after the wiping member has wiped off a surface of the housing and a surface of the connection port.

In an embodiment of the invention, the herein disclosed devices and systems are disposable. In an embodiment of the invention, the herein disclosed devices and systems are non-disposable. In an embodiment of the invention, the herein disclosed devices and/or systems are intended for single use.

In one or more embodiments, the wiping member is manufactured from or comprises a material including, but not limited to an elastic material, a rigid material, a flexible material, an expandable material, a polymer, and combinations thereof. In one or more embodiments, the wiping member is manufactured from or comprises an elastomeric material, such as, but not limited to a rubber material. In one or more embodiments, the wiping member is manufactured from or comprises a sponge or a sponge-like material. In one or more embodiments, the wiping member slides across the surface of the one or more vessel(s) under great friction, or under a friction sufficient to wipe off any contaminants from the surface, such as dirt or bacteria.

In one or more embodiments, the wiping member is covered, at least partially, by a sterilizing substance. As used herein the term “sterilizing substance” encompasses any substance that can eliminate or reduce the presence of microorganisms, air particles, or any of the alike. In one embodiment, the term “sterilizing substance” is interchangeable with the term “disinfecting substance”. In one or more embodiments, the sterilizing substance may be, but is not limited to, an antibacterial substance, an antiviral substance (e.g., an anti-HIV agent), an anti-tuberculin substance, an anti-fungal, and combinations thereof. In one or more embodiments, the sterilizing substance may be a bactericidal substance, a virucidal substance, and/or a fungicidal substance. Exemplary sterilizing substances include, without limitation, alcohol, isopropyl alcohol, and hydrogen peroxide. In one or more embodiments, the sterilizing or disinfecting substance may be a fluid or a liquid. In one or more embodiments, the sterilizing or disinfecting substance may be a gas. In one or more embodiments, the gas may be a pressurized gas. In one or more embodiments, the sterilizing substance may be housed within the housing of the decontamination device. In one or more embodiments, at least one of the openings of the decontamination device are sealed and/or covered by a removable sealing member thus preventing the sterilizing substance from escaping from inside the housing of the decontamination device. The seals and/or covers and/or removable sealing members maintain the sterility of the decontamination device until about the time of use of the decontamination device.

In one or more embodiments, the sterilizing substance is sodium hypochlorite. In one or more embodiments, the concentration of sodium hypochlorite is 0.55% or higher. In one or more embodiments, the concentration of sodium hypochlorite is below 0.55% concentration.

In one or more embodiments, the housing may be manufactured from various materials. Optionally, the housing is manufactured from an inert material, thus suitable for various types of pharmaceutical substances. The housing may be made from flexible or from rigid materials. Suitable materials include, without limitation a plastic, a glass, a rigid plastic, a flexible plastic, and combinations thereof. In one or more embodiments, the housing may be transparent. In one or more embodiments, the housing may be opaque.

In one or more embodiments, the housing may be a closed chamber which forms a tight, optionally, airtight connection between the vessels. In one or more embodiments, when the openings of the housing are connected to vessels or connection ports, or when the openings are covered by a cover or a seal or a removable sealing member, the housing may form an interior which is hermetically sealed/isolated from surrounding/environmental air. In one or more embodiments, the cover or the seal or the removable seal is frangible, breakable or rupturable. In one or more embodiments, the cover or the seal is pierceable by a piercing member such as a needle.

In one or more embodiments, the housing may optionally include one or more apertures, optionally covered by a filter, to thereby allow releasing air from the housing when actuating the wiping member. Optionally, the one or more apertures may be covered by a valve, to thereby allow releasing of air or pressure from within the housing when actuating the wiping member. In one or more embodiments, the valve may be a one-way valve, thereby allowing the release of air or pressure from within the housing when actuating the wiping member while preventing ambient or environmental air from entering the housing. In one or more embodiments, the filter or valve may be located in or on a wall of the housing's first, second or third compartment.

In one or more embodiments, the system or devices of the invention is provided when the housing of the device is already connected via the proximal end of the device to one or more medical vessels. In accordance with this embodiment, the one or more medical vessels may be integrally manufactured or assembled with the proximal end portion of housing and provided or distributed to consumers as such. In accordance with this embodiment, the system may be configured to allow a connection to additional one or more vessels. In one or more embodiments, the proximal end portion of the housing of the decontamination device with a wiper disposed inside the housing abuts a wall of a container. In one or more embodiments, the proximal end portion of the housing of the decontamination device with a wiper disposed inside the housing is flush mounted to a wall of a container. In one or more embodiments, the proximal end portion of the housing of the decontamination device with a wiper disposed inside the housing is surface mounted to a wall of a container. In one or more embodiments, the container is a bag. In one or more embodiments, the container is a bottle. In one or more embodiments, the container is a vial. In one or more embodiments, the vessel is a syringe. Alternatively, the device and/or system of the invention is provided without any attachment to a vessel. Alternatively, the device and/or system of the invention is provided without any attachment to a vessel and is configured to connect a plurality of vessels and decontaminate thereof.

In one or more embodiments, the housing of the device has an engagement mechanism configured or adapted to connect the at least one vessel and/or connection port to the housing. Various types of engagement mechanisms may be applicable and are contemplated. For example, the engagement mechanism is selected from, without limitation, a thread, a luer, a luer-lock, a luer-slip, snap-on, twist-on, a ratchet teeth mechanism, a retention member, a clamping rail, and an adhesive mechanism. The thread and the complementary thread may be selected from, without limitation, a luer, a smart-site mechanism and combinations thereof. In an embodiment, the engagement mechanism may be selected from a ratchet teeth mechanism, a clamping rail mechanism and/or an adhesive. In an embodiment of the invention, the engagement mechanism positions the surface of the second sealing member of the connection port at about same level as an interior wall of the housing of the device. In an embodiment of the invention, the engagement mechanism positions an exterior surface of the second sealing member of the connection port at the same level as an interior wall of a housing of the device. In an embodiment of the invention, the engagement mechanism positions the surface of the second sealing member of the connection port in a manner that protrudes into the housing of the device. In an embodiment of the invention, the protruding of the surface of the second sealing member connection port is a slight/minor protruding. In an embodiment of the invention, the protruding of the surface of the second sealing member of the connection port is less than one-eighth of an inch. In an embodiment of the invention, the engagement mechanism positions the surface of the second sealing member of the connection port in a manner that is below an inner surface of the housing. In an embodiment of the invention, the positioning of the surface of the second sealing member of the connection port ranges from less than one tenth of an inch to less than a half an inch from the inner surface of the housing. In an embodiment of the invention, the engagement mechanism positions the surface of the second sealing member of the connection port slightly below an inner wall/surface of the housing while allowing the wiping member of the device to wipe off the entire surface of the second sealing member of the connection port. Optionally, or alternately, the engagement mechanism may be configured or adapted to connect at least one opening of the housing of the housing of the device to a connection port In one or more embodiments, the connection port may be configured to couple/engage with an opening of the housing of the decontamination device. In one or more embodiments, the herein disclosed invention allows transferring medical substances in a contaminant-free, or in a substantially contaminant-free manner.

The herein systems and connectors may include one or more valves that restrict fluid flow or passageway. For example, one or more valves may be located at the opening of one or more vessels, at one or more openings of the housing of the device, and/or at one or more openings of the connection port. In a further exemplary embodiment one or more valves may be disposed at the aperture(s) of the vessels or at a location within a connection port or within a housing of the device. In one or more embodiments, the valve may be located at a proximal end portion of a device having a wiping member disposed inside the device and removable sealing member attached to a distal end portion of the device. In one or more embodiments, the valve may be located at a distal end portion of a device having a wiping member disposed inside the device and a removable sealing member attached to a distal end portion of the device. In one or more embodiments, the valve may be a one-way valve. In one or more embodiments, the one-way valve may be configured to allow a fluid to flow through the valve in one direction. In one or more embodiments, the one-way valve may allow fluid to flow from a proximal end portion of the device to a distal end portion of the device. In one or more embodiments, the one-way valve may allow fluid to flow from the distal end portion of the device to the proximal end portion of the device. In one or more embodiments, the connection port may have a one-way valve disposed inside the connection port, on the connection port, and/or over an opening/aperture of a/the connection port. In one or more embodiments, the valves are generally provided to seal and prevent fluid flow prior to any engagement of the housing of the device to the connection port and/or external displacement of connection interfaces. In an exemplary embodiment, when valves are provided to seal an aperture that provides for the external displacement of removable sealing members, the valves may establish resealing of the apertures at about the time, at the time, or following the external displacement of the removable sealing members, preventing any leakage of fluids from the engagement between the vessels. In certain embodiments, the resealing occurs at about the time or prior to the external displacement. In certain embodiments, the valves that provide for the passage and external displacement of the removable sealing members may be made of a rubber material, an elastomeric material, a plastic material, and combinations thereof. In an embodiment of the invention, the valve or aperture providing for the passage and external displacement of at least one or two removable sealing members maintains a hermetic seal between the device and the connection port by a force of the engagement between the distal end portion of the housing of the device and the proximal end portion of the connection port. In some embodiments the force of engagement that maintains the hermetic seal between the housing of the device and the connection port during the external displacement of the removable sealing members is provided by an engagement mechanism selected from a thread, a luer, a ratchet teeth mechanism, a snap-on mechanism, and combinations thereof. In certain embodiments, the aperture, sealed aperture, or valve providing for the passage and external displacement of at least one or at least two removable sealing members reseals prior to the removable sealing members fully displacing from the device and connection port. In some embodiments, the displacement of the removable sealing members occurs in the space between two ratchet teeth. In certain embodiments, the displacement of the removable sealing members occurs between threads of a thread/luer engagement mechanism. In certain embodiments, the valves may prevent backflow of fluids. In some embodiments, the valves may be one-way valves.

In an embodiment of the invention, the herein disclosed devices, systems and methods allow fluid communication in contaminant-free, or substantially contaminant-free manner. For example, between three or more, four or more, five or more or six or more vessels. In an embodiment of the invention, the herein disclosed devices, systems and methods allow fluid communication between three, four, five, six, or seven vessels. For example, the devices, systems, and methods of the invention allow fluid communication between two containers, between two bags, between a syringe and a bag, between a connector and a bag, between a vial and a bag, between a vial and a bottle, between a syringe and a vial or between a syringe and a connector.

A further purpose of this invention is to provide a device having a removable sealing member and a wiping member. Still a further purpose of this invention is to provide a system comprising a device having a wiping member and a removable sealing member, and a connection port having a removable sealing member, the device configured to engage/couple the connection port and the connection port configured to engage/couple the device thus providing a system that reduces the presence of non-purified air and/or air particles. This invention focuses on removable sealing members and wiping members that serve a decontamination/sterility function which substantially reduces of contaminants and ambient air particles and/or provides entirely contaminant-free and sterile engagement and/or fluid passageway between said device and said connection port. This invention focuses on a device, system, and method having a dual mechanism of action the first mechanism of action being external displacement of a removable sealing member from the device and the second mechanism of action being wiping off by the wiping member one or more surfaces of the device and optionally a connection port.

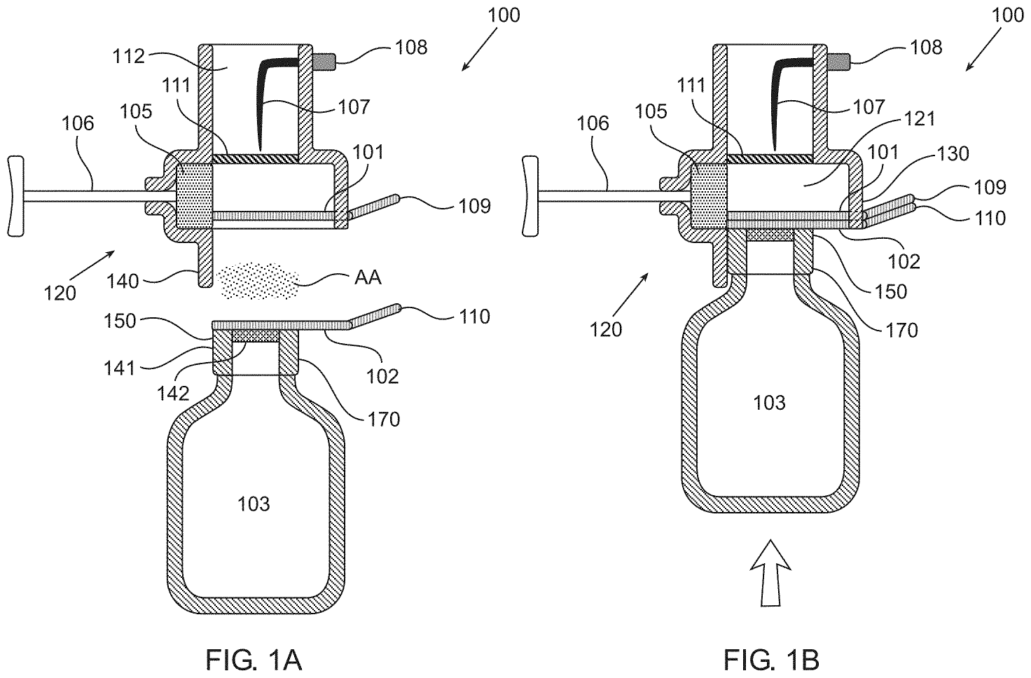

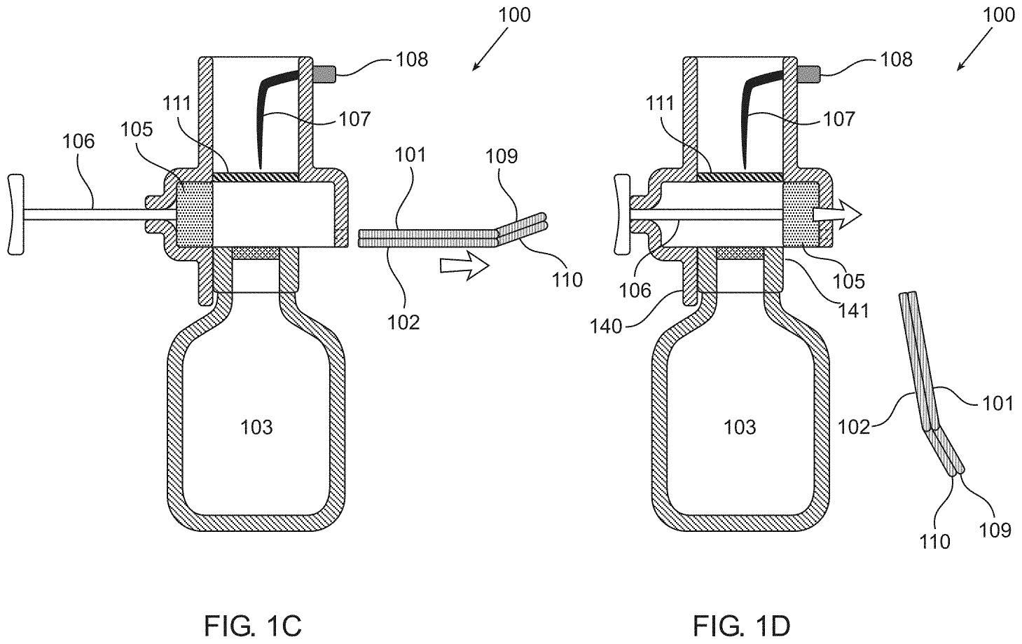

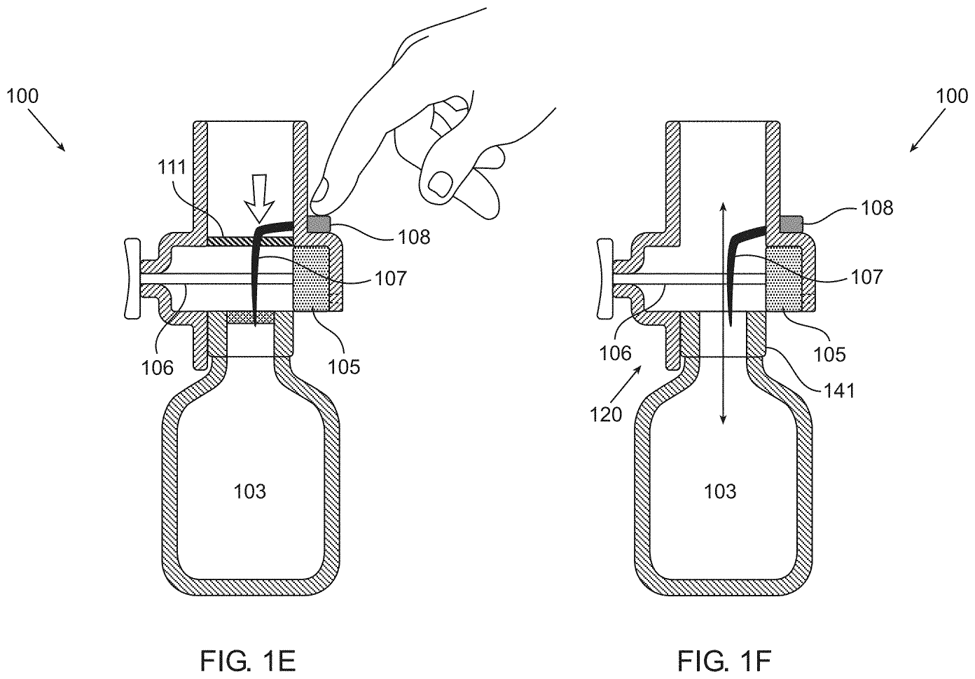

Referring now to the drawings, FIGS. 1A-1F illustrate an exemplary decontamination system 100 for the engagement of a device 120 and a connection port 141. Decontamination system 100 includes a decontamination device 120 with a housing 140 having a first removable sealing member 101 attached to the distal end portion 130 of the housing 140 of the device 120. The distal end portion 130 of the device 120 is configured to engage/couple a proximal end portion 150 of a connection port 141. The proximal end portion 160 of device 120 may be configured to engage/couple a vessel (not shown) such as a container, a bag, a bottle, a vial, a filter, a syringe, an infusion line, and/or any other type of medical/medicinal device. Distal end portion 170 of the connection port 141 may be attached, integrally attached, and/or form a unitary structure with a vessel such as container/vial/bottle 103 or any type of medical/medicinal device such as but not limited to an infusion line, a syringe, a filter, tubing, etc. Decontamination system 100 further includes a second removable sealing member 102 attached to a proximal end portion 150 of connection port 141. A vessel 103 in the form of a bottle/vial is shown attached to the distal end portion 170 of the connection port 141. First removable sealing member 101 of housing 140 of device 120 may be provided when coupled to housing 140 or optionally/alternately to a vessel (not shown) comprising/attached to housing 140. Second removable sealing member 102 may be provided when coupled to proximal end portion 150 of connection port 141, or optionally/alternately to second vessel 103. Optionally, the removable sealing member 101 may be integrally manufactured with housing 140 or vessel (not shown) comprising same. Removable sealing member 102 may be integrally manufactured with connection port 141. Further optionally, the removable sealing members 101 and 102 may be manufactured or assembled separately from the device and the connection port respectively, and coupled thereto at a later stage in a sterile manner. First removable sealing member 101 and second removable sealing member 102 may be similar or may be different in size and/or shape. First removable sealing member 101 and/or second removable sealing member 102 may be in the form of a film. The film may be manufactured from, for example, an elastic polymer (e.g., a rubber), an inelastic polymer, a plastic, or from a metal film (e.g., a metal alloy) or polymer and may be of a thickness of less than about 5 millimeters, less than about 3 millimeters, or less than about 1 millimeter. Removable sealing member 101 may be attached to device 120 via an adhesive or other type of mechanism that facilitates the removal, sliding off, and/or peeling off of removable sealing member 101 from device 120. Removable sealing member 102 may be attached to connection port 141 via an adhesive or other type of mechanism that facilitates the removal, sliding off, and/or peeling off of removable sealing member 102 from connection port 141. As herein shown second vessel 103 is a bottle/vial however other forms of vessels or containers are herein contemplated. For example, the vessel 103 may be a medical container chosen from a vial, a bag, a bottle, and the alike. The vessel 103 may also be chosen from a syringe, an infusion line, a connector, a filter, a manifold, a bag, a bottle, a vial, tubing, or any of the alike. Housing 140 of device 120 is attached to or accommodates first removable sealing member 101. Vessel 103 includes a connection port 141 which is attached to second removable sealing member 102. First removable sealing member 101 can be used to seal and/or cover distal end portion 130 of housing 140. Second removable sealing member 102 can be used to seal and/or cover proximal end portion 150 of connection port 141. The proximal end portion 160 of the device 120 may be attached to, connected to, integrally attached to, and/or form a unitary structure with a vessel (not shown) such as a container, a bag, a bottle, a vial, a syringe, a filter, tubing, an infusion line, tubing, a manifold, and/or any type of medical/medicinal device. First removable sealing member 101 and second removable sealing member 102 are configured to allow for an engagement between device 120 and connection port 141. The device 120 is shown having a wiping member 105 located inside the housing 140 of the device 120. The wiping member 105 is configured to move across the device 120. The wiping member is further configured to wipe off a surface of first sealing member 111 of device 120. In one or more embodiments, the wiping member 105 is configured to wipe off a surface of first sealing member 111 and a surface of second sealing member 142 of connection port 141. In one or more embodiments, wiping member 105 of device 120 is configured to wipe off a surface of second sealing member 142 when the wiping member 105 moves across device 120 when device 120 is engaged/coupled to connection port 141. Device 120 may also comprise, in one or more embodiments, a piercing member 107. Piercing member 107 may be a needle and/or a hollowed needle. Piercing member 107 is configured to move downwards and pierce through first sealing member 111, second sealing member 142 when device 120 is engaged/coupled to connection port 141, or piercing through both first sealing member 111 and second sealing member 142 when device 120 is engaged/coupled to connection port 141. An actuator 108 is provided and shown, the actuator configured to move the piercing member 107 downwards through at least one of first sealing member 111 and second sealing member 142 when device 120 is engaged/coupled to connection port 141. First sealing member 111 may be a bung, a rubber bung, a stopper, a rubber stopper, and/or combinations thereof. First sealing member 111 may, in one or more embodiments, be a film, may be made of a plastic and/or rubber material, and/or may be rupturable and/or frangible. Second sealing member 142 may be a bung, a rubber bung, a stopper, a rubber stopper, and/or combinations thereof. Second sealing member 142 may, in one or more embodiments, be a film, may be made of a plastic and/or rubber material, and/or may be rupturable and/or frangible. In one or more embodiments, first sealing member 111 may be configured to displace from shown position in FIG. 1A to a second position (not shown), wherein when in the second position the first sealing member 111 is located inside the interior 121 of housing 140 of device 120 or is located inside the interior 112 of a proximal end portion 160 of device 120. Optionally, or alternately, first sealing member 111 may be displaced into (the interior) of a vessel (not shown) attached/connected to the proximal end portion 160 of device 120. Such a displacement of first sealing member 111 may be termed internal displacement since first sealing member 111 is displaced internally into interior 121 of housing 140, interior 112 of proximal end portion 160 of device 120, and/or into the interior of a vessel (not shown) attached/connected to proximal end portion 160 of device 120. In accordance with this embodiment, first sealing member 111 is in the form of a plug that may be pushed/displaced upon application of sufficient force by a user. In one or more embodiments, second sealing member 142 of connection port 141 may also be in the form of a plug that may be pushed displaced upon application of sufficient force by a user, wherein second sealing member 142 may be displaced into the fluid passageway 117 of connection port 141, into the interior of vessel 103, and/or into the interior 121 of the housing 140 of device 120 when the connection port 141 is engaged/coupled to device 120.

FIG. 1A shows decontamination system 100 of the invention prior to engagement of the housing 140 of device 120 to connection port 141. Removable sealing member 101 is attached to housing 140 of device 120 and removable sealing member 102 is attached to connection port 141. Removable sealing member 101 of housing 140 of device 120 is shown in a first sealed position in which removable sealing member 101 is positioned to seal the distal end portion 130 of device 120 thus hermetically/airtight sealing the interior 121 of device 120 and preventing ambient air AA contaminants from entering into the interior 121 of device 120. The interior 121 of device 120 is the location of a fluid passageway that will be formed in FIGS. 1E and 1F after a piercing member 107 pierces through first sealing member 111 and second sealing member 142. Removable sealing member 102 of connection port 141 is shown located at the proximal end portion 150 of the connection port 141, wherein the removable sealing member 102 is shown in a first sealed position covering second sealing member 142 and also covering the location of the fluid passageway 117. When removable sealing member 102 is in the first sealed position as shown in FIG. 1A, removable sealing member 102 hermetically/airtight seals the proximal end portion 150 of connection port 141 thus preventing ambient air AA particles (contaminants) from contacting second sealing member 142 and/or the location of the fluid passageway 117. In FIG. 1A ambient air AA particles (contaminants) surround the removable sealing members 101 and 102. FIG. 1B shows decontamination system 100 of the invention following an engagement/coupling between the housing 140 of the device 120 with connection port 141. The distal end portion 130 of housing 140 of device 120 couples/engages with the proximal end portion 150 of connection port 141. Removable sealing member 101 of the device 120 comes in contact with the removable sealing member 102 of connection port 141. A guiding member (not shown) may be provided in some embodiments attached to housing 140, attached to connection port 141, or attached to both housing 140 and connection port 141. The guiding member (not shown) is configured to align removable sealing member 102 under removable sealing member 101 in a direction so that both removable sealing members 101 and 102 are directed in the same direction, and optionally aligning tab 110 under tab 109 so that both tabs 110 and 109 face the same direction and so that tab 110 is positioned under tab 109 as shown in FIG. 1B. In one or more embodiments, the guiding member (not shown) of either housing 140 and/or connection port 141 aligns and/or positions removable sealing member 101 is that removable sealing member 101 lies flat against removable sealing member 102 when the device 120 is coupled/engaged to connection port 141. The guiding member is important since when both removable sealing members are aligned properly as previously described said alignment facilitates the external displacement of both removable sealing members 101 and 102 via a pulling motion, optionally axial pulling, by a user's hand. An engagement between device 120 and connection port 141 involves entrapment of ambient air AA particles (contaminants) present between removable sealing member 101 and removable sealing member 102. FIG. 1C shows decontamination system 100 during external displacement of removable sealing members 101 and 102, respectively. In FIGS. 1C and 1D both of the removable sealing members 101 and 102 are shown in a second unsealed position in which both removable sealing member 101 and 102 are displaced from device 120 and connection port 141 respectively, and wherein both removable sealing members 101 and 102 are displaced external to the fluid passageway that will be established between device 120 and connection port 141.

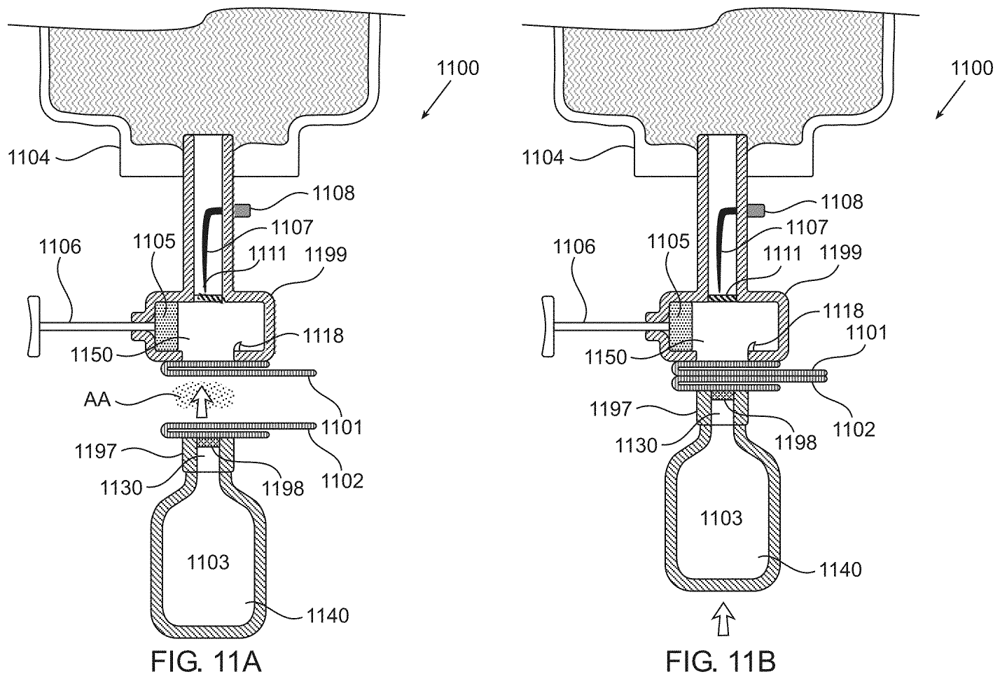

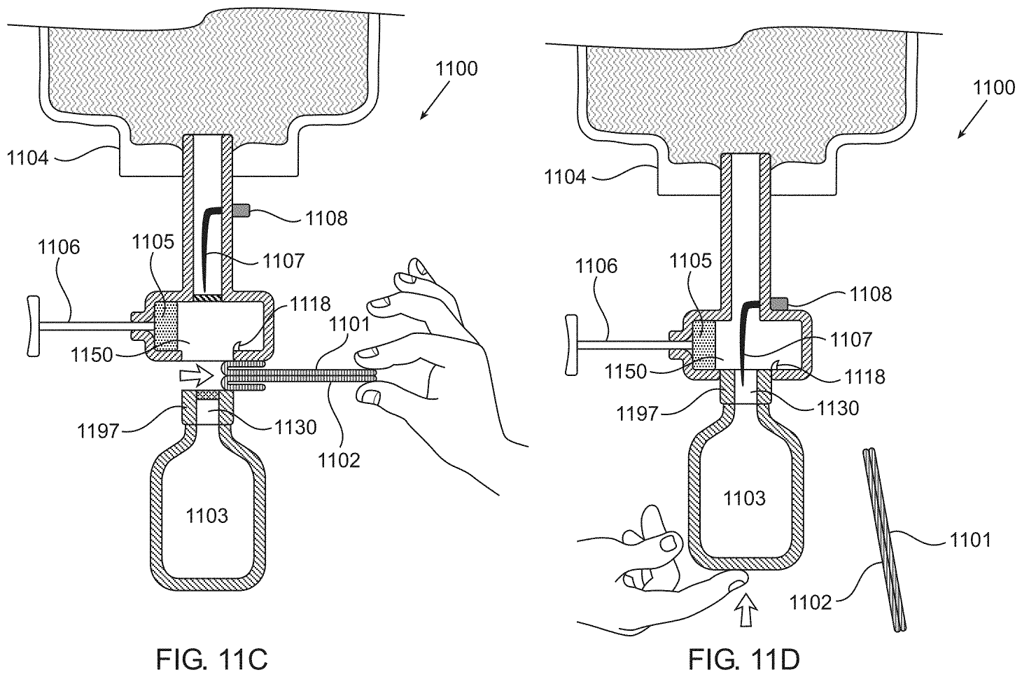

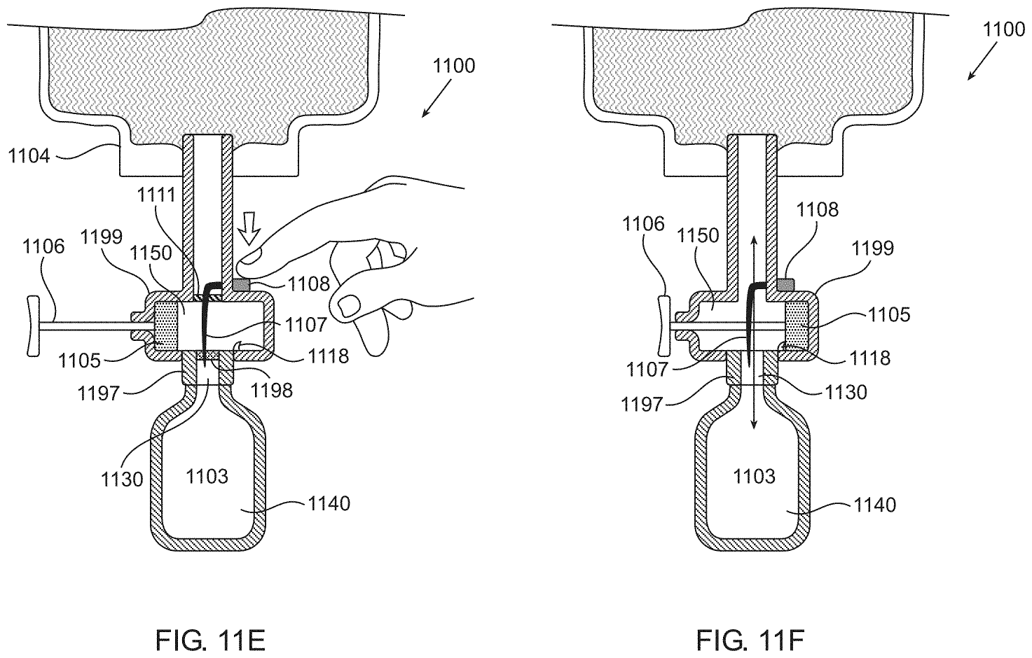

Decontamination system 100 may further comprise a tab 109 positioned on a longitudinal end of first removable sealing member 101 of device 120, and a tab 110 positioned on a longitudinal end of second removable sealing member 102 of connection port 141. Tabs 109 and 110 are configured to be pulled out axially by a user, the axial pulling of the tabs 109 and 110 is configured to allow for a displacement of the removable sealing members 101 and 102 external to device 120 and external to connection port 141. FIG. 1D shows another second step of decontaminating engagement/coupling and/or decontaminating location of fluidic communication between housing 140 and connection port 141 using a wiping member 105 disposed within interior 121 of housing 140 and configured to wipe off first sealing member 111 of housing 140 and optionally also wiping off second sealing member 142 of connection port 141. The wiping member 105 may be connected to an actuator in the form of a handle/tab 106 for mediating the axial push motion by a user. FIG. 1E shows a user actuating piercing member 107 movement by pushing actuator 108, to thereby pierce first sealing member 111 of housing 140 and second sealing member 142 of connection port 141. FIG. 1F shows the establishment of fluid communication between housing 140 of device 120 and connection port 141. The herein disclosed decontamination system 100 establishes a hermetically sealed connection between device 120 and connection port 141 which is maintained during and after the external displacement of the removable sealing members 101 and 102 and as long as the device 120 and the connection port 141 remain in contact/engaged/coupled to each other. In an embodiment of the invention, the engagement between the device 120 and the connection port 141, results in an ambient air AA particles-free engagement between the device 120 and the connection port 141. Ambient air AA particles interfacing/interacting/touching the first and second removable sealing members 101 and 102 are entrapped by the engaged first removable sealing member 101 of device 120 and the second removable sealing member 102 of connection port 141. Subsequently, removable sealing members 101 and 102 are externally displaced from the fluid passageway between device 120 and connection port 141. After said external displacement the wiping member 105 is moved across the housing 140 of device 120 to wipe off and remove any residual contaminants that may be present on the interior 121 of housing 140, on the surface of first sealing member 111, and/or on the surface of second sealing member 142. The dual decontamination mechanism of action of system 100 and/or device 120 provides a first decontamination mechanism comprising external displacement of removable sealing members 101 and 102 from the fluidic passageway and also provides for a wiping member 105 to wipe off any residual contaminants that may be present at the location of fluidic passageway between device 120 and connection port 141. Removable sealing members 101 and 102 are shown in FIGS. 1A-1F in an unfolded position, however it is important to note that removable sealing member 101 and 102 of FIGS. 1A-1F may be provided/configured in a folded position similar and/or identical to the configuration of removable sealing members 1101 and 1102 shown in FIGS. 11A-11D.

It is important to note that the interior 121 of housing 140 is the location of a fluid passageway of housing 140 and the interior 117 of connection port 141 is the location of a fluid passageway of connection port 141. Therefore, the fluidic passageway between housing 140 and connection port 141 is represented by either or both elements 121 and 117.

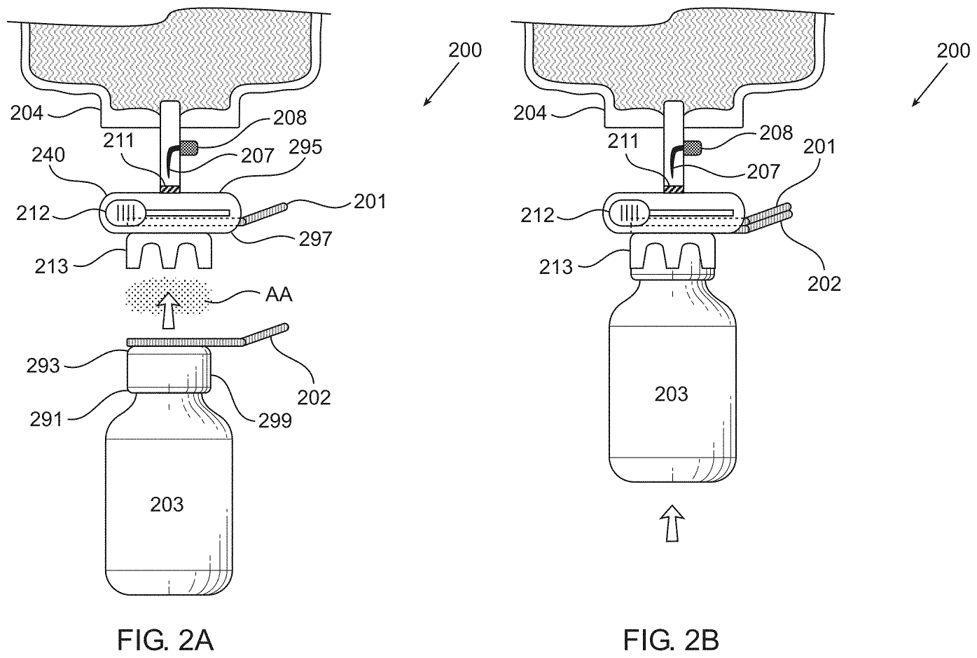

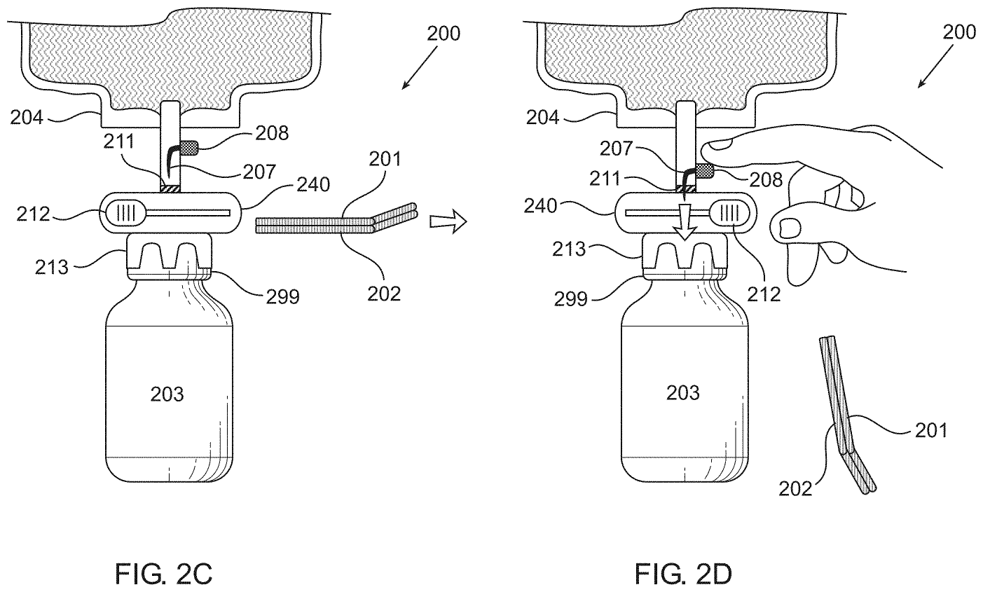

Reference is now made to FIGS. 2A-2D which illustrate yet another exemplary decontamination system for contaminant-free engagement/coupling between a device and a connection port. System 200 is similar to system 100 previously described. System 200 includes a housing 240 of a device, the housing 240 having a wiping member tab 212 which can be used by a user to actuate the wiping effect of a wiping member (not shown) disposed within/inside the housing 240. The system 200 further includes an engagement mechanism in the form of ratchet teeth 213 for allowing a secured engagement between the housing 240 and connection port 299. The engagement between housing 240 and connection port 299 is made by pushing connection port 299 towards housing 240. In an embodiment of the invention, the housing 240 is provided integrally attached with a vessel 204. Vessel 204 is shown as a container (bag or bottle), however other types of vessels are contemplated such as syringes, infusion lines, tubing, filters, connectors, etc. In accordance with this embodiment, the term housing is interchangeable with the term device. A first sealing member 211 is shown at the proximal end portion 295 of housing 240. Removable sealing member 201 is located at the distal end portion 297 of housing 240. Piercing member 207 in the form of a needle, optionally a hollowed needle, is shown connected to actuator button 208.

FIG. 2A shows housing 240 separated from connection port 299. Removable sealing member 202 of the connection port 299 is attached/engaged to proximal end portion 293 of the connection port 299. The removable sealing member 202 may be attached to connection port 299 via an adhesive or any other type of material that hermetically seals removable sealing member 202 to connection port 299. Removable sealing member 202 is configured to be peeled off and/or slid off connection port 299 by a force of a user's hand. Inside connection port 299 may be located/positioned a second sealing member (not shown). The second sealing member may be a plug, a bung, a rubber bung, a stopper, a rubber stopper, a pierceable seal, a frangible seal, a pierceable seal, and combinations thereof. When removable sealing member 202 is attached to proximal end portion 293 of connection port 299, the removable sealing member 202 hermetically/airtight seals the second sealing member (not shown) inside the connection port 299 resulting in a sterile second sealing member which is not in contact with ambient air AA contaminants present in the air. Removable sealing member 201 is attached to distal end portion of housing 297 via an adhesive or any other type of material that hermetically seals removable sealing member 201 to housing 240. Removable sealing member 201 is configured to be peeled off and/or slid off housing 240 by a force of a user's hand. When removable sealing member 201 is in FIG. 2A is shown in a first sealed position, wherein removable sealing member 201 hermetically seals the distal end portion 297 of housing 240 thus maintaining the sterility of the interior of housing 240. When removable sealing member 201 is in the first sealed position shown in FIG. 2A the ambient air AA contaminants are unable to enter into the interior of housing 240.

FIG. 2B shows distal end portion 297 of housing 240 engaged/coupled to proximal end portion 293 of connection port 299 via ratchet teeth mechanism 213. Removable sealing member 201 lies against, optionally lies flat against, removable sealing member 202 when housing 240 is engaged/coupled to connection port 299/

FIG. 2C shows both removable sealing member 201 of housing 240 displaced external to housing 240 and removable sealing member 202 of connection port 299 displaced external to connection port 299. Both removable sealing members 201 and 202 of FIG. 2C are shown in a second unsealed position displaced from a fluid passageway between the housing 240 and connection port 299. Both removable sealing members 201 and 202 are shown detached from the housing 240 and the connection port 299. Removable sealing members 201 and 202 are displaced from housing 240 and connection port 299 via manual manipulation by a user of removable sealing members 201 and 202. The manual manipulation may involve pulling the removable sealing members 201 and 202, axially pulling the removable sealing member 201 and 202, and/or peeling off the removable sealing members 201 and 202 from housing 240 and connection port 299, respectively. After both removable sealing members 201 and 202 are in the second unsealed position the engagement and location of fluidic communication between housing 240 and connection port 299 is decontaminated and/or sterile. In the event that any residual/remaining ambient air AA contaminants remain inside or on the surface of either or both housing 240 and connection port 299 a second decontamination action involving a wiping member follows as described in FIG. 2D.

FIG. 2D shows housing 240 engaged/coupled to connection port 299 after both removable sealing members 201 and 202 are in a second unsealed position. Wiping member tab 212 is actuated by moving/sliding wiping member tab 212 across the housing 240. In FIG. 2D wiping member tab 212 is moved/slid from the left side of housing 240 to the right side of housing 240. Wiping member tab 212 is connected to a wiping member (not shown) located/disposed inside housing 240 through an aperture in the wall of housing 240. Moving/sliding wiping member tab 212 across the housing moves the wiping member (not shown) across the housing, wherein the wiping member moves across/over a surface of first sealing member 211 and a second sealing member (not shown) of connection port 299 thus wiping off and decontaminating/sterilizing the surface of first sealing member 211 of the housing 240 and second sealing member (not shown) of the connection port 299. Additionally, movement of the wiping member (not shown) across the interior of the housing 240 pushes and quarantines any contaminants/ambient air AA contaminants to the right and away from the location of fluidic communication between housing 240 and connection port 299. Therefore, wiping off by the wiping member (not shown) of first sealing member 211 of housing 240 and second sealing member (not shown) of connection port 299 provides a second decontamination mechanism that ensures the sterility of a fluidic communication that will be established between housing 240 and connection port 299. Finally, FIG. 2D shows a user's finger pushing downwards on piercing member actuator 208 which moves piercing member 207 through first sealing member 211 and through second sealing member (not shown) of connection port 299 thus establishing a fluidic communication between the housing 240 and connection port 299. Now a decontaminated and sterile fluidic passageway has been established between housing 240 and connection port 299.

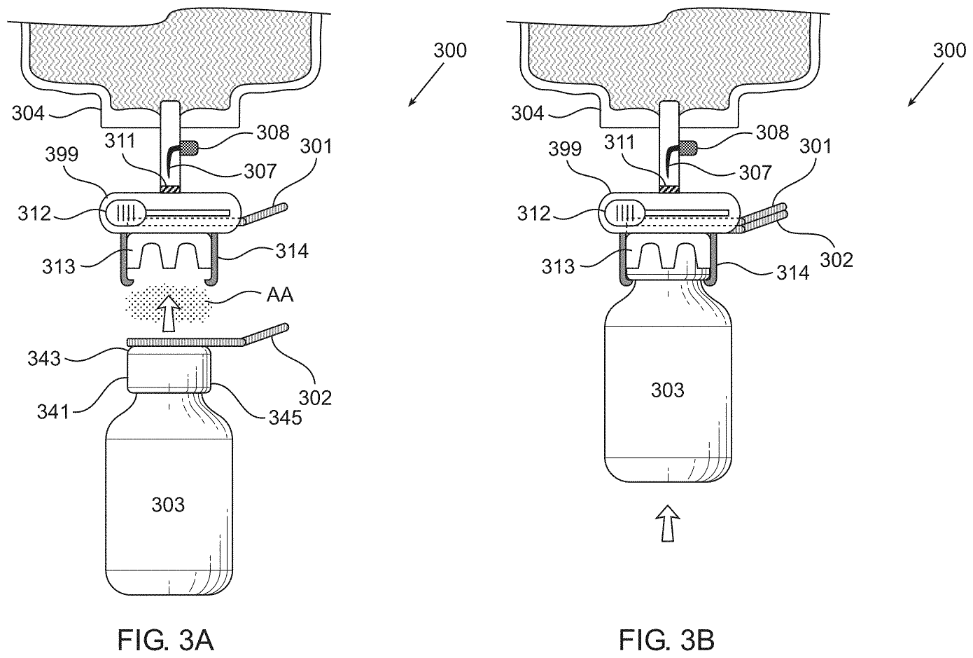

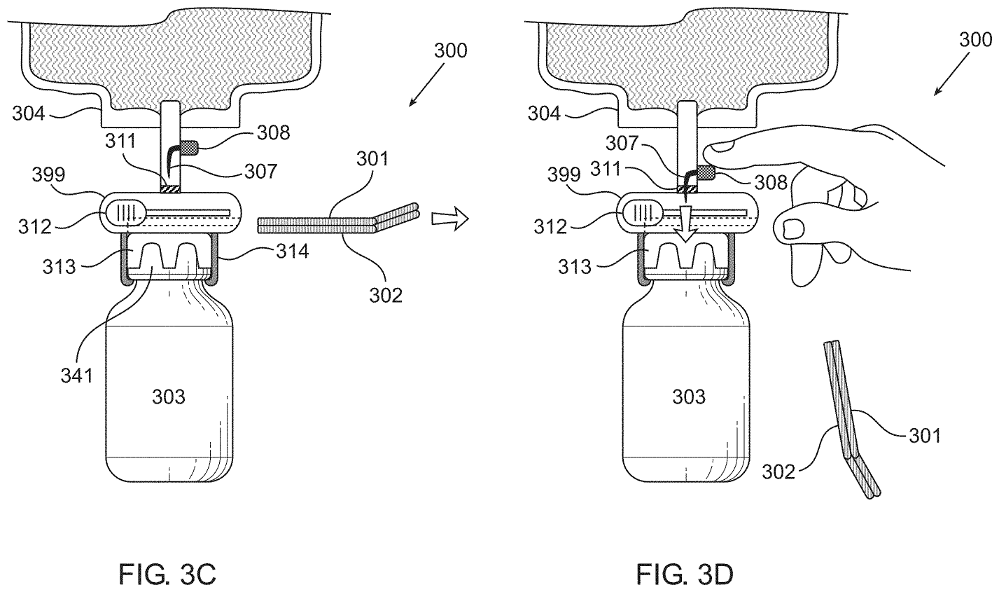

FIGS. 3A-3D show an exemplary decontamination system 300 which is similar to system 200 but further includes clamping arms 314 that are configured to attach connection port 341 and thereby further secure the engagement/coupling between device 399 and connection port 341. In one or more embodiments, connection port 341 may have clamping arms (not shown) similar to clamping arms 314 attached/manufactured with connection port 341 which are configured to engage/couple/clamp a retention member (not shown) located on device 399.

FIG. 3A shows device 399 having an actuator 312 connected to a wiping member (not shown) disposed inside device 399. First removable sealing member 301 is shown sealing the bottom (distal end portion) of device 399. Removable sealing member 301 is configured to be displaced from device 399 via manual manipulation of removable sealing member 301. The manual manipulation of removable sealing member 301 may involve a pulling of removable sealing member 301 so it slides off, optionally or alternately peels off the bottom (distal end portion) of device 399. Ratchet teeth 313 are present/attached to device 399, the ratchet teeth 313 configured to engage/couple with connection port 341. A clamping member 314 is also shown attached to device 399, optionally to ratchet teeth 313, the clamping member 314 configured to clasp connection port 341. Container 304 is shown in the form of a bag though other containers are contemplated such as a bottle, vial, syringe, etc. Container 304 may be replaced with any vessel and/or medical/medicinal device such as an infusion line, tubing, a filter, a connector, a manifold, etc. First sealing member 311 is shown at the proximal end portion of device 399, first sealing member 311 characterized in that it is pierceable and/or frangible. First sealing member 311 is positioned in such a way so as a surface of first sealing member facing/exposed to the interior of device 399 will be wiped off by the wiping member (not shown) located inside device 399 when the wiping member (not) moves across device 399. Actuator tab 312 is shown on an exterior of device 399, the actuator tab 312 is connected to wiping member (not shown) inside the device through a rod/handle that is positioned through device 399 (and/or through a wall of device 399). Piercing 307 in the form of a needle, optionally hollowed needle, is configured to pierce/puncture first sealing member 311 when actuator button 308 is pushed downward, optionally or alternately pressed. Connection port 341 is shown having second removable sealing member 302 attached to proximal end portion 343 of connection port 341. Removable sealing member 301 is attached to proximal end portion 343 of connection port 341 via an adhesive and/or any other type of seal that allows the removal, sliding off, and/or peeling off of removable sealing member 302 via manual manipulation of sealing member 302 by a user. Distal end portion of connection port 341 is attached to container 303. Container 303 is shown as a bottle/vial, however other types of containers may take the place of container 303 such as a bag or a syringe. Container 303 may be replaced by a variety of vessels/medicinal devices such as an infusion line, tubing, a filter, a manifold, a connector, etc. FIG. 3A shows device 399 separate from connection port 341 with ambient air AA contaminants/particles in between device 399 and connection port 341. Removable sealing member 301 is in a first sealed position in which removable sealing member 301 substantially seals an opening/aperture of device 399. The substantial sealing by removable sealing member 301 of opening of device 399 may be a hermetic/airtight sealing of the opening (not shown) at the bottom of the device 399. Removable sealing member 302 attached to the proximal end portion of connection port 341. The removable sealing member 302 is shown in a first sealed position in which removable sealing member substantially seals an opening (not shown) and/or substantially seals a second sealing member (not shown) of connection port 341. The substantial sealing by removable sealing member 302 may be a hermetic/airtight sealing of connection port 341. Removable sealing members 301 and 302 are shown in an unfolded position, however either or both removable sealing members 301 and 302 may be provided in a folded position similar to the removable sealing members 1101 and 1102 of FIGS. 11A-11C.

FIG. 3B shows connection port 341 engaged/coupled to device 399 via ratchet teeth 313 and/or clamping member 314. The removable sealing members 301 and 302 are shown still remaining in the first sealed position. FIG. 3C shows removable sealing members 301 and 302 being displaced

FIG. 3C shows removable sealing members 301 and 302 being pulled to the right and away from device 399 and connection port 341, respectively. The pulling may be the manual manipulation by a user's hand in the form of a pulling, optionally or alternately pulling axially, removable sealing members 301 and 302 away from the location of the fluidic passageway/fluidic communication that will be established between device 399 and connection port 341. Following external displacement of removable sealing members 301 and 302 the actuator tab 312 will be slid/moved to the right (not shown) which moves the wiping member (not shown) disposed inside device 399 across the interior of device 399 which serves as a second decontamination action/mechanism. Movement of the wiping member (not shown) across the device wipes off a surface of first sealing member 311. The surface of the first sealing member 311 that is wiped off by the wiping member (not shown) is the surface facing the interior of device 399. A surface of connection port 341 may also be wiped off by the wiping member (not shown) when the wiping member moves across the device.

FIG. 3D shows a user's finger moving a piercing member actuator tab 308 which moves a piercing member 307 downward and through sealing member 311 and optionally through a surface (not shown) of connection port 341 thus establishing a sterile and contaminant-free fluidic passageway between device 399 and connection port 341, and optionally a sterile and contaminant-free fluidic passageway between vessel/container 304 and vessel/container 303.

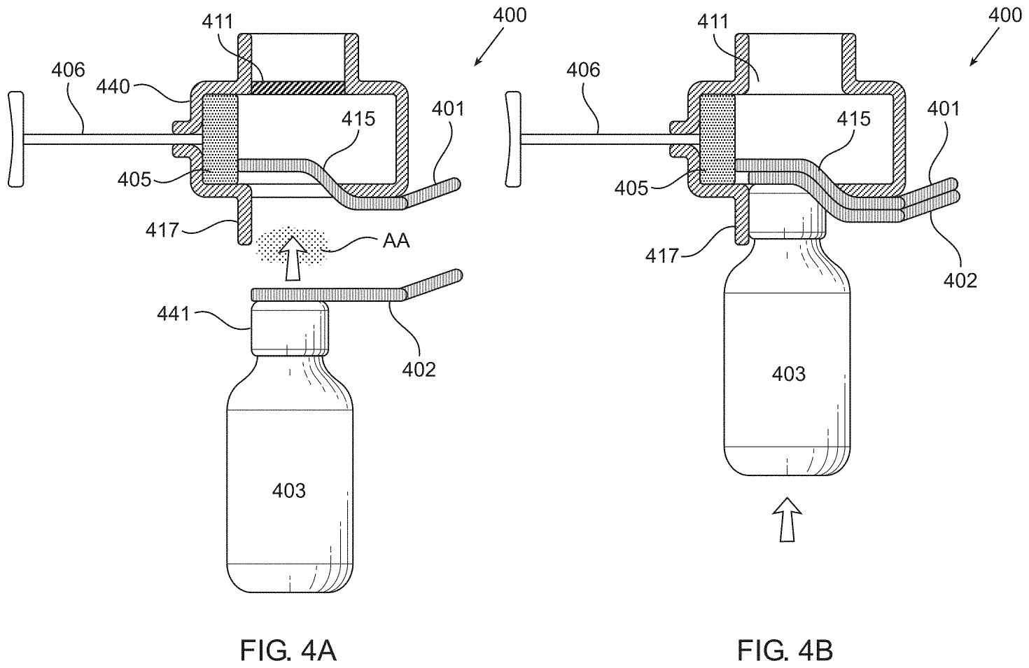

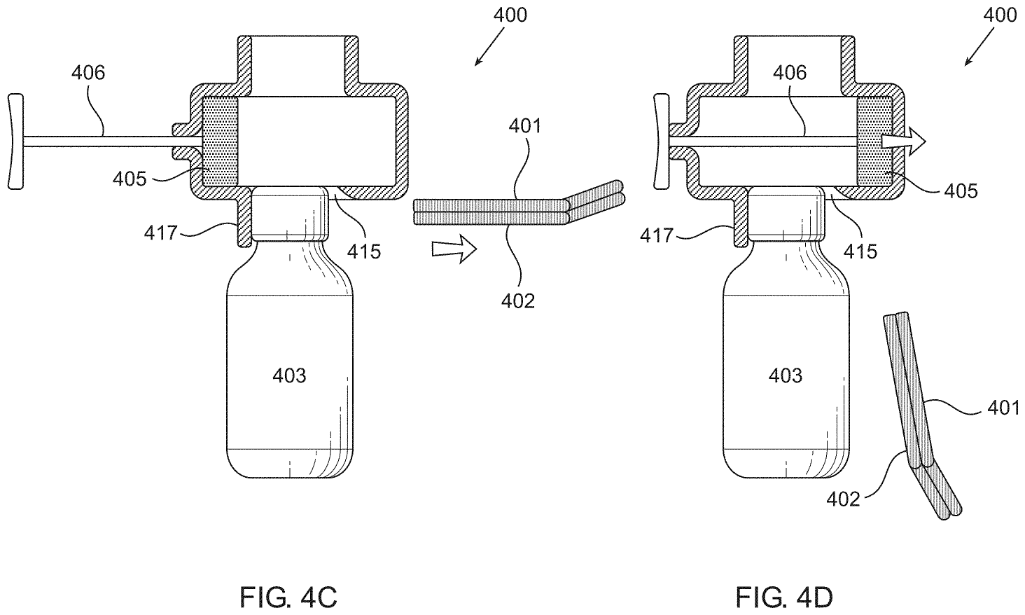

FIGS. 4A-4D illustrate an exemplary system 400 for decontaminated fluid passageway and/or contaminant-free fluid passageway between a device 440 and a connection port 441. In system 400, removable sealing members 401 and 402 extend from an internal location of device 440 to a location external to device 440 via an opening 415 at the bottom wall of device 440. When device 440 and connection port 441 engage, the removable sealing members 401 and 402 cling/adhere to each other as shown in FIG. 4B and can be pulled out via their tabs by a user (FIG. 4C). The wiping of the surfaces of first sealing member 411 and second sealing member (not shown) of connection port 441 can then be effected by axially pushing/moving wiping member 405 across the interior of device 440 via handle/rod/tab 406. Wiping member 405 is shown in FIGS. 4A-4C in a first/initial position in which the wiping member 405 has not moved across the housing of the device 440. Wiping member 405 is shown in FIG. 4D in a second/final position after wiping member 405 has moved across the interior of the housing to wipe off first sealing member 411 and a surface of connection port 441. Guiding member 417 is shown extending downward from housing of device 440. Guiding member 417 is configured to align the connection port 441 to device 440 in such a manner so that removable sealing member 402 of connection port 441 aligns, optionally aligns in an overlapping manner, with removable sealing member 401 of device 440 as is shown in FIGS. 4B and 4C. Such an overlapping alignment of removable sealing members 401 and 402 as is shown in FIGS. 4B and 4C facilitates the external displacement of both removable sealing members 401 and 402 at about the same, at the same time, and/or simultaneously via manual manipulation (pulling/axial pulling) by a user's hand. The guiding member 417 is shown as a downward extending extension of device 440, however various configurations/structures of the guiding member may be provided attached to device 440, connection port 441, or both device 440 and connection port 441. The guiding member 417 may have a rail mechanism disposed on a side, top, or bottom (not shown) of the guiding member which engages/couples with a complimentary retention member located the connection port 441 which guarantees the connection port 441 attaches/couples/engages device 440 in a manner that will align removable sealing members 401 and 402 in an overlapping manner as shown in FIG. 4B. One guiding member 417 is shown, however two or more guiding members may be provided attached to either or both device 440 and/or connection port 441.

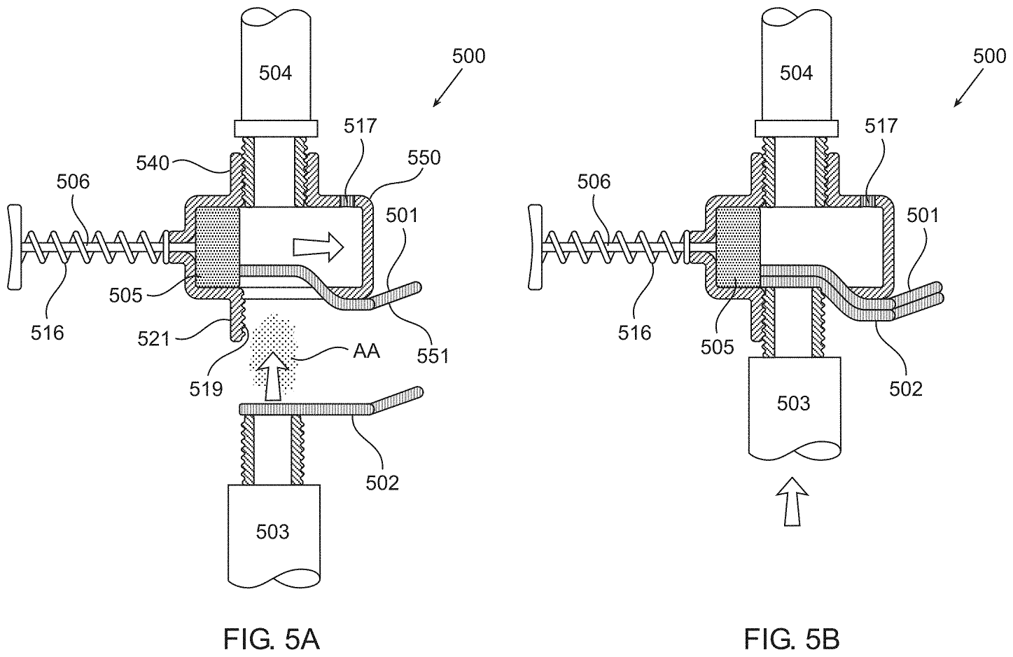

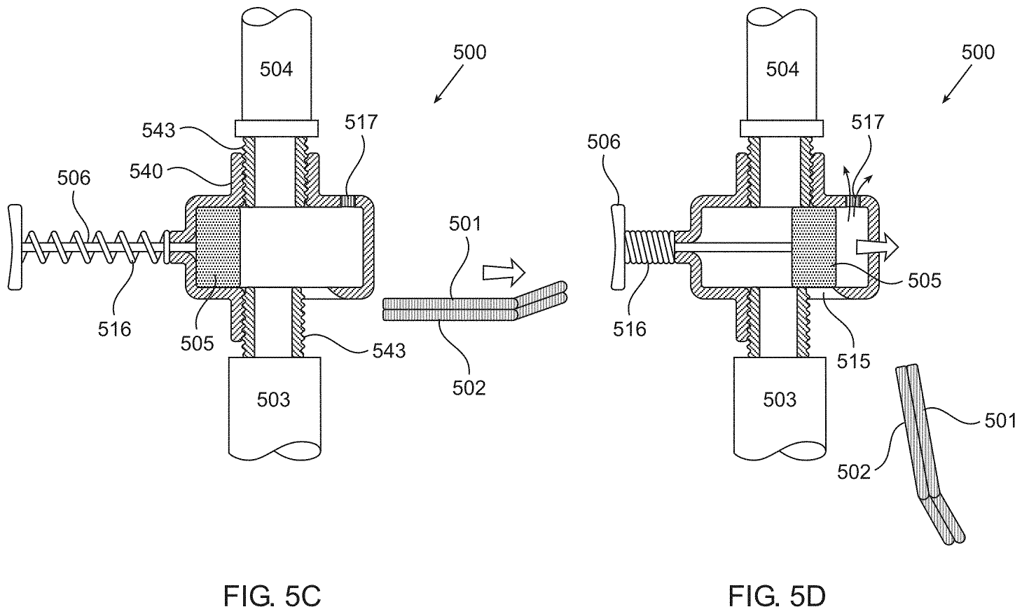

FIGS. 5A-5D illustrate yet another exemplary system 500 wherein housing 540 of device 550 has a wiping member 505 disposed inside the housing 540 and device 550 having removable sealing member 501 sealing the distal end portion 551 of housing 540. Guiding member 521 has threads 519 configured to engage complementary threads 543 of connection port 503. The system further includes a filter 517 disposed in an aperture in the housing 540. Filter 517 is configured to allow for releasing air from the housing 540 when actuating the wiping member 505. Accordingly, air pressure that can be built up during movement of wiping member 505 can be prevented. Filter 517 is located and devised so as to allow the pressurized air to pass therethrough, without allowing any contaminants inside. Filter 517 may have pores having a diameter of less than about a few microns and can push contaminants out of the housing 540. Filter 517 may have a porosity of about 0.22 microns or less. Optionally, filter 517 may be a valve (not shown) which may be a one-way valve allowing air to pass out of housing 540 while preventing ambient or environmental air from entering. Handle 506 may be spring loaded 516 for efficient pushing of wiping member 505. Guiding member 521 is shown extending downward from housing 540 of device 550, however in one or more embodiments of the invention the guiding member 521 may be located on connection port 503. In one or more embodiments of the invention, a plurality of guiding members 521 may be provided attached to either or both housing 540 or connection port 503. Guiding member 521 is characterized in that is guarantees aligning/overlapping of removable sealing members 501 and 502 when connection port 503 is engaged/coupled to device 550 as shown in FIG. 5B. FIG. 5C shows removable sealing members 501 and 502 in a second unsealed position displaced/removed from the fluid passageway that will be established between housing 540 and connection port 503. FIG. 5D shows wiping member 505 moving across the housing wiping off surface(s) of housing 540 and connection port 503 after removable sealing members 501 and 502 are in a second unsealed position.

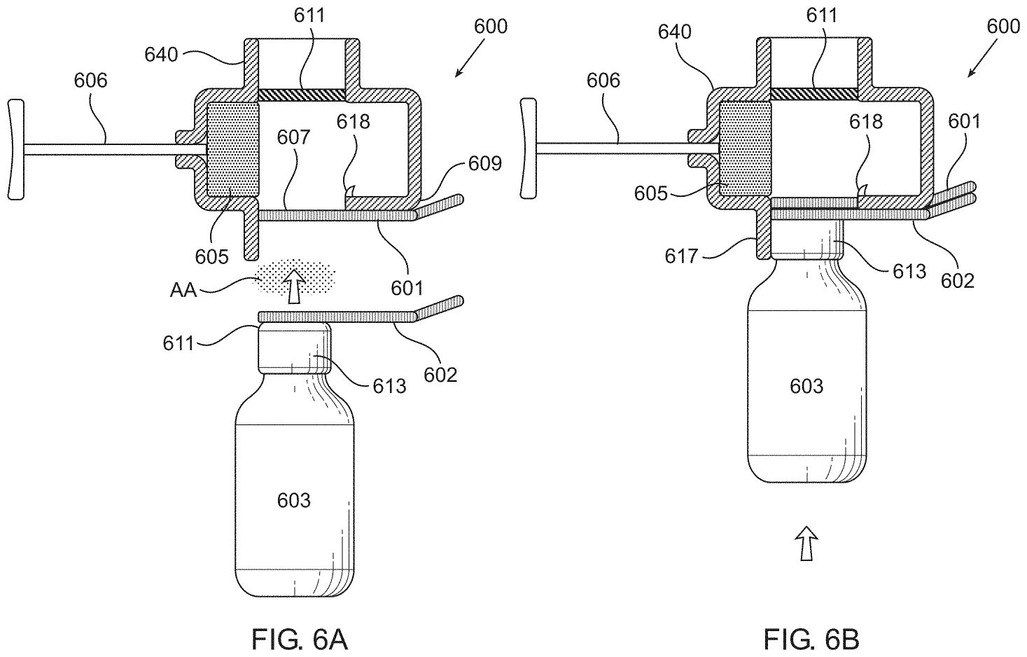

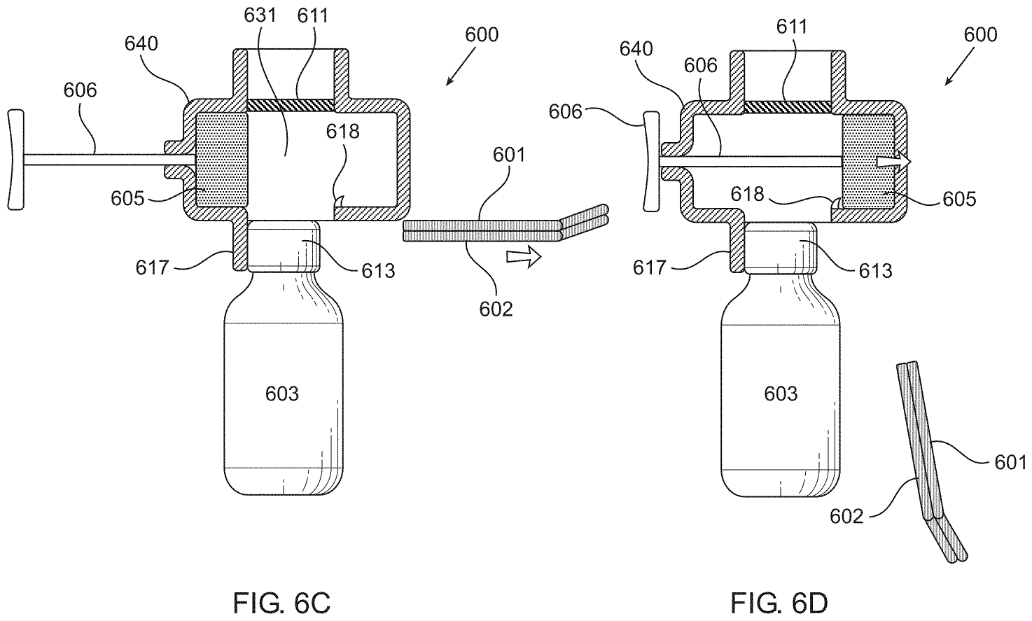

FIGS. 6A-6D illustrate exemplary system 600 which further comprises a locking member 618 disposed in housing 640. Locking member 618 is shown disposed on an interior surface of housing 640. The locking member 618 being configured for locking the wiping member 605 in the end position as shown in FIG. 6D, following actuation of wiping, to prevent the wiping member 605 from returning to an original or an initial position. Locking member 618 protrudes from the internal bottom surface of housing 640. When wiping member 605 passes across the surfaces of the vessels, it passes over locking member 618, which is angled away from wiping member 605 and optionally retractable and allows it to pass therethrough. Once wiping member 605 reaches the end position, shown in FIG. 6D, locking member 618 presses against wiping member 605, preventing it from returning backwards. The housing 640 has a wiping member 605 located inside the housing 640 and a removable sealing member 601 shown covering an opening 607 located at a distal end portion 609 of housing 640. Both removable sealing member 601 and removable sealing member 602 are shown in FIG. 6A hermetically sealing opening 607 of housing 640 and proximal end portion 611 of connection port 613, respectively. FIG. 6B shows connection port 613 engaged/coupled to housing 640. Guiding member 617 of housing 640 ensures alignment/overlapping of removable sealing members 601 and 602. FIG. 6C shows removable sealing members 601 and 602 being displaced simultaneously external to the housing 640 and the connection port 613, and also being displaced external to the location of fluidic communication 631 between the housing 640 and the connection port 613. FIG. 6D shows the wiping member 605 moving across the interior of the housing 640 due to actuation of actuator rod/handle 606.

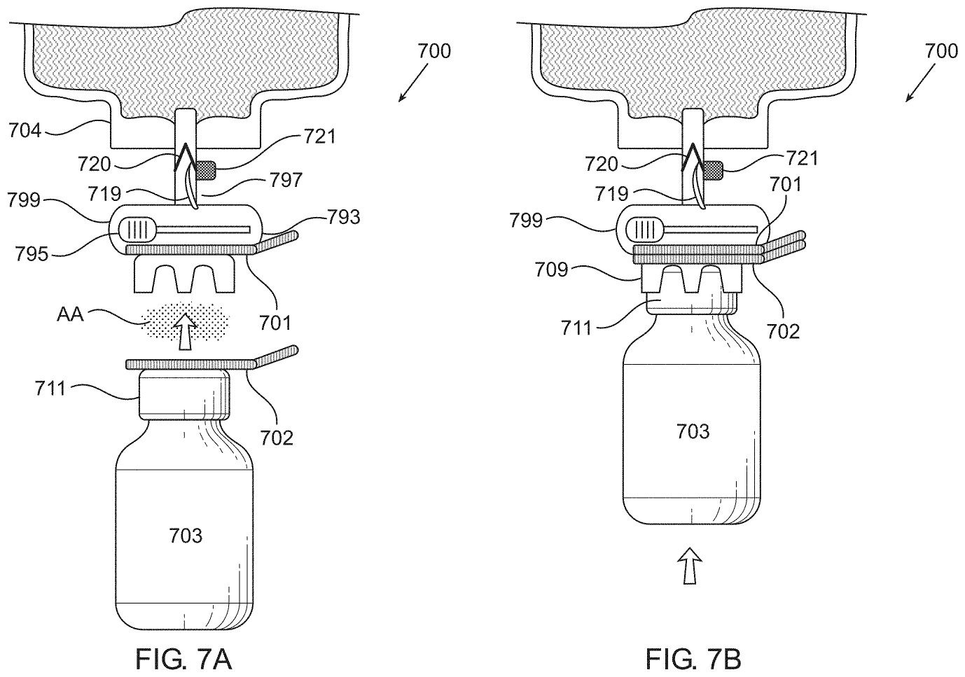

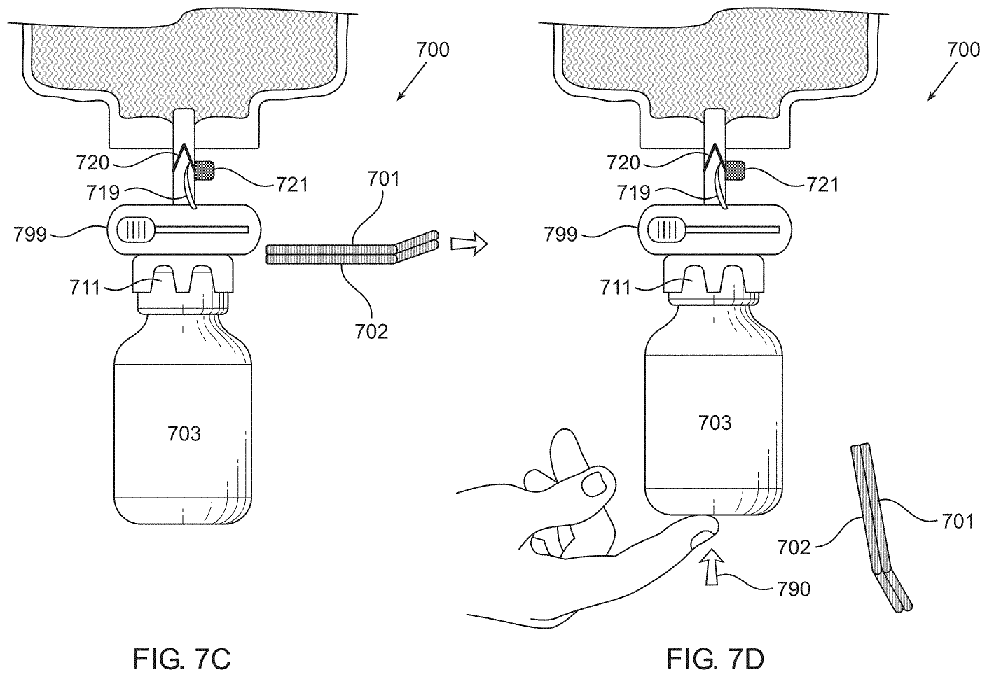

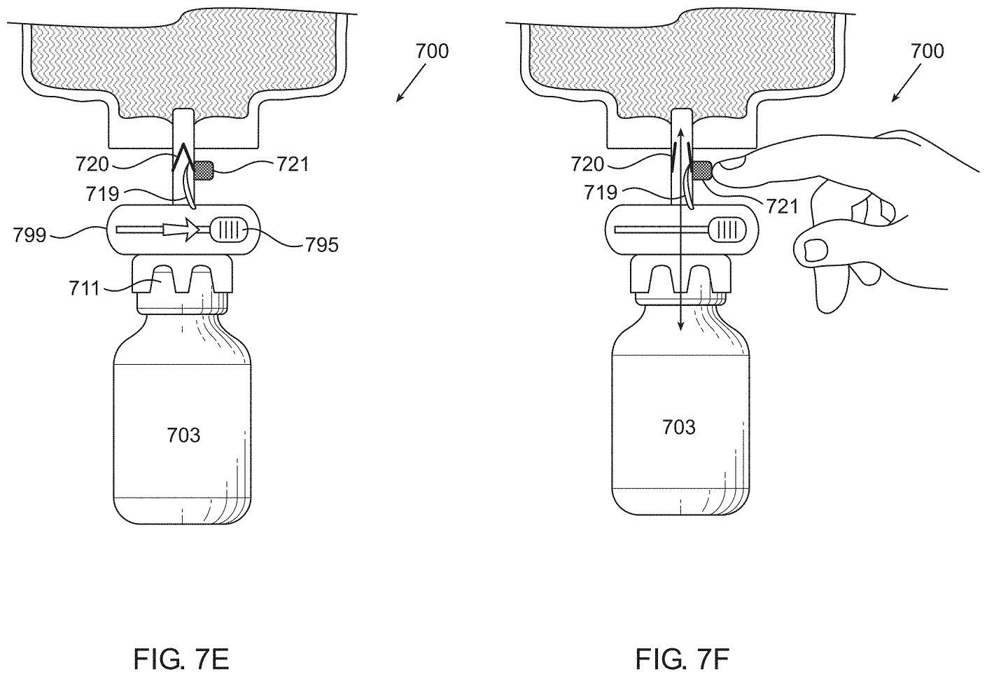

FIGS. 7A-7F illustrate yet another exemplary system 700 which is similar to systems 200 and 300 but further includes a valve 720 for blocking an opening of vessel 704 blocking an opening at the proximal end portion 797 of device 799. Valve 720 is kept closed and configured to be opened by a user pushing down on an actuator 721 (shown in FIG. 7F) which facilitates the valve unlock mechanism 719. Valve-unlocking mechanism 719 includes a hook which is movable and may release or allow opening of valve 720. Valve 720 may be a one-way valve. FIG. 7A shows device 700 having wiping member actuator tab 795, a wiping member (not shown) located inside device 799, and a removable sealing member 701 attached to distal end portion 793 of device 799. Connection port 711 is shown having a removable sealing member 702 sealing the top/opening of connection port 711. Device 799 and connection port 711 are shown separate from each other with ambient air AA contaminants present between device 799 and connection port 711. FIG. 7B shows device 799 attached/engaged/coupled to connection port 711 via engagement mechanism in the form of ratchet teeth 709. An airtight and hermetic engagement/coupling is now established between device 799 and connection port 711. The ratchet teeth 709 may have guiding member(s) (not shown) that ensure connection port 711 engages/couples with device 799 in a manner that removable sealing member 702 aligns underneath removable sealing member 701. Removable sealing member 702 is shown pressing against removable sealing member 701. Removable sealing members 701 and 702 are shown lying flat against each other (one another). An adhesive may be placed on a surface of at least one of removable sealing member 701 and 702 which adheres removable sealing members 701 and 702 to each other. FIG. 7C shows removable sealing members 701 and 702 displaced from device 799 and connection port 711, wherein the removable sealing members 701 and 702 are in a second unsealed position. Removable sealing members 701 and 702 may be pulled out at about the same time, optionally or alternately at the same time, by a force of a user's hand (manual manipulation is pulling). FIG. 7D shows vessel 703 in the form of a vial/bottle being pushed upwards (indicated by an upward pointing arrow 790) by a user's finger, said pushing upwards pushes the vial/bottle 703 slightly upwards towards device 799 which aligns a second sealing member (not shown) located inside and/or on a surface of connection port 711 with an interior wall of the device. The said alignment of the second sealing member (not shown) or surface (not shown) of the connection port 711 with an interior wall of the device allows/positions the second sealing member (not shown) or surface (not shown) of the connection port to be wiped off by the wiping member (not shown) located inside device 799 when the wiping member (not shown) moves across the device 799. FIG. 7E shows wiping member actuator in the form of a tab 795 being moved across (from left to right) device 799 which moves the wiping member (not shown) located inside the device 799 across the device. Movement of tab 795 and wiping member (not shown) across device wipes off at least one of and/or any combinations of an interior wall of the device 799, first sealing member of device 799, a second sealing member (not shown) of connection port 711, and a surface of connection port 711. A hermetic/airtight seal is maintained between device 799 and connection port 711 during movement of the wiping member (not shown) across the device 799. FIG. 7F shows actuator button 721 being pushed inwards/downwards to open valve 720 thus allowing for the establishment of a fluidic communication between device 799 and connection port 711.

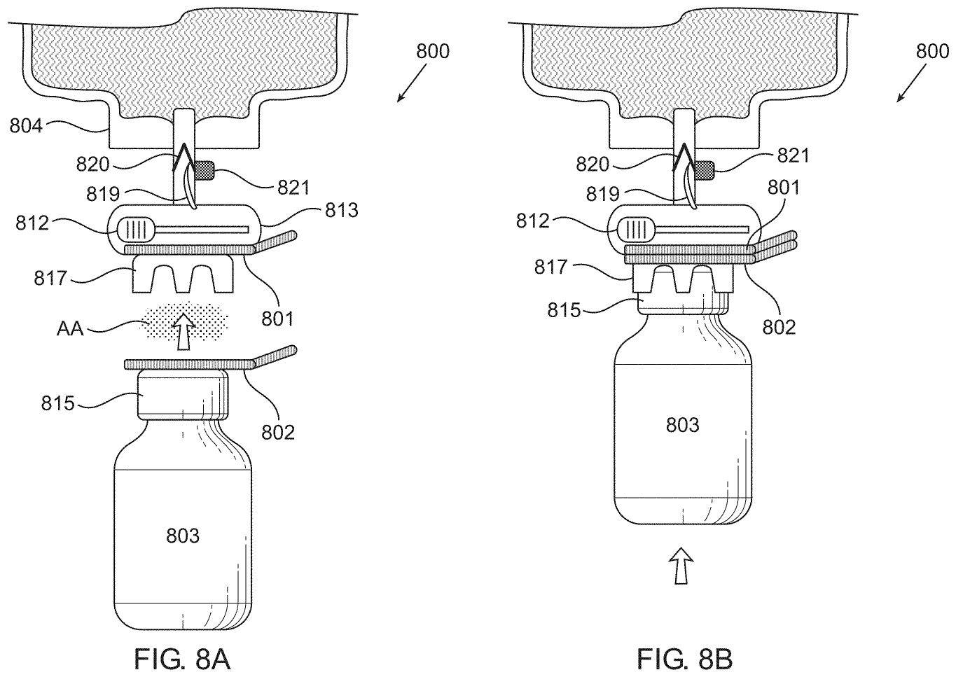

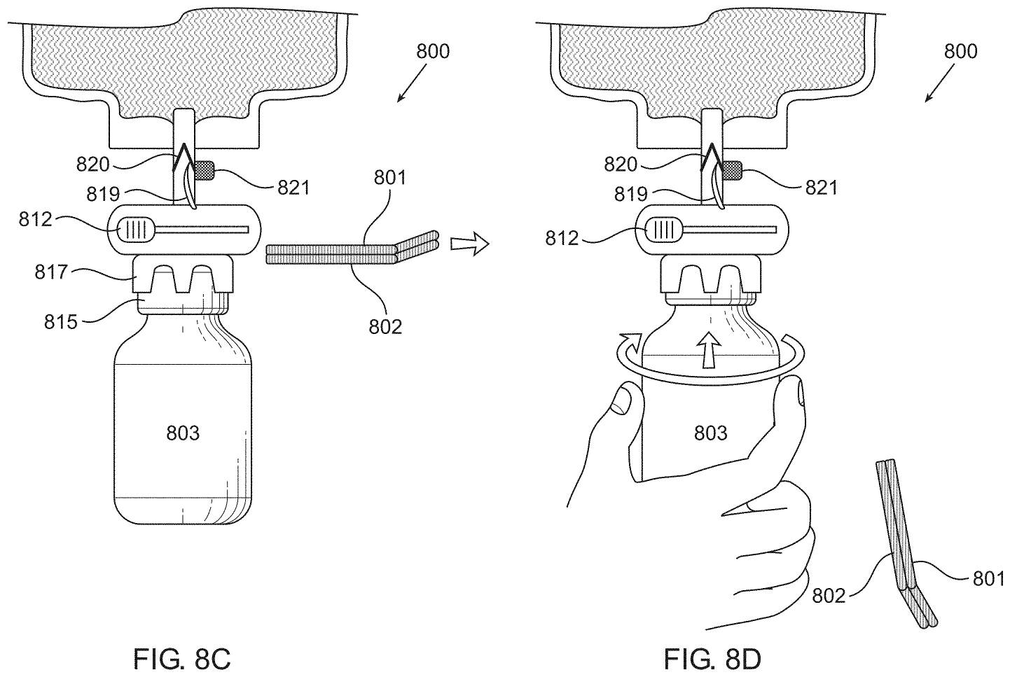

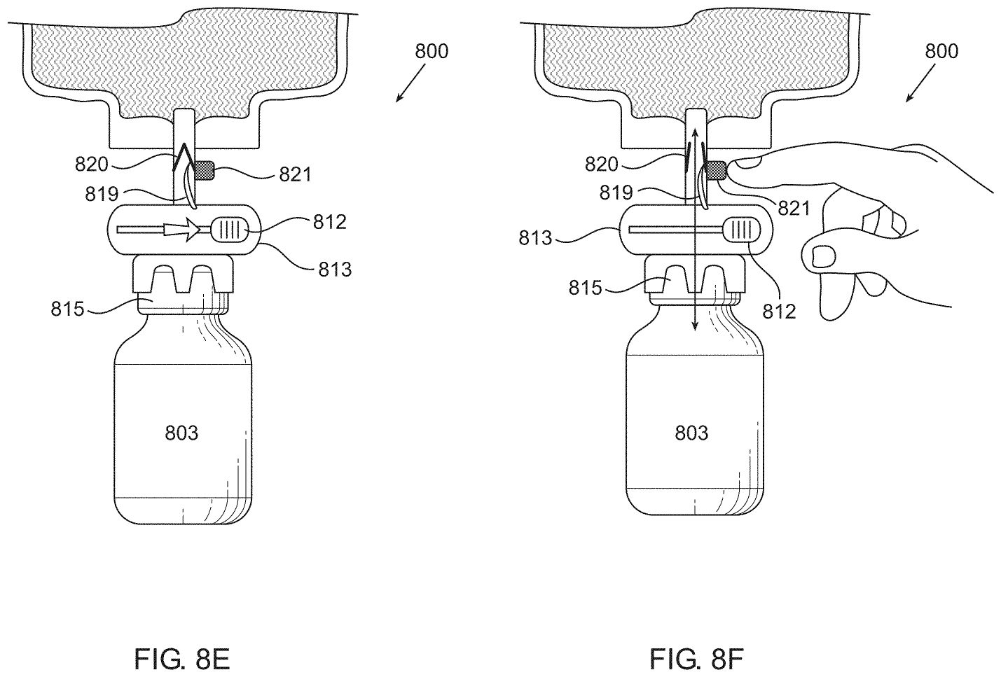

FIGS. 8A-8F illustrate yet another exemplary system 800 which is similar to system 700 but includes a mechanism for engagement between device 813 and connection port 815 that incorporates a twist motion mechanism (see FIG. 8D) whereas in system 700 the engagement mechanism incorporates a push motion mechanism (see FIG. 7D). FIG. 8A shows a device 813 having a removable sealing member 801 shown sealing distal end portion of device 813. Device 813 has a ratchet teeth mechanism 817 and a wiping member actuator tab 812 configured to move a wiping member (not shown) disposed inside device 813. Connection port 814 has a removable sealing member 802 shown in a first sealed position. Connection port 815 and device 813 are shown separated with ambient air AA particles located between removable sealing member 801 and removable sealing member 802. FIG. 8B shows connection port 815 hermetically/airtight engaged/coupled to device 813 wherein ambient air AA particles/contaminants are entrapped between removable sealing member 801 and removable sealing member 802. FIG. 8C shows both removable sealing members 801 and 802 displaced external to device 813 and connection port 815 and in a second unsealed position. FIG. 8D shows a user's hand turning/twisting vessel 803, optionally or alternately connection port 815 and/or device 813, the turning/twisting motion of connection port 815 relative to device 813 moves a second sealing member (not shown) and/or a surface (not shown) of the connection port 815 upwards towards device 813 to align the second sealing member and/or the surface of the connection port 815 with an interior wall of device 813 which positions the second sealing member and/or the surface of the connection port 815 to be wiped off by the wiping member (not shown) of device 813 when the wiping member moves across the interior of device 813. FIG. 8E shows wiping member actuator tab 812 moving across device 813 which moves the wiping member (not shown) across the interior of device 813. FIG. 8F shows valve actuator button 821 pushed inwards/downward/upwards to mediate the opening of valve 820 thus allowing for a sterile and decontaminated fluidic communication between device 813 and connection port 815.

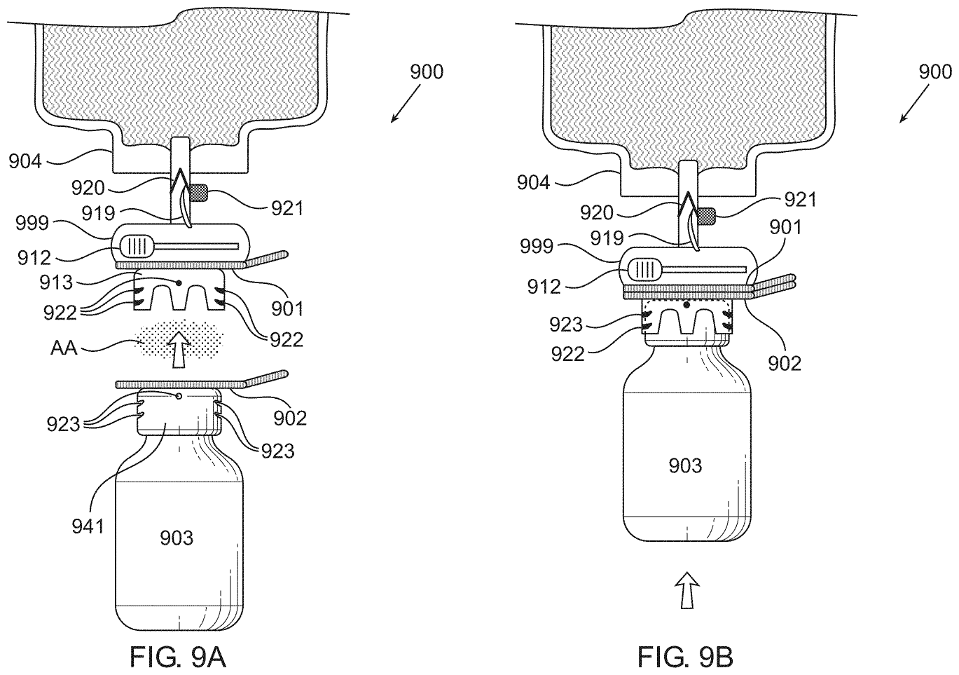

FIGS. 9A-9B illustrate yet another exemplary system 900 that further incorporates lock mechanisms for locking together device 999 and connection port 941 via retractable teeth 922 that can lock into holes or dents 923 of connection port 941. In one or more embodiments, retractable teeth 922 may be present/located on device 999, optionally on housing of device 999. In one or more embodiments, retractable teeth 922 may be located on a securing/engagement mechanism such as ratchet teeth/clamping member/retention member of device 999. In one or more embodiments, device 999, optionally or alternately a securing/engagement mechanism of device 999 may have holes/dents (not shown) similar/identical to those of connection port 941, the holes/dents configured to engage/couple retractable teeth (not shown) present/located on connection port 941. Retractable teeth (not shown) similar/identical to retractable teeth 922 may be present in some embodiments attached to device 999. Holes/dents (not shown) similar to the holes/dents 923 may in some embodiments be present/located on device 999. FIG. 9A shows device 999 having a wiping member actuator tab 912 connection to a wiping member (not shown) located inside device 999. Wiping member (not shown) is configured to move across device 999 when tab 912 is moved across the device by a user. First removable sealing member 901 of device 999 is provided and shown in a first sealed position. Device 999 has a ratchet teeth mechanism/clamping member 913 configured to engage/grasp/couple with connection port 941. Device 999 further comprises valve 920 and valve actuator button 921. Connection port 941 is shown having holes/dents 923 configured to engage/couple with teeth 922 of device 999. Teeth 922 may be retractable teeth or non-retractable teeth. Teeth 922, may in one or more embodiments, be flexible thus allowing connection port 941 to engage/couple with ratchet teeth/clamping member 913. Connection port 941 has a removable sealing member 902 in a first sealed position covering connection port 941. In FIG. 9A connection port 941 is separate from device 999 with ambient air particles AA located between removable sealing member 901 and removable sealing member 902. Connection port 941 is pushed/moved upwards towards device 999. Upon initial engagement/coupling of connection port 941 to ratchet teeth/clamping member 913, the bottom left and bottom right retractable teeth 922 will engage/couple the upper left and upper right holes/dents of connection port 923. At this point an airtight/hermetic engagement between connection port 941 and device 999 is established after which removable sealing members 901 and 902 may be displaced external to device 999 and connection port 941. After external displacement of removable sealing members 901 and 902 from device 999 and 941, connection port 941 may be pushed/moved upwards again towards device 999 as shown in FIG. 9B. FIG. 9B shows connection port 941 engaged/coupled to ratchet teeth/clamping member 913 of device 999 wherein both sets/all of the retractable teeth 922 of device 999 engage/couple with both sets/all holes/dents 923 of connection port 941. The gradual movement and/or engagement/coupling of connection port 941 upwards towards device 999 via retractable teeth 922 and holes/dents 923 provides for the external displacement of removable sealing members 901 and 902 while maintaining a hermetic engagement/coupling between device 999 and connection port 941, and further provides for the alignment of a second sealing member or a surface of the connection port 941 with an interior surface of the device 999 thus positioning the second sealing member or the surface of the connection port 941 to be wiped off by the wiping member of the device 999 when the wiping member (not shown) moves across the device. When the bottom left and bottom right retractable teeth 922 engage/couple the top left and top right holes/dents the connection port is engaged/coupled to the ratchet teeth 913 in an extended position. When the connection port 941 is further pushed/moved upwards towards device 999 the top left and top right retractable teeth 922 engage/couple the top left and top right holes/dents of connection port 941 and the bottom left and bottom right retractable teeth 922 engage/couple the bottom left and bottom right holes/dents 923 which represents an engagement/coupling of connection port 941 to ratchet teeth engagement mechanism 913 in a contracted position. In the/said extended position a surface or second sealing member of connection port 941 is not aligned with an interior surface of device 999. In the/said contracted position a surface or a second sealing member of connection port 941 is aligned with an interior wall of device 999 thus positioning the surface or the second sealing member of connection port 941 to be wiped off by the wiping member when the wiping member moves across the housing.

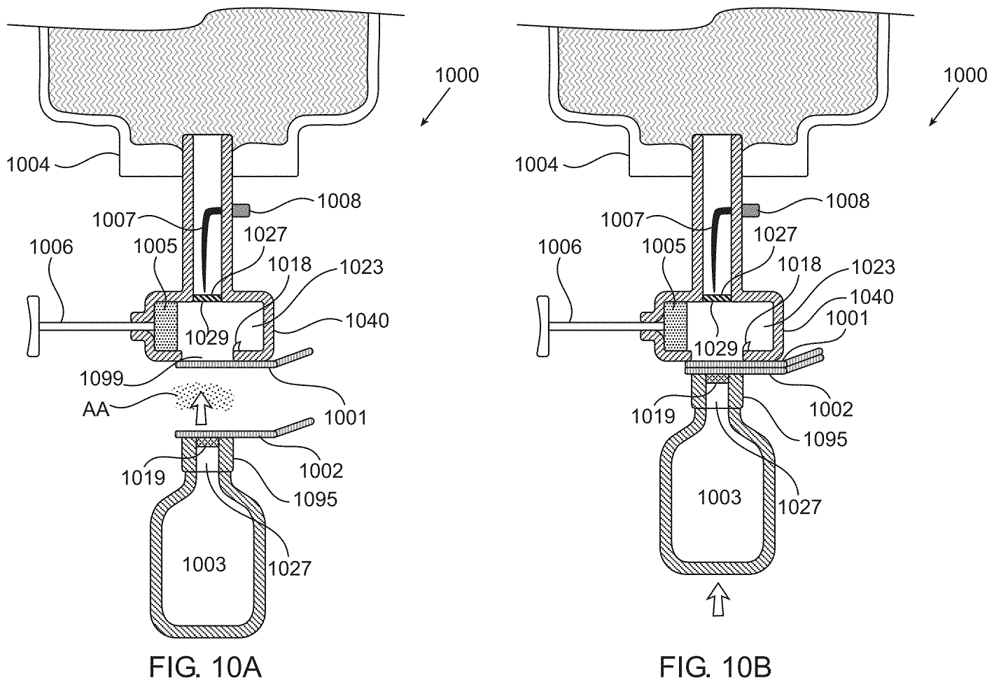

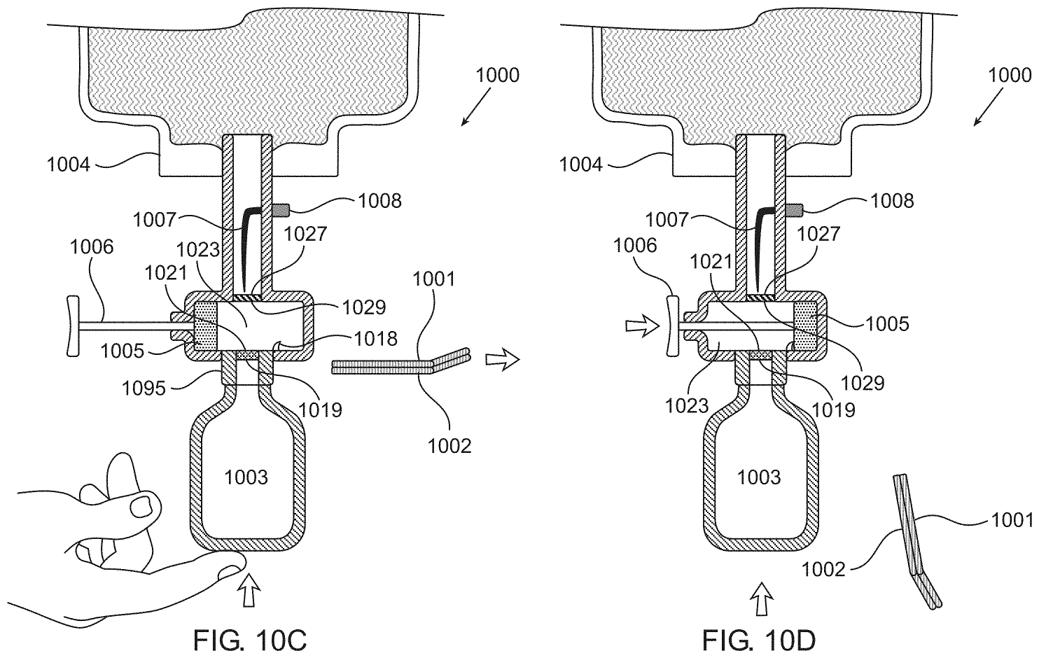

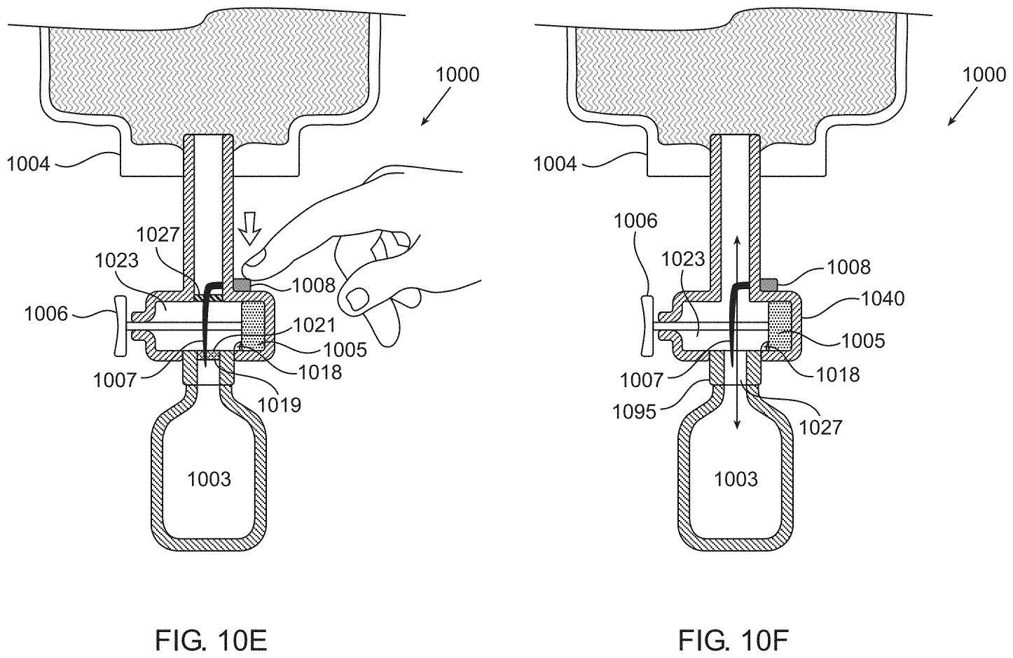

FIGS. 10A-10F illustrate system 1000 having a first vessel 1004 in the form of a container integrally connected to proximal end portion of housing 1040. Removable sealing member 1001 of housing 1040 substantially and/or completely covers and seals an opening 1099 of the housing as shown in FIG. 10A. Removable sealing member 1002 substantially and/or completely covers and seals an opening of connection port of connection port 1095 as shown in FIG. 10A. Ambient Air AA with contaminants surrounds the housing 1040 and connection port 1095. Ambient Air AA particles also surround the exterior surfaces of removable sealing members 1001 and 1002. Locking member 1018 is shown located on an inner surface of the housing 1040, optionally on an interior surface of a wall of housing 1040. Locking member 1018 may be a retractable pin configured to allow wiping member 1005 to pass over it when the wiping member moves across housing 1040. When removable sealing member 1001 is in a first sealed position as shown in FIG. 10A, removable sealing member hermetically/airtight seals opening 1099 of housing 1040 therefore isolating the interior 1023 of housing 1040 from ambient air AA contaminants. Ambient air AA contaminants are unable to enter into the interior 1023 of housing 1040 when removable sealing member 1001 substantially seals/covers opening 1099 of housing 1040. Likewise, when removable sealing member 1002 substantially seals/covers connection port 1095 as shown in FIG. 10A ambient air AA particles are unable to enter the interior of connection port 1095, and ambient air AA particles are unable to contact second removable sealing member 1019 of connection port 1095. FIG. 10B shows connection port 1095 engaged/coupled to housing 1040 wherein ambient air AA contaminants are entrapped between removable sealing members 1001 and 1002. When housing 1040 is engaged/coupled to connection port 1095 as shown in FIG. 10B an airtight/hermetic engagement/coupling is established between housing 1040 and connection port 1095. In FIG. 10B both removable sealing members 1001 and 1002 are in a first sealed position. FIG. 10C shows removable sealing members 1001 and 1002 displaced simultaneously and/or at about the same time via a pulling to the right by a user's hand which represents manual manipulation of the removable sealing members 1001 and 1002. In FIG. 10C removable sealing members 1001 and 1002 are both in a second unsealed position. A surface 1021 of second sealing member 1019 of connection port 1095 is now exposed to an interior 1023 of the housing 1040. The surface 1021 of the second sealing member 1019 is shown in FIG. 10C aligned with an interior surface (and/or an interior wall surface) of housing 1040, wherein the surface 1021 of second sealing member 1019 of connection port 1095 is positioned to be wiped off by the wiping member 1005 when the wiping member moves across the housing as shown in FIG. 10D. A surface 1029 of first sealing member 1027 is aligned with an interior wall of housing 1040, the surface 1029 of first sealing member 1027 is positioned to be wiped off by the wiping member 1005 when the wiping member moves across the housing 1040. FIG. 10D shows the wiping member 1005 which is disposed inside (in the interior 1023 of) the housing 1040 moving across the interior 1023 of housing 1040 thus wiping off via a force of friction surface 1029 of first sealing member 1029 and also wiping off via a force of friction surface 1021 of second sealing member 1019. Connection port 1095 or a securing/engagement/coupling mechanism (not shown) is in a second contracted position when surface 1021 of second sealing member 1019 is aligned with an inner wall of housing 1040. FIG. 10E shows a user's finger pushing downward piercing member actuator button 1008 which moves piercing member 1007 through first sealing member 1027 and second sealing member 1019 after the wiping member 1005 is in a second/final position. In FIGS. 10D-10F locking mechanism 1018 extends out from an interior 1023 wall of housing 1040 to press against wiping member 1005 thus preventing wiping member 1005 from retracting/returning back towards its initial/first position as was shown in FIG. 10A. FIG. 10F shows the establishment of a fluidic communication between housing 1040 and connection port 1095 via the interior 1023 of housing 1040 and interior 1027 of connection port 1095.

FIGS. 11A-11F illustrate system 1100 with folded removable sealing members 1101 and 1102. External ends of the removable sealing members 1001 and 1102 can be pulled out by a user to thereby unfold the removable sealing members and/or externally displace thereof. First sealing member of housing 1199 and second sealing member 1198 of connection port 1197 can then be pierced by piercing member 1107 to allow a fluid passageway between the housing 1199 and connection port 1197. FIG. 11A shows housing/device 1199 having a wiping member 1105 disposed inside the housing device 1199. Actuator/handle/rod 1109 is positioned through a wall of housing 1199 and is connected to wiping member 1105. Removable sealing member 1101 is shown in a folded configuration attached to housing 1199, removable sealing member 1101 is in a first sealed position. Removable sealing member 1102 is shown in a folded configuration attached to connection port 1197, removable sealing member 1102 covering second sealing member 1198 and preventing ambient air AA contaminants from contacting second sealing member 1198. FIG. 11B shows housing/device 1199 engaged/coupled to connection port 1197 via engagement mechanism/coupling mechanism/securing mechanism (not shown) which may be in the form of ratchet teeth, a clamping member, a retention member, a thread, and combinations thereof. FIG. 11C shows manual manipulation of removable sealing members 1101 and 1102, wherein a user's hand/fingers grasp removable sealing members 1101 and 1102 and pull the removable sealing members 1101, 1102 external to housing 1199 and connection port 1197, thus displacing removable sealing members 1101 and 1102 from the location of fluidic passageway between housing 1199 and connection port 1197. As the user's hand/fingers pull the removable sealing members as shown in FIG. 11C the removable sealing members 1101 and 1102 are unfolded and the engagement/coupling between housing 1199 and connection port 1197 is decontaminated, optionally sterilized. Next step is to push/move wiping member actuator handle/rod 1106 to move the wiping member 1105 across the interior of the housing 1199 (not shown). FIGS. 11D and 11E shows piercing member actuator 1108 pushed downward to move piercing member 1107 downwards through first sealing member 1111 and second sealing member 1198. FIG. F shows the wiping member 1105 in a second/final position and locked into the second/final position by the locking member 1118. When the wiping member 1105 is in the second/final position the wiping member has already wiped off a surface of first sealing member 1111 and second sealing member 1198. FIG. F shows the establishment of a fluidic communication between housing 1199 and connection port 1197. In one or more embodiments, second sealing member 1198 may be configured to remain and be unmovable in a position as is shown in Fig. A. In one or more embodiments, second sealing member 1198 of connection port 1197 may be configured to be movable thus displacing into the interior 1130 of connection port 1197, optionally or alternately displacing into the interior of vessel 1103, optionally or alternately displacing into the interior 1150 of housing 1199. In accordance with this embodiments, second sealing member 1198 is a plug, the plug may be a rubber plug and/or a plastic plug, and/or an elastomeric plug. The plug 1198 may be configured to displace upon application of pressure to the housing 1199 and/or application of pressure to container 1104/1103 which will push fluid from container 1104/1103 towards plug 1198 thus displacing plug 1198 and/or via application of pressure by piercing member 1107 which may push and displace plug 1198. In one or more embodiments, first sealing member 1111 may be configured to displace into the interior 1150 of housing 1199 or into the interior of container 1104. In accordance with this embodiment first sealing member 1111 may be a plug, 1111, the plug 1111 may be a rubber plug and/or a plastic plug, and/or an elastomeric plug. Plug 1111 may be configured to displace upon application of pressure to housing 1199 and or application of pressure to container 1104/1103 which will push fluid from container 1104/1103 towards plug 1111 thus displacing plug 1111 and/or via application of pressure by piercing member 1107 which may push and displace plug 1111.

In accordance with said embodiments wherein first sealing member 1111 and/or second sealing member 1198 are (internally) displaceable plugs, housing 1199 will engage/couple with connection port 1197, then removable sealing members 1101 and 1102 will displace external to housing 1199 and connection port 1197, and then either or both first sealing member 1111 and second sealing member 1198 will displace internally into the interior 1150 of housing 1199 or into the interior 1130 of connection port 1197.

In another embodiment wherein first sealing member 1111 and/or second sealing member 1198 are (internally) displaceable plugs, housing 1199 will engage/couple with connection port 1197, then removable sealing members 1101 and 1102 will displace external to housing 1199 and connection port 1197, then wiping member 1105 will move across housing 1199 via actuation of actuator handle/rod 1106 thus wiping off any residual contaminants from a surface of first sealing member 1111 and second sealing member 1198, and then either or both first sealing member 1111 and second sealing member 1198 will displace internally into the interior 1150 of housing 1199 or into the interior 1130 of connection port 1197.

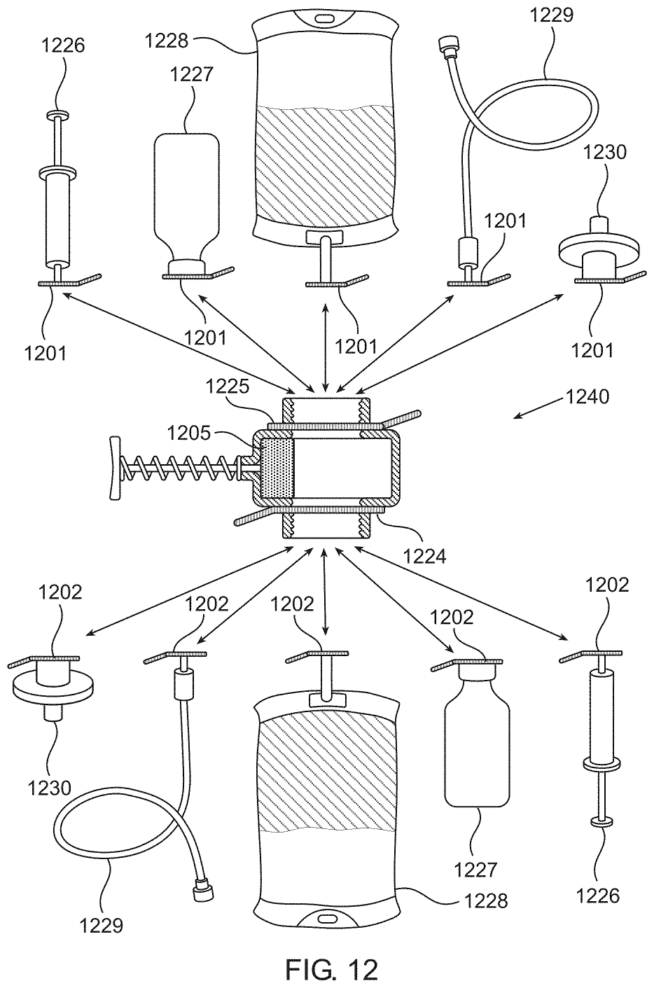

FIG. 12 illustrates an exemplary decontamination device 1240 configured for decontaminating a connection between the device 1240 and connection ports 1201 of various medical/medicinal vessels. The decontamination device 1240 includes at least two removable sealing members 1225 and 1224 that are shown in a first sealed position covering openings to the interior of the device 1240. The device 1240 can be attached to various vessel types, such as, but not limited to a syringe 1226, a bottle 1227, an infusion bag 1228, an infusion line 1229, and a filter 1230. The device 1240 includes removable sealing member 1225 and 1224 which can be engaged/coupled to removable sealing members 1201 of the various vessels, respectively. A wiping member 1205 is disposed within device 1240 and is configured to move across the interior of the device to wipe off any residual contaminants that may be present within device 1240 and/or on the surfaces of vessels 1226, 1227, 1228, 1229, and/or 1230.

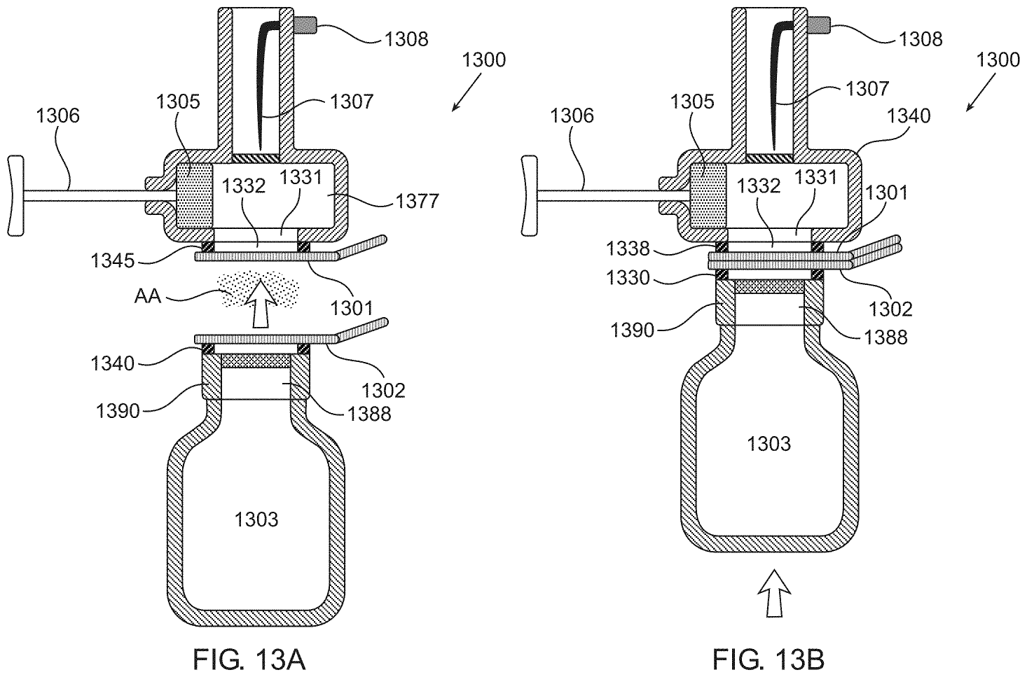

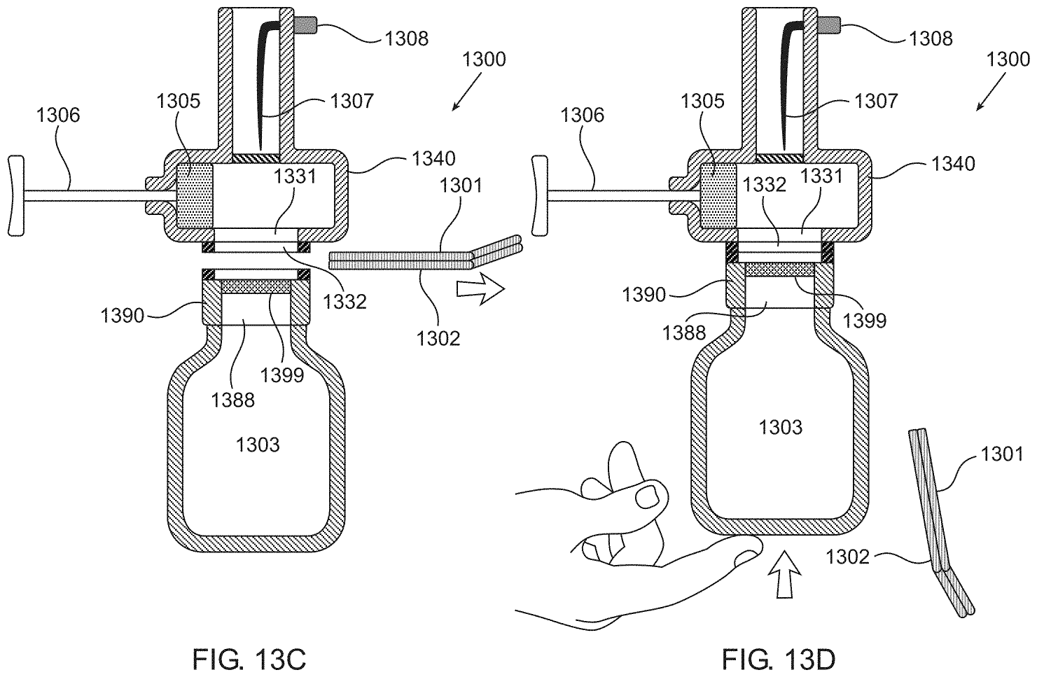

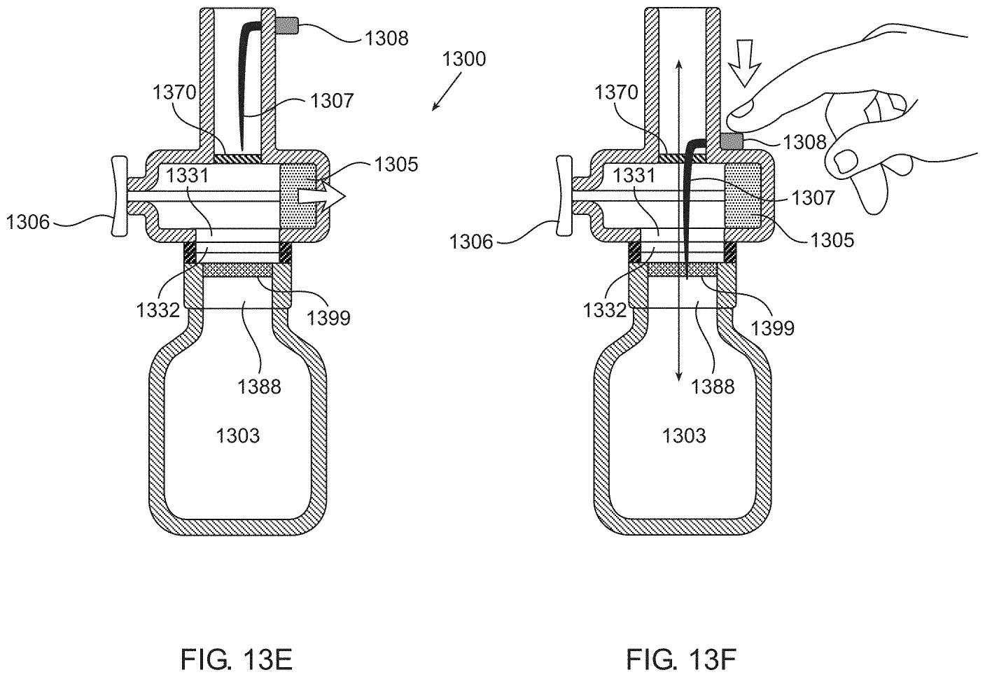

FIGS. 13A-13F illustrates yet another exemplary system 1300 that includes a sealing ring 1338 (“O-ring) about a surface of housing 1340 and a sealing ring 1330 about a surface of connection port 1390. The sealing rings 1338, 1330 may be made from a resilient material, such as a rubber and may be configured to maintain a sealed connection between housing 1340 and connection port 1390 at about the time and/or at the time of external displacement of removable sealing members 1301 and 1302 from housing 1340 and connection port 1390, respectively. Wiping member 1305 located inside housing 1340 is configured to move across the interior of the housing 1340 by pushing/sliding actuator handle 1306 which is connected to wiping member 1305 through a hole/aperture in a side wall of housing 1340. FIG. 13A shows housing 1340 separate from connection port 1390 with ambient air AA contaminants present between housing 1340 and connection port 1390. FIG. 13B shows housing 1340 engaged/coupled to connection port 1390 with removable sealing member 1301 and removable sealing member 1302 pressed against each other. Removable sealing member 1301 is attached to sealing ring 1338 and removable sealing member 1302 is attached to sealing ring 1330. Either or both of removable sealing members 1301 and/or 1302 may be provided in a folded (not shown) configuration as was shown in FIGS. 11A-11C. FIG. 13C shows removable sealing members 1301 and 1302 displaced externally to housing 1340 and connection port 1390, removable sealing members 1301 and 1302 shown in a second unsealed position. Connection port 1390 is intended to be engaged/coupled to housing 1340 in FIG. 13C, wherein a surface of the second sealing member 1399 of the connection port 1390 which was covered/sealed by the removable sealing member 1302 is now exposed to an interior of housing 1340. FIG. 13D shows a user's finger pushing vessel/container 13D upwards towards housing 1340 to align a surface of second sealing member 1399 with an interior surface of housing 1340 thus positioning the surface of second sealing member 1399 to be wiped off by the wiping member 1305 when the wiping member moves across housing 1340. FIG. 13E shows wiping member moving across the housing from left to right thus wiping off a surface of first sealing member 1370 and second sealing member 1399. FIG. 13F shows piercing member 1307 pushed downwards to pierce through, optionally or alternately displace, either or both first sealing member 1370 and/or second sealing member 1399. In one or more embodiments, element 1332 may be a plug 1332, plug 1332 may be configured to displace into the interior 1377 of housing 1340 or exterior to housing 1340. Plug 1332 of housing 1340 may be configured to engage/couple with second sealing member 1399 and after said engagement/coupling both plug 1332 and second sealing member 1399 may displace into the interior 1377 of housing 1340 or into the interior 1388 of connection port 1390. Said engagement/coupling of both plug 1332 and second sealing member 1399 into the interior 1377 of housing 1340 or into the interior 1388 of connection port 1390 is to occur after external displacement of removable sealing members 1301 and 1302 to a second unsealed position but prior to moving of the wiping member 1305 across the interior of housing 1340.

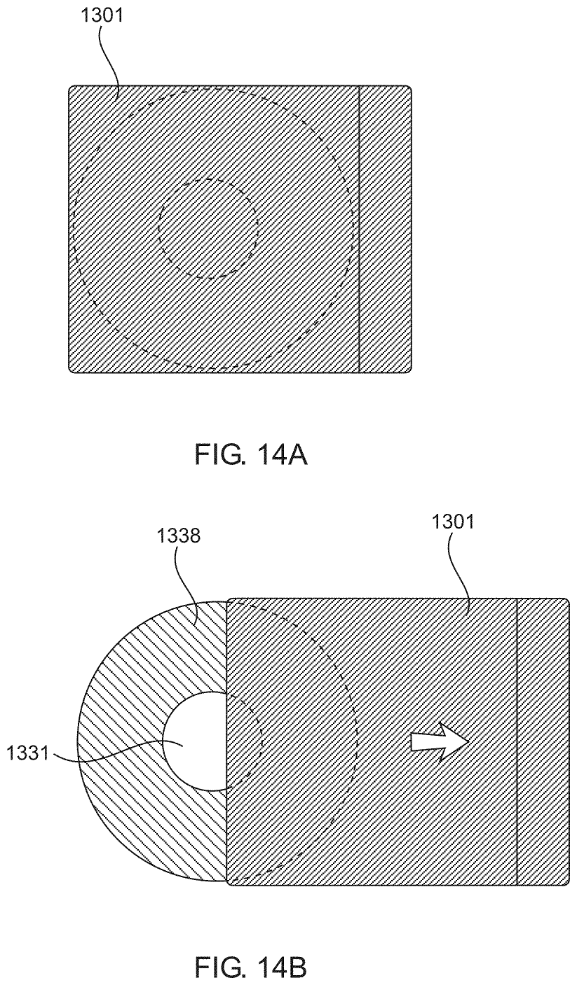

FIGS. 14A-14B are top views showing the removable sealing member 1301 of FIGS. 13A-13F that covers and seals an opening 1331 of housing 1340. The removable sealing member 1301 is shown to cover the entire opening 1331 thus hermetically sealing opening 1331 which prevents ambient air AA particles from entering into the interior of housing 1340. Sealing ring 1338 maintains the sealed connection following the external displacement of removable sealing member 1301 from housing 1340 (FIG. 14B). Press forces due to the engagement/coupling of housing 1340 to connection port 1390 via an engagement mechanism (not shown) such as ratchet teeth, thread-complimentary thread, and/or clamping member-retention member provides the press forces that maintain the airtight seal between sealing rings 1338 and 1330 during external displacement of removable sealing members 1301 and 1302.

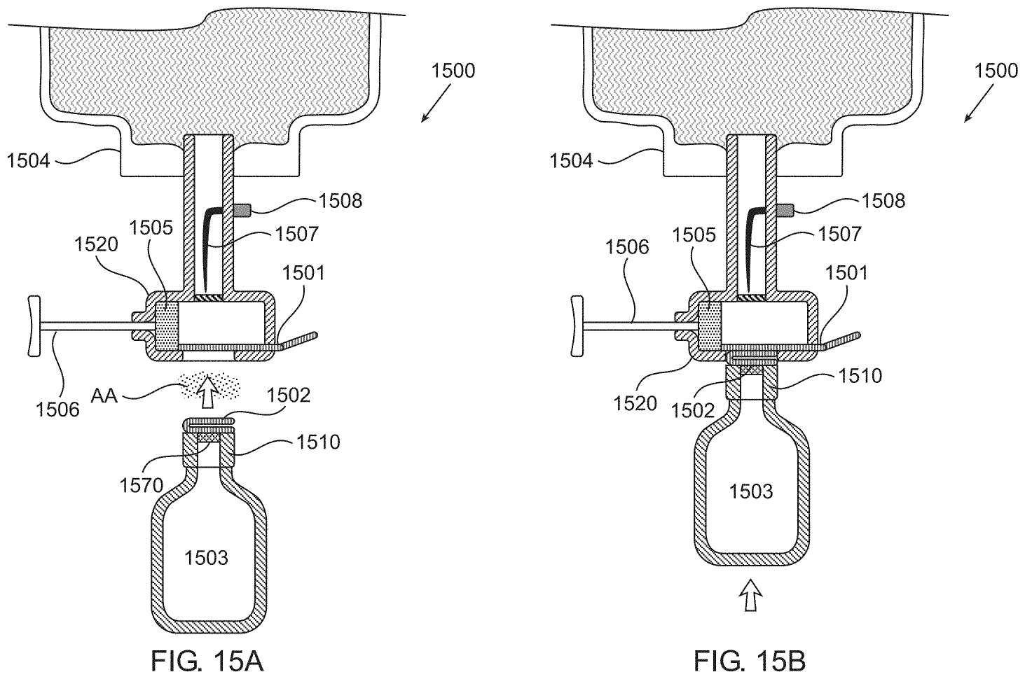

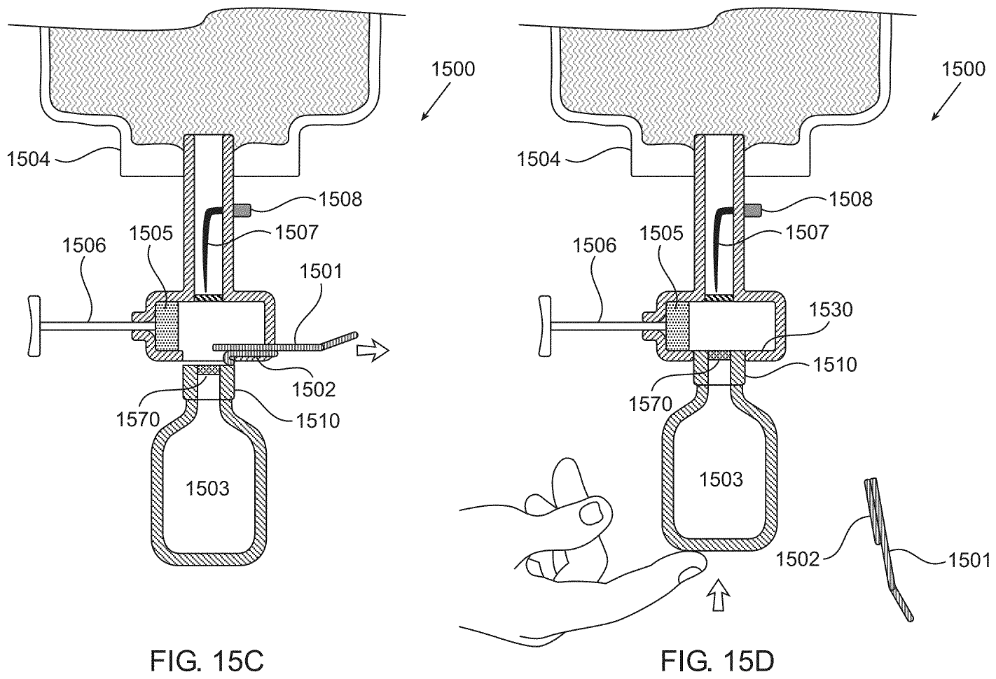

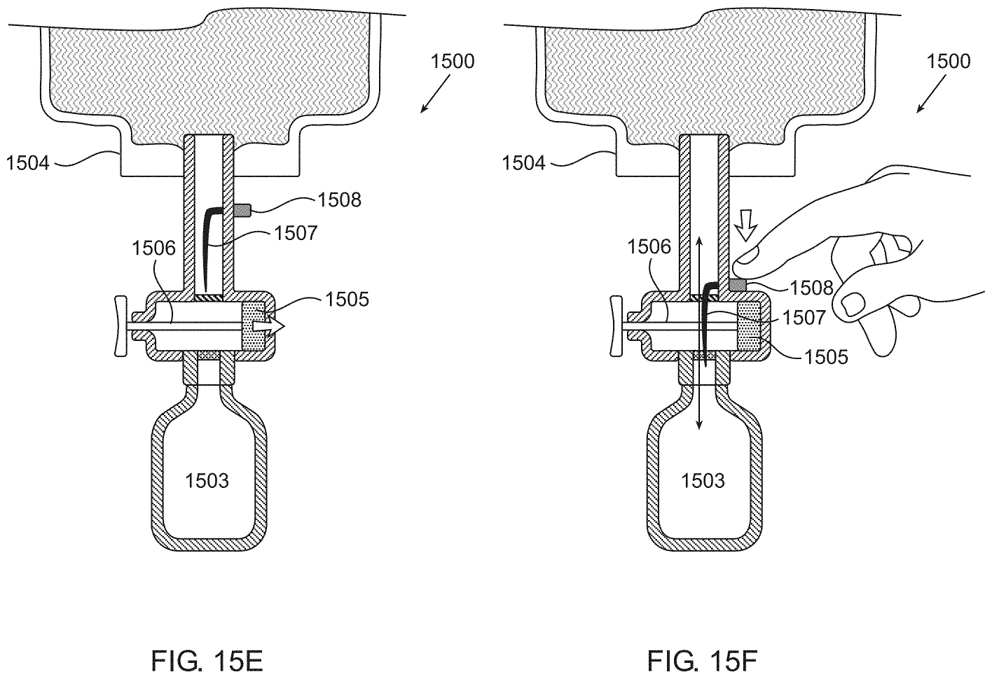

FIGS. 15A-15F illustrate a system 1500 which is similar to system 1100 but here removable sealing member 1502 is smaller in size than removable sealing member 1501 and does not protrude externally from an outer surface of connection port 1510. Removable sealing member 1501 is partially located on an interior of device 1520 and partially located exterior to device 1520. In one or more embodiments, a portion of removable sealing member 1501 is attached to an interior wall 1530 of device 1520 and a portion of removable sealing member 1501 positioned through an aperture/hole in the wall of device 1520 an extends exterior to device 1520. Wiping member 1505 is disposed inside device 1520 and is connected to actuator handle/rod/tab 1506 which is disposed through an aperture/hole in a side wall of device 1520. FIG. 15A shows device 1520 separated from connection port 1510. Removable sealing member 1501 in a first sealed position sealing an opening of device 1520. Removable sealing member 1502 is shown in a first sealed position covering and hermetically sealing top (proximal end portion) of connection port 1510. Removable sealing member 1502 is shown in a folded configuration. FIG. 15B shows connection port 1510 engaged/coupled to housing 1520 with removable sealing member 1501 lying flat against removable sealing member 1502. FIG. 15C shows removable sealing member 1501 being pulled to the right (shown by right pointing arrow) which displaces removable sealing member 1501 through an aperture/hole in the wall of device 1520. As removable sealing member 1501 is being displaced external to device 1520 it pulls along removable sealing member 1502 and displaces removable sealing member 1502 from connection port 1510. Both removable sealing members 1501 and 1502 move through the aperture/hole in the side of device 1420. After removable sealing member 1501 and 1502 are externally displaced to a second unsealed position as shown in FIG. 15D a user's finger/hand pushes, optionally or alternately twist, vessel 1503 and/or connection port 1510 towards device 1520 to align a surface of second sealing member 1570 with a surface of an interior wall 1530 of device 1520 thus positioning a surface of second sealing member 1570 to be wiped off by wiping member 1505 when wiping member moves across device 1520 as shown in FIG. 15E. FIG. 15F shows a user's finger moving piercing member actuator button 1508 to move a piercing member through a first sealing member of the device 1520 and second sealing member 1570 to establish a fluidic communication between housing/device 1520 and connection port 1570.

In one or more embodiments, at least one of the first and second vessels 1504 and 1503 is a container. In one or more embodiments, the containers 1503 and 1502 may be a bag or a bottle. In one or more embodiments, at least one of the first and second vessels 1504 and 1503 may be a syringe. In one or more embodiments, the container 1504 and/or 1503 has a flexible wall.

Each of the following terms: ‘includes’, ‘including’, ‘has’, ‘having’, ‘comprises’, and ‘comprising’, and, their linguistic, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of’.

Each of the phrases ‘consisting of’ and ‘consists of’, as used herein, means ‘including and limited to’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably.

The term ‘about’, is some embodiments, refers to ±30% of the stated numerical value. In further embodiments, the term refers to ±20% of the stated numerical value. In yet further embodiments, the term refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub combination in the context or format of a single embodiment, may also be illustratively described and presented in the context or format of a plurality of separate embodiments.

Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit and broad scope of the appended claims.

All publications, patents, and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A system for establishing a decontaminated fluid passageway between a device and a connection port, comprising:
a device, comprising:
a. a housing defining a fluid passageway, a distal end portion of the housing configured to couple a connection port, the distal end portion of the housing having a securing mechanism configured to engage the connection port;
b. a wiping member located inside the housing, the wiping member configured to move across the housing;
c. an actuator connected to the wiping member, the actuator configured for moving the wiping member across the housing, the actuator extending through an opening in the wall of the housing;
d. a removable sealing member for sealing the distal end portion of the housing, the removable sealing member positioned to seal the distal end portion of the fluid passageway, the removable sealing member configured to be movable between a first sealed position and a second unsealed position, the removable sealing member being moveable by manual manipulation of the removable sealing member, wherein the removable sealing member is displaced from the fluid passageway when the removable sealing member is in the second unsealed position;
a connection port, comprising:
a. a proximal end portion defining a fluid passageway, the proximal end portion of the connection port configured to couple the distal end portion of the housing of the device, the proximal end portion of the connection port having a securing mechanism configured to engage the securing mechanism of the distal end portion of the housing of the device; and
b. a removable sealing member for sealing the proximal end portion of the fluid passageway of the connection port, the removable sealing member of the connection port configured to be movable between a first sealed position and a second unsealed position, the removable sealing member of the connection port being movable by manual manipulation of the removable sealing member, wherein the removable sealing member is displaced from the fluid passageway of the connection port when the removable sealing member is in the second unsealed position.

2. The system of claim 1, wherein the removable sealing member of the device and the removable sealing member of the connection port are both configured to fluidly seal the fluid passageway between the device and the connection port when both the removable sealing member of the device and the removable sealing member of the connection port are in a first sealed position, and wherein the removable sealing member of the device and the removable sealing member of the connection port are configured to allow passage of fluid between the device and the connection port when the removable sealing member of the device and the removable sealing member of the connection port are both in the second unsealed position.

3. The system of claim 1, wherein at least one of the removable sealing member of the device and the removable sealing member of the connection port are in a folded configuration.

4. The system of claim 1, wherein the device further comprising a first sealing member, the first sealing member positioned so that a surface of the first sealing member can be wiped off by the wiping member when the wiping member moves across the housing.

5. The system of claim 1, wherein the actuator of the device is selected from a rod, a handle, and a tab.

6. The system of claim 1, wherein the housing of the device further comprising a locking member located on an interior wall of the housing, the locking member configured for locking the wiping member in an extended/second position to prevent the wiping member from returning to an original first position.

7. The system of claim 1, wherein the device further comprising a locking member in the form of a retractable pin configured to lock the wiping member in a final position after the wiping member has moved across the housing of the device.

8. The system of claim 1, wherein the connection port further comprising a second sealing member.

9. The system of claim 1, wherein the device further comprising a piercing member.

10. The system of claim 8, wherein the device further comprising a piercing member and a piercing member actuator, the piercing member actuator configured to move the piercing member through the second sealing member of the connection port.

11. The system of claim 8, wherein the second sealing member fluidly seals an opening of the connection port.

12. The system of claim 11, wherein a surface of the second sealing member of the connection port is characterized in that the surface of the second sealing member is positioned to be wiped off by the wiping member of the device when the wiping member moves across the housing of the device when the connection port is coupled/engaged to the device.

13. The system of claim 12, wherein the securing mechanism at the distal end portion of the housing is selected from a thread, a ratchet teeth mechanism, and a clamping member and the securing mechanism at the proximal end portion of the connection port is selected from a complimentary thread and a complimentary a retention member.

14. The system of claim 1, wherein the proximal end portion of the housing of the device is further configured to engage/couple a container and the distal end portion of the connection port is further configured to engage/couple a container.

15. The system of claim 1, wherein the removable sealing member of the device and the removable sealing member of the connection port are both configured to be displaced from the fluid passageway via a pulling motion.

16. The system of claim 1, wherein at least one of the device and the connection port further having at least one of a sealing member and an “O-ring”.

17. The system of claim 1, wherein the securing mechanism of the device having teeth or holes/dents configured to provide the connection port to be moveable between an extended position and a contracted position when the connection port is engaged/coupled to the device.

18. The system of claim 17, wherein when the device is coupled to the connection port and when the securing mechanism of the device is in the extended position, an exterior surface of the second sealing member of the connection is not aligned with an interior surface of the housing, and wherein when the device is coupled to the connection port and the securing mechanism of the device is in the second contracted position, an exterior surface of the second sealing member of the connection port is aligned with an interior surface of the housing of the device.

19. The system of claim 18, wherein when the exterior surface of the second sealing member of the connection port is aligned with an interior surface of the housing, the wiping member is able to wipe off the exterior surface of the second sealing member of the connection port when the wiping member moves across the housing.

20. The system of claim 1, wherein the device further comprising a first sealing member and wherein the connection port further comprising a second sealing member, wherein both the first and second sealing members are positioned to be wiped off by the wiping member when the wiping member moves across the housing and when the connection port is coupled to the device.
